SEQUENCE LISTING (57 sequences)

SEQ ID NO: 1 - DNA, 3730 bp, Arabidopsis thaliana
agaggactgtgagattgtgaattgcatagtcgtcgtcttctggcgggaaaagaagccctagaaaaagggtgaaaggtgaaaactctacttcttcttcttcttcttcttcagagtgtgagagagatgcattggattgctaccagaaacgccgtcgtttcattcccaaaatggcggttcttcttccgctcctcatatcgcacttactcttccctcaaaccctcctccccaattctacttaatagaaggtactctgaggggatatcttgtctcagagatggaaagtctttgaaaagaatcacaacggcttctaagaaagtgaagacgtcaagtgatgttctcactgacaaagatctctctcatttggtttggtggaaggagagattgcagacatgtaagaaaccatctactcttcagcttattgaaaggcttatgtacaccaatttacttggtttggaccctagcttgaggaatggaagtttaaaagatggaaacctcaactgggagatgttgcagtttaagtcaaggtttccacgcgaagttttgctctgcagagtaggagaattttatgaggctattggaatagatgcttgtatacttgttgaatatgctggtctcaatccttttggtggtcttcgatcagatagtattccaaaggctggctgcccaattatgaatcttcgacagactttggatgacctgacacgcaatggttattcagtgtgtattgtggaggaagttcaggggccaacaccagcacgctcccgtaaaggtcgatttatttcagggcatgcacatccaggaagtccttatgtatatgggcttgtcggtgttgaccatgatcttgactttcctgatcctatgcctgttgttgggatatctcgttcagcaagggggtattgtatgatatctattttcgagactatgaaagcatattcgctagatgatggtctaacagaagaagccttagttaccaagctccgcactcgtcgctgtcatcatcttttcttacatgcatcgttgaggcacaatgcatcagggacgtgccgctggggagagtttggggaagggggtctactctggggagaatgcagtagcaggaattttgaatggtttgaaggagatactctttccgagctcttatcaagggtcaaagatgtttatggtcttgatgatgaagtttcctttagaaatgtcaatgtaccttcaaaaaatcggccacgtccgttgcatcttggaacggctacacaaattggtgccttacctactgaaggaataccttgtttgttgaaggtgttacttccatctacgtgcagtggtctgccttctttgtatgttagggatcttcttctgaaccctcctgcttacgatattgctctgaaaattcaagaaacgtgcaagctcatgagcacagtaacatgttcaattccagagtttacctgcgtctcttctgctaagcttgtgaagcttcttgagcaacgggaagccaactacattgagttctgtcgaataaaaaatgtgcttgatgatgtattacatatgcatagacatgctgagcttgtggaaatcctgaaattattgatggatcctacctgggtggctactggtttgaaaattgactttgacacttttgtcaacgaatgtcattgggcgtctgatacaattggtgaaatgatctctttagatgagaatgaaagtcatcagaatgtaagtaaatgtgacaatgtcccgaacgaattcttttatgatatggagtcttcatggcgaggtcgcgttaagggaattcatatagaggaagaaatcactcaagtagaaaaatcagctgaggctttatctttagcagtagctgaggattttcaccctattatatcaagaattaaggccaccactgcttcacttggtggcccgaaaggcgaaatcgcatatgcaagagagcatgagtctgtttggttcaaggggaaacggtttacgccatctatctgggctggtactgcaggggaagaccaaataaaacagctgaaacctgccttagactcgaaaggaaaaaaggttggagaagaatggtttacgaccccaaaggtggaaattgctttagtcagataccatgaagctagtgagaatgcaaaagctcgggtgttggaactgttgcgcgagttatccgttaaattgcaaacaaaaataaatgttcttgtctttgcatctatgcttctggtcatttcaaaagcattattttcccatgcttgtgaagggagaaggcgaaagtgggtttttccaacgcttgtcggattcagtttagatgagggcgcaaaaccattagatggtgccagtcgaatgaagctgacaggcctgtcaccttattggtttgatgtatcttctggaaccgctgttcacaataccgttgacatgcaatcactgtttcttctaactggacctaacggtggtggtaaatcgagtttgctcagatcaatatgcgcagctgctctacttggaatttccggtttaatggttccagctgaatcagcttgtattcctcactttgattccatcatgcttcacatgaaatcatatgacagccctgtagacggaaaaagttctttccaggtagaaatgtcggaaatacgatctattgtaagccaggctacttcgagaagcctagtgcttatagatgagatatgccgagggacagagacagcaaaaggcacctgtatcgctggtagtgtggtagagagtcttgacacaagtggttgtttgggtattgtatctactcatctccatggaatcttcagtttacctcttacagcgaaaaacatcacatataaagcaatgggagccgaaaatgtcgaagggcaaaccaagccaacttggaaattgacagatggagtctgcagagagagtcttgcgtttgaaacagctaagagggaaggtgttcccgagtcagttatccaaagagctgaagctctttacctctcggtctatgcaaaagacgcatcagctgaagttgtcaaacccgaccaaatcataacttcatccaacaatgaccagcagatccaaaaaccagtcagctctgagagaagtttggagaaggacttagcaaaagctatcgtcaaaatctgtgggaaaaagatgattgagcctgaagcaatagaatgtctttcaattggtgctcgtgagcttccacctccatctacagttggttcttcatgcgtgtatgtgatgcggagacccgataagagattgtacattggacagaccgatgatcttgaaggacgaatacgtgcgcatcgagcaaaggaaggactgcaagggtcaagttttctataccttatggttcaaggtaagagcatggcttgtcagttagagactctattgattaatcaactccatgaacaaggctactctctggctaacctagccgatggaaagcaccgtaatttcggaacgtcctcaagcttgagtacatcagacgtagtcagcatcttatagtttgaaacattagctgtgtttgtagttgatcatctctatgtgcaattgaacaagtcagtttgctagaactagagtagattactaagaaaccatgccgtttttcattttgagattttgcaaaacggcatgcagttcgggtaagtcggatgccgcaattaccaattttgggtcagtctgtgtaattgtcgtttcataaatccgattaacgtgtactttgaacaaaactcagcagtaaacttctttattcatc

SEQ ID NO: 2 - protein, 1118 aa, Arabidopsis thaliana
MHWIATRNAVVSFPKWRFFFRSSYRTYSSLKPSSPILLNRRYSEGISCLRDGKSLKRITTASKKVKTSSDVLTDKDLSHLVWWKERLQTCKKPSTLQLIERLMYTNLLGLDPSLRNGSLKDGNLNWEMLQFKSRFPREVLLCRVGEFYEAIGIDACILVEYAGLNPFGGLRSDSIPKAGCPIMNLRQTLDDLTRNGYSVCIVEEVQGPTPARSRKGRFISGHAHPGSPYVYGLVGVDHDLDFPDPMPVVGISRSARGYCMISIFETMKAYSLDDGLTEEALVTKLRTRRCHHLFLHASLRHNASGTCRWGEFGEGGLLWGECSSRNFEWFEGDTLSELLSRVKDVYGLDDEVSFRNVNVPSKNRPRPLHLGTATQIGALPTEGIPCLLKVLLPSTCSGLPSLYVRDLLLNPPAYDIALKIQETCKLMSTVTCSIPEFTCVSSAKLVKLLEQREANYIEFCRIKNVLDDVLHMHRHAELVEILKLLMDPTWVATGLKIDFDTFVNECHWASDTIGEMISLDENESHQNVSKCDNVPNEFFYDMESSWRGRVKGIHIEEEITQVEKSAEALSLAVAEDFHPIISRIKATTASLGGPKGEIAYAREHESVWFKGKRFTPSIWAGTAGEDQIKQLKPALDSKGKKVGEEWFTTPKVEIALVRYHEASENAKARVLELLRELSVKLQTKINVLVFASMLLVISKALFSHACEGRRRKWVFPTLVGFSLDEGAKPLDGASRMKLTGLSPYWFDVSSGTAVHNTVDMQSLFLLTGPNGGGKSSLLRSICAAALLGISGLMVPAESACIPHFDSIMLHMKSYDSPVDGKSSFQVEMSEIRSIVSQATSRSLVLIDEICRGTETAKGTCIAGSVVESLDTSGCLGIVSTHLHGIFSLPLTAKNITYKAMGAENVEGQTKPTWKLTDGVCRESLAFETAKREGVPESVIQRAEALYLSVYAKDASAEVVKPDQIITSSNNDQQIQKPVSSERSLEKDLAKAIVKICGKKMIEPEAIECLSIGARELPPPSTVGSSCVYVMRRPDKRLYIGQTDDLEGRIRAHRAKEGLQGSSFLYLMVQGKSMACQLETLLINQLHEQGYSLANLADGKHRNFGTSSSLSTSDVVSIL

SEQ ID NO: 3 - DNA, 3765 bp, Glycine max
gtcagatacagagtccttccctcctcgtgtgtggactgtggcgggaactcattttgctagtttgcttcctctctctctctcgttcccattcaacgcaatgtacagggtagccacaagaaacgtcgccgttttcttccctcgttgctgttccctcgcgcactacactccttctctatttcccattttcacttcattcgctccctctcgtttccttagaataaatggatgtgtaaagaatgtgtcgagttatacggataagaaggtttcaagggggagtagtagggccaccaagaagcccaaaataccaaataacgttttagatgataaagaccttcctcacatactgtggtggaaggagaggttgcaaatgtgcagaaagttttcaactgtccagttaattgaaagacttgaattttctaatttgcttggcctgaattccaacttgaaaaatggaagtctgaaggaaggaacactcaactgggaaatgttgcaattcaagtcaaaatttccacgtcaagtattgctttgcagagttggggaattctatgaagcttggggaatagatgcttgtattcttgttgaatatgtgggtttaaatcccattggtggtctgcgatcagatagtatcccaagagctagttgtcctgtcgtgaatcttcggcagactttagatgatctgacaacaaatggttattcagtgtgcattgtggaggaggctcagggcccaagtcaagctcgatccaggaaacgtcgctttatatctgggcatgctcatcctggaaatccctatgtatatggacttgctacagttgatcatgatcttaactttccagaaccaatgcctgtagtaggaatatctcattctgcgaggggttattgcattaatatggtactagagaccatgaagacatattcttctgaagattgcttgacagaagaagcagttgttacgaagcttcgtacttgccaatatcattacttatttttgcatacatccttgaggcggaattcttgtggaacctgcaactggggagaatttggtgagggagggctattatggggagaatgtagttctagacattttgattggtttgatggcaaccctgtctccgatcttttggccaaggtaaaggaactttatagtattgatgatgaggttacctttcggaacacaactgtgtcttcaggacatagggctcgaccattaactcttggaacatctactcaaattggtgccattccaacagaaggaataccttctttgttgaaggttttacttccatcaaattgcaatggattaccagtattgtacataagggaacttcttttgaatcctccttcatatgagattgcatccaaaattcaagcaacatgcaaacttatgagcagtgtaacgtgttcaattccagaatttacatgtgtttcgtcagcaaagcttgtaaagctacttgaatggagggaggtcaatcatatggaattttgtagaataaagaatgtactggatgaaattttgcagatgtatagtacctctgagctcaatgaaatattgaaacatttaatcgagcccacatgggtggcaactgggttagaaattgactttgaaaccttggttgcaggatgtgagatcgcatctagtaagattggtgaaatagtatctctggatgatgagaatgatcagaaaatcaactcgttctcttttattcctcacgaattttttgaggatatggagtctaaatggaaaggtcgaataaaaagaatccacatagatgatgtattcactgcagtggaaaaagcagctgaggccttacatatagcagtcactgaagattttgttcctgttgtttctagaataaaggctattgtagcccctctcggaggtcctaagggagaaatatcttatgctcgggagcaagaagcagtttggttcaaaggcaaacgctttacaccgaatttgtgggctggtagccctggagaggaacaaattaaacagcttaggcatgctttagattctaaaggtagaaaggtaggggaggaatggtttaccacaccaaaggtcgaggctgcattaacaaggtaccatgaagcaaatgccaaggcaaaagaaagagttttggaaattttaaggggactcgctgctgagttgcaatacagtataaacattcttgtcttttcttccatgttgcttgttattgccaaagctttatttgctcatgcaagtgaagggagaagaaggagatgggtctttcccacgcttgtagaatcccatgggtttgaggatgtgaagtcattggacaaaacccatgggatgaagataagtggtttattgccatattggttccacatagcagaaggtgttgtgcgtaatgatgttgatatgcaatcattatttctgttgacaggaccgaatggtggtgggaaatcaagttttcttaggtcaatttgtgctgctgcactacttgggatatgtggactcatggttcctgcagaatcagccctaattccttattttgactccatcacgcttcatatgaagtcatatgatagtccagctgataaaaagagttcctttcaggttgaaatgtcagaacttcgatccatcattggcggaacaaccaacaggagccttgtacttgttgatgaaatatgccgaggaacagaaactgcaaaagggacttgcattgctggtagcatcattgaaacccttgatggaattgggtgtctgggtattgtatccactcacttgcatggaatatttactttgcccctaaacaaaaaaaacactgtgcacaaagcaatgggcacaacatccattgatggacaaataatgcctacatggaagttgacagatggagtttgtaaagaaagtcttgcttttgaaacggctaagagggaaggaattcctgagcatattgttagaagagctgaatatctttatcagttggtttatgctaaggaaatgctttttgcagaaaatttcccaaatgaagaaaagttttctacctgcatcaatgttaataatttgaatggaacacatcttcattcaaaaaggttcctatcaggagctaatcaaatggaagttttacgcgaggaagttgagagagctgtcactgtgatttgccaggatcatataaaggacctaaaatgcaaaaagattgcattggagcttactgagataaaatgtctcataattggtacaagggagctaccacctccatcggttgtaggttcttcaagcgtctatgtgatgttcagaccagataagaaactctatgtaggagagactgatgatctcgagggacgggtccgaagacatcgattaaaggaaggaatgcatgatgcatcattcctttattttcttgtcccaggtaaaagcttggcatgccaatttgaatctctgctcatcaaccaactttctggtcaaggcttccaactgagcaatatagctgatggtaaacataggaattttggcacttccaacctgtatacataactagtctatagacattgatattatctacctcaatcgcgtatttttgcctcttttaaatggctcaaagacttcaatcatcgatgttaagtttaggaaacaatgtctgcagcatttttgttagaattagttgctgcagctgcatttatgtccacatcttcaagtgtggaaattcttgttcattagcttgtaagtacaaaagtgtttgtgtacgtttggagtcccgagagaatatacaagtacaaatgaacaaatatattagtaatgaatgcactaga

SEQ ID NO: 4 - DNA, 3642 bp, Zea mays
gcgcactaccccgagaaacgtgcgacgggaacctccgcggttccccaagttcgcctccttcactactctcgcgccccggcacgcctgaaaaaccccacccctcctgccgctccgcctctcccatcacttcccacgcccctcgccgcctcccattccagcgtggacacgacgccactcgccagcacggagacgcgcgcctcgaagcactactgcactagccagccgtcgttcttccgcgccggcgccatgcaccgggtgctcgtgagctcgcttgtggccgccacgccgcgatggctgcccctcgccgactccatcctccggcgccgccggccgcgctgctcccctcttcccgtgctgatgttcgatcggagggcttggtccaagccaaggaaggtctcacgaggcatttcagtggcgtccaggaaagctaacaaacagggagaatactgtgatgaaagtatgctgtcgcatatcatgtggtggaaagagaaaatggagaggtgcagaaaaccatcatccatacaattgactcagaggcttgtgtattcaaatatattagggttggatccgaatttaagaaacggaagcttgaaagatggaaccctgaacatggagattttggtatttaaatcaaaatttcctcgtgaggttctactttgcagagtaggagatttctatgaagctatcggttttgatgcctgtattctcgtagagcatgcaggcttaaatccttttggaggtttgcgttccgacagtattcctaaagctgggtgtccagtcgtgaatttacggcagacattggatgatttgactcgatgtggttattccgtgtgcatagtcgaggaaattcaaggcccaactcaagcccgtgctcggaaaagtcgatttatttctgggcatgcccatcctggtagtccttatgtatttggtcttgctgaagtagaccatgatgtagagttccctgatccgatgcctgttgttgggatttcacattctgcaaaaggttattgcttgatatctgtgctagagacaatgaaaacttattcagctgaggagggcttaacagaggaggctattgttactaagctccgcatatgtcgttatcaccatctataccttcacaattctttgaagaataattcttcagggacatcacgctggggtgaattcggtgaaggtgggctcttgtggggagagtgcagtgggaagtcctttgagtggtttgacggttcacctattcaagaacttttatgcaaggtacgggaaatatatggccttgatgagaaaacggtttttcgcgatgtcaccgtctcattggaaggcaggccccaacctcttcatcttgggactgctactcaaattggagtcataccaactgagggaataccgagtttgttaagaatggtgcttccttcaaattgtggcgggcttccatcaatgtatattagagatcttcttcttaatcctccatcatttgaggttgcagcagcgatccaagaggcttgcaggcttatgggcaacataacctgctccattcctgaatttacatgcatatcagcagcaaagcttgtgaaactacttgagtcgaaaggggtcaatcacattgaattttgtagaataaaaaatgtccttgatgagattatgctcatgaacagggatgctgagctttctgcaatcctgcatgaattactggtacctgcttctgtggctactggtttcaaagttgaagctgatatgctaatgaacggatgtagcattatttcacaacgaatagctgaagtgatttctttaggtgttgaaagtgatcaggcaataacttcattggaatatattccaaaggagttcttcaatgatatggagtcatcttggaaggggcgcgtgaaaaggatccatgctgaagaagagtttgcaaatgttgatagggctgctgaggcattatcaattgcggtcattgaagattttatgccaattatttcgagggtgaaatctgtagtgtcctcgaatggaggtttgaaaggagaaatcggttatgcaaaagaacatgaagctgtttggtttaaaggaaagagattcataccaaatgtatgggctaacacacctggtgagcagcaaataaaacaactgaagcctgcaattgattcaaaaggcagaaaggttggggaggaatggtttacaacaagcaaagttgagaatgctttagccaggtaccatgaagcttgtgataatgcaagaaataaagttcttgagctgttgagaggcctttctagtgaattgcaggacaaaattaacatacttgtcttttgctcaacactgctcatcattgcaaaagcactttttggtcatgttagtgaggctcgaagaagaggttggatgcttcctactatatctcccttatcaaaggactgtgttgtggaggaaagttcaagtgcaatggatttagtaggactatttccttactggcttgatgttaatcaaggaaatgcaatattgaatgatgtccacatgcactctttatttgttcttactggcccaaatggtggtggtaaatctagcatgttgcgatcagtctgtgcagctgtgcttcttggaatatgtggcctgatggtaccttcaacttcagctgtaatcccacattttgattccattatgctgcatatgaaagcctatgatagcccagcagatgggaaaagttcatttcagattgaaatgtcggagatacgtgctttagtcagccgagctactgctaggagtcttgttctgattgatgaaatatgtagaggcacagaaactgcaaaaggaacatgtatagctggtagcatcattgaaagacttgataatgttggctgcctaggcatcatatcaactcacctgcatgggattttcgacctgcctctctcacttagcaacactgatttcaaagctatgggaactgaagtggtcgatggatgcattcatccaacatggaaactgattgatggcatatgtagagaaagccttgcttttcaaacagcaaggagggaaggcatgcctgacttgataatcaccagggctgaggagctatatttgagtatgagtacaaataacaagcagggagcatcagtggcgcacaatgagcctcctaatggcagccccagtgtaaatggcttggttgaggagcctgaatctctgaagaacagactagaaatgctgcctggtacctttgagccgctgcggaaggaagttgagagtgctgttactacgatgtgtaagaaaatactgtcggacctttacaacaaaagtagcatcccagaactggtcgaggtggtctgcgttgctgtaggtgctagagagcaaccaccgccttccactgttggcagatctagcatctacgtgattatcagaagcgacaacaggctctatgttggacagacggacgatcttctggggcgcttgaacgcccacagatcgaaggaaggcatgcgggacgctacggtattatacgtcttggtccctggcaagagcgttgcctgccagctggaaacccttctcataaaccagctcccttcgaggggcttcaagctcatcaacaaggcagacgggaagcacaggaacttcggtatatctcgaatctctggcgaggcagttgctactggacggaactag

SEQ ID NO: 5 - DNA, 3373 bp, Lycopersicon esculentum
atgtattgggttacggcaaaaaacgtcgtcgtttcagttccccgttggcgttcactgtcccttttcctccgtccaccacttcgccggcgtttcttatctttctctccacatactctgtgccgagagcagatacgttgcgtgaaggagcggaagttttttgccacaacggcaaaaaaactcaaacaaccaaaaagtattccagaggaaaaagactatgttaatattatgtggtggaaagagagaatggaattcttgagaaagccttcttccgctcttctggctaagaggcttacatattgtaacttgctgggtgtggatccgagtttgagaaatggaagtcttaaagagggaacacttaactcggagatgttgcagttcaagtcaaaatttccacgtgaagttttgctctgtagagtaggtgatttttatgaagctattggattcgatgcttgtattcttgtggaatatgctggtttaaatccatttggtggcctgcactcagatagtataccaaaagctggttgtccagttgtgaatctaagacagacgcttgatgatctcacacgtaatggtttctctgtgtgcgtcgtggaggaagttcagggtccaactcaagctcgtgctcgtaagagtcgatttatatcagggcatgcacatccaggcagtccctatgtttttggccttgttggagatgatcaagatcttgattttccagaaccaatgcctgttgttggaatatcccgttcagcgaaggggtattgcattatctctgtttacgagactatgaagacttactctgtggaagatggcctaactgaagaagccgtagtcaccaaacttcgtacttgtcgatgccatcatttttttttgcataattcattgaagaacaattcctcaggaacatcgcgttggggagagtttggtgaaggtggacttttgtggggagaatgtaatgctagacagcaggaatggttggatggcaatcctatcgatgagcttttgttcaaggtaaaagagctttatggtctcaatgatgacattccattcagaaatgtcactgttgtttcagaaaataggccccgtcctttacaccttggaactgccacacaaattggtgctattccaaccgaagggattccatgtttgttaaaggtgttgcttcctcctcattgcagtggtctaccagtcctgtatattagggatcttcttttaaatccaccagcctatgagatttcttcagacattcaagaggcatgcagacttatgatgagtgtcacatgttcaattcctgattttacctgtatttcatctgcaaagctggtcaagctgcttgagttgagggaggcaaatcacgttgagttctgcaaaataaagagcatggtcgaagagatactgcagttgtatagaaattcagagcttcgtgctattgtagagttactgatggatcctacttgggtggcaactgggttgaaagttgattttgatacactagtaaatgaatgtggaaagatttcttgtagaatcagtgaaataatatccgtacatggtgaaaatgatcaaaagattagttcctatcctatcatcccaaatgatttctttgaagatatggagttgttgtggaaaggccgtgtcaagaggatccatttggaggaagcatatgcagaagtagaaaaggctgcggatgctttatctttagccataacagaagatttcctacctattatttcaagaataagggccacgatggccccacttggaggaactaaaggggagattttgtatgcccgtgagcatggagctgtatggtttaagggaaagagatttgtaccaactgtttgggctggaaccgctggagaagaacaaattaagcaactcagacctgctctagattcaaaggggaagaaggttggagaagaatggttcactacaatgagggtggaagatgcaatagctaggtatcacgaggcaagtgctaaggcaaagtcaagggtcttggaattgctaaggggactttcttctgaattactatctaagatcaatatccttatctttgcatctgtcttgaatgtgatagcaaaatcattattttctcatgtgagtgaaggaagaagaagaaattggattttcccaacaatcacacaatttaacaaatgtcaggacacagaggcacttaatggaactgatggaatgaagataattggtctatctccttattggtttgatgcagcacgagggactggtgtacagaatacagtagatatgcagtccatgtttcttttaacaggtccaaatggtgggggcaaatcaagcttgctgcgttcgttgtgtgcagctgcattgctaggaatgtgtgggttcatggttccagctgaatcagctgtcattcctcattttgactcaattatgctgcatatgaaatcatatgatagtcctgttgatggaaaaagttcatttcagattgaaatgtctgaaattcggtctctgattactggtgccacttcaagaagtcttgtacttatagatgaaatatgtcgaggaacagaaacagcaaaagggacatgtattgctggaagtgtcatagaaaccctggacgaaattggctgtttgggaattgtatcaacccacttgcatggaatatttgatttacccctgaaaatcaagaagaccgtgtataaagcaatgggagctgaatatgttgacggtcaaccaataccaacttggaaactcattgatgggatctgtaaagagagtctagcatttgaaacagctcagagagaaggaattccagaaatattaatccaaagagcagaagaattgtataattcagcttacgggaatcagataccaaggaagatagaccaaataagacctctttgttcagatattgacctcaatagcacagataacagttctgaccaattaaatggtacaagacaaatagctttggattctagcacaaagttaatgcatcgaatgggaatttcaagcaagaaacttgaagatgctatctgtcttatctgtgagaagaagttaattgagctgtataaaatgaaaaatccgtcagaaatgccaatggtgaattgcgttcttattgctgccagggaacagccggctccatcaacaattggtgcttcaagtgtctatataatgctaagacctgacaaaaagttgtatgttggacagactgatgatcttgagggcagagtacgtgctcatcgcttgaaggagggaatggaaaacgcgtcattcctatatttcttagtctctggcaagagcatcgcctgccaattggaaactcttctaataaatcaacttcctaatcatggttttcagctaacaaacgttgctgatggtaagcatcgtaattttggca

SEQ ID NO: 6 - DNA, 3180 bp, Sorghum bicolor
atgcaccgggtgctcgtgagctcgctcgtggccgccacgccgcggtggctccccctcgccgactccatcctccggcgccgccgcccgcgctgctctcctcttcccatgctgctattcgaccggagggcttggtccaagccaaggaaggtctcacgaggcatctcagtggcgtctaggaaagctaacaaacagggagaatattgtgatgaaagcatgctatcgcatatcatgtggtggaaagagaaaatggagaagtgcagaaaaccatcatccgtacaattgactcagaggcttgtgtattcaaatatattagggttggatccaaatctaagaaatggaagcttgaaagatggaaccctgaacatggagattttgctatttaaatcaaaatttcctcgtgaggttctactttgcagagtaggagacttctatgaagctattggttttgatgcctgtattctcgtagagcatgcaggcttaaatccttttggaggtttgcgttctgacagtatccctaaagctgggtgtccagtcgtgaatttacggcagacattggatgatttgactcgatgtggttattctgtgtgcatagttgaggaaattcaaggcccaacacaagcccgttcccggaaaagtcgatttatttctgggcatgcccatcctggtagtccttatgtatttggtcttgctgaagtagaccatgatgtagagttccctgatccgatgcctgttgttgggatttcacattctgcaaaaggttattgcttgatatctgtgctagagacaatgaaaacttattcagctgaggagggcttaacagaagaggctattgttactaagctccgcatatgtcgttatcatcatctataccttcacaattctttgaagaataattcttcagggacatcacgctggggtgaattcggtgaaggagggctcttgtggggagagtgcagtgggaagtcctttgagtggtttgatggtttacctattgaagaacttttatgcaaggtacgggaaatatatggccttgatgagaaaactgtttttcgcaatgtcaccgtctcattggaaggcaggccccaacctctttatcttggaactgctactcaaattggagtcataccaactgagggaataccgagtttgctaaaaatggcactcccttcaagttgtggcgggcttccatcaatgtatattagagatcttcttcttaatcctccatcatttgatgttgcggcagcggtccaagaggcttgcaggcttatggggagcataacttgttctgttcctgaatttacttgcatatcacttgtgaagctacttgagtctaaagaggtcaatcacattgaattttgtagaataaaaaatgtccttgatgagattatgctcatgaacaggaatgctgagctttctgcaatcctgaacaaattgctggtacctggttctgtggctactggtttgaaagttgaagctgatatgctagtcattgaagattttatgccaattatttcaagggtgaaatctgtagtgtcctcaaatggaggttcgaaaggagaaatctgttatgcaaaagaacatgaagctgtttggtttaaaggaaagcgattcacaccaactgtatgggctaacacacctggtgagcagcaaataaaacaactgaagcctgcaattgattcgaaaggcagaaaggttggggaggaatggtttacaacaagcaaagttgagaatgctttagccaggtaccatgaagcttgtgataatgcaagaaataaagttgttgagctgttgagagggctttcaagtgaattgcaggacaaaattaacatacttgtcttttgctcaacactgctcatcattgcaaaagcactttttggtcatgttagtgaggctcggagaagaggctggatgcttcctactatatttcccttgtcaaaggactgtgttgcagaggaaagttcaaatgcaatggatttagtaggactctttccttactggcttgatgttaatcaaggaaatgcaatattgaatgatgtccacatgcactctttatttgttcttactggtccaaatggtggtggtaaatctagtatgttgcgatcagtctgtgcagctgcgctgcttggaatatgtggcctgatggtaccttcaacttcagctgtaatcccgcattttgattccattatgctgcatatgaaagcctacgatagcccagccgatgggaaaagttcatttcagattgaaatgtcggagatacgtgctttagtcagccgagctactgctaggagtcttgtcctgattgatgaaatatgtaggggcacagaaactgcaaaaggaacctgtattgctggtagcatcatcgaaaggctggataatgttggctgcctaggcatcatatcaactcacctgcatgggatttttgacttgcctctctcactcagcactactgatttcaaagctatgggaactgaagtggtcgacgggtgcattcatccaacatggaaactgatggatggcatctgtagagaaagccttgcttttcaaacagccaggagggaaggcatgcctgagttcataatcagaagggctgaggagctatatttgactatgagtacaaataacaagcagaccgcatcaatggtccacaatgagcctcgtaatgacagccccagtgtaaatggcttggttgagaagcctgaatatctgaaatacagactagaaattctgcctggtacctttgagccgttgcggagggaagttgagagtgctgttactatgatatgcaagaaaaaactgttggatctttacaataaaagtagcatcccagaactggttgaggtggtctgtgttgctgtaggtgctagagagcaaccaccaccttccactgttggcaggtctagcatctatgtgattatcagaagcgacaacaagctttatgttggacagacggatgatcttctggggcgccttcacgcccacagatcgaaggaaggcatgcaggatgctacgatattatacatcttggttcctggcaagagcgttgcctgccagctggaaacccttctcataaatcagcttccttcgaggggcttcaagctcatcaacaaggcagacggaaagcataggaacttcggtatatctcgaatctctggagaggcaatcgccacccagctaaactaa

SEQ ID NO: 7 - DNA, 3399 bp, Oryza sativa
atggccattcagcggctgctcgcgagctcgctcgtggccgccacgccgcggtggcttcccgtcgccgccgactcgtttctccggcgccgccaccgccctcgctgctccccgctccccgcgctgctatttaacaggaggtcctggtctaaaccaaggaaagtctcacgaagcatttccattgtgtctaggaagatgaacaaacaaggagatctctgtaatgaaggcatgctgccacatattctgtggtggaaagagaaaatggagaggtgcaggaaaccatcatcaatgcaattgactcagagacttgtgtattcaaatattttaggattggatccaactttaagaaatggaagcttgaaggatggaagcctgaacacggaaatgttgcaattcaaatcgaagtttcctcgtgaagttctactttgcagagtgggagatttctacgaggctgttgggtttgatgcatgtatccttgtggagcatgcaggcttaaatccttttggaggcttgcgttctgatagtattccaaaagctggatgtccagtcatgaatttgcggcagacattggatgatttgactcgatgtggttactctgtgtgcatagttgaagaaattcaaggcccaacccaagctcgtgctaggaaaggccgatttatttctggccatgcacatcctggtagtccttatgtatttggtcttgctgaagtagaccatgatgttgagttccctgatccaatgcctgtagttgggatttcacgatctgcaaaaggctattgcctgatttctgtgctagagacaatgaaaacatattcagctgaggagggcttaacagaggaagcagttgttactaagcttcgcatatgccgttatcatcatctataccttcatagttctttgaggaacaattcttcaggcacatcacgctggggagaatttggcgaaggtgggctattgtggggagagtgcagtggaaaatcttttgagtggtttgatggtaatcctattgaagaactgttatgcaaggtaagggaaatatatgggcttgaagagaagactgttttccgtaatgtcagtgtctcattggaagggaggcctcaacccttgtatcttggaacagctactcaaattggggtgataccaactgagggaatacccagtttgctaaaaattgttctccctccaaactttggtggccttccatcattgtatattagagatcttcttcttaaccctccatcttttgatgttgcatcatcagttcaagaggcttgcaggcttatgggtagcataacttgctcgattcctgaatttacatgcataccggcagcaaagcttgtgaaattactcgagtcaaaagaggttaatcacatcgaattttgtagaataaagaatgtcctcgatgaggtgttgttcatgggtagcaatgctgagctttctgctatcctgaataaattgcttgatcctgccgccatagttactgggttcaaagttgaagccgatatactagtgaatgaatgtagctttatttcacaacgtatagctgaagtaatctctttaggtggtgaaagtgaccaggcaataacttcatctgaatatattccgaaagagttcttcaatgatatggagtcatcttggaagggacgtgtaaaaagggtgcatgctgaagaggagttctcaaatgttgatatagctgctgaggcactgtcaacagcggtcattgaagattttctgccaattatttcaagagtaaaatctgtgatgtcctcaaatggaagttcgaagggagaaatcagttatgcaaaagagcatgaatctgtttggtttaaagggaggcgattcacaccaaatgtgtgggccaacactcctggtgaactacagataaagcaattgaagcctgcaattgactcaaaaggtagaaaggtcggagaagaatggttcaccactatcaaagttgagaatgctttaaccaggtaccatgaagcttgtgataatgcaaaacgtaaagttcttgagttgttgagaggactttcaagtgaattgcaggacaagattaatgtccttgtcttttgctcaacgatgctcatcataacaaaagcactttttggtcatgttagtgaaggacgaagaaggggttgggtgcttcctactatatctcccttgtgtaaggataatgttacagaggaaatctcaagtgaaatggaattgtcaggaacttttccttactggcttgatactaaccaagggaatgcaatactgaatgatgtccatatgcactctttgtttattcttactggtccaaacggtggtggtaaatccagtatgctgagatcagtctgtgctgctgcattacttggaatatgtggcctgatggtgccagctgcttcagctgtcatcccacatttcgattccatcatgctgcatatgaaagcatatgatagcccagctgatggtaaaagttcgtttcagattgaaatgtcagagatacgatctttagtctgccgagctacagctaggagtcttgttctaattgatgaaatatgtaggggcacagaaacagcaaaaggaacatgtatagctggtagcatcattgaaagactcgataatgttggctgcataggcatcatatcaactcatttgcatggcatttttgaccttccactgtcactccacaatactgatttcaaagctatgggaaccgaaatcatcgataggtgcattcagccaacatggaaattaatggatggcatctgtagagagagtcttgcttttcaaacagccaggaaagaaggtatgcctgacttgataattagaagagctgaggaactatatttggctatgagcacaaacagcaagcagacatcatcagctgtccaccatgaaatatccatagccaactctactgtaaatagcttggttgagaagcctaattacctgagaaatggactagagcttcaatctggttccttcggattactaagaaaagaaattgagagtgttgttaccacaatatgcaagaagaaactgttggatctctacaacaaaaggagcatctcagaactgattgaggtggtctgtgttgctgtgggtgctagggagcaacccccaccttcaactgttggcaggtccagcatttatgtaattatcagacgtgacagcaagctctatattggacagacggatgatcttgtgggtcgacttagtgctcacagatcgaaggaaggtatgcaggatgccacgatattatatattttggtacctgggaagagcattgcatgccaactggaaactcttctcataaatcagctacctttgaaaggtttcaagctcatcaacaaggcagatggcaagcatcgaaatttcggtatatctcttgtcccaggagaggcaattgccgcatag

SEQ ID NO: 8 - DNA, 3381 bp, Brachypodium distachyon
atgcagcggcttctggcgagcacgatcgtggccgccacgccgcgttggctccccctcgccgactctatcgtccggcgccgccgcccgcgccgttccccgctccccgtcctgctattccacagatcattgtacaaaccaaggaaggtttcacgaggcattacaatggtgtctaataaggtgaacaaacagggagatctctgcaatgaaggcatgctgtcacatattatgtggtggaaagagaaaatggagagctgcaggaaaccatcatctgtgcagttgactcagagacttgtgtactctaatatattagggttggatccaactttaaggaatggaagcttaaaagatggaaccctgaacatggagatgttacaatttaaatcaaagtttccacgtgaggtcctactttgcagagtaggagatttctatgaagccattgggtttgatgcctgcattcttgtagagcatgcaggcctaaatccttttgggggcttgcgttctgacagtattccaaaagctggatgtccaatcatgaatttgcggcaaacattggatgatttgactcggtctggttattctgtgtgcatagttgaggaaattcaaggcccaactcaagcccgtgctcggaaaggtcgatttatctctggccatgcgcatcctggcagtccttatgtatttggtcttgctgaagtagatcatgatcttgagtttcctgacccaatgcctgtagttgggatttcacgctctgcaaaaggctattgcttgatttctgtgctagagacgatgaaaacttattcagctgaggagggcctaacagaagaagctgtagtgactaagctgcgcatatgccgttatcatcatctataccttcacagttctttgaggaataattcttcagggacatcacgctggggggaattcggagagggaggactcttgtggggagagtgcagtggaaagtgttttgaatggtttgatggttctcctattgaggaacttttatgcaaggtaagggagatatatgggctggatgagaaaactaatttccgcaatgtcactgtctcattggaagggaggcctcaacctttatatcttggaactgctactcaaattggagtgatacaaacggagggaattcccagtttactaaaaatgctactccctccaaactatggcgggcttccatcaatgtatatcagagatcttcttcttaatcctccatcttttgatgtcgcgtctgcaattcaggaggcttgcaggcttatgggcagcataacttgttcgattcctgaatttacttgcataccatcagcgaagcttgtgaaattactcgagtcaaaagaggttaatcacattgaattttgtagaataaagaatgtccttgatgacattatattaatgaatggaaacactgagctttctgctatcatggacaaattgctcgaacctgcttcggtggttactggtttgaaagttgatgctgatatactaattagagaatgtagccttatctcacaacgtataggtgaagtcatctctttaggtggggaaagcgatcaggcaataacttcatcggaatatattcccaaggagttctttaatgatatggagtcatcttggaaggggcgtgtgaaaagggttcatgctgaagaagagttcacaaatgtcgatgtagctgctgaagcattatcaaccgcggtaactgaagattttctgccaattattgtaagagttaaatctgtgatatcttcacatggaggttctaaaggggaaatctcttatgcaaaagaacacgaagctgtttggtttaaagggaagcgattcacaccaaatgtctgggcgaacacacctggtgaacaacagataaaacaactaaagcctgcgattgattcaaaaggtagaaaagttggggaggaatggtttacaacaatcaaagttgagaatgctttagccaggtatcatgaagcttgtgatagtgcaaaaggcaaagttcttgagctgttgagaggtctttcaagtgaattgcaggacaagattaatatacttgtcttctgctcgacgctgctcatcatagcaaaagcactttttggtcatgttagcgagggtcttagaaggggttgggtgcttcctgccatatctcccctatctaaggactatagtactgaagaaggctcaagtgaaatggatttattgagactctttccttactggcttgacagtaatcaagggaatgcaatactgaatgatgtcaatatgcactctttgtttattctgactggcccaaatggtggaggtaaatccagtatgttgcgatcagtctgtgcagctgcattgcttggaatatgtggtctgatggtgccagctgcttcagctgtcatcccacactttgattccatcatgctgcatatgaaggcctatgatagcccagctgatgggaaaagttcgtttcagattgaaatgtcagagatccgatctttagtcagccgtgctactggtaggagtcttgttctcattgatgaaatatgtaggggcacagaaactgcaaaaggaacttgtatagctggtagcatcatcgaaaggctcgacgatgttggctgcctaggcatcatatcaacccatttgcatggcatttttgacttgcctctgtcactcggcaatactgatttcaaagctatgggaacagaagttgtcaatgggtgcattcagccaacatggagattaatggatggtatctgtagagaaagccttgcttttcaaacagcaaggaaggaaggtatgcctgacttgataattaaaagagcagaggagctatacagtactatgggcagaagcaagacgtcatcaacagtccaccatggtccatccgttgctaagtctaaagcaagtggattggttgatatgcctgatggtctgggaaatggattagaacttccatctggtgcttttgcactgctgcgaaaggatgtcgaaataattgtgaccgcaatatgcaaggataaattgttggatctctacaacaaaagaagcatctcagagctggttgaggtggtttgtgttactgtaggtgctagggagcaaccgccaccttcaactgttggcaggtccagcatctacatagttatcaggcgtgacaacaagctctatgttggacagacggatgatcttgttggccgtcttgctgttcatagatccaaggaaggtatgcagggtgccacaatattatatatcgtggttcctggcaagagcgttgcgtgccagctggagacacttctcataaaccagcttccctcgaaaggttttaagctcacgaacaaggcagatggcaagcatcggaacttcggcatgtctgttatctctggagaagccattgctgcacactga

SEQ ID NO: 9 - DNA, 3520 bp, Vitis vinifera
atgtactggctgtcaaccaaaaacgtcgtcgtttcattccctcgattctactctctcgctcttcttctccgttcccctgcctgcaaatacacttcatttcgttcttctacacttctactccaacagtttgagaagagccgatgtctcaacgaaaggagggttttgaaaggagctggaagaatgacaaaaaatgttataggattgcaaaatgagctagatgaaaaggatctttctcacataatgtggtggaaggagaggatgcaaatgtgtaaaaagccgtccactgtccaccttgttaaaaggcttatatattccaatttgctaggagtggatcctaacttgaaaaatgggaatctaaaagaaggaacgctgaactgggagatgttgcagttcaagtcaaagtttcctcgtgaagttttactctgcagagtaggggatttttatgaagccatcggaattgatgcttgtattcttgttgaatatgctggtttgaatccttttggtggtttgcgctcagacagtataccaagagctggctgcccagtcatgaatctacgacaaactttggatgacctgacacgtagcgggtattcagtttgcatagtggaggaagttcagggtccaactcaagctcgttctcgtaaaggtcgttttatctctgggcatgcgcatccgggtagtccttatgtatttggacttgttggggttgatcatgatcttgattttccagaaccaatgcctgtagttggaatttctcgttctgcgaagggttattctataattttagtccttgagactatgaagacgttttcagtagaggatggtctgacagaagaggctttagttaccaagcttcgcacttgtcactaccatcatttattgctgcatacatctctgagacgcaactcctcaggtacttgtcgttggggagaatttggtgagggaggactattatggggagaatgtagtgctagacactttgaatggtttgaaggggatcctgtatctcaacttttgtttaaggtgaaggagctctatggttttgatgatcaagttacatttagaaatgtcactgtgtcttcagagaaaagaccccgttctttacaccttggcacagctacacaaattggtgccataccaacagagggcataccgtgtttgttaaaggtgttgcttccatcaaattgcactggtctacctcttttgtatgttagagatcttcttctcaaccctcctgcttatgagattgcatccataattcaagcaacatgcagactcatgaacaatgtaacgtgctcgattcctgagtttacttgtgtttcccctgcaaagcttgtgaagctacttgagcttagggaggctaatcatattgagttctgcagaataaaaagtgtacttgatgaaatattgcagatgcatagaaactctgatcttaacaaaatccttaaattattgatggatcctacctgggtggcaactggattgaagattgactttgacacattggtgaacgaatgtgaatggatttcagctagaattggtaaaatgatctttcttgatggtgaaaatgatcaaaagataagttaccatcctatcattccaaatgacttttttgaggacatggaatctccttggaagggtcgtgtgaagaggatccatgtagaagaagcatttgctgaagtggaaagagcagctgaggcattatctttagctatctccgaagattttctacctattatttcaagaataaaagctaccacagccccacttggaggtccaaaaggagaagttgtatatgctcgagagcatgaagctgtttggttcaagggaaaacgttttgcaccagttgcatgggcaggtactccaggggaagaacaaattaagcagcttagacctgctatagattcaaaaggtagaaaggttggattggaatggtttaccacagtgaaggtggaggatgcactaacaaggtaccatgaggctggggacaaggcaaaagcaagggtcttggaattgttgaggggactttctgcggagttacaaactaaaattaacatccttatctttgcttccatgttgcttgtcattgcaaaggcattatttgctcatgtgagtgaagggagaagaaggaaatgggttttcccctctcttgtagagttgcataggtctaaggacatggaacctctggatggagctaattggatgaagataactggtttatcaccatattggttggacgtggcacaaggcagtgctgtgcataatacagttgatatgaaatcattgtttcttttgacaggacctaatgggggtggtaaatcaagtttgcttcgatcaatttgtgcagccgcattacttggaatatgtggatttatggtgcctgcagaatcggccttgattcctcattttgattctattatgcttcacatgaaatcttatgatagcccagctgatggaaaaagttcatttcagattgaaatgtcagagatgcgatccataatcactggagccacttcaagaagcctggtgctgatagatgaaatctgccgaggaacagaaacagcaaaggggacatgtattgctggtagcatagttgaaactcttgataagattggttgtctgggtattgtatccactcacttgcatggtatatttaccttgggactgaatactaagaatgctatttgtaaagcaatgggaactgaatatgttgatggcaaaacaaaaccgacctggaagttgatagatggaatctgtagagaaagccttgcctttgaaacagctcagaaggagggaattcctgaaacaattatccgaagagcagaagagctgtatctttcaatccattcaaaagacttaattacagggggaactatttgtcctaaaattgagtcaacaaatgaaatggaagtcttacataagaaagttgagagtgcagtcaccattgtttgccaaaagaagctgaaggagctctataagcagaaaaacacgtcaaaacttccagagataaactgtgtggccattttgccaggggaacagccgccgccatcaacaattggtgcttcaagtgtgtatgtgttgtttagcactgataagaaactttatgttggagagacagatgatcttgaaggcagagtccgtgcgcatcgatcaaaggaaggaatgcagaaggcctcattcctttattttgtggtcccagggaagagcttggcatgccaactcgaaacgcttctcatcaaccagctccctgtccaggggttccaactggtcaatagagctgatggtaaacatcgaaattttggcacattggatcactccgtggaagttgtgaccttgcatcaatgagcctgcgctccttgccacccattttgtagaatggttccatctttgaaatatgtacttgaatgacaaaaaccagatgaaagtggctgcagcaattttggttttttgatgtacgttgctccacttgcattagtattatctacctgatgaaatatgcattgatattgcttgctctaca

SEQ ID NO: 10 - DNA, 3615 bp, Cucumis sativus
atggaaatatccatctatgtcgatgtggcattgtggcgggaagtatcggaaaccaagggttttctgttccggcgacgacgagttacaaacaccctcctcatttcaaaccaaaacgctttaaaacttccaatcacaacaagattgaagctcacaaaccatccatttttatccaccgccatgtactgggcggcaacacgaaccgttgtttctgcttcccggtggcgttttctggctcttttgattcgcttccctccgcgtaacttcacctcagttactcattcgccggcatttatagaaaggcaacagcttgaaaagttgcactgttggaaaagcagaaaaggttcaagaggaagcatcaaagctgctaagaagtttaaggataataatattctccaagacaataagtttctttctcacattttatggtggaaagagacggtggaatcatgcaagaagccgtcatctgtccagctggttaagaggcttgacttttccaacttgctaggtttagatacaaacctgaaaaatgggagtcttaaagaaggaactcttaactgtgagattctacagttcaaggcaaagtttcctcgagaagttttgctctgtagagttggagatttttatgaagcaattggaatagatgcttgcatacttgtggaatatgctggtttaaatccttttggaggtcagcgtatggatagtattccaaaagctggttgccccgttgtgaatcttcgtcaaactttggatgatctgacacgcaatgggttctcagtgtgcatagtggaagaagttcagggcccaattcaagctcgttctcgcaaaggacgttttatatctgggcatgcacacccaggcagtccctatgtttttgggcttgtcggggttgatcacgatcttgactttccagaaccgatgcctgtgattggaatatctcgatccgcaaggggctattgcatgagccttgtcatagagaccatgaagacatattcatcagaggatggtttgacagaagaggccttagttactaaactgcgcacttgtcaataccatcatttatttcttcacacgtcattaaggaacaactcctcaggcacttgccgctggggtgaatttggtgagggtggccggctatggggggaatgtaatcccagacattttgagtggttcgatggaaagcctcttgataatcttatttctaaggttaaagagctttatggtcttgatgatgaagttacatttagaaatgttacaatatcgtcagaaaataggccacatccgttaactctaggaactgcaacacagattggtgccataccaacagagggaataccttgtttgctgaaggttttgcttccatccaattgtgctggccttcctgcattgtatatgagggatcttcttctcaatcctcctgcttatgagactgcatcgactattcaagctatatgcaggcttatgagcaatgtcacatgtgcaattccagacttcacttgctttcccccagccaagcttgtgaagttattggaaacgagggaggcgaatcatattgaattctgtagaatgaagaatgtacttgacgaaatattacaaatgcacaaaaattgcaagctaaacaatatcctgaaattgctgatggatcctgcatctgtggcaactgggttgaaaattgactatgatacatttgtcaacgaatgtgaatgggcttccagtagagttgatgaaatgatttttcttggtagtgaaagtgaaagtgatcagaaaatcagttcttatcctattattcctaatggttttttcgaggacatggaattttcttggaaaggtcgtgtgaagaggattcacattgaagaatcttgtacagaagttgaacgggcagctgaagcactctcccttgcagttactgaagattttgtcccaatcatttctagaatcagggctactaatgcaccactaggaggtccaaagggagaaatattatatgctcgggaccatcaatctgtctggttcaaaggaaaacggtttgcaccatctgtatgggctggaagccctggagaagcagaaattaaacaactgaaacctgctcttgattcaaagggaaaaaaagttggggaggagtggtttaccacgaagaaggtggaggattctttaacaaggtaccaagaggccaataccaaagcaaaagcaaaagtagtagatctgctgagggaactttcttctgaattgttagctaaaattaacgtcctaatatttgcttccatgctactcataattgccaaggcgttatttgctcatgtgagtgaagggaggaggaggaaatgggtttttcccacccttgctgcacccagtgataggtccaaggggaaagttgcgatgaagctggttggtctatctccctattggtttgatgttgtcgaaggcaatgctgtgcagaatactattgagatggaatcattatttcttttgactggtccaaatgggggtggaaaatctagtttgcttcgatcgatttgtgctgctactttgcttgggatatgtggatttatggtaccggcagagtccgccctgattccccacttcgactcaattatgcttcatatgaaatcttttgatagtcctgctgatggaaaaagttcttttcaggtggaaatgtcagagatgagatccattgtcaatagagtaacggagagaagtcttgtacttatcgatgaaatctgtcgtggaacagaaacagcaaaaggaacttgtattgccgggagcattattgaagctcttgataaagcaggttgtcttggcattgtctccactcacttgcatggaatatttgatttgcctttagatacccaaaacattgtgtacaaagcaatgggaactgtttctgcggaaggacgcacggttcccacttggaagttgattagtggaatatgtcgagagagccttgcctttgaaacagcaaagaatgaaggaatctctgaagctataattcaaagggctgaagatttgtatctctcaaattatgctaaagaagggatttcaggaaaagagacgacagatctgaacttttttgtttcttctcatccaagccttaatggtaatggcactggaaaatccaatctcaagtcaaacggtgtgattgtaaaggctgatcagccaaaaacagagacaactagcaaaacaggtgtcttgtggaagaaacttgagagggctatcacaaagatatgccaaaagaagttgatagagtttcatagagataaaaacacattgacacctgctgaaattcaatgtgttctaattgatgcaagagagaagccacctccatcaacaataggtgcttcgagcgtatatgtgattcttagaccggatggcaaattctatgttggacagactgatgatctggatggtagggtccaatcacatcgtttaaaggaaggaatgcgggatgctgcattcctttatcttatggtgcctgggaagagcttagcttgccaacttgaaactcttctcatcaatcgacttcctgatcacgggttccagctaactaacgttgctgatggaaagcatcggaattttggcacagccaatctcttatccgacaatgtgactgtttgctcatga

SEQ ID NO: 11 - DNA, 660 bp, Gossypium hirsutum
ggcacgaggttgctattgctgcaagggaacagccacctccatcaactatcggtgcttcttgcgtatatgtcatgttcagacctgataagaaactatacattggagagacggatgatcttgatggtcgaattcgttcgcatcgttcaaaggacgggatggaaaatgcttctttcctatatttcacagttccagggaagagtattgctcgccaactcgaaactcttctaatcaaccaactcttaagtcaaggcttcccgatcgccaacttggctgacggtaagcatcagaattttggcacatccagtctctcatttgacggcataaccgtagcctaacgagttaaaatgtatatcaatacgtaatttatatcgaaattgacatagaagtggcggcagcaattttgcctttgatctcggttgctccacttgctttgtacatgcatcacccttttaaccaagggtaaagttttctagtcataatttaatagcatgtatctattaagtccattttgaggtttatatgaatcaggttttcatcattaattggttaaattctgttattagctcctctactttactaaagttgtagatttagttcttatactttaattagattatttttactctatacttttcgaatgataaaattttagtcttcatt

SEQ ID NO: 12 - DNA, 2225 bp, Oryza sativa
agaaaagctaaaatggatgaaaaaaacagagaaaaagaaatcctaccctccatagcgagcagtggtggtctgcactctgcaggcaggcaggcacccaggcctgcagctcgatggcgccgccgccgccgtcgccgccgcgatctctcacctccgtctccctccggactcctctgagccctctcctcttcctacggccagcttcttgcaatccatcggcggtctccggctcctgcagcagcggagcatgccgcggcgtgcgctgctcggcggcgaacaagccttctccttccaccgctccgggcaccgaggtaaggtagccggctagccgccccccatattcttgtttctgtgttgatcggagctcgatggctggggtgctctgggctcgtcgtcgtcggtcgatcgtcatggcttgcttcgtttcttgcagctcgacctccatggcaaagataaggagtgaggtgctgtccccgttccgctccgtgcggatgttcttctacctcgcgttcatggccagcgccgggctcggggccctcatcgcgctcacgcagctcatcccggcgctgtccagcccggcgagggcggccgccgcgggggagacgctcaagggcctgggcatcgacgtcgcggcggtctccgtcttcgcgttcctctactggcgcgagagcaaggccaaggacgcgcaggtggcgaagctcacgcgggaggagaacctgtccaggctcaggatccgcgccggcgagggccgcccgcccgtcccgctcggcgagctgaggggcaccgcgcggctcgtcatcgtcgccggccccgcggcgttcgtcaccgagtcgttccgccggagcaagccgttcttgaaggacctcatggagcgcggcgtgcttgtcgtgcccttctcgacggacggcaacgcgccggacctgcagttcgacgaggccgacgaggaggaggaggaggcggcggcggcggctgggaagatgaagcggaggctctggcagctcactccggtttacacttctgaatgggccaagtacgcgcaaagccgggatcccatgaatttagctgcttaaatttcttcttcatgtcaatcgaaattcaaatgcaaattagtatctcattttcaaatcgattgctgcttcttgcagatggctagatgagcagaagaagctagccaacgtgtcacctgattcccccgtgtgagtatcaaaaactactctgaatttgtctgaaaatataactgaagtttctgcagctgctgaactgaaaccgcatcactcttgcaggtatctctcgctccggctggacggccgcgtccgtggcagcggcgtcgggtacccgccgtggcaagcgttcgtggcgcagctgccgccggtgaaggggatgtggtccggcctccttgatgggatggacgggagggtgctttgaatatttgactgatacagaccgtgaaaacattagttgattggagaaaaaaaaggacggccgggttcgatctatagcttatactagaacaagaacaggaagagtttgatgattgctttaacttctgtggggttgattttgcttcctgcatcccagcgacatcgcccaagtgaatgtgatatgccatgtgcccatgtacatgttgttttgcagcctacgtgacttgattattaacgagaatcctgtgtcaaagatcgctttttccgtggtaggcttctccattttattttatttttgaatatatatacgaaccgtgacaaatctgatggaacactggaccatgggggtaatgatactgtagtcgcctggtctttttatcaggcgctaaatgcaaacaatcagacagcttaaacaacctgaggttgttcagccagcccagaatacaaaaagcccatggaccgtgagcccgtgaaaccatggcccacccatcagtcacgtcacgtcgggacgtgtgcgcactaccccgagaagcgcgcggccgtaagccacaaccacaaccccacaccgcctcttggctgctcgcgccctgacacttcccaaaaccccaaaccgcccatatctctctcctcttctccgctcctcgcttcccccaaaaccctcgcggtggcggtgggccgccggtctcattccgccggcgtccacggaggcggccggagccaggcgctccgcgggccggggagccaccggaaggctggaaccctagcggcggcgggtctcctccccctccccgcgcgccgggcggcgcc

SEQ ID NO: 13 - DNA, 2000 bp, Solanum lycopersicum
cacgcatcaacatgtactagctaactttgtccaccaaggaatatctattaccttcaaccagcaacaagtatcagctaactttgtccatttaggttaaaaccttgactaaatccattgaaaacctattacctcccaccagcaacttatgccaactaacattgtctaccaagggatatccgttgcctcaacattgtccatcgagggatatccattgcctcccagcagcaacaagtaccagctaacattgtccaatcgagggacatccataccaaataatttttgagaaagtaattatttagggtgatgtttatttgtgcttggaaaacaaatagccattgaattagtaaaatttgcaattaaatcacattacaaaaatgtctaaggtaatttatcctaaatacaatagtgatatcaataataatatatttgagtgtaatgtcataatcaagaatttataaaaataatatgtcagtaggtttaattactgatatattaaaattgtttcgataaattcttgatttaaagagtacaatcaacaaataaaagaacaaatagtaaaatataagatacgtatatttttaaatagtagtgtacagtaggactagatgacaaaataaaattgtaagaagaagaggatagtctttaccattctcacacaaaatcatatctcgtcacaaatgaatttacagttactcataaatatatttaataaaatgatagtattagcaaaacataattgatgtgttagttcatcagttattatgcacttaaagttatttataaaaaaaaaaggtcaaatgccccccctaatctgttgtccgatttctaagtacgtacgtaaactttagaggggtcatatcacccctgaactgtttaaaattgaaatttttgtactcctaaaaagccataaccatatttatgttgatgagtgaaactcacgcgttttgacacatgaaaaatccattagaaaaagtttttcttcaattttttcattattctttattcttttcttctattttttcaccatctctccaacacaaaaatattttcactattttctccattatcataaacaccaaaggttcactaatcactacaatcttcaaaacgaaaaggtcggcagtgattttgattcttatttgtcatttcgcccactacttagttggtgctaagatcattttcatgaatacatgtgtcaaatttctcaaattagagtaaatgaataacaatggaggaaagtcgaatgtgggttttattaaaaatttaaaattcttcgtttgtttgaattaaaattacaaatttggagctttgaagaaccaaaaaaagaatttgccgacctaattgaccttaatttgtcgtttgaccatgtacaaggatgattaacaactccaataatatcgataaagagttgattttcaaaaactcttaattccatacacaaattaaaatcaagaaaaatgcgaaaattttatgaacaaatctaataacaaagaacgaaaaaatttctacgaagaacaaagaaaagaataaggaagaagataaaatatttgttttggtgctcactctatcagggagtgaaatacattcactatggagatgataatttcaattttaaacagttcaggaaggtgatatgcgtaaagtttaagttaatagataatcaaagatatctgttgacgtattgattcaatgttaaatatacagttgagttacttataacttataaatacaataagtaatttttttccttctaccatttgaaaaaaaataacgcgtacgtcattgtcttcaatcatcaacgatttattattttcaatgtgattatattattaagtaattaattcgtcccaaataccaatatctactaacattctttgcctaatgtttaattgtaattcctacagattttatttttttgaaaataatataaagtacaccatttctctgccgggaagaacaaatacacagagagagagtgtattgtgcactgatatcgagca

SEQ ID NO: 14 - DNA, 2000 bp, Sorghum bicolor
tggcacataagtaaaattgctgacttggtagggtcatttaatgaaggaatttcatgagatgagagagaagtttcatccccatgaaactcatatggctcggttacttggtaactgtgtcatcaaactatgcattgagactggcctgcatgtttcatgagagtgtcatgcacattaaataagatatcacataagcaaaattgctgacttagctgggtcattaaatgaaggagtttcatcagatgagagaggagtttcatcttcataaaactcttgtggctcggttacctagtttttagtctcggtaactgtgtcatgaaactataatacatgttttatggaagtgtcacgcatattatagggtgccacataagtaaaattgttaacttgacagagtcattaaatgaagaagtttcatcagatgagagaggagtttcatccccataaaattcatgtggctcagttacctagtttatagtcttggtaactgtgccatgaaactatgcattgagactagcctaacaaggtgataaggccagtctcaatgcatgtttcataagagtgtcatgcacattaaataagatgccacataagcaaaattgctgacttgacagggtcattaaatgaaggagtttcgttagatgagagaggagtttcatccccatgaaactcttatggctcgattacctagtttatagtcttgataactgtgtcatgaaactatgtattaagactggagactcaaatacaaattgtatatagcctatggctctatttgtttccgcgaccggccaacccgtgaccacgattaagcaaacacgactcgagatcgtgtatgtataaatttggatcttcggtggtttaattttagtttttttagctaagagaatttatatacattttccattattaaaccatgtttcaaggtttctaattccatgctctaaaataatatattattttatggaaaatagatatttttgaaattaaatgtatgtataaattttcaagactatataaaataataaaagctctaatctattgatgtagttcaatttttatctccctagagctgatgtggcaccacatacggtgccacctaggttaaaactaacctcaaaacccggctaggtttgtgatttgcctcgtatttgatagtttagggtgtactccgtataagattatctgtatattcagagattagaaaatcaaacacatctactaaattttagaacaagcatcaccctgtttggcggctagtcgtaaaagtaactgatgttaatttattgtgagagtaaaatactgttgtttgataataaaacttttacttataatttcaagctaatagagcccataaagacaaacctgtctcacaagttagaattcttacaacttttcgtctaatcacatcgaatatttgaacacatgtatatagtattaaatataataaaaatatttaattgtacagtttacctatatttacaagacgaatattttaaatataattagtttattattaaacactaattactataattataaatatactacaatataaaaaaaaaactttcgtttttgcgagctaaacaccgggacacaaaacgctcctgtctcctgggcggcttgggcctgccagagcccgggaaccgtgagcccgtgaccccagcgggcccaaccagtcagacgagagtcgaaaggcgtgcgcactaccccgagaaacgtgcgacaggaacctcccccttcccggcctgggagcggcctggcgagtggcgactggcgtcttccgcggttccccccagttcgcctccttcactgccgcccgcgcgccccgacacgcctgaaaaaccccacctctcccctccgctccgcctctctcgccctccacttcccacgccccacgccgcctgccattccagttccagcgtggactcgacgccagcgcggagacgcgcgtctcgaagcactagccccccgttgttctgccgcgccggcgcgccggcgcc

SEQ ID NO: 15 - DNA, 2049 bp, Arabidopsis thaliana
catatgaaacatttcctgtagcagtgagatggttacaagaggatccatacccgagtgctgtgcaatcagacaaactacaagcatagtcgatattatcaggcaagtcatcgaggttatatgcattagggtcaagaatacaccaagttttgggcagatacttcacatcttccacaggaaccaaaggcttgtcatttcctttacctgacaagtcaagctcatatttcggtctcccatcaaactcaaagatcccccagtgcctctcaaaagtccccggtgctatactcttggcgtcctcatcaacaaggctgaaaaggtaaacatccataatcactcctttcctcgcaggcgttccatttccagacatagcatgctttaccattccctgattgaatctctttgcactctttacattagcgttcttgtctccatccgtaggccacccgacctctcctacaatgatcttcatccccaagaagctatatctctccatagcacaaatcaaagtgtcgagattcgcatcaaacacattggtgtagaccaaatttccatctctcaaagacttgttagtcccatcaaagaaggcaaaatccaaaggaaagtaagcatttccatagagactaagaaaagggtatatgttaaccgtgaaaggcgaatcatgtgaatacaagaaattgattatctcaatcgttgcatcccttagctcaggtctaaagtctccagctgatggaacagggttcgcttcaggggaaaaatagatgtctgcgttgaaaggaacagtgactttcacatttttcagatcagcttcctctagtgctcgttggatgttgataagagctggtaatgtgaactcaacgtaagttccattatatgtctgaaggaaaggctcgtttccaacagctatgtacttgatgttgactccaccgttgtaagaataagcagtaacgttttcttcaacccatgaagctgctacagatgtatcttgagccatttctttaagaaaccggtttggtattcctatcatgacttcaatgtctgagccaattagagcgtctaagatgttttggtcggcttcaaatagtttcagcttagtgaaactattgtccattagcatcttcacaaccttttctggtggaagctggtgactcgccattattccccagttaactcctacattactcgtgttgcttgaggcaatggaaacttgtgagatgataagaaagtaacaaagaataatctgatgattataaaagtggtttgttgttaactttgatctctctcctgccatttttttctctgtttatgagtcttttcttctcttttctttatggagtctttgttaagggagaagatgaaatgtgattggatatttgtgatttgtacttagttcagttaaagaagcagacacaacatgcaaaatagccattggtgaaacactttgtgcatgcctatctgataaatccattgactcaccacaaattcttatgtaattctagatgtttcgtatttgttgtgccaaacaaacacacacactcacacactgcactgagtctagacatttagtggttttgttttcttattattaatactcattagagtattaagtttgtatagaattcagaaacaactgatagtcattttaagatttctaattacaaaacttttgatcctctttgaaaagcagagaaattacaatctttacaaacaaaactgagagattagagatgtgttcatagagatgggttctttgttagacattccaaaaagatacaaaactagccgatgattaattttggtaaattaatgaacaagaatgtaatttgaaacattatagggagcaaatgagaaattactctttttaaaaggctaaaatcctaattacctttaaactaagaagacaagaagagaagagaaaacatgttttccattagaggactgtgagattgtgaattgcatagtcgtcgtcttctggcgggaaaagaagccctagaaaaagggtgaaaggtgaaaactctacttcttcttcttcttcttcttcagagtgtgagagag

SEQ ID NO: 16 - DNA, 946 bp, Arabidopsis thaliana
aaaagttgaaacagcaaagtagcgatagatttcgtgaaaacagagaagcggacatatcttgaaacacatggcagcgatttctccatggttatcttctcctcagagcttttcgaatccccgcgttaccattacagattccagaagatgttcatcaatttctgcggcaatctctgttcttgacagctccaacgaggaacaacatcgaatttcgtctagagatcatgttgggatgaagagaagagacgtcatgttacagatagcttcctctgttttcttccttccattggccatttcacctgcatttgcagagacaaatgcatcagaagctttccgtgtgtacacagatgaaacgaacaaattcgagatatcaatcccacaagattggcaagtcgggcaagcagaacctaatggattcaagtcaatcacagctttttacccacaagaaacttcaacttccaatgtgagtatagcgatcactggactaggtccagacttcaccaggatggaatcattcggaaaggtcgaagctttcgccgaaacattggtcagtggattggatagaagctggcaaaaaccagtaggagtgactgcaaagctaatcgatagcagagcttctaagggattctattacatcgagtacaccttacaaaaccctggagaagctcgcaagcatttgtactctgcaattggaatggcaacaaacggatggtacaaccgtttatacactgtcacaggacagtttacagatgaagaatctgctgaacaaagctctaagatccagaagacagtcaagtctttcagattcatctgagaatgtcattcatatctatcagcggaactaaattatagaattgatcaaacaatttgtttactgaacaattacttttttgcaatgaaattctgagaaaagagcctactccatactttgaagtaagcttcagtaaac

SEQ ID NO: 17 - DNA, 807 bp, Arabidopsis lyrata
tccagaagatgttcatcaattcctgtagcaatctcagctctagacagctccaacgaggaacaacatcgaatttcgtctagagatcatgtggggattaaaagaagagaagccatgttacagatagcttcctctgttttcttccttccattggccgtttcacctgcatttgcagagacaaatgcatcagaagctttccgtgtgtacacagatgaagcgaacaaattcgagatatcaatcccacaagaagattggcaagtcgggcaagcagaacctaatggattcaagtcaatcacagccttttaccctcaggaaacttcaacttccaacgtgagcatagcgatcactggactaggtccggacttcaccaggatggaatcttttggaaaggtcgaagctttcgctgaaacactggtcagtggattggatagaagttggcaaaaaccagcaggagtgactgcaaagctaatcgatagcagatcttccaagggattctattacatcgagtacaccttacaaaaccctggagaagctcgcaagcatctgtactctgcaattggaatggcaacaaacggttggtacaaccgcttatacactgtcacaggacagtttacagatgaagaatctgctgaacaaagctccaagatccaaaagacagtcaagtctttcagattcatctgagaatgttattcatatctatcagcggaactatattattgaattgatcaagcaatttgtttactgaacaatcacttttttcaatgaaattctgagaaaagagccaactccatactttgaagtaagcttcag

SEQ ID NO: 18 - DNA, 1054 bp, Cucumis sativus
ggagcgcattgtacaaagaaaatccatctctaatctttgagtggactacaagcatggcgatggcgtcccttctttcacccagcgctgtaatcctacgccctcactcattccgcttctcacaatcatcactctccaatggattctccattattcctatccgctcaacacttcgtgttttctgctctgccaatggcaacagcatccacacttctaacaaaaacccagttatttggcgagcggggtcaacagacgagaaattatgctagggattggattcactgcattttcatttcaagaagttgtttctaatgccctagctgagagtgttgtggttgctgaagattatcggacgtatacagacgaagcgaataagttcagcttggtgattcctcaagattggcaagtgggtaatggtgaaccgaatggattcaagtcggttacggcattttttcctcaagaaacttcaacttccaatgtcagtgttgtaatctcggggcttggtcctgattacacgaggatggaatcctttggcaaggttgaggaatttgctgatacattggtgagtggactggacagaagctggaaaaggccaccaggtgtggcggcgaaacttatcgactgtagatcatctaaagggatatattacatagagtacacactgcagaatccaggtgaaagccgcaaacatttatactcagcaattgggatgtcatccaatggctggtacaatagactttacaccataacaggacagtatgcagatgaagaatcggagagctatagctccaaaatcgagaaggttgtcaattccttcgctttcatttgatgattgccacagaattggcctccaccacactatcataatggttaaatgttttccacatctctctctaattatagttctcttttgttattattattattattattttttgtaatgagttctaaacataatattgaattgtctttgatgcatctatatttttacattttcacgaggaatgaattcacatttctattaattcataaaagaatccacaaaacagaaaaaaaa

SEQ ID NO: 19 - DNA, 795 bp, Ricinus communis
atggcttcaatttctttactctgttgcaattgctacttcacatctttctccaacaagacacctctccatcttttgaaacctaacttaaacttcctctctgcttcaccttcttttcgatttaacagttgcagaaagcaacatcttccatgttgcaccaactctttcccagacgaagaccaacaccaaccattattctgtcgttttaggcttcaagaaccatatggaagaagagaagctttgttcagcgtggcatttaccactgggtttacttttccagggcttatttctaatgcatttgcagagattgatgacttccgcctttatactgatgatgccaacaagttccaaatatcgattccccaagactggagagtaggtgctggagaacctaatgggttcaaatcagtgaccgctttctacccagaagaagcttcaggctctagtgtcagtgtagtgatcacaggactcggtccggattttactagaatggagtcttttggcaaagtggaagccttcgccgaaactctggttagtggattggacagaagctggcaaaggcccccaggcgttgcagcaaaacttatcgactgtaaagcgactaaagggatttactacattgagtacacattacaaaacccaggcgaaggtcgcaaacatctgttttctgctcttgggatggctttcaatggttggtataacagactgtatacagtgacagggcagtttgtggaagaggagtcagagaattatggatcaaaggttcagaaggttgtttcatcattcaagttcatctga

SEQ ID NO: 20 - DNA, 1028 bp, Vitis vinifera
caatcaaaaaaagcatggctctgtattttccacttcctctccgttctgggtcctgcgacttctcagcttattcgagtaaaaaaggttatgggtcaagaaccgggaaatgtggaaaaaagcaacgtgttgtcttctgcaagaatgagaacaaggaagaagaaaaaacaagttttgggattaaagaacaacatggaggtggaagaagggaggttgtgctacagatggtgttcagtacaatttcccttcaggcaattgttcctaacgcactggccgatactgaggtgccagaggatttcaaggtttactcagatgaggtcaacaagttcaaaatacagattccccaagattggcaggtgggttcaggagaaccaagtggatttaaatcagtgacagcattctacccagaagaagcttctggttcaaatgtcagcgtagttatcactgggcttggcgcggattttaccagactcgagtcttttggcaaagttgatgcttttgcagagaatctggtaaatggattggatagaagctggcaaaggccccctggtattgctgcaaaactcattgactgcagagctgctaatgggttttattacattgagtattggcttcagaatcctggggaaagtcgtagacatttattttcagctgttgggatggcaaacaacggttggtacaacaggctttatactgtgaccggacagtatttggaagaagaatcagaaaaattcagttctaaaattgagaaggttgttgcatccttcaggtttatttgaagaaaaatttgcatgttcaggatataaactgaggctgaagattactggttcagcaactctgtggatttcacaatgcacacgaattggcattgtgcaaaaagatgagatgatttatatactcagattgcatcaggtgtcttttgttgtaaaattgtaaggaaggggaagggaaattatctctatgctaccattgaaaattttttccacacctttgcagttgcttcacattcatttgcagaattgatggatgag

SEQ ID NO: 21 - DNA, 726 bp, Medicago truncatula
atggcatccatttcatggttcagctgtctacacatccgaccaacagccactgccggcgacaaaggtttatcatctcccataaccgtggaacatcataaaacaagaccacaaaatttactctcatcctcggaagaaggacttgcgattaatagaagacaactaattctttacacatccactgcagcaattgcagcttcatctactgactcaaatgcattggcactcaatgatgtatctgaggattttagtatctacactgatgatgagaacaagttcaagatagatattccacaagagtggcaaattggaacaggagagtctgcagggttcaaatcattaactgctttctacccaaaagagcaatctaattccaatgtgagcgttgtgatcacaggagtgggtccagatttcactaagatggaatcattcggcaaagttgaagaatttgctgacactctggttagtgggttggatagaagctggaaaaaaccacctggtgtggctgctaaactcatagattgtaaatcatctaaaggattttatttcattgagtatacgctgcaaagtcctggtgagggtcgcaaacatctatattcagctattgggatgttaacaaatggctggtataacagactgtatacagtgacaggacagtatggggaagaggaaacagacaagtatgcttccaaaattcagaaggcagttcgatcgtttaagttcatataa

SEQ ID NO: 22 - DNA, 579 bp, Populus trichocarpa
ggaactaaaagaagagaagctttattcaatatggtatttactgcttttactttccctgcaattgcctctactgcattggcagccacaggcgtggcagaggattcacgtgtttataccgatgatgcgaacaagtttaagatatctattccccaaggctggcaagtaggtgcaggagaaccaagtggatacaaatccgtcactgctttctatccagaagaagcttctaattcaagtgtcagcgttgtgatcaccgggcttggtccagattttactagattggaatcatttggcaaagttgatgcctttgctgagactctggtgggtggattggacaggagctggcagaggcccccgggcgtggcagcaaaacttatagactctaaagctgctaatgggctttactacatcgagtatacgctgcaaaatccaggcgaaagtcgcagacatttgctttcagcacttggagttacattcaatggttggtacaacagactatatacggtgacagggcagtttgtcgatgaagaatcagagaaattcggcaccgagatcaggaaggtatatcagaactcttcattt

SEQ ID NO: 23 - DNA, 951 bp, Lotus japonicus
ggaatggcgtccatttcctggtcttgttgtctgcgttggcgaccaacaatatccgaccgcacagcctctgcggccgacaaaggtttctcacctcccataacattggagcatcataaaaaaacaccatgtttactatcagcacgcaattcctccattgaagaaggacatgcggttaacagaagacaacttgttttctacacgtcactagctgcatttgcagctgccccatctactgtcctgaaggcattggcactcaatgatgtggttgaggatgttcgtatctacattgatgatgagaacaagttcaagatagagattccccaagattgggaagtaggaacaggagactctagtgggttcaaatcattaactgcattctaccccaaagaggcatctagttccaatgtgagtgttgctatcacagggttggggccggatttcactaagatggagtcgtttggcaaggttgatgagtttgctgagactctggttagtgggctggacagaagctggagaaaaccgcctggtgtagctgctaaactcataaatagtaaaccatctaaaggaatttattatatcgagtactcgttgcaaaatcctggtgagagtcgcagacatctatattcagctatagggatggcaacaaatggttggtataacagactgtatactgtgacaggacagtatgtggaagaggaaacagacaagtatgcttccgaaattcagaaggcggtcacatcatttaagttcatataaagaaatgctcatgatgaaggagaaatttccccacagccatctttcctatataaatacagatttgtgccttcctacagtgtaggattcttatgagcaagagaggattcttatatttgtctttatgagcaaaatggaatacttcattatttcattcctctcttatgtctcttgctcctcagattatgtatattgtat

SEQ ID NO: 24 - DNA, 866 bp, Fragaria vesca
atggcttctgttgcgtcttggcatcctctgttccttcgacctcgcacatcccatttcaccacgacctcctacaacacaggcaccgccatatgtagaaagagctatctgcaatgttgcaacaacaaagaacaagaaccacaaccagaacaagaagaaaaatcggtttttgggatgcaatgccaagccaagagaagacaagttttgcttgggactacttttgctgcattttcttttccggaaatttattccaacattgcattggccgagaatgacgattttcgtgttttcaccgatgatgtcaacaagttccagatatcaattcccctagactggcaagtaggcgcaggggaaccaagtgggttcaagtcagttactgctttttacccggaagagggatctagctcaattagtgtcgtaatcacggggcttggtccggattttacgaagatggaatcctttggcaaagttgacgaattcgctgagactctggtcagtggactagataggagctggcaaaggacagcaggagttgcagcaaaactcatagattgcaaatcatctaaagggatttactacattgagtattcgctacagaaacctggtgaaagtatcaagcacctctattcagctcttgggatggcaaacaacggctggtacaacagactatataccgtcactggccagtttggagaggaggaagcggataaatacagatccaaaattgagaaggctgtaaaatccttcaagttcatatgataaacaacctccagaggggcagagtttgaattgtgaactacggtttaccaattttgattgggtcagttgtacacaaatttttcatcgtaatctaatgtaatacatttgaa

SEQ ID NO: 25 - DNA, 1015 bp, Glycine max
gtgaataataatcagaacaaaaccaaccatttaaaaaaaaaaaaaaaagaaatggtggcagagaagctgatctgaaaggaatggcgttcatttggcggttctgtggtgtgtctctatgcaacttcacagcctctaatgcccagaaaggtccttctccttctctgcccataaccttggacttggagcatcatataacaaccccatctttactttcttccatcgaagaagaagaaggacgcgcggttaataggagacaacttattcttcacacgccagtagctgcagcagctgcatttgcagtcccaaatgcattggcactcaatgatgtgtctgaggatgttcgtgtctacactgacgatgagaacaagttcaagattgagattcccgaagagtggcaagtgggaacaggagacggagaatctagtgggtttaaatccataactgctttctacccaacacaggcatccaattccaatgtgagcgttgtgatcacagggctgggaccggatttcaccaggatggaatcctttggcaaagttgacgagtttgctcagactctagttagtgggcttgacagaagctggcgaaaacccccgggtgtggctgctaaactcatagattgtaaatcatctaatgggatttattacatcgagtatttgctgcaaaatcctggtgagagtcgcaggtatttgtattcagctattgggatggcatcaaatggttggtataacagactgtataccgtgacaggacagtatgtggaagaggacacagacaagtatgcttcaaaagttcagaaggtagttgcatcatttaggttcatatgaagaaaatggtcatgacgaggaagaatttttatcacagcacttcatctattctatttcattatggattttcctggcattgttctttaagctagatatggcattctagatcggactggtatgataaaaaccatgacatttccttcgagattgttgaatgaaagtaatatacttagtggccataattgaca

SEQ ID NO: 26 - DNA, 1159 bp, Solanum lycopersicum
ggctgtgaattggatacaccaatatctctgcttcttcaaagaaaacaaaaaaaataaaaacagaaatggcgactctttcatcttcatcttcatcttcatcttcatcttcatctccatgtttgaaccagtaccagtatcaagctattcttcgcttgccacgtgtccctttaatttcctctcatcttcttaaagttcccaagaaaaatcgaaactcacttattttctgctgcaacaacactgtgcctgattcaagaacaggtgagcaagttaaaggagaatgcttaaccaagagaagagagctcctgctacaggcaggctctgttgcattttctctgtccgcctttacatcgattgcattggcagagaaggatgtcccggaggagtttcgtgtttattcagatgatgtcaacaagtttaagatcatgatacctagtgattggcaaataggcgcgggagaaggtgatggagtaaggtcactcttagctttctatcctccagaagcttctaactcaaatgtcagcatagtaatcacaagccttggtgctgatttcaccaagttggaatctttcgggaaagttgatgcttttgctgagaatctggtcagcggatttgatagaagctggcaaaggcctccgggagtgaaagcaaaactcatagatagcaaagcttctaaagggttgtattacatcgagtacactctccaaaatcccggtgaaagtctcagacatctattttcagtgcttgggatagcaaacaatgggatttacaacagactgtatactctcactggacagtttgtagacgaggaggcagagaaatatggtgccaaaatacagaaggctgtttcttctttcagattaatatgatgacatgaacagagagcgcgatatcgcaaattttggcttgagcttctggtttttctcgtttggtgaatggtaaacataattgagagcgcgatatcacagattcaagttctggttaaggtatattatgacgactcgagaaaaaactggagttgtaagtatgaactagcaacttgatcaatgttagagttagtatttgcatatatcgttatataccaaaactgtatcgattttttgataaaaatatgaccttagtgcaaataatttgatgctcaagttttgattatatatttgta

SEQ ID NO: 27 - DNA, 1680 bp, Cicer arietinum
tctcgtaaaagaatgagattcagttgaagttatcgaagaagaagaatgcagcagaggaaaaggcaaagtatggatgggcctagcatctatgcgtggttgttttgtgtcattggaatgacatcacttttcggtgctgctttcctaccaccagatatagggattgtgttttttctcagacaaatatgtctgtttgtgtttcgatctatgtggatgacaaggttggtgttttttctagctgcagctgcacatgtcattgaggctatctatgcttggtgcttggctagaagactggatccttccaattcaagggcttggttttggcaaacattggctctaggctttttttcattgcgttttctattgaaattgaaaagatcaaaggaataactttgaaataatcacttcttgtaccattgttattttcagaaatcaattatctttcccatcaattgtatgccttttttttatgtaaaattaatatttttacaagttgttattagttatcactgtcgaataaatttaccatgacagtacatatcttttaattttgaaaattgtccattttgttttcttatctataatggttttcttctctatttaattttggatttaattaaattagaaatagtgtcttctctgtggaggataaaagtgtaattaaatagataataataataaaggaaatgaagggtgagaaagagaagctgatctaaaaaggaatggcatcaatttcatggttcagctgtttacacattccaccaacatcctctgctgccgataaaggtttatcatcatctcccataaccgtggaacatcataaaacaacaacacgtttaatctcttcctttgaaggacaacaacatgttgttaatagaagacaactgattctttatacatccacagcagcaattgcagcactatctactgtcccaaatgcattggcactaaatgatgtgtctgaggatgttagtatctacactgatgatgagaacaagttcaagatagaaattcctcaagagtggcagataggaacaggagagtctgcagggtttaaatccttaactgctttctacccaaaagatgaatctaattccaacgtgagcgttgtgatcacaggggtcggaccggatttcactaagatggaatcattcggcaaagttgaagaatttgctgacactctggtaagtgggcttgacagaagctggaaaagaccccctggtgtggctgctaaactcataaattgtaaatcatctaaaggattttattacattgagtatacgctgcaaaatcccggcgagagtcgcaagcatctatattcagttattgggatgtcaacagttggctggtataacagactgtatactgtgacaggacagtttgtggaagaggaaacagaaaagtatgcttccaaaattttgaaggcggttgcatcgtttaagttcatataaagaaatgcttgtgacgggagagaaatgttctcattggtttctttcatgggctgccgtttaatgttttcatgacattttttgtaagctagaaatggcgtctaaatgttataaatatgatatttgctatggtacttccttcaaaaactgataaaccagagtaggctaaatgaatggcacaaattgatgtaatggataagatattttgcagtgaatatcagcaccttcagttaaa

SEQ ID NO: 28 - DNA, 1068 bp, Triticum aestivum
gaagcacagcagcgtcgtcgaccaccatcgagccgtactccatggctgccgtgaccaccgcctctctctgtcccggcctcggcaagccccgccgagaccacgcgaagccaccgagaaccacggtctgccattgcctccctgctcggaggacggaggagggggtgaagcggcgggacgccctgctcggcgtcctcctctccgctaccgccgcgtcgtcggcgccgctgctcgtccccgccgaggctttcgccgaggtcgccgatgcgcaggaggggttcaccgcgtacgaggacgaggccaacaagttcaccctcgtgattccacaaggctggcaggtcggcgcaggtgaacgcagcggcttcaagaacgtgacagcgttcttccccgagcaaaaccccaactccagcgtcagcgtcgtgatcaccgggatcgggccggacttcaccagcctcaagtccttcggtaacgtcgacgagttcgccgaaaacctggtgaccggcctggacaggagctggcagaggccggcggggctcgcagcgaagctcatcgactccaaggcatcaaacggcttgtactacatcgagtacacgctgcagaaccccggcgagaagcgccgccacatcgtctccgccatcgggatggcattcaacggctggtacaatcggctgtacacggtgacagggcagtacatcgacgacgacgaggagtcagccatatacaaacctgagatagagaagtctgtcaagtcgttcaagttcacatgaaatgcccccaaaaaggaagttcaggtgagaacaagtatagagtgacagagaagagagagtatacaaagctagtagctcctgatgtcaagttcaattagtgagtatgcatatgtttgtcgaatttaccggaaagaaaagatgaacaccagatgttcgaagacttcgatggcgtagcttggctgagaacagcattggcagcatgagtgtgagatagagcatgagtgtcgttggttctaagaaaattgctagaactctgttacaaggaaactaaaattgctctgatgtaaaaaaaaaaaaaaaacga

SEQ ID NO: 29 - DNA, 1037 bp, Oryza sativa
gcaaggcaggcagcagcgaccagacgaagcagagagagcgcccgcgccgcgccggccatggctgccgccgtgaccaccaccaccaccgccacaaccacccatctctgccgtggcctctcctcctcctccgccgccgccgccaagccgcggcgagcgacgacgctcagatgcggcgccgctgctcgggtggaagggctggggcggagggaggcgttgctcggcgtgctcctctccacggcgacggcggcgtccgcgcccgtcgccgccgtggccgcgaccgccgagttgcaggaggggttccgcacgtacgaggatgaggccaacaagttcagcatcgccattccacaagactggctgatcggcgccggcgaggtcagcggcttcaagtccgtcacggcgttctaccctgaccaagtcgccgactccaatgtcagcgtagccatcaccggaatcggccccgatttcaccagcctcaagtcgttcggcgacgtcgacgccttcgcagagaccctggtgaacggcctggacaggagctggaaacggccgccgggggtcgccgcgaagctcatcaactccagggcagccaacgggttttactacatcgagtacacgctgcagaaccccggcgagcagcgccggcacattgtctcggccatcgggatggcgttcaacggctggtacaaccggctctacacggtgacaggccagtacatcgatgaggacggggatgtagacaagtacagggctcagatagagaagtgtgttcagtcattcaggttcacatgaaagaggagcatcctacacaacatccaacaaggcgaggacgaaaaacattttgtaaaccaacgtatttcgttataattgtaaatcaatcagtatattcatgtcatcagttcaaccaactaaatgtacaccaattgttccgagattttgacgatgcggccttgccgaggccaacatgagctaattatgttgtggcaagtcataagcattgttttctatgcatttttaagggagaaaaaacaggtgtatttgtt

SEQ ID NO: 30 - DNA, 1269 bp, Brachypodium distachyon
atggcccggctcggcccggcccacgtgttttacagccgttgggctgggccgtactgccaaaacgtgtccaaatggcccacgtcgaagacgaactctcacgggccgacgtccttggcgcgcggccatggcccatgggtaagtaagatacgagggggccgaagcaatgtgcggcggaagttcccggacgacgacacgacggccggccaccgtacaaagcttctcagcaaaatatcctccccctcgaaagccaccagcgcagcagcagcgcagagccattcgctcgccatggctgccgtgaccaccgcctcctccgccatctgccccggcttcagcagcaacccccgccgaggccacgcgaagccgcggagatccacggcctgggcctgccattgccgccgctcccctgccctgcgcgagcaacaacctacggcggccgttgccgggacggcggaggaggggctcaggcggagggatgccctgctcggcgtcgtcttctcggccggcacggcgacgctgctcgctagtcccgccggtgctctcgccgaggccgctgccgaggtgcaggaggggttcagcgagtaccaggacgaggccaacaagttcagcatcgtggttccgcaaggatggcagatgggcgctggtgagggcagcggcttcaagaacgtcacggcgttcttcccggacaaggccgccgactcgagcgttagcgtggtcatcaccgggatcgggccggacttcaccagcctcaagtccttcggcgacgtcgacgccttcgccgagaacctggtgaccgggctggacaggagctggcagcggcctgcgggggtcaccgcgaagctcatcgactccagggcgtccaacggcatgtactacatcgagtacacactgcagaaccccggcgataagcgccggcacatcgtctccgccatcggcatggcgttcaacggctggtacaaccggctctacacggtgacagggcagtacatcgaagatgacgaggagtccgtcaagttcaagcctcagattgagaagtctgtcaaatcattcaagttcacatgaaatgccttcaaaacaaaggtcacatgaaaataagtactgctactacttttgaatgaagtactatatctaagcagagaagagaaggtatataaaggcagcttccggtaatgtgtgcagaacgaaatgaactaaacctttgtgaatgtaagggttgtgagctttgagaatatatatgtttgtcaattttactgaatacatagctctagactt

SEQ ID NO: 31 - DNA, 1024 bp, Phyllostachys edulis
atagtgccatacgccatggccgccgtaactaccgcctccctctttcctggcctctcctcctgcagccccaagcccagaagccacaggaagctgcagagaacgacggtctgccaatgccgccctgctcggatggaggggatgaaacggagggaggccttgcttagcatcctcctctccactgccgcttcggcgccgccgcttgctcctgccgaagctttggccgagaccaccgagttgcaggagggcttccgtacgtacgaggacgaggctaacaagtttagcattgcggttccacaagactggatggtcggcgcaggcgagggcagcggcttcaagtccgtcacggcgttctaccctgaaggcgccgactcgagcgtcagcgtcgtgatcaccggaatcggaccggatttcaccagcctcaagtccttcggcgacgtcgacgccttcgccgagagcctggtgaacggcctggacaggagctggcagaggccgccggggctcgccgcgaagctcatcgactccagggcagcgaacggtctgtactacgtcgagtacacgctgcagaaccccggcgaaaagcggcggcatatcgtctcggccgtcgggatggcgttcaacggctggtacaacaggctctacacggtgacagggcagtacatcgatgacgacgacgagccaggcaagtacaagcctcagatagagaagtctgtcctatcgttcaggttcacatgaaagaactaaactacagtctacccagagtgcaacaatatgcagagaagataaagtagataaaagcccttccgcagataagttcagaacggaagatacgttgtgatttttgtcaatcagtgagcatatgtttgtcgatttgaccaaataaaatatgtactccacatgttcgacgacttgctgtgcccagcatgagttaattgtaagagaagttaccatgcgccggacctgtcattctgaaactgtgatgagtgacattctgaaactgtaacatagtaaacgtatgttcagtttt

SEQ ID NO: 32 - DNA, 1053 bp, Hordeum vulgare
agagccgtactccatggccgccgtgaccaccgcctccctctgcccgggcctcggcaagacccaacgaggccacgcgaagccgccgagaacaacggtctgccattgcctccctgctcggaggacggaggagggggtgaagcggcgggacgccctgctcggcgtcctcctctccgctaccgccgcgtcgtcggcgccactgctcgtccccgccgaggctttcgccgaggccgccgaggcgcaggaggggttcaccgcgtacgaggatgaggccaacaagttcaccctcgcgattccacaaggctggcaggtcggcgcaggtgaacgcagcggcttcaagaacgtgacggcgttcttccccgagcaaaaccccaactcgagtgtcagcgtcgtgatcaccggaatcgggccagacttcaccagcctcaagtccttcggtaatgtcgacgagttcgccgagaacctggtgacaggcctggacaggagctggcagcggccggcggggctcaccgcgaagctcatcgactccaaggcagcaaacggtctctactacatcgagtacacgctgcagaaccccggcgagaagcgccgccacatcgtgtccgccatcgggatggcgttcaacggctggtacaaccggctctacacggtgacaggacagtacatcgatgacgacgaggattcagccatatacaagcctgagatagagaagtctgtcaagtctttcaagttcacatgaaatgcctccaaaaaggaagttcaggtgagaacaagtatagaggaacagagaagagaaagtatacaaaactggtagctcttcatgttaagttcaattagtgagtgtgtatatgtttgtcgaatttaccggaagaaaatatgaacaccaaatgttcaaagacttcgatggcgttgcttggctgaggacagcaatggcagcatgagggtatgagatagagcatgagaatgtcgtttgttctgagaacattgctagaactccttataagaaactaaaattgctccgatgtaaacttcttcctagcatctatttttgggctc

SEQ ID NO: 33 - DNA, 1138 bp, Zea mays
cccgttgccacacatacggatccaacaaaatctcccgcaaagacaacggcgagccaaccaccaccagcgtcccgcgctagctgcggcaccgcatggctgccgtgaccagcaccgcctccatctgcccggccgcagccggcgccctctcttcgctgccgtccttcatcacgcgcaagcccaccagcggcagcaggaggttgcagcaggcagcagcgacgacagtctgccactgccgctctgctcgggtagaggaggggctgctgggccggagggacgccttattgctcggcatcgtcttctccgccgcgacgccgccgctgctcgcccctgccggcgctctggcggacgaggccaccgccgagtcgcaggagggcttcactacgtacgaggatgaggccaacaagttcagcattcaagttccgcaaggctggctggtcggcgccggcgaggccagcggcatcaagtctgtcacggcgttctaccccgagcaggccgccaccgactccaatgtcagcgtcgccatcaccgggatcgggccggacttcaccagcctcaagtccttcggcgacgtcgatgccttcgccgagggtctggtgaacggcctggacaggagctggcagaggccgccggggctcgccgccaagctcatcgactccagggcggcaaacggcctgtactacctggagtacacgctgcagaaccccggcgagcgacggcgccacatcgtctcggccatcgggatggccttcaacggctggtacaaccgcctctacactgtgacgggccagtacatcgacgacgatgactcggagaagtacaggcctcagatagagaaggctgttggatcgttcaggctgacatgaaagatgcgatgtcatccagcaccagcagcagcagccgcccacggtacataaaccctaaatatgtatgcggagaggtccagcaacatgttgtgcccgaaaattgacaccttgccatttcgatgagacaagacaaggcatgtgcctattgccctattccaattcttgagcactgtaacactgccaatatgcagagtatatgttttctgcctgttgaggtggatacaaatgcatgcttttttttttattaataactcatgtgtaacactgctgccttt

SEQ ID NO: 34 - DNA, 1162 bp, Sorghum bicolor
agcgcgagcctgccagccaaccaccgccaccggctttcatcccgcgcgtgcgtcctgcgctagctgcgcccatggctgccgtgaccagcaccgcctccctctgcccggccgcagccggcggcctctctgcctcgtcgtcgtcgccgttcacgcgcaagcccagcagcagcaggaggctgcaggcagcgtccacggcctgccactgccgccctgctcgggtagtagaggggctggaccggagggacgccttgctcggcatcgtcctctccgccgcggtggcgccgctgctcgcccctgccggtgctctagcggacgagcccaccaccgagtcgcaggagggcttcactacgtacgaagatgaggccaacaagttcagcattcaagttccacagggctggctggtcggcgccggcgaggccagcggcatcaagtcggtcacggcgttctacccggagcaggcagccgccgattccaacgtcagcgtcgccatcaccgggatcgggccggacttcaccagcctcaagtccttcggcgacgtcgactccttcgccgagggccttgtgaacggcctggacaggagctggcagaggccgccggggctcgccgccaagctcatcgactccagggcggcaaacggtttgtactacctggagtacacgctgcagaaccccggcgagcggcggcggcacatcgtctcggccatcgggatggcgttcaacggctggtacaaccgcctctacacggtgacaggccagtacatcgacgacgatgacgattccgaaaagtacaggcctcagatagagaaggctgttcgatcgttcaggctgacatgaaagatgccatgtcattcagcagaggtcttgtgcctgaaaattgacaccttgccatttccatgagatgagacaagacaagacatgtatgccaattcttgagcactgtaacactgcaagtatgcgaatatattttctcctttttgaggtggatataaatatgttttttgtaactcttgtgtaacgttgctgcggtgtttttttggttgtgtatatgtaatgtttagagggtcgggctgaaggagcaactatgtgacctttattctctttttaaggcaaagttcgtgtcacttcttttcaaaacaagcaaatggttttgtttcttgagctgg

SEQ ID NO: 35 - DNA, 1062 bp, Setaria italica
cggtgcctaccaaaacacacggctggaacaaaatatcccccacgaaaacaaacggcgagccaaccgcaaccaccactcggcaccggctggcctgcggcgcgcgccatggctgccgtgaccagcaccgccttcctctgcccggccgccggcggcctctccccctcgccgcccttcaggcgcaatcccggcagcagcagcagccgcaggaggctgcagctgcaggtctgccactgccgccctgctcgggtagaggggctggaccggagggaggccttgctcggcgtcgccctctccgccgccgcgccggcgctcttcgcccccgcggctgctctggcggccgaggccaccggtactttgcaggagggcttcaccacgtacgaggatgaggccaacaagttcagcattgtggttccacaaggctggctgatcggcgccggcgagtccagcggcatcaagtccgtcacggcgttctaccccgagcaggccgccgactccaacgtcagcgtcgcgatcaccggcatcgggccggacttcaccagcctcaagtccttcggcgatgtcgacgccttcgccgagggcctggtgaacggcctggacaggagctggcagaggccgccggggctcgccgcgaagctcatcgattccaaggcggcaaacggtctgtactacgtggagtacacgctgcagaaccccggcgagcggcggcggcacatcctctctgcaatcgggatggcgttcaacggctggtacaaccgcctctacacggtgacaggccagtacatcgacgacgaggagtcggagaagttcaggcctcagattgagaaggcggttcgatcgttcaggctgacatgagagtgcttcgcactgtgtagcattcagagatgcacggtatgcaggtggacgcctgtaaattgaccaactcgtcttccacattattaagtttttttttaagcaagtctcacggtatgttggaaagtacattgctacacctcaacaatcccatagatcgtctcatgtaatgccacatataatttttgctggtgtggaggaaggagggtgatg

SEQ ID NO: 36 - DNA, 1091 bp, Picea glauca
acttcaaaatcccaaacccatcagaggagggagttccaacagatgactatcgtggtgaagatgggcatgcttggcctggtgtcatcaccatcaattcccactactcacaatctatgctctccagtccaaccattcagaaggaatttcaagaatttcaagcaggggaaaaagcaagtcacaggctgtttcgaaatccagaacaatctgtcctcccatgaattgtcaagaaatggaagaaggcaggccatatgtcagattgctgctttgttctcagcgattccttgtactgtttcagcggcaagggcagcagaaactgagcttcaagaagattacgagttgtataaggacgagacagacaaattttcactactagttcctcgagactggataaagggtgaaggaaaaacagatggacagagagcagtgactgccttctaccctgaaagcggcatagtttctaatgtgaatgtaataataacaggactttctgctgactatacaaaaatggaatcatttggcactgttgatgcatttgctgagaccctggttaattctctagatagaagctggaaaagaccgccagggcaagcagcaaagctgcttaatgcaaaatccaaaaacggcttgtattatatagagtattcattgcaaaagcctggggagagtaagatccatcttctctctgcgatcggaatggcaatgaatggttggtacaacaggctttacactgtaacggggcagtatctagaagacgatgctggcaaatatggctcaaagattgaaaagtccatttcatctttcagattagtttgaaagattaattaccttccatgtgaggcatcaagtatgttgggaaaagacttataatatacaagagcataaaggtgataaatattaaataattaaaaatcccccattttattcatcttcaattatgtctggaataaacttgatttaccttgtaatatataatgtatgacctaatatcttatttggaactaagtgtgaaaccactcatagttattccaactaaattttagtttagacaagggaataaaatacattcaatgtccttattgtttactaaaaaa

SEQ ID NO: 37 - DNA, 355 bp, Arachis diogoi
ggggtgggtccggattttactaagatggaatcgtttggtaaagtggaagaatttgccgagactctgattggtggattggacagaagctggcaaagaccaccgggtgtggctgccaaactcatagattgtaaatcatccaaggggttttattacatagagtattcactgcaaaatccgggtgagagtcgcagaaccttatattctgctattggaatggcatcaaatggttggtataacagactctacacggtgacaggacagtttgtagaagaggaaactgacaagtatgcttccaaagtgaaaaaggctgttgcatcatttaggttcatatgaacaaagagttcatgagggagat

SEQ ID NO: 38 - DNA, 1089 bp, Physcomitrella patens
gcaggagcaatggacgctgtcgttggtcgcacctcatgccccttgtctctgtcttcctcgtatcaatggattgctgggtcgccatctgcttctcgtgctacagtcgttgttagaggtacaagccggcgtgacggtaaacacaaagcagtgcgttgcgagcaggttccagaatgcagcaccagcaattgtcaaacaatgcagagacgagaggttatcggtcaagctctattagccatgtccatgagctttgctcctccagctcgttcggccacagacacagatgctgctactgaatttacgacttacgaggatgcagccgataaattcacattgctcgtgccacaagcctggaacagaggcgaagggaaaacgtccgggcaaaggaaagtcacggctttctatcctgcggatggcggtcttaccaatgtaaatatagtcataacaggactcggagcagatttcacgagtttaggatcctttggcacggccgacaatttcgcggagaatttggtgaacagtttggacaggagttggcagaaacccccgggccagaaagcaaggcttgtggattgtaaatcaagagcagataaatactatgtagaatacactatacagagactcggagagcagcagcggcacttagtctcagttgttgggattggaaacaatggatgggtcaacagattatacaccgtcacgggccagtactttgaggaagactcagccaaatataaacaagacattaacaagatcatctcctctttcaaaatactgtagttcatagatcgaagactcggggcacagactgcagaccatggagtttatgactgaccagcattgtggtaaaacctgggattttcgttcctcatgcttcgtgtcacagagaagctcattgagttgggagaactgaagtggttgtgtaaacacgctggggttgttttgcatgtggtgagagcagatgtccttcgagcagccattcataaatctcaagatgagtttattctgttctgcagaaactgccgaacctggattcttgtaatagaaccattcaataatttccaaggtcaccattgggcctgagatatcatc

SEQ ID NO: 39 - DNA, 2425 bp, Selaginella moellendorffii
atgggggaggagaagcgcgagaatctcatggagtcgctcttcggggaggagtcggatgacgacgccgatgccgctgcctcctactctgaggaggagggcgaaggcgaaggcgaggcagagggcggtggtgccgagagcggtggcgacagggaagagagcgagggcgagcgggaagccagtgagggcgacaaggaggagagcgagcatgagagagtgtccaggaggagcagtgatgacggaggtggtgaagaagggagcgccgccagcggctctgagaacgatggacaagtccaggaaagggaagcaactcgcagcgaggaggtggaggagcagcgtggtgctagaattcgtgcagcattgggtgattctgatgacgacgatcaggaggaaggaggagttgacggccctcgcaagccatcgagtccagagggggagggctcggacttggagggaagctaccacaaagatagtaagcggggtgcagaggatgacgaggagcagtattactctgatgaagagcgcgcagaaatcaagaaacccaagggtgaacctcttacagtagaagttccactcaggcaacctccaactcaggctcacaacgcaagtagtttggcacttgtttctttttcattatctcgtgagcttacgggcctttgctttcagatgaatctcgtcaggatctccaacattatggatatagagcaaaggccttttgatccaaagacatacgtcgaagaggatggatttgttgatgaaactggccgacgccgtatacgtatagaggagaacgtggtgcgctggagatacgtcaggaatcgggatggctcgcgctcggccgagagcaacgcccggtttgtgaagtggtccgatggtagcatgcaacttcttctcggcaatgaagttctggatcttgctgttcaagatgggcaacaagacgaatcacacttgtttatccgtcagcccaagggattattacaagcccaagggaggctggcacgtaagatgagattcatgccttcgtcgttgagatcgaagtcgcaccgtctcttaactgctctcgtggattctacgcacaagaaggtgttcaaagtgaagaatgtgatcacggacttcgatcccgagaaggacaaagaacaaaaagagaaggcagcagaacagaggattaaaagcaaagaagatctccaaaaaaagcaggagaagacgatgcgtaaatacccccctacacgggagagagaacctcagctttctcctggataccttgaaggggcgcttgaagaggaggatgaagattatgacgacgaggggcgtagcaaccgacggtaccaagaccagcttgatgccgaaagcagggcggagagacgaatcaaccaggttaagcggcaaccaccaaaggcaatggagcgacgcccttcttcgcggagcaggagggatattttggaagacgaggattcagacgagagcgaccgcaatagaggtgcgcgaggtgatgaaggtgaggaagagcaggaagatgacgagggggaggaagaagaggaggaagtggaggcaagggatagccgccgcaagaggaaagacagggacagggaacagctgcagcagcagcaaaattcgcctcccagaaagcagcaaacgcacaggcggagagcagtagtttggtttctcgccacaggttgcaagagtagaatagataggggcagccagggaagtgtggcgtcatggcaagtgtgcatacaaactgctatgttcgtccagcagttccagccagttcaagtcgaccgccattcaatgctcctgctactgctgcctgtccaccgctgagcaagagaaatctcttattgctggtgcctctgttggcgatgccggcgacgcctgtgtttgctgctggtattgtacttgtcaatcgttcgttaaactttgatgcaagacaggagcctcagatgaatatcaagtatacgaggaacaagacaagttctccctcactgtacccaaagactggataaaaggcgaggggaaagtcggatccagaagagtggtggcattccatccatccaaggctactttcccaaacgtgaacgtgatcatcacgaacctgggtgccgatttcaccgggatcggctcactgggatccgtggactcgttcgccgcgagcgtggttggcagcatggatagaagctacaagcggccgcctggaacagcggctcggctggtgaatgctgtgtcgagaaacggcatgtattatctcgactacaccgtccagacgcccggggaagcccagcgccatttcttctcggtggccggtgttggcgagacacagttttacaagcagctctacacggcaaccggccagtactgggaggctgatggagacagggacaggaaagcattgcaagaggcgatagagtcattccggattgttcacaagtga

SEQ ID NO: 40 - DNA, 832 bp, Camellia sinensis
  misc_feature (90)..(90): n is a, c, g, or t
atacccaaagttgccaagtggggccgggggaaataaaggagatcaagtcagtaacggagttgtacccgatgggacctctgatgaaagtcncgtggtaatcgcggcctggtgcgggttttaccagaatagaaatcttttggtaaagatgatgcttttgcagagaatctgctacgggctgggacaagaagctggcaaaggccaccggggggtaagagcaaaactcatagactctaaaactgctaatggtttgtattacattgaatatacactgcaaaatcctggccaaagttgcagacatttatttcagtgcttgggatccgaacaatggttggtatccagactatataccgtcactggacagtttgttgatgaggattcagaaaaatatggctccaaaattgagaaggctgtttcatcgttcagattaaactgagattttgaggatcctttccatttttgctttcaacatcggctctcatcgctgcaacatgtccaattgaagtcaagtttactaaaggaagcaaagcattgaatgatgtttgcatcctggccgaggatttccatgcacctggagccagctgtttggaagacaccaagaggcggctagattgtggcatatttactctctgttttacttttgttattcctagcctttcttgcaacttttcttgaagtatgctggaacctttattatttatttgactaggaaatttattcttaataccaccagaaagagatagacaaaaactaccttggtgattgcattgaattaacataaagtgcccaaaaatattttgctaagaaaaaaaaaaaaaaaaaaaaaaaaaaaaaa

SEQ ID NO: 41 - DNA, 2246 bp, Zea mays
tttggtgtttctcgcagtgcgcagagggcttcagtcattatcgtctcgtgctctctattgatggtagttttttgcttgggaagtataggggcacattgttggttgccatttcatgtgacgtggacaacgcattggttcttttggcatttgctttggtggtgagggagaacagagatagttcgttttggttcttgcgacttgttcggatccatgtcgtgggccctggtcgggagattggtgtcatatatgacagaaaccagggtattcttaatgtagtgcaagagcagattcctggctacacacccatgcaccacagatggagcactcgacacctagtagaaaatctttttcgaaagggttgtaccaagaataattcccccctttttgaggaggtctgtcgacagttggaggtttcgttattcgaggataagttgaaggaattaaaagatacaacaaatgatgaaggtgaaaaatggattgctagatttcctaactgtatttcctcgtctcctacacaaaaaacagtaagtacgaatgcaacattacgactgatgctatcaaaaagcaatatcaacctcatcgtaatctaccacatgtccaaaaataacccacaccaagatcagaaggaactaatccatacttagcttcattaaaaccgccactcattttttctttttctttgcctctggctcaggccaatgggacctaggaccatacaaccactctatgaaactacacttacgcccaccgtatggccgtaggggaataaattagtaaatttgtgaaaaaataacaaaattttggacatttgttatgcactcacataattactgttgggacacacgaagtacttcacagtgtccttacataagacaacacgatctccgcaatcacaccgaggcggagacttcagtcgtctttctgcgactaagttcttcttctcatccgtcattggtgtcgtgtcgggggaggaggcacccaacgcttaaaacactgtatattccttctggacaaccaattagcgaaaaggagatatcgatggtcaaatttatgaggaccgtcgatccattggaaaacacatcaagtgatcctacagaaatggaatgaagcattagtacacatgtagtaaacatcaataacacacaagtcataagctacatatgttaaaaacgccacaagtgtagaagcagcgagcagccgtgtctagatatttgaattgacatacccaaggcggtctgccacagtcacaattaggtacaggaagatcaggaggaacatgagcatccttactagatacatccagatacaactcccgaggacgatctctcttctgccacaacgactcctaatacatgtcttttcatctaacaaacgattatatattaacacaactatatagaaataacagaaatgattttaacttataaatacatgtatttcataaattatctaacaaactgtagtcattatatgaaccatatatcataggattcataactaatagcaatatgagcaacaatcaaaactaattaatcgaaaacatacaacgcaatatacgtaacaaatgaatcagtgagacactataccttggtcttcaaagttttccaactcgaatacgaagattggagcacctctgggtggtatggaagaagatacaaatggttgggtgcagctctcggctaggaaacaaccgatttataggccttcctagctcggcgccacagatccatggcgccgagctagtgccatgtcggtgctgcatcagccctcgacgctagcggccttgtgttaccttggtgctacaacttatgatattgagcaacgagtccaaatgcagaaataagttatctaggggtctaaacattaaacacgatttttttcttaaaaaggaccaacataaaaaaaactcgggctggcggcctgccagcgcccggggaccgtgagcccgtgaccccagcgggcccaaccagtcaaaaccgcaagagagagtcgtgaggcgtgcgcactaccccgagaaacgtgcgacgggaacctccgcggttccccaagttcgcctccttcactactctcgcgccccggcacgcctgaaaaaccccacccctcctgccgctccgcctctcccatcacttcccacgcccctcgccgcctcccattccagcgtggacacgacgccactcgccagcacggagacgcgcgcctcgaagcactactgcactagccagccgtcgttcttccgcgccggcgcc

SEQ ID NO: 42 - DNA, 27 bp, Artificial
  synthetic
tttttaggaattattgagtattattga

SEQ ID NO: 43 - DNA, 24 bp, Artificial
  synthetic
aaataaaaatcatacccacatccc

SEQ ID NO: 44 - DNA, 26 bp, Artificial
  synthetic
tgttgaattattaagatatttaagat

SEQ ID NO: 45 - DNA, 25 bp, Artificial
  synthetic
tcaaccaataaaaattaccatctac

SEQ ID NO: 46 - DNA, 30 bp, Artificial
  synthetic
taagttttttttaagagtttgtatttgtat

SEQ ID NO: 47 - DNA, 25 bp, Artificial
  synthetic
taaaaataatcaaaacctaacttac

SEQ ID NO: 48 - DNA, 25 bp, Artificial
  synthetic
attgtttattaaatgttttttagtt

SEQ ID NO: 49 - DNA, 24 bp, Artificial
  synthetic
ctaacaattcccaaaacccttatc

SEQ ID NO: 50 - DNA, 24 bp, Artificial
  synthetic
gtgtactcatctggatctgtattg

SEQ ID NO: 51 - DNA, 25 bp, Artificial
  synthetic
ggttgaggagcctgaatctctgaac

SEQ ID NO: 52 - DNA, 1000 bp, Arabidopsis thaliana
acagaaaaaaaaaaactaatttcgattccacaatgccgaaacccagaaaaattctcaaaacgaagtcactcaagaagataatcaacaaagcgaaaacggagaagggtttgggtttaaggtaccgtaacgaactccggtaagttccgatgatggtgttgacagcgaagcttcgtggaacagcgtgaccgtcgttactcactggatttgccggtgaatttgatgagtcaaaaatataactagcagctggtcaaagtaaccactctgaataagacacgtgtcagaaactttgttgaccattctgactaaagaaaccttgtaaccaaaaacatccacatttttacatgctctgtcatatttcaactgtatactaaagttgaaatatgactatggtaacttatgtttttttagttgaaaactaaggtaaactattctatgccagcaggtaaaagatgtggatgaccaactactgctggcataatttattctatgactaaggtaaaactaaaagttaatatcactcgaaatatttacaaaagtattagataaagtactaaaaattttaagttagaaatgatgaaattaaaagatatatgtggcttatttaggaaaatagatacttttaatatgatggttgggtagttttattattgtttgggattgaaaaaaactatgaaagtgtctccaagtacagtaatatttaacaactaactaggaaggtgtacattgttcagtattcatcattctaattaatacaaataattttaaaaatttggggtcgtcaattgctcaaagatattcattattttataattttctacattttatcaaaaacagaattgtgtaaatgttgtgtcattatgtgtcgacttggatgtatgcattacataattttattttatatatgtttttaattgttagagcatttattcgaaaattataaatattgaattttaatcctcagtcacttaataaaaacattattttaggactaaaaaataat

SEQ ID NO: 53 - DNA, 1000 bp, Oryza sativa
caacgacggcttgcaggaatttttaaaaaactataaaaaaaatcatatcaataatacggatcttaccggtgtagtgagatgacaaagaagagaagcaatgggcaggtgagcgtgcgcaaacaaatgagataggaggcagttgccacaggagggcatgccgagggtggtaaagtaaataaaaaaataattataagagttcaagtagaattacaatttataaatgaaagaaatatcacaataagaaatatttatttttgaaaaaaatagtccatacagaccacaacatgacaaattataataaaaaaacaagagaatagacccataaaaaattagagtggtgtaggtagatgggacatctaaaaactataaacaaaaccttaaatagaatatcaatgataattaagaggagggacaacgagccggtcgttaagcagcacgatctcgacgattggagggatttctagaaagtaaaaaaaaactctcaattataatcatgttcgattttcaaatctcaacgataataaaaatgggaagcaatggacgggccaaagaggagtatagtggcggcgtttagcgagacttataaaaattataaaaatgaaacccaacaatacaatgaactctaaaaccacaaggtcaaatttgtagaggttccaggaaggatgaatgaaagcattggtagatcgagcaagtaaataaagcgatgataaagaaataatgcgtagtgattagcatgactattaaaagcaccgggatgataaagtcggtttctaacatatagttatttaacaaattttaagtaaaatcatatgtaaaaaatagataattttgtatgggtgctagcccgcgcaattgcgcgggccacctagctagtttttttaaaaatgtaaatataaaaaattcgaccaccacgcatcgcctgctctgccgcggcgacgcgagccacaaagccgtcgaccaccgcaacaacaaaatatcctcctcccgacgcaaaccggtt

SEQ ID NO: 54 - DNA, 1000 bp, Solanum lycopersicum
atgtttaatttatgggtaatgtagaagtgtatttaaaaaaaaaaaattgataaatatgtttatattatatgtgtaatgtgtgttaatattttcggtgatggaataatatatgataaatcatttagactcatgaagctcttcttaaatataatgaaaagacttccaatgtaaaaaattataatcggaagcggagccaatatatatttaggggttcatttgaattctctttagcgaaaaatatagtactatttatatatgatcagttatttttttttatgcatatgtagtagatattaaatctccttcgattatttcgtgtgtttacttcttgagattttaaattccctattcaaaaatcctagcctcgccattgtccgtaacaaagagatactaaatcgtatcttgtactccctgtgtcccaatttatgcgacttacgttgctttttagtcagtccaaaaataatgacacaattctatattaagtaacaatttaactataaaacgtcgattttatccttaatgaaacgatttaccaccacacaaatttctcattttagactgcaagttttttaaaaattttcatttctttcttaaaactctgtgccgaataaaactactttacgtaaaatagaaaggaggaaatatttatttacacatcataaaaagtctcgaggaaaatcaaaaaaacgtcacaacaaacataatattttcattgaaaaaatcgtttcatacctatttcttttgttgtctctttttcatccagtgttcagtactcactataaacactgaccaatataaaactattcgcgctccaaaataccacattaaataaaagcaacttcatacataaaactcgaactcataatctcccgttataaaaggataccacaactttttagtgctctttttcaataataactttttttttataaaaaaaaactaactgccagagtggaatttccaactgtatctatttgatgaaaatagcagaaaactggtt

SEQ ID NO: 55 - DNA, 21 bp, Artificial
  synthetic
acggaaaaagttctttccagg

SEQ ID NO: 56 - DNA, 21 bp, Artificial
  synthetic
gctttccatcggctaggttag

SEQ ID NO: 57 - DNA, 34 bp, Artificial
  synthetic
tagcatctgaatttcataaccaatctcgatacac

(19) United States
(12) Patent Application Publication
(10) Pub. No.: US 2015/0052630 A1
(43) Pub. Date: Feb. 19, 2015

(54) Methods and Compositions for Obtaining Useful Plant Traits

(71) Applicant: Board of Regents of the University of Nebraska, Lincoln, NE (US)

(72) Inventors: Sally Mackenzie, Lincoln, NE (US); Michael Fromm, Lincoln, NE (US); Kamaldeep Virdi, Lincoln, NE (US); Yashitola Wamboldt, Lincoln, NE (US); Jiantao Yu, Lincoln, NE (US); Mon-Ray Shao, Lincoln, NE (US)

(21) Appl. No.: 14/454,518

(22) Filed: Aug. 7, 2014

Related U.S. Application Data
(60) Provisional application No. 61/970,424, filed on Mar. 26, 2014.
(60) Provisional application No. 61/863,267, filed on Aug. 7, 2013.

(51) Int. Cl. A01H 1/02; C12Q 1/68; A01H 5/10; A01H 1/04; A01H 1/06; C12N 15/82

(52) U.S. Cl. CPC A01H 1/02; A01H 1/06; C12N 15/8222; A01H 5/10; A01H 1/04; C12Q 1/6895; C12Q 2600/13; C12Q 2600/154
USPC 800/266; 800/276; 435/320.1; 800/298; 506/7

(57) ABSTRACT

Methods for obtaining plants that exhibit useful traits by perturbation of plastid function in plants are provided. Methods for identifying genetic loci that provide for useful traits in plants and plants produced with those loci are also provided. In addition, plants that exhibit the useful traits, parts of the plants including seeds, and products of the plants are provided as well as methods of using the plants. Recombinant DNA vectors and transgenic plants comprising those vectors that provide for plastid perturbation are also provided.

### CROSS-REFERENCE TO RELATED APPLICATIONS

This application claims the benefit of U.S. Provisional Patent Application No. 61/970,424, filed Mar. 26, 2014, and U.S. Provisional Patent Application No. 61/863,267, filed Aug. 7, 2013, which are each incorporated herein by reference in their entireties.

### STATEMENT REGARDING FEDERALLY SPONSORED RESEARCH OR DEVELOPMENT

This invention was made with Government Support under a grant from the Department of Energy (DE-FG02-10ER16189) and the National Science Foundation (IOS 1126935). The government has certain rights to this invention.

### INCORPORATION OF SEQUENCE LISTING

The sequence listing contained in the file named “46589_{—}133998_SEQ_LST.txt”, which is 110,868 bytes in size (measured in operating system MS-Windows), contains 57 sequences, and which was created on Aug. 7, 2014, is contemporaneously filed with this specification by electronic submission (using the United States Patent Office EFS-Web filing system) and is incorporated herein by reference in its entirety.

### BACKGROUND

Evidence exists in support of a link between environmental sensing and epigenetic changes in both plants and animals (Bonasio et al., Science 330, 612, 2010). Trans-generational heritability of these changes remains a subject of active investigation (Youngson et al. Annu. Rev. Genom. Human Genet. 9, 233, 2008). Previous studies have shown that altered methylation patterns are highly heritable over multiple generations and can be incorporated into a quantitative analysis of variation (Vaughn et al. 2007; Zhang et al. 2008; Johannes et al. 2009). Earlier studies of methylation changes in Arabidopsis suggest amenability of the epigenome to recurrent selection and also suggest that it is feasible to establish new and stable epigenetic states (F. Johannes et al. PLoS Genet. 5, e1000530 (2009); F. Roux et al. Genetics 188, 1015 (2011). Manipulation of the Arabidopsis met1 and ddmt mutants has allowed the creation of epi-RIL populations that show both heritability of novel methylation patterning and epiallelic segregation, underscoring the likely influence of epigenomic variation in plant adaptation (F. Roux et al. Genetics 188, 1015 (2011)). In natural populations, a large proportion of the epiallelic variation detected in Arabidopsis is found as CpG methylation within gene-rich regions of the genome (C. Becker et al. Nature 480, 245 (2011), R. J. Schmitz et al. Science 334, 369 (2011).

Induction of traits that exhibit cytoplasmic inheritance (Redei Mutat. Res. 18, 149-162, 1973; Sandhu et al. Proc Natl Acad Sci USA. 104:1766-70, 2007) or that exhibit nuclear inheritance by suppression of the MSH1 gene has also been reported (WO 2012/151254; Xu et al. Plant Physiol. Vol. 159:711-720, 2012).

Plant genomes contain relatively large amounts of 5-methylcytosine (5 meC; Kumar et al. 2013 J Genet 92(3): 629-666). Other than silencing transposable elements and repeated sequences, the biological roles of 5 meC are still emerging. Intercrossing a low methylation mutant plant with a normally methylated plant resulted in heritable changes in DNA methylation in the plant genome that affected some plant phenotypic traits (Cortijo et al. 2014 Science. 2014 Mar. 7; 343(6175):1145-8).

Over expression of Arabidopsis MET1, a DNA methyltransferase, in Arabidopsis resulted in plants that flowered earlier (U.S. Pat. Nos. 6,011,200 and 6,444,469). This method focused specifically on MET1 type of DNA methyltransferases, which predominantly use CG as their DNA methylation substrate. Further, U.S. Pat. Nos. 6,011,200 and 6,444,469 only describes progeny plants expressing transgenic MET1. U.S. Pat. No. 5,750,868 describes the use of a bacterial DAM methylase to cause male sterility in plants.

### SUMMARY

Methods for producing a plant exhibiting useful traits, methods for identifying one or more altered chromosomal loci in a plant that can confer a useful trait, methods for obtaining plants comprising modified chromosomal loci that can confer a useful trait, plants exhibiting the useful traits, parts of those plants including cells, leafs, stems, flowers and seeds, methods of using the plants and plant parts, and products of those plants and plant parts, including processed products such as a feed or a meal are provided herein. Also provided herein are recombinant DNA constructs that provide for selective expression of heterologous sequences in specific plastid subpopulations, as well as transgenic plants and plant cells comprising those recombinant DNA constructs. Seed lots comprising seed or progeny plants grown from the seed that exhibit the traits and methods for obtaining such seed lots are also provided.

Methods for producing a plant exhibiting a useful trait comprising the steps of (a) perturbing plastid function in a first parental plant or plant cell; (b) screening a population of progeny plants obtained from the parental plant or plant cell for the useful trait, wherein plastid function has been recovered in at least a portion of the progeny plants; and, (c) selecting one or more progeny plants that exhibit(s) the useful trait and have recovered plastid function, wherein the trait exhibits nuclear inheritance are provided. In certain embodiments, the perturbing does not comprise direct suppression of MSH1 gene expression. In certain embodiments, the perturbed plastid function is selected from the group consisting of a sensor, photosystem I, photosystem II, NAD(P)H dehydrogenase (NDH) complex, cytochrome b6f complex, and plastocyanin function. In certain embodiments, the photosystem II function and/or sensor function is perturbed by suppressing expression of a gene selected from the group consisting of a PPD3 gene, a PsbO-1, a PsbO-2, PsbY, PsbW, PsbX, PsbR, PsbTn, PsbP1, PsbP2, PsbS, PsbQ-1, PsbQ-2, PPL1, PSAE-1. LPA2. PQL1, PQL2 and a PQL3 gene. In certain embodiments, the sensor function is perturbed by suppressing MSH1 gene expression. In certain embodiments of any of the aforementioned methods, the plastid function is selectively inhibited in cells containing sensory plastids. In certain embodiments, the selective inhibition is effected with a transgene comprising a promoter that is selectively expressed in cells containing sensory plastids and that is operably linked to a sequence that perturbs plastid function. In certain embodiments, the promoter that is selectively expressed is a MSH1 promoter or a PPD3 promoter. In certain embodiments of the methods, the methylation status of one or more genes of said nuclear chromosome is monitored. In certain embodiments, the monitored nuclear genes are selected from the group consisting of plant stress genes, plant defense genes, regulatory genes, protein turnover genes, and kinase genes. In certain embodiments, the methylation status of Msh1 and/or a pericentromeric region of a chromosome is monitored. In certain embodiments, a first and/or second generation progeny plant obtained from the first parental plant or plant cell thereof exhibits Msh1-dr traits as compared to a control plant that had not been subjected to the plastid perturbation. In certain embodiments, a first and/or second generation progeny plant obtained from the first parental plant or plant cell exhibits CG hypermethylation of a region encompassing a MSH1 locus in comparison to a control plant that had not been subjected to the plastid perturbation. In certain embodiments, a first, second, and/or third or later generation progeny plant obtained from the first parental plant or plant cell exhibits pericentromeric CHG hyper-methylation in comparison to a control plant that had not been subjected to the plastid perturbation. In certain embodiments, the pericentromeric CHG hyper-methylation is heritable. In certain embodiments, the perturbation provides for increased levels of plastoquinol in comparison to a control plant that had not been subjected to the plastid perturbation. In certain embodiments of any of the aforementioned methods, the method further comprises the step of producing seed from: i) a selfed progeny plant or plants; ii) an out-crossed progeny plant or plants; or, iii) both of a selfed and an out-crossed progeny plant or plants. In certain embodiments of any of the aforementioned methods, the method further comprises the step of producing seed from: (i) a selfed progeny plant or plants selected in step (c); or from (ii) an out-crossed progeny plant or plants selected in step (c). In certain embodiments of any of the aforementioned methods, the method comprises: (i) outcrossing or selfing the first parental plant or progeny thereof to obtain an F1 generation of plants, wherein the first parental plant or progeny thereof exhibits one or more Msh1-dr traits; (ii) screening the population of plants obtained from the outcross for the presence of the useful trait and the absence of Msh1-dr traits; (iii) selecting a population of plants exhibiting the useful trait and recovered plastid function; and (iv) obtaining seed from the selected population of step (iii) or, optionally, repeating steps (iii) and (iv) on a population of plants grown from the seed obtained from the selected population. In certain embodiments of any of the aforementioned methods, the useful trait is selected from the group consisting of improved yield, delayed flowering, non-flowering, increased biotic stress resistance, increased abiotic stress resistance, enhanced lodging resistance, enhanced growth rate, enhanced biomass, enhanced tillering, enhanced branching, delayed flowering time, and delayed senescence in comparison to a control plant that had not been subjected to the plastid perturbation. In certain embodiments of the methods, the useful trait is associated with one or more epigenetic changes in one or more nuclear chromosomes. In certain embodiments of any of the aforementioned methods, the selected progeny plant(s) or progeny thereof exhibit an improvement in the trait in comparison to a plant that had not been subjected to the plastid perturbation but was otherwise isogenic to the first parental plant or plant cell. In certain embodiments of any of the aforementioned methods, the plant is a crop plant. In certain embodiments, the crop plant is selected from the group consisting of corn, soybean, cotton, canola, wheat, rice, tomato, tobacco, millet, and sorghum. In certain embodiments, the crop plant is sorghum. In certain embodiments where the crop plant is sorghum, the trait can be selected from the group consisting of panicle length, panicle weight, dry biomass, and combinations thereof. Also provided is a plant or population of plants produced by the aforementioned methods, wherein the plant or population of plants exhibits an improvement in at least one useful trait in comparison to a plant that had not been subjected to the plastid perturbation but was otherwise isogenic to the first parental plant or plant cell and wherein the plant or at least 25%, 50%, 70%, 80%, 90%, or 95% of the population of plants exhibit the trait. In certain embodiments, the plant or plant population is an inbred plant or plant population. Also provided are seed obtained from the plant or plant populations, wherein the seed or a plant obtained therefrom exhibits the improvement in at least one useful trait. Also provided are processed products from the plant or population of plants or from the seed therefrom, wherein the product comprises a detectable amount of a nuclear chromosomal DNA comprising one or more epigenetic changes that were induced by the plastid perturbation. In certain embodiments, the product is oil, meal, lint, hulls, or a pressed cake. Also provided is a method for producing a seed lot, comprising the steps of selfing a population of plants of claim 25, and harvesting a seed lot therefrom, wherein at least about 25%, 50%, 70%, 80%, 90%, or 95% of harvested seed or plants obtained therefrom exhibit the improvement in at least one useful trait.

Also provided are methods for identifying one or more altered chromosomal loci in a plant that can confer a useful trait comprising the steps of: (a) comparing DNA methylation status of one or more nuclear chromosomal regions in a reference plant that does not exhibit the useful trait to one or more corresponding nuclear chromosomal regions in a test plant that does exhibit the useful trait, wherein the test plant was obtained by any of the aforementioned methods; and, (b) selecting for one or more altered nuclear chromosomal loci present in the test plant with a DNA methylation status that is distinct from the DNA methylation status in the reference plant, wherein the selected chromosomal loci are associated with the useful trait. In certain embodiments of the methods, the DNA methylation status comprises CG hypermethylation and/or CHG hypermethylation. In certain embodiments of the methods, the selection comprises isolating a plant or progeny plant comprising the altered chromosomal locus or obtaining a nucleic acid associated with the altered chromosomal locus. In certain embodiments of the methods, the reference plant and the test plant are both obtained from a population of progeny plants obtained from a parental plant or plant cell wherein plastid function had been perturbed. In certain embodiments of the methods, the reference plant and the parental plant or plant cell were isogenic prior to perturbation of plastid function in the parental plant or plant cell. In certain embodiments of the methods, the useful trait is selected from the group consisting of increased yield, male sterility, non-flowering, increased biotic stress resistance, increased abiotic stress resistance, enhanced lodging resistance, enhanced growth rate, enhanced biomass, enhanced tillering, enhanced branching, delayed flowering time, and delayed senescence in comparison to a control plant that had not been subjected to the plastid perturbation. Also provided is an altered chromosomal locus of a plant identified by any of the aforementioned methods. Also provided is a plant comprising the altered chromosomal locus.

Methods for producing a plant exhibiting a useful trait comprising the steps of: a. introducing a nuclear chromosomal modification associated with a useful trait into a plant, wherein the chromosomal modification comprises an epigenetic change induced by any of the aforementioned methods and that is associated with the useful trait, a transgene that provides for the same genetic effect as an epigenetic change induced by any of the aforementioned methods, or a chromosomal mutation that provides for the same genetic effect as an epigenetic change induced by any of the aforementioned methods; and, b. selecting for a plant or plants that comprise the nuclear chromosomal modification and exhibit the useful trait. In certain embodiments, the method further comprises the step of producing seed from: i) a selfed progeny plant of the selected plant or plants of step (b), ii) an out-crossed progeny plant of the selected plant or plants of step (b), or, iii) from both of a selfed and an out-crossed progeny plant of the selected plant or plants of step (b). In certain embodiments, the chromosomal modification comprises CG hypermethylation and/or CHG hypermethylation. In certain embodiments, the chromosomal modification comprises the transgene or the chromosomal mutation and wherein the plant is selected by assaying for the presence of the transgene or the chromosomal mutation. In certain embodiments, the plant is selected by assaying for the presence of the useful trait. In certain embodiments, the epigenetic change has a genetic effect that comprises a reduction in expression of a gene and wherein the chromosomal modification comprises a transgene or a chromosomal mutation that provides for a reduction in expression of the gene. In certain embodiments, the transgene reduces expression of the gene by producing a small inhibitory RNA (siRNA), a microRNA (miRNA), a co-suppressing sense RNA, and/or an anti-sense RNA directed to the gene. In certain embodiments, the altered chromosomal locus has a genetic effect that comprises an increase in expression of a gene and wherein the chromosomal modification comprises a transgene or a chromosomal mutation that provides for an increase in expression of the gene. In certain embodiments of any of the aforementioned methods, the useful trait is selected from the group consisting of increased yield, male sterility, non-flowering, increased biotic stress resistance, increased abiotic stress resistance, enhanced lodging resistance, enhanced growth rate, enhanced biomass, enhanced tillering, enhanced branching, delayed flowering time, and delayed senescence in comparison to a control plant that had not been subjected to the plastid perturbation. Also provided is a plant made by any of the aforementioned methods.

Recombinant DNA constructs comprising a promoter that is selectively expressed in cells containing sensory plastids and that is operably linked to a heterologous sequence that perturbs plastid function are also provided. In certain embodiments, the promoter is selected from the group consisting of a Msh1 promoter and a PPD3 promoter. In certain embodiments, the perturbed plastid function is selected from the group consisting of a sensor, photosystem I, photosystem II, NAD(P)H dehydrogenase (NDH) complex, cytochrome b6f complex, and plastocyanin function. In certain embodiments, the photosystem II and/or sensor function is perturbed by suppressing expression of a gene selected from the group consisting of a Msh1, PPD3 gene, a PsbO-1, a PsbO-2, PsbY, PsbW, PsbX, PsbR, PsbTn, PsbP1, PsbP2, PsbS, PsbQ-1, PsbQ-2, PPL1, PSAE-1, LPA2, PQL1, PQL2, and a PQL3 gene. In certain embodiments of any of the aforementioned constructs, the heterologous sequence that perturbs plastid function comprises a sequence selected from the group consisting of a small inhibitory RNA (siRNA), a microRNA (miRNA), a co-suppressing sense RNA, and/or an anti-sense RNA that suppresses expression of a gene that provides a plastid function. In certain embodiments, the construct further comprises minichromosome sequences and/or sequences that provide for removal for the recombinant DNA construct from a chromosome. Also provided is a transgenic plant or plant cell comprising the recombinant DNA constructs. In certain embodiments, the transgenic plant exhibits Msh1-dr traits as compared to a non-transgenic control plant that lacks the recombinant DNA construct.

Methods for producing a seed lot comprising: (i) selecting a first sub-population of plants exhibiting a useful trait associated with an epigenetic change at one or more nuclear chromosomal loci and recovered plastid function from a first population of plants that are segregating for the useful trait; and (ii) obtaining a seed lot from the first selected sub-population of step (i) or, optionally, repeating steps (i) and (ii) on a second population of plants grown from the seed obtained from the first selected sub-population of plants are also provided. In certain embodiments, the epigenetic change was induced by plastid perturbation. In certain embodiments, the epigenetic change was induced by suppressing expression of a gene selected from the group consisting of an Msh1 gene, a PPD3 gene, a PsbO gene, a PsbO1, a Psb02, and a Psb03 gene. In certain embodiments, wherein the epigenetic change is associated with CG hyper-methylation and/or CHG hyper-methylation at one or more nuclear chromosomal loci in comparison to a control plant that does not exhibit the useful trait. In certain embodiments, wherein the epigenetic change is associated with CG hyper-methylation and/or CHG hyper-methylation and/or CHH hyper-methylation at one or more nuclear chromosomal loci in comparison to a control plant that does not exhibit the useful trait. In certain embodiments, the first subpopulation is also segregating for recovered plastid function. In certain embodiments, a plurality of plants in the first sub-population exhibit heritable pericentromeric CHG hyper-methylation. In certain embodiments, a plurality of plants in the first sub-population exhibit heritable CHG and/or CHH hyper-methylation of one or more regions comprising pericentromeric or transposable element or repeated sequences. In certain embodiments of any of the aforementioned methods, at least 25%, 50%, 60%, 70%, 80%, 90%, or 95% of progeny plants grown from the seed lot obtained in step (ii) exhibit the useful trait associated with an epigenetic change. In certain embodiments, the seed or progeny plants grown from the seed comprise a mixture of inbred and hybrid germplasm that is epigenetically heterogenous. Also provided is a seed lot produced by the method of any.

Also provided is a seed lot comprising seed wherein at least 25%, 50%, 60%, 70%, 80%, 90%, or 95% of progeny plants grown from the seed exhibit a useful trait associated with one or more epigenetic changes, wherein the epigenetic changes are associated with CG hyper-methylation and/or CHG hyper-methylation at one or more nuclear chromosomal loci in comparison to a control plant that does not exhibit the useful trait, and wherein the seed or progeny plants grown from said seed that is epigenetically heterogenous. In certain embodiments, the epigenetic changes are induced by plastid perturbation. In certain embodiments, the epigenetic changes are induced by suppression of MSH1 gene expression or by suppression of PPD3 gene expression. In certain embodiments, the epigenetic changes are associated with CG hyper-methylation and/or CHG hyper-methylation and/or CHH hyper-methylation at one or more nuclear chromosomal loci in comparison to a control plant that does not exhibit the useful trait. In certain embodiment, the useful trait is selected from the group consisting of increased yield, male sterility, non-flowering, increased biotic stress resistance, increased abiotic stress resistance, enhanced lodging resistance, enhanced growth rate, enhanced biomass, enhanced tillering, enhanced branching, delayed flowering time, and delayed senescence in comparison to a control plant that lacks the epigenetic change(s). In certain embodiments, the seed comprise a mixture of inbred and hybrid germplasm.

Methods for producing a plant exhibiting new combinations of altered chromosomal loci useful for breeding comprising the steps of: (a) crossing a plant comprising altered chromosomal loci induced by plastid perturbation to produce progeny; and, (b) assaying the DNA methylation of said progeny to identify and select individuals with new combinations of altered chromosomal loci, thereby producing a plant exhibiting new combinations of altered chromosomal loci useful for breeding are provided herein. Methods for producing a plant exhibiting new combinations of altered chromosomal loci useful for breeding comprising the steps of: (a) crossing a plant comprising altered chromosomal loci induced by MSH1 or PPD3 suppression to produce progeny; and, (b) assaying the DNA methylation of said progeny to identify and select individuals with new combinations of altered chromosomal loci, thereby producing a plant exhibiting new combinations of altered chromosomal loci useful for breeding are also provided herein. In some embodiments altered chromosomal loci are selected from the group consisting of MSH1, pericentromeric regions, CG enhanced genes, CG depleted genes, transposable elements, transposable elements containing genes, and transposable elements in pericentromeric regions. In certain embodiments DNA methylation of altered chromosomal loci occurs at CHG or CHH sites within one or more DNA regions selected from the group consisting of MSH1, pericentromeric regions, transposable elements, transposable elements containing genes, and transposable elements in pericentromeric regions. In certain embodiments DNA methylation of altered chromosomal loci occurs at CG sequences near or within one or more CG altered genes.

Methods for producing a plant exhibiting new combinations of altered chromosomal loci useful for breeding comprising the steps of: (a) crossing a plant comprising altered chromosomal loci induced by plastid perturbation to produce progeny; and, (b) assaying one or more sRNAs of said progeny to identify and select individuals with new combinations of altered chromosomal loci, thereby producing a plant exhibiting new combinations of altered chromosomal loci useful for breeding are provided. Methods for producing a plant exhibiting new combinations of altered chromosomal loci useful for breeding comprising the steps of: (a) crossing a plant comprising altered chromosomal loci induced by MSH1 or PPD3 suppression to produce progeny; and, (b) assaying one or more sRNAs of said progeny to identify and select individuals with new combinations of altered chromosomal loci, thereby producing a plant exhibiting new combinations of altered chromosomal loci useful for breeding are also provided. In certain embodiments one or more sRNAs assayed have sequence homology to the group consisting of MSH1, pericentromeric regions, CG enhanced genes, CG depleted genes, transposable elements, transposable elements containing genes, and transposable elements in pericentromeric regions. 100171 Methods for identifying a plant with altered chromosomal loci useful for plant breeding comprising the steps of: (a) assaying DNA methylation of one or more plants comprising altered chromosomal loci induced byplastid perturbation; and, (b) identifying one or more plants from step (a) comprising one or more altered chromosomal loci selected from the group consisting of MSH1, pericentromeric regions, CG enhanced genes, CG depleted genes, transposable elements, transposable elements containing genes, and transposable elements in pericentromeric regions, thereby identifying a plant with altered chromosomal loci useful for plant breeding are provided. Methods for identifying a plant with altered chromosomal loci useful for plant breeding comprising the steps of: (a) assaying DNA methylation of one or more plants comprising altered chromosomal loci induced by MSH 1 or PPD3 suppression; and, (b) identifying one or more plants from step (a) comprising one or more altered chromosomal loci selected from the group consisting of MSH1, pericentromeric regions, CG enhanced genes, CG depleted genes, transposable elements, transposable elements containing genes, and transposable elements in pericentromeric regions, thereby identifying a plant with altered chromosomal loci useful for plant breeding are also provided. In certain embodiments DNA methylation of altered chromosomal loci occurs at CHG or CHH at DNA sequences selected from the group consisting of MSH1, pericentromeric regions, transposable elements, transposable elements containing genes, and transposable elements in pericentromeric regions. In certain embodiments DNA methylation of altered chromosomal loci occurs at CG sequences near or within one or more CG altered genes.

Methods for identifying a plant with altered chromosomal loci useful for plant breeding comprising the steps of: (a) assaying one or more sRNAs of one or more plants comprising altered chromosomal loci induced byplastid perturbation; and, (b) identifying one or more plants from step (a) comprising one or more increases or decreases in one or more sRNAs with homology at DNA sequences selected from the group of altered chromosomal loci consisting of MSH1, pericentromeric regions, CG enhanced genes, CG depleted genes, transposable elements, transposable elements containing genes, and transposable elements in pericentromeric regions, thereby identifying a plant with altered chromosomal loci useful for plant breeding are provided herein. Methods for identifying a plant with altered chromosomal loci useful for plant breeding comprising the steps of: (a) assaying one or more sRNAs of one or more plants comprising altered chromosomal loci induced by MSH1 or PPD3 suppression; and, (b) identifying one or more plants from step (a) comprising one or more increases or decreases in one or more sRNAs with homology at DNA sequences selected from the group of altered chromosomal loci consisting of MSH1, pericentromeric regions, CG enhanced genes, CG depleted genes, transposable elements, transposable elements containing genes, and transposable elements in pericentromeric regions, thereby identifying a plant with altered chromosomal loci useful for plant breeding are also provided herein.

Methods for producing a plant exhibiting new combinations of altered chromosomal loci useful for breeding comprising the steps of: (a) selfing a plant comprising altered chromosomal loci induced by plastid perturbation to produce progeny; and, (b) assaying the DNA methylation at altered chromosomal loci of said progeny to identify and select individuals with new combinations of altered chromosomal loci are provided herein. In certain embodiments altered chromosomal loci are selected from the group consisting of MSH1, pericentromeric regions, CG enhanced genes, CG depleted genes, transposable elements, transposable elements containing genes, and transposable elements in pericentromeric regions are also provided herein. Methods for producing a plant exhibiting new combinations of altered chromosomal loci useful for breeding comprising the steps of: (a) selfing a plant comprising altered chromosomal loci induced by MSH1 or PPD3 suppression to produce progeny; and, (b) assaying the DNA methylation at altered chromosomal loci of said progeny to identify and select individuals with new combinations of altered chromosomal loci are provided herein. In certain embodiments altered chromosomal loci are selected from the group consisting of MSH1, pericentromeric regions, CG enhanced genes, CG depleted genes, transposable elements, transposable elements containing genes, and transposable elements in pericentromeric regions are also provided herein. In certain embodiments DNA methylation of altered chromosomal loci occurs at CHG or CHH sites within a DNA region selected from the group consisting of MSH1, pericentromeric regions, transposable elements, transposable elements containing genes, and transposable elements in pericentromeric regions. In certain embodiments DNA methylation of altered chromosomal loci occurs at CG sequences near or within one or more CG altered genes.

Methods for producing a plant exhibiting new combinations of altered chromosomal loci useful for breeding comprising the steps of: (a) selfing a plant comprising altered chromosomal loci induced by plastid perturbation to produce progeny; and, (b) assaying one or more sRNAs of said progeny to identify and select individuals with new combinations of altered chromosomal loci are also provided herein. In certain embodiments one or more sRNAs assayed have sequence homology to the group of altered chromosomal loci consisting of MSH1, pericentromeric regions, CG enhanced genes, CG depleted genes, transposable elements, transposable elements containing genes, and transposable elements in pericentromeric regions are provided herein Methods for producing a plant exhibiting new combinations of altered chromosomal loci useful for breeding comprising the steps of: (a) selfing a plant comprising altered chromosomal loci induced by MSH1 or PPD3 suppression to produce progeny; and, (b) assaying one or more sRNAs of said progeny to identify and select individuals with new combinations of altered chromosomal loci are also provided herein. In certain embodiments one or more sRNAs assayed have sequence homology to the group of altered chromosomal loci consisting of MSH1, pericentromeric regions, CG enhanced genes, CG depleted genes, transposable elements, transposable elements containing genes, and transposable elements in pericentromeric regions are also provided herein.

Methods for selecting a plant comprising one or more altered chromosomal loci useful for plant breeding comprising the steps of: a) comparing the DNA methylation status of one or more nuclear chromosomal regions in a reference plant to one or more corresponding nuclear chromosomal regions in a candidate plant, wherein said candidate plant or one or more of its progenitors was obtained by plastid perturbation; and, b) selecting a candidate plant comprising one or more nuclear chromosomal regions present in the candidate plant with a DNA methylation status that is distinct from the DNA methylation status in the reference plant, thereby selecting a plant comprising one or more altered chromosomal loci useful for plant breeding are provided herein. Methods for selecting a plant comprising one or more altered chromosomal loci useful for plant breeding comprising the steps of: a) comparing the DNA methylation status of one or more nuclear chromosomal regions in a reference plant to one or more corresponding nuclear chromosomal regions in a candidate plant, wherein said candidate plant or one or more of its progenitors was obtained by suppression of MSH1 or PPD3; and, b) selecting a candidate plant comprising one or more nuclear chromosomal regions present in the candidate plant with a DNA methylation status that is distinct from the DNA methylation status in the reference plant, thereby selecting a plant comprising one or more altered chromosomal loci useful for plant breeding are also provided herein.

Methods for selecting a plant comprising one or more altered chromosomal loci useful for plant breeding comprising the steps of: a) comparing one or more sRNAs with homology to one or more nuclear chromosomal regions in a reference plant to one or more sRNAs from corresponding nuclear chromosomal regions in a candidate plant, wherein said candidate plant or one or more of its progenitors was obtained byplastid perturbation; and, b) selecting a candidate plant comprising one or more sRNA with abundances or sequences that are distinct from the sRNAs in the reference plant, thereby selecting a plant comprising one or more altered chromosomal loci useful for plant breeding are provided herein. Methods for selecting a plant comprising one or more altered chromosomal loci useful for plant breeding comprising the steps of: a) comparing one or more sRNAs with homology to one or more nuclear chromosomal regions in a reference plant to one or more sRNAs from corresponding nuclear chromosomal regions in a candidate plant, wherein said candidate plant or one or more of its progenitors was obtained by suppression of MSH1 or PPD3; and, b) selecting a candidate plant comprising one or more sRNA with abundances or sequences that are distinct from the sRNAs in the reference plant, thereby selecting a plant comprising one or more altered chromosomal loci useful for plant breeding are also provided herein.

In certain embodiments of any of the aforementioned methods, the plant is a crop plant. In certain embodiments the crop plant is from the group consisting of corn, wheat, rice, sorghum, millet, tomato, potato, soybean, tobacco, cotton, canola, alfalfa, rapeseed, sugar beets, and sugarcane.

In certain embodiments of the methods, the DNA methylation status comprises at least one of CG hypermethylation, CHG hypermethylation, or CHH hypermethylation. In certain embodiments of the methods, the DNA methylation status comprises at least one of CG hypomethylation, CHG hypomethylation, or CHH hypomethylation. In certain embodiments of the methods, the DNA methylation status comprises hypermethylation and hypomethylation in chromosomal regions comprising sequences selected from the group of CG, CHG, and CHH DNA sequences.

In certain embodiments of any of the aforementioned methods, the selection comprises isolating a plant or progeny plant comprising the altered chromosomal locus. Also provided is an altered chromosomal locus of a plant identified by any of the aforementioned methods. Also provided is a plant, plant part, plant seed, or processed plant product comprising the altered chromosomal locus. Also provided is a plant made by any of the aforementioned methods as well as seed therefrom.

In certain embodiments of any of the aforementioned methods, the plants or progeny thereof can be self pollinated, outcrossed or crossed to an isogenic line. In certain embodiments progeny can be vegetatively propagated. Clonal propagates obtained from the plants, the progeny thereof, or from the plant parts are also provided.

In certain embodiments, the plant is selected from the group consisting of a crop plant, a tree, a bush, a grass, and a vine. In certain embodiments, the crop plant is selected from the group consisting of corn, soybean, cotton, canola, wheat, rice, tomato, tobacco, millet, potato, sugarbeet, cassava, alfalfa, barley, oats, sugarcane, sunflower, strawberry, and sorghum. In certain embodiments, the tree is selected from the group consisting of an apple, apricot, grapefruit, orange, peach, pear, plum, lemon, coconut, poplar, eucalyptus, date palm, palm oil, pine, and an olive tree. In certain embodiments, the bush is selected from the group consisting of a blueberry, raspberry, and blackberry bush. Also provided are plants or progeny thereof obtained by any of the aforementioned methods. Also provided are plant parts obtained from the plant or progeny thereof that were made by any of the aforementioned methods. In certain embodiments, the plant part is selected from the group consisting of a seed, leaf, stem, fruit, and a root. Also provided are clonal propagates obtained from the plant or progeny thereof that were made by any of the aforementioned methods.

### BRIEF DESCRIPTION OF THE DRAWINGS

The accompanying drawings, which are incorporated in and form a part of the specification, illustrate certain embodiments of the present invention. In the drawings:

FIG. 1A-J illustrates that MSH1 is located in distinct epidermal and vascular parenchyma plastids. (A) Laser confocal micrograph of the leaf lamina of an Arabidopsis MSH1-GFP stable transformant. Mesophyll chloroplasts autofluoresce red. (B) Laser confocal Z-scheme perpendicular rotation to allow simultaneous visualization of optical sections. Note the lack of GFP fluorescence below the top (epidermal) layer. (C) Enlargement from panel A to allow discrimination of the smaller sized plastids containing MSH1-GFP. (D) Laser confocal micrograph of the midrib region of an Arabidopsis MSH1-GFP stable transformant. Note the dense population of smaller sized plastids with GFP signal. (E) Confocal Z-scheme perpendicular rotation of the midrib section. Note the dense GFP signal through all layers. (F) MSH1-GUS localization to plastids in the vascular parenchyma of the leaf midrib. (G) Floral stem cross-section of an Arabidopsis MSH1-GUS stable transformant. Note the intensity of GUS staining within the vascular parenchyma cells. (H) MSH1-GUS expression in a cleared root of an Arabidopsis stable transformant. (I) MSH1-GUS localization pattern in a cleared Arabidopsis leaf. Note the intense staining of the vascular tissue and epidermal trichomes. (J) Leaf cross-section showing MSH1-GFP localization by laser confocal microscopy. Yellow arrow indicates vascular bundle.

FIG. 2A-G shows that MSH1 is expressed predominantly in reproductive tissues and in vascular tissues throughout the plant. (A) MSH1-GUS expression in an Arabidopsis stable transformant seedling. MSH1 expression at the meristem (B) and root tip (C). (D) MSH1-GUS expression in the ovule; note enhanced expression evident in the funiculus. (E) MSH1-GUS localization in developing pollen within a cleared anther. (F) MSH1-GFP expression within a petal, showing enhanced localization within vascular tissues. (G) MSH1-GUS localization within the Arabidopsis flower.

FIG. 3A-E shows that MSH1 is located in a specialized plastid type. (A) Sensory plastids in vascular parenchyma adjacent to mesophyll cell chloroplasts in Arabidopsis. (B) Enlargement of a sensory plastid and adjacent mesophyll chloroplast. Note difference in size and grana organization. (C) Tobacco leaf epidermal and mesophyll chloroplasts, red channel (arrow indicates stomate) (D) green channel image, showing MSH1-GFP localization. (E) Merged image showing association of MSH1-GFP with smaller epidermal plastids. Note the punctate appearance of GFP signal within the smaller organelles.

FIG. 4A-C shows that MSH1 co-purifies with the thylakoid membrane fraction. (A) Total Col-0 plastid preparations were separated to stromal and thylakoid fractions for protein gel blot analysis, with antibodies specific for MSH1, Rubisco and PsbO proteins. The lower panel is a Coomassie-stained gel sample of the preparations. (B) Total plastid preparations from a MSH1-GFP stable transformant were fractionated for immunoblot analysis that included milder detergent washes. (C) Influence of increased concentration salt washes on membrane association of MSH1, PsbO and PsbP. In each case, experimental results shown are spliced from single experiments.

FIG. 5A-C shows that MSH1 interacts with components of the photosynthetic electron transport chain. (A) MSH1 coIP assay products, with msh1 negative control in lane 1 and wildtype in lane 2. Arrow indicates MSH1 protein. This assay produced PsbA and PetC as putative interaction partners to MSH1. (B) Yeast 2-hybrid assay with full-length MSH1 as bait in one-on-one assay with PsbA and PetC, allowed to incubate for one week, suggesting weak interaction. (C) Yeast 2-hybrid experiments with MSH1 full-length or individual domains as bait in combination with various components of the PSII oxygen evolving complex (PsbO1/O2, PPD3), D1 (PsbA) and PetC from the neighboring B6F complex. Note the weak signal observed for PsbA and PetC.

FIG. 6A-F shows that MSH1 and PPD3 appear to be co-expressed in the vascular parenchyma and epidermal cell plastids. (A) Floral stem cross-section showing xylem (blue) and chloroplast autofluorescence (red). (B) Floral stem cross-section showing MSH1-GFP expression localized to the parenchyma of phloem and xylem, epidermal cells and in the pith. (C) Floral stem cross-section showing PPD3-GFP expression localized to plastids in a similar pattern to MSH1. (D) Confocal micrograph of leaf epidermal cells showing PPD3-GFP localization to plastids. (E) Enlargement showing GFP signal for MSH1 in the vascular tissue. Note that the signal is localized within small plastids. (F) MSH1 (GFP, green) and the nucleoid protein MFP1 (RFP, red) localization in epidermal plastids. Larger sized chloroplasts of the underlying mesophyll cells are shown in blue. Note that MSH1 and MFP1 do not completely co-localize (co-localization signal is yellow).

FIG. 7A-D shows that the ppd3 mutant resembles the msh1 dr phenotype. (A) Diagram of the PPD3 gene in Arabidopsis and the T-DNA insertion mutation site. (B) PCR-based genotyping of three PPD3 T-DNA insertion mutants. (C) RT-PCR assay of PPD3 expression in three T-DNA insertion mutants. (D) ppd3-gabi mutant phenotype under conditions of 10-hour day length, displaying aerial rosettes similar to msh1-dr.

FIG. 8A-B shows that the msh1 mutant displays altered plastid redox features. (A) Plastoquinone (PQ9) levels, reduced and oxidized) in Arabidopsis were assayed in wild type (Col-0) and the msh1 mutant, testing both leaf (where mesophyll chloroplasts predominate and MSH1 levels are very low) and in stem (where sensory plastids are in greater abundance and MSH1 levels are higher). (B) Plastochromanol-8 (PC8) levels were measured in both leaf and stem. The observation of changes in plastoquinone level, redox state (becoming more highly reduced), and increases in PC-8 levels in the stem of the msh1 mutant suggests that the changes we observe may be more pronounced in the sensory plastids of the msh1 mutant. Note the difference in Y-axis scales to allow more detailed evaluation of stem effects.

FIG. 9A-B shows that sensory plastids comprise ca 2-3% of the plastids derived from crude plastid extractions. Fluorescence-activated cell sorting (FACS) analysis was carried out with total leaf crude plastid extractions derived from (A) Arabidopsis and (B) tobacco plants stably transformed with the Arabidopsis full-length MSH1-GFP fusion construct, comparing to wildtype as negative control for plastid autofluorescence. Plots show GFP fluorescence (X axis) over background auto-fluorescence of chlorophyll. The percentage in each plot of GFP sorted chloroplasts in wildtype and transgenic lines is indicated at the bottom of each plot.

FIG. 10A-D shows that MSH1 and PPD3 show evidence of protein interaction by co-immunoprecipitation. Stable double transformants for MSH1-GFP and PPD3-RFP fusion genes (PPD3×MSH1 OE) were used for coIP analysis. In each experiment, the left lane is a marker. (A) Immunoblot with anti-MSH1 antibodies on blotted total protein. (B) Immunblot with anti-RFP antibodies on total protein. (C) CoIP from incubation of total protein with anti-MSH1 beads, probed with anti-GFP and anti-RFP antibodies. (D) Coomassie stained gel of the coIP precipitate from panel C.

FIG. 11 shows that PsbO2-GFP expression in a cross-section of the floral stem. Xylem is visualized as blue, chloroplast autofluorescence is in red (in plastids that are not disturbed by sectioning. The PsbO2 protein is a lumenal protein. We presume that the chloroplasts that appear green are those that have been disrupted by sectioning, while those below that appear red likely are intact. Under photosynthetically active wavelengths, the lumen is likely to maintain a very low pH, which would prevent visualization of GFP.

FIG. 12A-C shows that the msh1 and ppd3 mutants are similar in non-photochemical quenching (NPQ) properties of their plastids. Fluorometric measurements of chlorophyll fluorescence for calculation of NPQ was carried out in Arabidopsis wildtype (Col-0), two msh1 mutants, chm1-1 and 17-34, and two ppd3 mutants, ppd3-Gabi and ppd3-Sail. Both the msh1 and ppd3 mutants develop NPQ faster than WT in the light. The NPQ in these mutants then decays slower in the dark, with differences significant at the P<0.05 level.

FIG. 13A-G shows the enhanced growth phenotype of MSH1-epi lines in Arabidopsis. (A) Crossing and selection procedure to derive early generation msh1 materials for methylome analysis. (B) First-generation msh1 phenotypes for segregating progeny from a single hemizygous plant. Null msh1 plants are marked with triangles. Plants shown are 33 days old. (C) Segregating second generation siblings from a single null msh1 first generation parent. Note the size variation and extensive variegation in the second generation. Plants are 33 days old. (D) Crossing strategy for epiF3 and epiF4 families. (E) Enhanced growth phenotype of the epiF4. (F) Arabidopsis epiF4 plants show enhanced plant biomass, rosette diameter and flower stem diameter relative to Col-0. Data are shown as mean±SE from >6 plants. (G) The Arabidopsis epiF4 phenotype at flowering.

FIG. 14A-F shows MSH1-epi enhanced growth in Arabidopsis is associated with chloroplast effects. (A) Mitochondrial hemi-complementation line AOX-MSH1×Col-0 F1. (B) Plastid-complemented SSU-MSH1×Col-0 F2 appears identical to Col-0 wildtype. (C) Rosette diameter and fresh biomass of SSU-MSH1-derived F2 lines relative to Col-0. (D) Mitochondrial-complemented AOX-MSH1×Col-0 F2 showing enhanced growth. (E) Rosette diameter and fresh biomass of AOX-MSH1-derived F2 lines is significantly greater (P<0.05) than Col-0. (F) Enhanced growth phenotype in the F2 generation of AOX-MSH1×Col-0.

FIG. 15A-D shows Genome-wide 5-methyl-cytosine CG patterns in Arabidopsis. Distribution of CG-DMPs (red) and CG-N-DMPs (blue) along each chromosome in a comparison of first and second-generation msh1/msh1 versus a wildtype sib MSH1/MSH1, advanced-generation msh1 versus Col-0, and epiF3 versus Col-0, with data normalized across all chromosomes. The arrow indicates the position of MSH1 on Chromosome 3.

FIG. 16A-D shows hypermethylation trends in first, second and advanced generation msh1 and epiF3 lines (A) Relative contributions of CG, CHG and CHH methylation to differential methylated positions (DMPs) and non-differential methylated positions (NDMPs) of the genome in the msh1 and epiF3 lines relative to Col-0. (B) Relative distribution of DMPs within genes in the msh1 and epiF3 lines. (C) Relative proportion of hyper- and hypomethylation CG and CHG changes in early generation msh1 versus a MSH1/MSH1 sib, and advanced generation msh1 and epiF3 relative to wildtype Col-0. (D) Heat map of CHG analysis. The heatmap values represent the DMP number within the sliding windows along each chromosome (window size=100 kb, moving distance=5 kb). The arrow to the right of each shows approximate location of centromere.

FIG. 17A shows the distribution of flowering time in Arabidopsis Col-0, epiF4 and epiF5 lines. Each distribution is plotted based on 15-20 plants.

FIG. 17B shows the distribution of msh1 SNPs and indels versus Col-0 across the genome. Each dot represents the number of SNPs and indels found in a window of 50 kbp. Note that the Y-axis has been synchronized with the maximum number found on chr4 to enable comparisons between chromosomes. The region 7,800,000-9,850,000 bp on chr4, a likely introgressed segment from Ler, contains 8582 of the total 12,771 SNPs and indels. The overlap between these data and the known SNPs and small indels of Ler vs. Col-0 (17) is 72% and 67% for SNPs and indels, respectively.

FIG. 17C shows Arabidopsis F1 plants resulting from crosses of the msh1 chloroplast hemi-complementation line×Col-0 wildtype. Transgene-mediated chloroplast hemi-complementation of msh1 restores the wildtype phenotype. However, crossing of these hemi-complemented lines to Col-0 results in range from 10% to 77% of the plants displaying leaf curl in independent F1 progenies (F1). The cause of this phenotype is not yet known, but it is heritable in derived F2 populations (F2).

FIG. 18A-D shows the Venn Diagrams of the overlapping DMRs for CG (A)(B)(C), and CHG (D).

FIG. 19 shows an example of CG DMP distribution plotted by hypermethylation versus hypomethylation along Chromosome 3. Pink arrows show regions where the asymmetry is particularly pronounced in the msh1 second generation dwarfed (dr) lines.

FIG. 20 shows the Gene ontology distribution of genes with significantly altered expression levels in msh1 versus those in epiF3 based on transcript profile analysis.

FIG. 21A-G. Phenotypically variable msh1 mutants produce enhanced progeny upon crossing to wild type. a, Scheme to derive early generation msh1 materials for methylome analysis. b, Segregating progeny from a single hemizygous plant. First generation msh1 −/− plants are marked with triangles. c, Second generation siblings from a single first generation msh1 −/− parent exhibited variegation and size variation. d, Crossing scheme for creating epi-lines. e, Enhanced growth phenotype of the epiF₄. f, The epiF₄ plants show enhanced plant biomass, rosette diameter and floral stem diameter relative to Col-0. g, The epiF₄ phenotype at maturity.

FIG. 22A-C. Pair-wise DMP patterns of MSH1+/− and early msh1 mutants when compared to wild type segregants, and of advanced msh1 mutants and epiF₃ when compared to stock Col-0. a, Distribution of CG, CHG, and CHH-DMPs along chromosome 2. Top window, distribution of transposons; arrow indicates centromere. b, Comparison of whole genome, gene, and transposon pair-wise DMP counts. c, Distribution of hypermethylated pair-wise DMPs over genes and transposons.

FIG. 23A-D. Partition of the set of samples into subsets based on genome-wide methylation patterns. a,b, Discriminatory information conserved in two linear discriminate (LD) functions reveals the existence of genome-wide CG and CHG methylation patterns that discriminate the epiF3 lines from the subsets of mutants and wild types. c,d, Loadings of group-wise DMRs in the LD functions indicate which DMRs have a relevant contribution in discerning between samples.

FIG. 24A-C. Graft transmission of the msh1-associated enhanced-growth phenotype. a, Representative plants of the first generation of progeny from grafts, designated by scion/rootstock in each case. b, Rosette diameter and fresh biomass of CoI-0/Col-0 control graft compared to msh1 and the first generation of progeny from independent grafts. c, Rosette diameter, leaf number and fresh biomass of the second generation of progeny from the indicated grafts. All grafts involved floral stems and progeny measurements were taken at a single time point. The msh1 mutant shown is the advanced mutant chm1-1.

FIG. 25A-B. a, Arabidopsis F₁ plants resulting from crosses of the msh1 chloroplast hemi-complementation line×Col-0 wild type. Transgene-mediated chloroplast hemi-complementation of msh1 restores the wild type phenotype′. However, crossing of these hemi-complemented lines to Col-0 results in a variable proportion of plants displaying leaf curl (at varying intensities) in the F₁. The cause of this phenotype is not yet known, but it is heritable in derived F₂ populations. b, Analysis of phenotype data from individual Arabidopsis F₂ families derived by crossing hemi-complementation lines×Col-0 wild type. SSU-MSH1 refers to lines transformed with the plastid-targeted form of MSH1; AOX-MSH1 refers to lines containing the mitochondrial-targeted form of the MSH1 transgene. In all genetic experiments using hemi-complementation, presence/absence of the transgene was confirmed with a PCR-based assay.

FIG. 26A-F. MSH1-mediated enhanced growth from crossing is associated with plastid effects. a, Mitochondrial hemi-complementation line AOX-MSH1×Col-0 F₁. b, Mitochondrial-complemented AOX-MSH1×Col-0 F₂ showing enhanced growth. c, Rosette diameter and fresh biomass of AOX-MSH1-derived F₂ lines is significantly greater than Col-0 (* p<0.05). d, Plastid-complemented SSU-MSH1×Col-0 F₂ appears similar to wild type Col-0. e, Rosette diameter and fresh biomass of SSU-MSH1-derived F₂ lines compared to Col-0. f, Enhanced growth phenotype in the F₂ generation of AOX-MSH1×Col-0.

FIG. 27A-C. a, Distribution of msh1 SNPs and indels versus Col-0 across the genome. Each dot represents the number of SNPs and indels found in a window of 50 kbp. Note that the Y-axis has been synchronized with the maximum number found on chr4 to enable comparisons between chromosomes. The region 7,800,000-9,850,0000 bp on chr4, a likely introgressed segment from Ler, contains 8582 of the total 12,771 SNPs and indels. The overlap between these data and the known SNPs and small indels of Ler vs. Col-0³² is 72% and 67% for SNPs and indels, respectively. Paired-end genome-wide sequencing, alignment and de novo partial assembly of the chm1-1 genome produced 14,416 contigs (n50=40,761 bp) containing 118.5 Mbp; mapping these contigs against Col-0 covers 72 Mbp. Alignment of paired-end reads to the Col-0 public reference sequence produced 95% alignment and identified 12,771 SNPs and indels, with the one 2-Mbp interval, on chromosome 4, accounting for 8,582 and the second on Chromosome 3 accounting for 2200. The chm1-1 mutant used in this study is a Col-0 mutant once crossed to Ler (Redei, G. P. Mutat. Res. 18, 149-162 (1973), and the Ler introgressed segment on Chromosome 3 was identified genetically during positional cloning of MSH1 (Abdelnoor, R. V. et al. Proc. Natl. Acad. Sci. USA 100, 5968-5973 (2003)). Comparing SNPs and indels in the chromosome 4 interval with those in a recent study of Ler×Col-0 Lu, P. et al. Genome Res. 22, 508-518 (2012) accounts for 5060 of 6985 SNPs (72%) and 1073 of 1597 indels (67%), consistent with a Ler introgressed segment. Of the remaining 1988 SNP/indels, about 70% reside in non-genic regions. This SNP mutation rate appears consistent with natural SNP frequencies Becker, C. et al. Nature 480, 245-249 (2011)). b, For treatment of seedlings with the methylation inhibitor 5-azacytidine, seeds were alternately arranged as shown to minimize the effect of spatial variation. c, Increased epi-line root length is abolished by 50 μM 5-azacytidine. To assess the significance of the differences between the lines under control treatment versus 5-azacytidine, root length data was fit to the linear model Yᵢⱼₖ=lineᵢ+treatmentⱼ+(line*treatment)ᵢⱼ+εᵢⱼₖ; two-way ANOVA then indicated that the line*treatment interaction term was significant (F=6.60, df=2, p-value=0.002).

FIG. 28. Chromosomal distributions of pair-wise CG-DMPs (red) and CG-NDMPs (blue), in a comparison of MSH1+/−, first generation msh1, second generation variegated msh1, and second generation dwarf msh1 versus wild type segregant (normalized together), as well as advanced msh1 and epiF₃ versus Col-0 (normalized together). Arrow on msh1_gen1 indicates the position of the MSH1 gene on chromosome 3.

FIG. 29A-C. a, Proportion of pair-wise DMPs composed of each cytosine context within genes, transposons, and the whole genome. msh1 second generation dwarf and epi-F3 show disproportionately high levels of CHG hypermethylation, particularly within transposons. b, Separate plots by cytosine context for comparison of relative hypermethylated pair-wise DMPs and hypomethylated pair-wise DMPs. msh1 and epiF₃ mutants showing higher a trend of hypermethylation, except for transposon CG methylation in epiF₃. c, Distribution of hypomethylated pair-wise DMPs across genes and transposons, by cytosine context.

FIG. 30A-B. a, Heat maps of pair-wise CHG-DMPs by chromosome using pooled samples (left), and individual samples with cross-comparisons in the order: mutant_rep1 vs wildtype_rep1, mutant_rep2 vs wildtype_rep1, mutant_rep1 vs wildtype_rep2, mutant_rep2 vs wildtype_rep2 (middle). Heat map of pair-wise CHH-DMPs by chromosome using pooled samples (right). Approximate location of centromere is indicated by arrows. b, Pair-wise CHG-DMP numbers in the cross-comparisons for msh1_gen2_dwf and epiF3, maintaining the same order as in the heat map.

FIG. 31A-B. a, By count, Gypsy-like retrotransposons are highly enriched among transposons overlapping group-wise CG and CHG-DMRs in our material. To a lesser degree LINE and (for CHG) Copia-like elements are also enriched. This superfamily distribution generally resembles that of transposons which are associated with an intact transposable element gene. Bottom right: enrichment of particular superfamilies is unlikely to be an artifact from the amount of sequence space occupied by those superfamilies across the genome. b, Table of counts for each transposon superfamily, with Benjamini-Hochberg adjusted p-values for significantly under or over-represented superfamilies (based on Wallenius' non-central hypergeometric distribution) overlapping with group-wise DMRs. For this test, transposons were weighted by median superfamily sequence length to counter potential length bias in DMR overlap.

FIG. 32A-B. Distribution of relative (a) hypermethylated and (b) hypomethylated pair-wise DMP frequencies across transposable elements that do not contain or overlap with a TE gene (n=26317) and those that do contain or overlap with a TE gene (n=4872), by cytosine context. Unsurprisingly, transposons not associated with a TE gene are typically shorter than those that are (median by length=255 and 1332.5, respectively; Wilcoxon test p-value <2.2 e-16). Fluctuations in CG-DMP frequencies within bodies of transposons that are not associated with a TE gene are likely due to the relatively small number of such transposons that are long in sequence length.

FIG. 33A-F. Clustering based on the LDA coordinates of the samples. Hierarchical clustering for (a) CG and (b) CHG methylation of the LDA presented in FIGS. 3A and B of the main text, respectively. LDA for (c) CG and (d) CHG methylation regions of window size of 340 bp with at least 20 cytosine coveraged sites; panels (e) and (f) are their corresponding hierarchical clustering, respectively. In all the cases, the four first PCA components were used as new variables in the LDAs and the proportion of conserved variance was greater than 0.8.

FIG. 34A-B. Enhanced growth progeny from Col-0 scions grafted to msh1 mutants. a, Second generation of progeny from grafts derived by self-pollination of first generation of progeny from grafts. These grafts involved chm1-1 and were used for measurements presented in FIG. 24. b, msh1 mutant rootstocks from the SAIL_{—}877_F01 T-DNA line influenced Col-0 scions to produce enhanced growth progeny. “Gen1” and “Gen2” indicates rootstock was from first or second generation msh1 mutants, respectively, as described in FIG. 21 a. Rosette diameter at the time of floral stem bolting was measured for Col-0 and progeny from Col-0 scions grafted to Gen1 and Gen2 plants.

### DESCRIPTION

As used herein, the terms “useful for plant breeding” or “useful for breeding” refer to plants that are useful in a plant breeding program for the objective of developing improved plant traits.

As used herein, the terms “pericentromeric” or “pericentromere” refer to heterochromatic regions containing abundant repeated sequences, transposable elements, and retrotransposons that physically flank the centromeric regions. At the sequence level, a functional definition for pericentromeric sequences are repeated sequences that contain transposable elements and retrotransposons embedded in said repeated sequences. When known, centromeric repeats can be computationally removed from the repeated sequences, but their presence is not detrimental if not computationally removed. When available, chromosomal positioning information about the location of sequences that are located adjacent to the centromere can be used as additional criteria for pericentromeric sequences.

As used herein, the terms “CG altered gene” or “CG altered genes” refer to a gene or genes with increased or decreased levels of DNA methylation (5 meC) at CG nucleotides within or near a gene or genes. The region near a gene is within 5,000 bp, preferably within 1,000 bp, of either the 5′ or 3′ end of the gene or genes.

As used herein, the terms “CG enhanced genes” refers to CG altered genes with higher levels of DNA methylation or sRNA derived from said CG enhanced genes relative to the levels from a reference plant.

As used herein, the phrase “CG depleted genes” refers to CG altered genes with lower levels of DNA methylation or sRNA derived from said CG enhanced genes relative to the levels from a reference plant.

As used herein, the phrase “chromosomal modification” refers to any of: a) an “altered chromosomal loci” and an “altered chromosomal locus”; b) “mutated chromosomal loci”, a “mutated chromosomal locus”, “chromosomal mutations” and a “chromosomal mutation”; or c) a transgene.

As used herein, the phrases “altered chromosomal loci” (plural) or “altered chromosomal locus (singular) refer to portions of a chromosome that have undergone a heritable and reversible epigenetic change relative to the corresponding parental chromosomal loci. Heritable and reversible genetic changes in altered chromosomal loci include, but are not limited to, methylation of chromosomal DNA, and in particular, methylation of cytosine residues to 5-methylcytosine residues, and/or post-translational modification of histone proteins, and in particular, histone modifications that include, but are not limited to, acetylation, methylation, ubiquitinylation, phosphorylation, and sumoylation (covalent attachment of small ubiquitin-like modifier proteins). As used herein, “chromosomal loci” refer to loci in chromosomes located in the nucleus of a cell.

As used herein, the phrase “new combinations of altered chromosomal loci” refers to nuclear chromosomal regions in a progeny plant with one or more differences in altered chromosomal loci when compared to altered chromosomal loci of a parental plant if derived by self-pollination, or if derived from a cross, when compared to either parental plant, each compared separately to said progeny plant.

As used herein, the term “progeny” refers to any one of a first, second, third, or subsequent generation obtained from a parent plant if self pollinated or from parent plants if obtained from a cross. Any materials of the plant, including but not limited to seeds, tissues, pollen, and cells can be used as sources of RNA or DNA for determining the status of the RNA or DNA composition of said progeny.

As used herein, the phrases “suppression” or “suppressing expression” of a gene refer to any genetic, nucleic acid, nucleic acid analog, environmental manipulation, grafting, transient or stably transformed methods of any of the aforementioned methods, or chemical treatment that provides for decreased levels of functional gene activity, including inhibition of the protein activity produced from the gene, in a plant or plant cell relative to the levels of functional gene activity that occur in an otherwise isogenic plant or plant cell that had not been subjected to this genetic or environmental manipulation.

As used herein, the phrases “assaying” or “assayed” refer to methods for determining the amounts, or sequences, or both, of DNA methylation or sRNA, corresponding to one or more nuclear chromosomal regions for DNA or with homology to one or more nuclear chromosomal regions for sRNA. The nuclear chromosomal regions assayed for DNA methylation can be a single nucleotide position or a region greater than this. Preferably the DNA methylation is from a region comprising one or more CG, CHG, or CHH sites and is compared to the corresponding parental chromosomal loci prior to MSH1 suppression. sRNA can be measured for a single type of sRNA, one or more sRNAs, or a whole population of sRNAs by methods known to those skilled in the art.

As used herein, the phrases “epigenetic modifications” or “epigenetic modification” refer to heritable and reversible epigenetic changes that include, but are not limited to, methylation of chromosomal DNA, and in particular, methylation of cytosine residues to 5-methylcytosine residues. Changes in DNA methylation of a region are often associated with changes in sRNA levels with homology to the region and are derived from the region.

As used herein, the phrases “increased DNA methylation” or “decreased DNA methylation” refer to nucleotides, regions, genes, chromosomes, and genomes located in the nucleus that have undergone a change in 5 meC levels in a plant or progeny plant relative to the corresponding parental chromosomal loci prior to MSH1 suppression or to a parental plant not subjected to MSH1 suppression.

As used herein, the term “comprising” means “including but not limited to”.

As used herein, the phrases “mutated chromosomal loci” (plural) (plural), “mutated chromosomal locus” (singular), “chromosomal mutations” and “chromosomal mutation” refer to portions of a chromosome that have undergone a heritable genetic change in a nucleotide sequence relative to the nucleotide sequence in the corresponding parental chromosomal loci. Mutated chromosomal loci comprise mutations that include, but are not limited to, nucleotide sequence inversions, insertions, deletions, substitutions, or combinations thereof. In certain embodiments, the mutated chromosomal loci can comprise mutations that are reversible. In this context, reversible mutations in the chromosome can include, but are not limited to, insertions of transposable elements, defective transposable elements, and certain inversions. In certain embodiments, the chromosomal loci comprise mutations are irreversible. In this context, irreversible mutations in the chromosome can include, but are not limited to, deletions.

As used herein, the term “discrete variation” or “V_{D}” refers to distinct, heritable phenotypic variation, that includes traits of male sterility, dwarfing, variegation, and/or delayed flowering time that can be observed either in any combination or in isolation.

As used herein, the phrase “heterologous sequence”, when used in the context of an operably linked promoter, refers to any sequence or any arrangement of a sequence that is distinct from the sequence or arrangement of the sequence with the promoter as it is found in nature. As such, an MSH1 promoter can be operably linked to a heterologous sequence that includes, but is not limited to, MSH1 sense, MSH1 antisense, combinations of MSH1 antisense and MSH1 sense, and other MSH1 sequences that are distinct from, or arranged differently than, the operably linked sequences of the MSH1 transcription unit as they are found in nature.

As used herein, the term “MSH-dr” refers to leaf variegation, cytoplasmic male sterility (CMS), a reduced growth-rate phenotype, delayed or non-flowering phenotype, increased plant tillering, decreased height, decreased internode elongation, plant tillering, and/or stomatal density changes that are observed in plants subjected to suppression of plastid perturbation target genes. Plastid perturbation target genes that can be suppressed to produce an MSH-dr phenotype include, but not limited to, MSH1 and PPD3.

As used herein, the phrase “quantitative variation” or “V_{Q}” refers to phenotypic variation that is observed in individual progeny lines derived from outcrosses of plants where MSH1 expression was suppressed and that exhibit discrete variation to other plants.

As used herein, the phrase “reference plant” refers to a parental plant or progenitor of a parental plant prior to MSH1 suppression, but otherwise isogenic to the candidate plant to which it is being compared. In a cross of two parental plants, a “reference plant” can also be from a parental plant wherein MSH1 suppression was not used in said parental plant or one of its progenitors.

As used herein, the phrases “suppression” or “suppressing expression” of a gene refer to any method that provides for decreased levels of functional gene activity, including inhibition of the protein activity produced from the gene, in a plant or plant cell relative to the levels of functional gene or protein activity that occur in an otherwise isogenic plant or plant cell that had not been subjected to the method. Methods for “suppression” or “suppressing expression” of a gene include, but are not limited to, genetic, nucleic acid, nucleic acid analog, environmental manipulation, grafting mediated, transient transformation, stably transformation, chemical treatment methods, and combinations thereof.

As used herein the terms “microRNA” or “miRNA” refers to both a miRNA that is substantially similar to a native miRNA that occurs in a plant as well as to an artificial miRNA. In certain embodiments, a transgene can be used to produce either a miRNA that is substantially similar to a native miRNA that occurs in a plant or an artificial miRNA.

As used herein, the phrase “obtaining a nucleic acid associated with the altered chromosomal locus” refers to any method that provides for the physical separation or enrichment of the nucleic acid associated with the altered chromosomal locus from covalently linked nucleic that has not been altered. In this context, the nucleic acid does not necessarily comprise the alteration (i.e. such as methylation) but at least comprises one or more of the nucleotide base or bases that are altered. Nucleic acids associated with an altered chromosomal locus can thus be obtained by methods including, but not limited to, molecular cloning, PCR, or direct synthesis based on sequence data.

The phrase “operably linked” as used herein refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter is linked such that the resulting translation product is in frame with the translational open reading frame that encodes the protein desired. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators), sequences that provide DNA transfer and/or integration functions (i.e., site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences, homologous recombination sequences), and sequences that provide replication functions (i.e., bacterial origins of replication, autonomous replication sequences, centromeric sequences).

As used herein, the term “transgene” or “transgenic”, in the context of a chromosomal modification, refers to any DNA from a heterologous source that has been integrated into a chromosome that is stably maintained in a host cell. In this context, heterologous sources for the DNA include, but are not limited to, DNAs from an organism distinct from the host cell organism, species distinct from the host cell species, varieties of the same species that are either distinct varieties or identical varieties, DNA that has been subjected to any in vitro modification, recombinant DNA, and any combination thereof.

As used herein, the term “non-regenerable” refers to a plant part or plant cell that can not give rise to a whole plant.

As used herein, the phrase “crop plant” includes, but is not limited to, cereal, seed, grain, fruit, vegetable, tuber, and tree crop plants.

As used herein, the term “commercially synthesized” or “commercially available” DNA refers to the availability of any sequence of 15 bp up to 1000 bp in length or longer from DNA synthesis companies that provide a DNA sample containing the sequence submitted to them.

As used herein, the phrase “loss of function” refers to a diminished, partial, or complete loss of function.

As used herein, the phrase “mutated gene” or “gene mutation” refers to portions of a gene that have undergone a heritable genetic change in a nucleotide sequence relative to the nucleotide sequence in the corresponding parental gene that results in a reduction in function of the gene's encoded protein function. Mutations include, but are not limited to, nucleotide sequence inversions, insertions, deletions, substitutions, or combinations thereof. In certain embodiments, the mutated gene can comprise mutations that are reversible. In this context, reversible mutations in the chromosome can include, but are not limited to, insertions of transposable elements, defective transposable elements, and certain inversions. In certain embodiments, the gene comprises mutations are irreversible. In this context, irreversible mutations in the chromosome can include, but are not limited to, deletions.

As used herein, the term “heterotic group” refers to genetically related germplasm that produce superior hybrids when crossed to genetically distinct germplasm of another heterotic group.

As used herein, the term “genetically homogeneous” or “genetically homozygous” refers to the two parental genomes provided to a progeny plant as being essentially identical at the DNA sequence level.

As used herein, the term “genetically heterogeneous” or “genetically heterozygous” refers to the two parental genomes provided to a progeny plant as being substantially different at the sequence level. That is, one or more genes from the male and female gametes occur in different allelic forms with DNA sequence differences between them.

As used herein, the term “isogenic” refers to the two plants that have essentially identical genomes at the DNA sequence levels level.

As used herein, the term “F1” refers to the first progeny of two genetically or epigenetically different plants. “F2” refers to progeny from the self pollination of the F1 plant. “F3” refers to progeny from the self pollination of the F2 plant. “F4” refers to progeny from the self pollination of the F3 plant. “F5” refers to progeny from the self pollination of the F4 plant. “Fn” refers to progeny from the self pollination of the F(n−1) plant, where “n” is the number of generations starting from the initial F1 cross. Crossing to a isogenic line (backcrossing) or unrelated line (outcrossing) at any generation will also use the “Fn” notation, where “n” is the number of generations starting from the initial F1 cross.

As used herein, the term “S1” refers to a first selfed plant. “S2” refers to progeny from the self pollination of the S1 plant. “S3” refers to progeny from the self pollination of the S2 plant. “S4” refers to progeny from the self pollination of the S3 plant. “S5” refers to progeny from the self pollination of the S4 plant. “Sn” refers to progeny from the self pollination of the S(n−1) plant, where “n” is the number of generations starting from the initial S1 cross.

As used herein, the phrases “self”, “selfing”, or “selfed” refer to the process of self pollinating a plant.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definitions will be used herein.

Methods for introducing heritable and epigenetic and/or genetic variation that result in plants that exhibit useful traits are provided herewith along with plants, plant seeds, plant parts, plant cells, and processed plant products obtainable by these methods. In certain embodiments, methods provided herewith can be used to introduce epigenetic and/or genetic variation into varietal or non-hybrid plants that result in useful traits as well as useful plants, plant parts including, but not limited to, seeds, plant cells, and processed plant products that exhibit, carry, or otherwise reflect benefits conferred by the useful traits. In other embodiments, methods provided herewith can be used to introduce epigenetic and/or genetic variation into plants that are also amenable to hybridization.

In most embodiments, methods provided herewith involve suppressing expression of plant plastid perturbation target genes, restoring expression of a functional plant plastid perturbation target gene, and selecting progeny plants that exhibit one or more useful traits. In certain embodiments, these useful traits are associated with either one or more altered chromosomal loci that have undergone a heritable and reversible epigenetic changes.

In certain embodiments, methods for selectively suppressing expression of plant plastid perturbation target genes in sub-populations of cells found in plants that contain plastids referred to herein as “sensory plastids” are provided. Sensory plastids are plastids that occur in cells that exhibit preferential expression of at least the MSH1 promoter. In certain embodiments, MSH1 and other promoters active in sensory plastids can thus be operably linked to a heterologous sequence that perturbs plastid function to effect selective suppression of genes in cells containing the sensory plastids. In addition to the distinguishing characteristic of expressing MSH1, such cells containing sensory plastids can also be readily identified as their plastids are only about 30-40% of the size of the chloroplasts contained within mesophyll cells. Other promoters believed to be active in sensory plastids include, but are not limited to, PPD3 gene promoters. Selective suppression of plastid perturbation target genes in cells containing sensory plastids can trigger epigenetic changes that provide useful plant traits. Suppression of plant plastid perturbation target genes including but not limited to, photosynthetic components, in specific sub-sets of plant cells that contain the sensory plastids is preferred as suppression of those genes in most other plant cell types is detrimental or lethal to the plant due to impairment of its photosynthetic or other capabilities.

Plastid perturbation target genes that can be suppressed by various methods provided herein to trigger epigenetic or other changes that provide useful traits include, but are not limited to, genes that encode components of plant plastid thylakoid membranes and the thylakoid membrane lumen. In certain embodiments, the plastid perturbation target genes are selected from the group consisting of sensor, photosystem I, photosystem II, the NAD(P)H dehydrogenase (NDH) complex of the thylakoid membrane, the Cytochrome b6f complex, and plastocyanin genes. A non-limiting and exemplary list of plastid pertubation targets is provided in Table 1.

TABLE 1
Exemplary Plastid Perturbation Target Genes

| | | Exemplary Genes Database |
|---|---|---|
| | | Accession Numbers and/or |
| Category | Gene name(s) and/or Activity | SEQ ID NO |
| Sensor | MSH1 | SEQ ID NO: 1, 3-11. |
| Sensor | PPD3 | AT1G76450; SEQ ID NO: |
| | | 16-40 |
| Photosystem I | PHOTOSYSTEM I SUBUNIT | PSAG AT1G55670.1 |
| | G, PSAG | |
| Photosystem I | PHOTOSYSTEM I SUBUNIT | PSAD-2 AT1G03130.1 |
| | D-2, PSAD-2 | |
| Photosystem I | PHOTOSYSTEM I SUBUNIT | PSAO AT1G08380 |
| | O, PSAO | |
| Photosystem I | PHOTOSYSTEM I SUBUNIT | PSAK AT1G30380.1 |
| | K, PSAK | |
| Photosystem I | PHOTOSYSTEM I SUBUNIT | PSAF AT1G31330.1 |
| | F, PSAF | |
| Photosystem I | Photosystem I PsaN, reaction | PsaN AT1G49975.1 |
| | centre subunit N | |
| Photosystem I | PHOTOSYSTEM I SUBUNIT | PSAH-2, PSAH2, PSI-H |
| | H-2, PHOTOSYSTEM I | AT1G52230.1 |
| | SUBUNIT H2, PSAH-2, | |
| | PSAH2, PSI-H | |
| Photosystem I | PHOTOSYSTEM I SUBUNIT | PSAE-2 AT2G20260.1 |
| | E-2, PSAE-2 | |
| Photosystem I | PHOTOSYSTEM I P | PSAP AT2G46820.1 |
| | SUBUNIT, PLASTID | |
| | TRANSCRIPTIONALLY | |
| | ACTIVE 8, PSAP, PSI-P, | |
| | PTAC8, THYLAKOID | |
| | MEMBRANE | |
| | PHOSPHOPROTEIN OF 14 | |
| | KDA, TMP14 | |
| Photosystem I | PHOTOSYSTEM I SUBUNIT | PSAH-1 AT3G16140.1 |
| | H-1, PSAH-1 | |
| Photosystem I | PHOTOSYSTEM I SUBUNIT | PSAD-1AT4G02770 |
| | D-1, PSAD-1 | |
| Photosystem I | PHOTOSYSTEM I SUBUNIT | PSAL AT4G12800 |
| | L, PSAL | |
| Photosystem I | PSAN | PSAN AT5G64040 |
| | LHCA5, PHOTOSYSTEM I | LHCA5 AT1G45474 |
| | LIGHT HARVESTING | |
| | COMPLEX GENE 5 | |
| Photosystem II | PsbY | PsbY AT1G67740 |
| Photosystem II | PsbW | PsbW AT2G30570 |
| Photosystem II | PsbW-like | PsbW-like AT4G28660 |
| Photosystem II | PsbX | PsbX AT2G06520 |
| Photosystem II | PsbR | PsbR AT1G79040 |
| Photosystem II | PsbTn | PsbTn AT3G21055 |
| Photosystem II | PsbO-1 | PsbO-1 AT5G66570 |
| Photosystem II | PsbO-2 | PsbO-2 AT3G50820 |
| Photosystem II | PsbP1 | PsbP1 AT1G06680 |
| Photosystem II | PsbP2 | PsbP2 At2g30790 |
| Photosystem II | PsbS | PsbS AT1G44575 |
| Photosystem II | PsbQ-1 | PsbQ-1, AT4G21280 |
| Photosystem II | PsbQ-2, | PsbQ-2, AT4G05180 |
| Photosystem II | PPL1 | PPL1 At3g55330 |
| Photosystem II | PSAE-1 | PSAE-1 AT4G28750 |
| Photosystem II | LPA2 | LPA2 AT5G51545 |
| Photosystem II | PsbQ-like PQL1 | PQL1 AT1G14150 |
| Photosystem II | PsbQ-like PQL2 | PQL2 AT3G01440, |
| Photosystem II | PsbQ-like PQL3 | PQL3 AT2G01918 |
| NAD(P)H dehydrogenase | PHOTOSYNTHETIC NDH | PPL2 At2g39470 |
| (NDH) Complex | SUBCOMPLEX L 1, PNSL1, | |
| | PPL2, PSBP-LIKE PROTEIN | |
| | 2 | |
| NAD(P)H dehydrogenase | NAD(P)H | NDH48 AT1G15980 |
| (NDH) Complex | DEHYDROGENASE | |
| | SUBUNIT 48, NDF1, NDH- | |
| | DEPENDENT CYCLIC | |
| | ELECTRON FLOW 1, | |
| | NDH48, PHOTOSYNTHETIC | |
| | NDH SUBCOMPLEX B 1, | |
| | PNSB1 | |
| NAD(P)H dehydrogenase | NDF6, NDH DEPENDENT | NDF6 AT1G18730 |
| (NDH) Complex | FLOW 6, | |
| | PHOTOSYNTHETIC NDH | |
| | SUBCOMPLEX B 4, PNSB4 | |
| NAD(P)H dehydrogenase | NAD(P)H | NDH45 AT1G64770 |
| (NDH) Complex | DEHYDROGENASE | |
| | SUBUNIT 45, NDF2, NDH- | |
| | DEPENDENT CYCLIC | |
| | ELECTRON FLOW 1, | |
| | NDH45, PHOTOSYNTHETIC | |
| | NDH SUBCOMPLEX B 2, | |
| | PNSB2 | |
| NAD(P)H dehydrogenase | NDF5, NDH-DEPENDENT | NDF5 AT1G55370 |
| (NDH) Complex | CYCLIC ELECTRON FLOW | |
| | 5 | |
| NAD(P)H dehydrogenase | CHLORORESPIRATORY | NDHL AT1G70760 |
| (NDH) Complex | REDUCTION 23, CRR23, | |
| | NADH DEHYDROGENASE- | |
| | LIKE COMPLEX L, NDHL | |
| NAD(P)H dehydrogenase | NAD(P)H:PLASTOQUINONE | NDHO AT1G74880 |
| (NDH) Complex | DEHYDROGENASE | |
| | COMPLEX SUBUNIT O, | |
| | NADH DEHYDROGENASE- | |
| | LIKE COMPLEX), NDH-O, | |
| | NDHO | |
| NAD(P)H dehydrogenase | PIFI, POST-ILLUMINATION | PIFI AT3G15840 |
| (NDH) Complex | CHLOROPHYLL | |
| | FLUORESCENCE | |
| | INCREASE | |
| NAD(P)H dehydrogenase | NDF4, NDH-DEPENDENT | NDF4AT3G16250 |
| (NDH) Complex | CYCLIC ELECTRON FLOW | |
| | 1, PHOTOSYNTHETIC NDH | |
| | SUBCOMPLEX B 3, PNSB3 | |
| NAD(P)H dehydrogenase | NADH DEHYDROGENASE- | NDHM AT4G37925 |
| (NDH) Complex | LIKE COMPLEX M, NDH-M, | |
| | NDHM, SUBUNIT NDH-M OF | |
| | NAD(P)H:PLASTOQUINONE | |
| | DEHYDROGENASE | |
| | COMPLEX | |
| NAD(P)H dehydrogenase | FK506-BINDING PROTEIN | AT4G39710 |
| (NDH) Complex | 16-2, FKBP16-2, | |
| | PHOTOSYNTHETIC NDH | |
| | SUBCOMPLEX L 4, PNSL4 | |
| NAD(P)H dehydrogenase | CYCLOPHILIN 20-2, , | PNSL5 AT5G13120 |
| (NDH) Complex | CYCLOPHILIN 20-2, CYP20- | |
| | 2, PHOTOSYNTHETIC NDH | |
| | SUBCOMPLEX L 5, PNSL5 | |
| NAD(P)H dehydrogenase | CHLORORESPIRATORY | NDHU AT5G21430 |
| (NDH) Complex | REDUCTION L, CRRL, | |
| | NADH DEHYDROGENASE- | |
| | LIKE COMPLEX U, NDHU | |
| NAD(P)H dehydrogenase | CHLORORESPIRATORY | CRR7 AT5G39210 |
| (NDH) Complex | REDUCTION 7, CRR7 | |
| NAD(P)H dehydrogenase | NAD(P)H | NDH18 AT5G43750 |
| (NDH) Complex | DEHYDROGENASE 18, | |
| | NDH18, PHOTOSYNTHETIC | |
| | NDH SUBCOMPLEX B 5, | |
| | PNSB5 | |
| NAD(P)H dehydrogenase | NADH DEHYDROGENASE- | NDHN AT5G58260 |
| (NDH) Complex | LIKE COMPLEX N, NDHN | |
| Cytochrome b6f complex | Rieske iron-sulfur protein | PetC At4g03280 |
| | containing a [2Fe—2S] cluster, | |
| | OetC | |
| Cytochrome b6f complex | ferredoxin: NADP- reductase | FNR1 AT5G66190 |
| | [FNR1 and FNR2] | FNR2 AT1G20020 |
| plastocyanin | PETE1, PLASTOCYANIN 1 | PETE1 AT1G76100 |
| plastocyanin | PETE2, PLASTOCYANIN 2 | PETE2 AT1G20340 |
| other | PPD1, PSBP-DOMAIN | PPD1 At4g15510 |
| | PROTEIN1 | |
| other | PPD2, PSBP-DOMAIN | PPD2 At2g28605 |
| | PROTEIN2 | |
| other | PPD4, PSBP-DOMAIN | PPD4 At1g77090 |
| | PROTEIN4 | |
| other | PPD5, PSBP DOMAIN | PPD5 At5g11450 |
| | PROTEIN 5 | |
| other | PPD6, PSBP-DOMAIN | PPD6 At3g56650 |
| | PROTEIN 6 | |
| other | PPD7, PSBP-DOMAIN | PPD7 At3g05410 |
| | PROTEIN 7 | |
| MSH1 interacting proteins | CAD9 (CINNAMYL ALCOHOL | CAD9 AT4G39330 |
| identified by Yeast Two Hybrid | DEHYDROGENASE 9); binding/ | |
| | catalytic/oxidoreductase/zinc | |
| | ion binding | |
| MSH1 interacting proteins | KAB1 (POTASSIUM | KAB1 AT1G04690 |
| identified by Yeast Two Hybrid | CHANNEL BETA SUBUNIT); | |
| | oxidoreductase/potassium | |
| | channel | |
| MSH1 interacting proteins | GOS12 (GOLGI SNARE 12); | GOS12 AT2G45200 |
| identified by Yeast Two Hybrid | SNARE binding | |
| MSH1 interacting proteins | ELI3-1 (ELICITOR- | ELI3-1 AT4G37980 |
| identified by Yeast Two Hybrid | ACTIVATED GENE 3-1); | |
| | binding/catalytic/ | |
| | oxidoreductase/zinc ion binding | |
| | (CAD7), response to bacterium, | |
| | plant-type hypersensitive response | |
| MSH1 interacting proteins | STT3B (staurosporin and | STT3B AT1G34130 |
| identified by Yeast Two Hybrid | temperature sensitive 3-like b); | |
| | oligosaccharyl transferase | |
| MSH1 interacting proteins | tRNA synthetase beta subunit | AT1G72550 |
| identified by Yeast Two Hybrid | family protein, FUNCTIONS IN: | |
| | phenylalanine-tRNA ligase | |
| | activity, RNA binding, | |
| | magnesium ion binding, | |
| | nucleotide binding, ATP binding | |
| | (unknown to date) | |
| MSH1 interacting proteins | high mobility group (HMG1/2) | AT4G23800 |
| identified by Yeast Two Hybrid | family protein, FUNCTIONS IN: | |
| | sequence-specific DNA binding | |
| | transcription factor activity; | |
| | LOCATED IN: nucleus, | |
| | chloroplast | |
| MSH1 interacting proteins | Protein kinase superfamily | AT3G24190 |
| identified by Yeast Two Hybrid | protein, FUNCTIONS IN: protein | |
| | kinase activity, ATP binding; | |
| | INVOLVED IN: protein amino | |
| | acid phosphorylation; LOCATED | |
| | IN: chloroplast | |
| MSH1 interacting proteins | Protein kinase superfamily | AT1G64460 |
| identified by Yeast Two Hybrid | protein, FUNCTIONS IN: inositol | |
| | or phosphatidylinositol kinase | |
| | activity, phosphotransferase | |
| | activity (interacts with SNARE | |
| | At2G45200) | |
| MSH1 interacting proteins | RNA-binding (RRM/RBD/RNP | AT1G20880 |
| identified by Yeast Two Hybrid | motifs) family protein; | |
| | FUNCTIONS IN: RNA binding, | |
| | nucleotide binding, nucleic acid | |
| | binding; (interactomes map) | |
| MSH1 interacting proteins | unknown protein, LOCATED IN: | AT5G55210 |
| identified by Yeast Two Hybrid | chloroplast | |
| MSH1 interacting proteins | ATPase, F0/V0 complex, subunit | AT4G32530 |
| identified by Yeast Two Hybrid | C protein; FUNCTIONS IN: | |
| | ATPase activity; INVOLVED IN: | |
| | ATP synthesis coupled proton | |
| | transport (vacuole) | |
| MSH1 interacting proteins | RNA binding: FUNCTIONS IN: | AT3G11964 |
| identified by Yeast Two Hybrid | RNA binding; mRNA processing, | |
| | RNA processing | |

Exemplary plastid perturbation target genes from Arabidopsis with the accession number for the corresponding sequences in the Arabidopsis genome database (on the world wide web at the address “Arabidopsis.org”) are provided in Table 1. Orthologous genes from many crop species can be obtained through the BLAST comparison of the protein sequences of the Arabidopsis genes above to the genomic databases (NCBI and publically available genomic databases for specific crop species), as well as from the specific names of the subunits. Specifically the genome, cDNA, or EST sequences are available for apples, beans, barley, Brassica napus, rice, Cassava, Coffee, Eggplant, Orange, sorghum, tomato, cotton, grape, lettuce, tobacco, papaya, pine, rye, soybean, sunflower, peach, poplar, scarlet bean, spruce, cocoa, cowpea, maize, onion, pepper, potato, radish, sugarcane, wheat, and other species at the following interne or world wide web addresses: “compbio.dfci.harvard.edu/tgi/plant.html”; “genomevolution.org/wiki/index.php/Sequenced_plant_genomes”; “ncbi.nlm.nih.gov/genomes/PLANTS/PlantList.html”; “plantgdb.org/”; “arabidopsis.org/portals/genAnnotation/other_genomes/”; “gramene.org/resources/”; “genomenewsnetwork.org/resources/sequenced_genomes/genome_guide_pl.shtml”; “jgi.doe.gov/programs/plants/index.jsf”; “chibba.agtec.uga.edu/duplication/”; “mips.helmholtz-muenchen.de/plant/genomes.jsp”; “science.co.il/biomedical/Plant-Genome-Databases.asp”; “jcvi.org/cms/index.php?id=16”; and “phyto5.phytozome.net/Phytozome_resources.php”. The main protein complexes involved in photon capture and electron transport of photosystem II (PSII), NAD(P)H dehydrogenase (NDH), Cytochrome b6f complex, plastocyanin, photosystem I (PSI), and associated plastid proteins that represent certain plastid perturbation targets are also described in Grouneva, I., P. J. Gollan, et al. (2013) Planta 237(2): 399-412 Ifuku, K., S. Ishihara, et al. (2010). J Integr Plant Biol 52(8): 723-734.

In general, methods provided herewith for introducing epigenetic and/or genetic variation in plants simply require that plastid perturbation target gene expression be suppressed for a time sufficient to introduce the variation and/or in appropriate subsets of cells (i.e cells containing sensory plastids). As such, a wide variety of plastid perturbation target gene suppression methods can be employed to practice the methods provided herewith and the methods are not limited to a particular suppression technique.

Sequences of plastid perturbation target gene genes or fragments thereof from Arabidopsis and various crop plants are provided herewith. In certain embodiments, such genes may be used directly in either the homologous or a heterologous plant species to provide for suppression of the endogenous plastid perturbation target gene in either the homologous or heterologous plant species. A non-limiting, exemplary demonstration where an exemplary MSH1 plastid perturbation target gene from one species was shown to be effective in suppressing the endogenous MSH1 gene in both a homologous and a heterologous species is provided by Sandhu et al. 2007, where a transgene that provides for an MSH1 inhibitory RNA (RNAi) with tomato MSH1 sequences was shown to inhibit the endogenous MSH1 plastid perturbation target gene genes of both tomato and tobacco. A transgene that provides for a plastid perturbation target gene inhibitory RNA (RNAi) with maize plastid perturbation target gene sequences can be used in certain embodiments to inhibit the endogenous plastid perturbation target gene genes of millet, sorghum, and maize. Plastid perturbation target gene genes from other plants including, but not limited to, cotton, canola, wheat, barley, flax, oat, rye, turf grass, sugarcane, alfalfa, banana, broccoli, cabbage, carrot, cassava, cauliflower, celery, citrus, a cucurbit, eucalyptus, garlic, grape, onion, lettuce, pea, peanut, pepper, potato, poplar, pine, sunflower, safflower, soybean, strawberry, sugar beet, sweet potato, tobacco, cassava, cauliflower, celery, citrus, cotton, a cucurbit, eucalyptus, garlic, grape, onion, lettuce, pea, peanut, pepper, potato, poplar, pine, sunflower, safflower, strawberry, sugar beet, sweet potato, tobacco, cassava, cauliflower, celery, citrus, cucurbits, eucalyptus, garlic, grape, onion, lettuce, pea, peanut, pepper, poplar, pine, sunflower, safflower, soybean, strawberry, sugar beet, tobacco, Jatropha, Camelina, and Agave can be obtained by a variety of techniques and used to suppress expression of either the corresponding plastid perturbation target gene in those plants or the plastid perturbation target gene in a distinct plant. Methods for obtaining plastid perturbation target genes for various plants include, but are not limited to, techniques such as: i) searching amino acid and/or nucleotide sequence databases comprising sequences from the plant species to identify the plastid perturbation target gene by sequence identity comparisons; ii) cloning the plastid perturbation target gene by either PCR from genomic sequences or RT-PCR from expressed RNA; iii) cloning the plastid perturbation target gene from a genomic or cDNA library using PCR and/or hybridization based techniques; iv) cloning the plastid perturbation target gene from an expression library where an antibody directed to the plastid perturbation target gene protein is used to identify the plastid perturbation target gene containing clone; v) cloning the plastid perturbation target gene by complementation of an plastid perturbation target gene mutant or plastid perturbation target gene deficient plant; or vi) any combination of (i), (ii), (iii), (iv), and/or (v). The DNA sequences of the target genes can be obtained from the promoter regions or transcribed regions of the target genes by PCR isolation from genomic DNA, or PCR of the cDNA for the transcribed regions, or by commercial synthesis of the DNA sequence. RNA sequences can be chemically synthesized or, more preferably, by transcription of suitable DNA templates. Recovery of the plastid perturbation target gene from the plant can be readily determined or confirmed by constructing a plant transformation vector that provides for suppression of the gene, transforming the plants with the vector, and determining if plants transformed with the vector exhibit the characteristic responses that are typically observed in various plant species when MSH1 expression is suppressed that include leaf variegation, cytoplasmic male sterility (CMS), a reduced growth-rate phenotype, and/or delayed or non-flowering phenotype. The characteristic responses of MSH1 suppression have been described previously as developmental reprogramming or “MSH-dr1” (Xu et al. Plant Physiol. Vol. 159:711-720, 2012).

In certain embodiments, plastid perturbation target genes or fragments thereof used in the methods provided herein will have nucleotide sequences with at least 50%, 60%, 70%, 80%, 90%, 95%, 98%, 99%, or 100% nucleotide sequence identity to one or more of the plastid perturbation target genes or fragments thereof provided herein that include, but are not limited to, genes provided in Table 1 and orthologs thereof found in various crop plants. In certain embodiments, plastid perturbation target genes or fragments thereof used in the methods provided herein encode plastid perturbation target gene proteins or portions thereof will have amino acid sequences with at least 50%, 60%, 70%, 80%, 90%, 95%, 98%, 99%, or 100% amino acid sequence identity to one or more of the plastid perturbation target gene proteins provided herein that include, but are not limited to, the plastid perturbation target gene proteins encoded by genes provided in Table 1. In certain embodiments, plastid perturbation target genes or fragments thereof used in the methods provided herein will have nucleotide sequences with at least 50%, 60%, 70%, 80%, 90%, 95%, 98%, 99%, or 100% nucleotide sequence identity to one or more of the PPD3 plastid perturbation target genes fragments thereof, orthologs thereof, or homologs thereof, provided herein that include, but are not limited to, SEQ ID NO:16-40. In certain embodiments, plastid perturbation target gene genes or fragments thereof used in the methods provided herein encode plastid perturbation target gene proteins or portions thereof will have amino acid sequences with at least 50%, 60%, 70%, 80%, 90%, 95%, 98%, 99%, or 100% amino acid sequence identity to one or more of the PPD3 plastid perturbation target gene proteins or plastid perturbation target gene homologs provided herein that include, but are not limited to, the proteins encoded by SEQ ID NO:16-40. PPD3 plastid perturbation target gene genes from plants other than those provided herein can also be identified by the encoded regions with homology to the PsbP1 and PsbP2 gene domains that characterize many PPD3 genes.

It is anticipated that plastid perturbation target gene nucleic acid fragments of 18 to 20 nucleotides, but more preferably 21 nucleotides or more, can be used to effect suppression of the endogenous plastid perturbation target gene. In certain embodiments, plastid perturbation target gene nucleic acid fragments of at least 18, 19, 20, or 21 nucleotides to about 50, 100, 200, 500, or more nucleotides can be used to effect suppression of the endogenous plastid perturbation target gene. Regions of 20, 50, 100, 500, or more by are suitable for this purpose, with lengths of 100 to 300 bases of the target gene sequences preferable, and lengths of 300 to 500 bp or more being most preferable. For use in a hairpin or inverted repeat knockdown design, a spacer region with a sequence not related to the sequence of the genome of the target plant can be used. A hairpin construct containing 300 to 500 bp or more of a target gene sequence in the antisense orientation, followed by a spacer region whose sequence is not critical but can be a intron or non-intron. If the spacer is an intron, the caster bean catalase intron which is effectively spliced in both monocots and dicots (Tanaka, Mita et al. Nucleic Acids Res 18(23): 6767-6770, 1990), is known to those skilled in the art and is useful for the present embodiment. After the spacer the same target gene sequence in the sense orientation is present, such that the antisense and sense strands can form a double stranded RNA after transcription of the transcribed region. The target gene sequences are followed by a polyadenylation region. 3′ polyadenylation regions known to those skilled in the art to function in monocots and dicot plants include but are not limited to the Nopaline Synthase (NOS) 3′ region, the Octapine Synthase (OCS) 3′ region, the Cauliflower Mosaic Virus 35S 3′ region, the Mannopine Synthase (MAS) 3′ region. Additional 3′ polyadenylation regions from monocotyledonous genes such as those from rice, sorghum, wheat, and maize are available to those skilled in the art to provide similar polyadenylation region and function in DNA constructs in the present embodiments. In certain embodiments, a transgene designed to suppress a target gene in dicots is designed to have the following order: promoter/antisense to target gene/catalase intron/sense gene A/polyadenylation region. In embodiments where a gene is designed to suppress a target gene in monocots can have the following order: promoter/intron for monocots/antisense to target gene/catalase intron/sense gene A/polyadenylation region.

Sequences that provide for suppression of a plastid perturbation target gene can include sequences that exhibit complementarity to either strand of the promoter, 5′ or 3′ untranslated region, intron, coding regions, and/or any combination thereof. A target gene promoter region for gene suppression can include the transcription start site, the TATA box, and upstream regions. The promoter region for gene silencing can be about 20, 50, 80, or 100 nucleotides in length, and more preferably is about 100 to 500 nucleotides in length. The promoter region used for such suppression can be from different regions in the upstream promoter, preferably containing at least about 500 nucleotides upstream from the start of transcription, and most preferably containing at least about 500 nucleotides upstream from the start of translation of the native coding region of the native gene. This would include the UTR which may or may not be part of the promoter. A description of various recombinant DNA constructs that target promoter and/or adjoining regions of target genes are described in U.S. Pat. No. 8,293,975, which is incorporated herein by reference in its entirety.

For gene targets with closely related family members, sense, antisense or double hairpin suppression designs can include sequences from more than one family member, following the designs described above. In certain embodiments, a transgene to suppress two genes, target gene A and target gene B, is designed to have the following order: promoter/optional intron/antisense to target gene A/antisense to target gene B/spacer sequence/sense target gene B/sense gene A/polyadenylation region. In certain embodiments, this spacer sequence can be an intron. Exemplary embodiments include, but are not limited to, the following combinations of gene family members that can each be arranged in a single recombinant DNA construct any order that provides for hairpin formation and suppression of the gene targets:

(a) Construct 1: PsbQ-like PQL1, PsbQ-like, PsbQ-like PQL3, and any combination thereof;

### (b) Construct 2: PsbO-1 and PsbO-2;

### (c) Construct 3: PsbP1 and PsbP2;

### (d) Construct 4: PsbQ-1 and PsbQ-2;

### (e) Construct 5: FNR1 and FNR2;

(f) Construct 6: PETE1 and PETE2; and,

### (g) Construct 7: PsbW and PsbW-like.

In certain embodiments, suppression of plastid perturbation target gene in a plant is effected with a transgene. Transgenes that can be used to suppress expression of plastid perturbation target gene include, but are not limited to, transgenes that produce dominant-negative mutants of a plastid perturbation target gene, a small inhibitory RNA (siRNA), a microRNA (miRNA), a co-suppressing sense RNA, and/or an anti-sense RNA that provide for inhibition of the endogenous plastid perturbation target gene. U.S. patents incorporated herein by reference in their entireties that describe suppression of endogenous plant genes by transgenes include U.S. Pat. No. 7,109,393, U.S. Pat. No. 5,231,020 and U.S. Pat. No. 5,283,184 (co-suppression methods); and U.S. Pat. No. 5,107,065 and U.S. Pat. No. 5,759,829 (antisense methods). In certain embodiments, transgenes specifically designed to produce double-stranded RNA (dsRNA) molecules with homology to the plastid perturbation target gene can be used to decrease expression of the endogenous plastid perturbation target gene. In such embodiments, the sense strand sequences of the dsRNA can be separated from the antisense sequences by a spacer sequence, preferably one that promotes the formation of a dsRNA (double-stranded RNA) molecule. Examples of such spacer sequences include, but are not limited to, those set forth in Wesley et al., Plant J., 27(6):581-90 (2001), and Hamilton et al., Plant J., 15:737-746 (1998). One exemplary and non-limiting vector that has been shown to provide for suppression of plastid perturbation target gene in tobacco and tomato has been described by Sandhu et al., 2007 where an intron sequence separates the sense and antisense strands of the plastid perturbation target gene sequence. The design of recombinant DNA constructs for suppression of gene expression are also described in Helliwell, C. and P. Waterhouse (2003). “Constructs and methods for high-throughput gene silencing in plants.” Methods 30(4): 289-295.

In certain embodiments, transgenes that provide for plastid perturbation target gene suppression can comprise regulated promoters that provide for either induction or down-regulation of operably linked plastid perturbation target gene inhibitory sequences. In this context, plastid perturbation target gene inhibitory sequences can include, but are not limited to, dominant-negative mutants of plastid perturbation target gene, a small inhibitory RNA (siRNA), a microRNA (miRNA), a co-suppressing sense RNA, and/or an anti-sense RNA that provide for inhibition of the endogenous plastid perturbation target gene of a plant. Such promoters can provide for suppression of plastid perturbation target gene during controlled time periods by either providing or withholding the inducer or down regulator. Inducible promoters include, but are not limited to, a PR-1a promoter (U.S. Patent Application Publication Number 20020062502) or a GST II promoter (WO 1990/008826 A1). In other embodiments, both a transcription factor that can be induced or repressed as well as a promoter recognized by that transcription factor and operably linked to the plastid perturbation target gene inhibitory sequences are provided. Such transcription factor/promoter systems include, but are not limited to: i) RF2a acidic domain-ecdysone receptor transcription factors/cognate promoters that can be induced by methoxyfenozide, tebufenozide, and other compounds (U.S. Patent Application Publication Number 20070298499); ii) chimeric tetracycline repressor transcription factors/cognate chimeric promoters that can be repressed or de-repressed with tetracycline (Gatz, C., et al. (1992). Plant J. 2, 397-404), and the like.

In certain embodiments, a promoter that provides for selective expression of a heterologous sequence that suppresses expression of the target gene in cells containing sensory plastids is used. In certain embodiments, this promoter is an Msh1 or a PPD3 promoter. In certain embodiments, this promoter is an Msh1 or a PPD3 promoter and the operably linked heterologous sequence suppresses expression of a target gene provided in Table 1 (above). Msh1 promoters that can be used to express heterologous sequences in cells containing sensor plastids include, but are not limited to, the Arabidopsis, sorghum, tomato, and maize promoters provided herewith (SEQ ID NO:11, 12, 13, 14, and 41) as well as functional derivatives thereof that likewise provide for expression in cells that contain sensor plastids. In certain embodiments, deletion derivatives of the Msh1 promoters comprising about 1500 Bp, 1000 Bp, or about 750 Bp of SEQ ID NO:11, 12, 13, 14, and 41 can also be used to express heterologous sequences. PPD3 promoters that can be used to express heterologous sequences in cells containing sensor plastids include, but are not limited to, the Arabidopsis, rice, and tomato promoters provided herewith as SEQ ID NO:52, 53, and 54 as well as functional derivatives thereof that provide for expression in cells that contain sensor plastids. In certain embodiments, deletion derivatives of the Msh1 promoters comprising about 800 Bp, 600 Bp, or about 500 Bp of SEQ ID NO: 52, 53, and 54 can also be used to express heterologous sequences. In certain embodiments, PPD3 promoters comprising SEQ ID NO:52, 53, and 54 and an additional 200, 500, or 1000 basepairs of the endogenous 5′PPD3 promoter sequences can be used to express heterologous sequences. Additional 200, 500, or 1000 basepairs of the endogenous 5′PPD3 promoter sequences can be obtained by methods including, but not limited to, retrieval of sequences from databases provided herein and recovery of the adjoining promoter DNA by PCR amplification of genomic template sequences or by direct synthesis. In certain embodiments, recombinant DNA constructs for suppression of dicot target genes can comprise a MSH1 or PPD3 promoter from a dicotyledonous species such as Arabidopsis, soybeans or canola, is attached to a hairpin construct containing 300 to 500 bp or more of a target gene sequence in the antisense orientation, followed by a spacer region whose sequence is not critical but can be a intron or non-intron. The caster bean catalase intron (Tanaka, Mita et al. Nucleic Acids Res 18(23): 6767-6770, 1990), can be used as a spacer in certain embodiments. After the spacer the same target gene sequence in the sense orientation is present, such that the antisense and sense strands can form a double stranded RNA after transcription of the transcribed region. The target gene sequences are followed by a polyadenylation region. Various 3′ polyadenylation regions known to function in monocots and dicot plants include but are not limited to the Nopaline Synthase (NOS) 3′ region, the Octapine Synthase (OCS) 3′ region, the Cauliflower Mosaic Virus 35S 3′ region, the Mannopine Synthase (MAS) 3′ region. In certain embodiments recombinant DNA constructs for suppression of monocot target genes can comprise MSH1 or PPD3 promoter from a monocot species such as rice, maize, sorghum or wheat can either be attached directly to the hairpin region or to a monocot intron before the hairpin region. Monocot introns that are beneficial to gene expression when located between the promoter and coding region are the first intron of the maize ubiquitin (described in U.S. Pat. No. 6,054,574, which is incorporated herein by reference in its entirety) and the first intron of rice actin 1 (McElroy, Zhang et al. Plant Cell 2(2): 163-171, 1990). Additional introns that are beneficial to gene expression when located between the promoter and coding region are the maize hsp70 intron (described in U.S. Pat. No. 5,859,347, which is incorporated herein by reference in its entirety), and the maize alcohol dehydrogenase 1 genes introns 2 and 6 (described in U.S. Pat. No. 6,342,660, which is incorporated herein by reference in its entirety).

In still other embodiments, transgenic plants are provided where the transgene that provides for plastid perturbation target gene suppression is flanked by sequences that provide for removal for the transgene. Such sequences include, but are not limited to, transposable element sequences that are acted on by a cognate transposase. Non-limiting examples of such systems that have been used in transgenic plants include the cre-lox and FLP-FRT systems.

Any of the recombinant DNA constructs provided herein can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. Aforementioned methods of introducing transgenes are well known to those skilled in the art and are described in U.S. Patent Application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton). Plant transformation methods for producing transgenic plants include, but are not limited to methods for: Alfalfa as described in U.S. Pat. No. 7,521,600; Canola and rapeseed as described in U.S. Pat. No. 5,750,871; Cotton as described in U.S. Pat. No. 5,846,797; corn as described in U.S. Pat. No. 7,682,829. Indica rice as described in U.S. Pat. No. 6,329,571; Japonica rice as described in U.S. Pat. No. 5,591,616; wheat as described in U.S. Pat. No. 8,212,109; barley as described in U.S. Pat. No. 6,100,447; potato as described in U.S. Pat. No. 7,250,554; sugar beet as described in U.S. Pat. No. 6,531,649; and, soybean as described in U.S. Pat. No. 8,592,212.

In certain embodiments, plastid perturbation target gene suppression, including but not limited to suppression of an MSH1 or PPD3 gene, can initiate epigenetic modifications to produce useful traits (see U.S. Patent Application Publication No. US 2012/0284814, U.S. Provisional Patent Application 61/882,140, and U.S. Provisional Patent Application 61/901,349, each of which is incorporated by reference in its entirety). Plastid perturbation target gene suppression, including but not limited to suppression of an MSH1 or PPD3 gene, can be accomplished by any of the aforementioned suppression methods or by techniques including, but not limited to, topical RNA (U.S. Patent Application Publication No. US 2014/0018241 A1), promoter silencing (Deng et al., Plant Cell Physiol. 2014 Feb. 2), or site directed methods such as CRISPR/CAS9 methods (Jiang et al., Nucleic Acids Res. 2013 Nov. 1; 41(20):e188. doi: 10.1093/nar/gkt780. Epub 2013 Sep. 2).

Plastid perturbation target gene suppression can be readily identified or monitored by molecular techniques. In certain embodiments where the endogenous plastid perturbation target gene is intact but its expression is inhibited, production or accumulation of the RNA encoding plastid perturbation target gene can be monitored. Molecular methods for monitoring plastid perturbation target gene RNA expression levels include, but are not limited to, use of semi-quantitive or quantitative reverse transcriptase polymerase chain reaction (qRT-PCR) techniques. The use of semi-quantitive PCR techniques to monitor plastid perturbation target gene suppression resulting from RNAi mediated suppression of plastid perturbation target gene has been described (Sandhu et al. 2007). Various quantitative RT-PCR procedures including, but not limited to, TaqMan™ reactions (Applied Biosystems, Foster City, Calif. US), use of Scorpion™ or Molecular Beacon™ probes, or any of the methods disclosed in Bustin, S. A. (Journal of Molecular Endocrinology (2002) 29, 23-39) can be used. It is also possible to use other RNA quantitation techniques such as Quantitative Nucleic Acid Sequence Based Amplification (Q-NASBA™) or the Invader™ technology (Third Wave Technologies, Madison, Wis.).

In certain embodiments where plastid perturbation target gene suppression is achieved by use of a mutation in the endogenous plastid perturbation target gene of a plant, the presence or absence of that mutation in the genomic DNA can be readily determined by a variety of techniques. Certain techniques can also be used that provide for identification of the mutation in a hemizygous state (i.e. where one chromosome carries the mutated msh1 gene and the other chromosome carries the wild type plastid perturbation target gene). Mutations in plastid perturbation target DNA sequences that include insertions, deletions, nucleotide substitutions, and combinations thereof can be detected by a variety of effective methods including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entireties. For example, mutations can be detected by hybridization to allele-specific oligonucleotide (ASO) probes as disclosed in U.S. Pat. Nos. 5,468,613 and 5,217,863. U.S. Pat. No. 5,210,015 discloses detection of annealed oligonucleotides where a 5′ labelled nucleotide that is not annealed is released by the 5′-3′ exonuclease activity. U.S. Pat. No. 6,004,744 discloses detection of the presence or absence of mutations in DNA through a DNA primer extension reaction. U.S. Pat. No. 5,468,613 discloses allele specific oligonucleotide hybridizations where single or multiple nucleotide variations in nucleic acid sequence can be detected by a process in which the sequence containing the nucleotide variation is amplified, affixed to a support and exposed to a labeled sequence-specific oligonucleotide probe. Mutations can also be detected by probe ligation methods as disclosed in U.S. Pat. No. 5,800,944 where sequence of interest is amplified and hybridized to probes followed by ligation to detect a labeled part of the probe. U.S. Pat. Nos. 6,613,509 and 6,503,710, and references found therein provide methods for identifying mutations with mass spectroscopy. These various methods of identifying mutations are intended to be exemplary rather than limiting as the methods of the present invention can be used in conjunction with any polymorphism typing method to identify the presence of absence of mutations in a plastid perturbation target gene in genomic DNA samples. Furthermore, genomic DNA samples used can include, but are not limited to, genomic DNA isolated directly from a plant, cloned genomic DNA, or amplified genomic DNA. The use of mutations in endogenous PPD3 genes is specifically provided herein.

Mutations in endogenous plant plastid perturbation target gene genes can be obtained from a variety of sources and by a variety of techniques. A homologous replacement sequence containing one or more loss of function mutations in the plastid perturbation target gene and homologous sequences at both ends of the double stranded break can provide for homologous recombination and substitution of the resident wild-type plastid perturbation target gene sequence in the chromosome with a msh1 replacement sequence with the loss of function mutation(s). Such loss of function mutations include, but are not limited to, insertions, deletions, and substitutions of sequences within an plastid perturbation target gene that result in either a complete loss of plastid perturbation target gene function or a loss of plastid perturbation target gene function sufficient to elicit alterations (i.e. heritable and reversible epigenetic changes) in other chromosomal loci or mutations in other chromosomal loci. Loss-of-function mutations in plastid perturbation target gene include, but are not limited to, frameshift mutations, pre-mature translational stop codon insertions, deletions of one or more functional domains that include, but are not limited to, a DNA binding (Domain I), an ATPase (Domain V) domain, and/or a carboxy-terminal GIY-YIG type endonuclease domain, and the like. Also provided herein are mutations analogous the Arabidopsis msh1 mutation that are engineered into endogenous plastid perturbation target gene plant gene to obtain similar effects. Methods for substituting endogenous chromosomal sequences by homologous double stranded break repair have been reported in tobacco and maize (Wright et al., Plant J. 44, 693, 2005; D'Halluin, et al., Plant Biotech. J. 6:93, 2008). A homologous replacement msh1 sequence (i.e. which provides a loss of function mutation in an plastid perturbation target gene sequence) can also be introduced into a targeted nuclease cleavage site by non-homologous end joining or a combination of non-homologous end joining and homologous recombination (reviewed in Puchta, J. Exp. Bot. 56, 1, 2005; Wright et al., Plant J. 44, 693, 2005). In certain embodiments, at least one site specific double stranded break can be introduced into the endogenous plastid perturbation target gene by a meganuclease. Genetic modification of meganucleases can provide for meganucleases that cut within a recognition sequence that exactly matches or is closely related to specific endogenous plastid perturbation target gene sequence (WO/06097853A1, WO/06097784A1, WO/04067736A2, U.S. 20070117128A1). It is thus anticipated that one can select or design a nuclease that will cut within a target plastid perturbation target gene sequence. In other embodiments, at least one site specific double stranded break can be introduced in the endogenous plastid perturbation target gene target sequence with a zinc finger nuclease. The use of engineered zinc finger nuclease to provide homologous recombination in plants has also been disclosed (WO 03/080809, WO 05/014791, WO 07014275, WO 08/021207). In still other embodiments, mutations in endogenous plastid perturbation target gene genes can be identified through use of the TILLING technology (Targeting Induced Local Lesions in Genomes) as described by Henikoff et al. where traditional chemical mutagenesis would be followed by high-throughput screening to identify plants comprising point mutations or other mutations in the endogenous plastid perturbation target gene (Henikoff et al., Plant Physiol. 2004, 135:630-636). The recovery of mutations in endogenous PPD3 genes is specifically provided herein.

Any of the recombinant DNA constructs provided herein can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. Aforementioned methods of introducing transgenes are well known to those skilled in the art and are described in U.S. Patent Application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton), each of which are incorporated herein by reference in their entirety. Methods of using bacteria such as Rhizobium or Sinorhizobium to transform plants are described in Broothaerts, et al., Nature. 2005, 10; 433(7026):629-33. It is further understood that the recombinant DNA constructs can comprise cis-acting site-specific recombination sites recognized by site-specific recombinases, including Cre, Flp, Gin, Pin, Sre, pinD, Int-B13, and R. Methods of integrating DNA molecules at specific locations in the genomes of transgenic plants through use of site-specific recombinases can then be used (U.S. Pat. No. 7,102,055). Those skilled in the art will further appreciate that any of these gene transfer techniques can be used to introduce the recombinant DNA constructs into the chromosome of a plant cell, a plant tissue or a plant.

Methods of introducing plant minichromosomes comprising plant centromeres that provide for the maintenance of the recombinant minichromosome in a transgenic plant can also be used in practicing this invention (U.S. Pat. No. 6,972,197 and U.S. Patent Application Publication 20120047609). In these embodiments of the invention, the transgenic plants harbor the minichromosomes as extrachromosomal elements that are not integrated into the chromosomes of the host plant. It is anticipated that such mini-chromosomes may be useful in providing for variable transmission of a resident recombinant DNA construct that suppresses expression of a plastid perturbation target gene.

In certain embodiments, it is anticipated that ppd3 suppression can be effected by exposing whole plants, or reproductive structures of plants, to stress conditions that result in suppression of an endogenous PPD3gene. Such stress conditions include, but are not limited to, high light stress, and heat stress. Exemplary and non-limiting high light stress conditions include continuous exposure to about 300 to about 1200 μmol photons/m2.s for about 24 to about 120 hours. Exemplary and non-limiting heat stress conditions include continuous exposure to temperatures of about 32° C. to about 37° C. for about 2 hours to about 24 hours. Exemplary and non-limiting heat, light, and other environmental stress conditions that can provide for MSH1 suppression are also disclosed for heat (Shedge et al. 2010), high light stress (Xu et al. 2011) and other environmental stress conditions (Hruz et al. 2008) and can also be adapted to effect PPD3 suppression

Methods where plastid perturbation target gene suppression is effected in cultured plant cells are also provided herein. In certain embodiments, plastid perturbation target gene suppression can be effected by culturing plant cells under stress conditions that result in suppression of endogenous plastid perturbation target gene. Such stress conditions include, but are not limited to, high light stress. Exemplary and non-limiting high light stress conditions include continuous exposure to about 300 to about 1200 μmol photons/m2.s for about 24 to about 120 hours. Exemplary and non-limiting heat stress conditions include continuous exposure to temperatures of about 32° C. to about 37° C. for about 2 hours to about 24 hours. Exemplary and non-limiting heat, light, and other environmental stress conditions also that can provide for plastid perturbation target gene suppression are also disclosed for heat (Shedge et al. 2010), high light stress (Xu et al. 2011) and other environmental stress conditions (Hruz et al. 2008). In certain embodiments, plastid perturbation target gene suppression is effected in cultured plant cells by introducing a nucleic acid that provides for such suppression into the plant cells. Nucleic acids that can be used to provide for suppression of plastid perturbation target gene in cultured plant cells include, but are not limited to, transgenes that produce a small inhibitory RNA (siRNA), a microRNA (miRNA), a co-suppressing sense RNA, and/or an anti-sense RNA directed to the plastid perturbation target gene. Nucleic acids that can be used to provide for suppression of plastid perturbation target gene in cultured plant cells include, but are not limited to, a small inhibitory RNA (siRNA) or a microRNA (miRNA) directed against the endogenous plastid perturbation target gene. RNA molecules that provide for inhibition of plastid perturbation target gene can be introduced by electroporation. Introduction of inhibitory RNAs to cultured plant cells to inhibit target genes can in certain embodiments be accomplished as disclosed in Vanitharani et al. (Proc Natl Acad Sci USA., 2003, 100(16):9632-6), Qi et al. (Nucleic Acids Res. 2004 Dec. 15; 32(22):e179), or J. Cheon et al. (Microbiol. Biotechnol. (2009), 19(8), 781-786). The suppression of endogenous PPD3 genes in cultured plant cells is specifically provided herein.

Methods where plastid perturbation target gene suppression is effected in vegetatively or clonally propagated plant materials are also provided herein. Such vegetatively or clonally propagated plant materials can include, but are not limited to, cuttings, cultured plant materials, and the like. In certain embodiments, recovery of such plant or clonally propagated plant materials that have been subjected to plastid perturbation can be accomplished by methods that allow for transient suppression of the plastid perturbation target gene. In certain non-limiting examples, plant or clonally propagated plant materials that have been subjected to plant plastid perturbation are recovered by placing recombinant DNA constructs that suppress a plastid perturbation target gene in vectors that provide for their excision or segregation. In certain embodiments, such excision can be facilitated by use of transposase-based systems or such segregation can be facilitated by use of mini-chromosomes. In certain embodiments, such excision or segregation can be facilitated by linking a transgene that provides for a “conditional-lethal” counter selection to the transgene that suppresses a plastid perturbation target in the recombinant DNA construct. Vegetatively or clonally propagated plant materials that have been subjected to plastid perturbation and lacking recombinant DNA constructs that suppress a plastid perturbation target gene can then be screened and/or selected for useful traits. Also provided are methods where vegetatively or clonally propagated plant materials are obtained from a plant resulting from a self or outcross or from a cultured plant cell, where either the plant or plant cell had been subjected to suppression of a plastid perturbation target gene. Such vegetatively or clonally propagated plant materials obtained from such plants resulting from a self or outcross or from a plant cell that have been subjected to plastid perturbation can also be screened and/or selected for useful traits. Also provided herein are methods where a sexually reproducing plant or plant population comprising useful traits is vegetatively or clonally propagated, and a plant or a plant population derived therefrom is then used to produce seed or a seed lot. In certain embodiments of any of the aforementioned methods, the plastid perturbation target gene can be a MSH1 or a PPD3 gene.

Plastid perturbation target gene suppression can also be readily identified or monitored by traditional methods where plant phenotypes are observed. For example, plastid perturbation target gene suppression can be identified or monitored by observing organellar effects that include leaf variegation, cytoplasmic male sterility (CMS), a reduced growth-rate phenotype, and/or delayed or non-flowering phenotype. Phenotypes indicative of MSH1 plastid perturbation target gene suppression in various plants are provided in WO 2012/151254, which is incorporated herein by reference in its entirety. These phenotypes that are associated with plastid perturbation target gene suppression are referred to herein as “discrete variation” (V_{D}). Plastid perturbation target gene suppression can also produce changes in plant phenotypes including, but not limited to, plant tillering, height, internode elongation and stomatal density (referred to herein as “MSH1-dr”) that can be used to identify or monitor plastid perturbation target gene suppression in plants. Other biochemical and molecular traits can also be used to identify or monitor plastid perturbation target gene suppression in plants. Such molecular traits can include, but are not limited to, changes in expression of genes involved in cell cycle regulation, Giberrellic acid catabolism, auxin biosynthesis, auxin receptor expression, flower and vernalization regulators (i.e. increased FLC and decreased SOC1 expression), as well as increased miR156 and decreased miR172 levels. Such biochemical traits can include, but are not limited to, up-regulation of most compounds of the TCA, NAD and carbohydrate metabolic pathways, down-regulation of amino acid biosynthesis, depletion of sucrose in certain plants, increases in sugars or sugar alcohols in certain plants, as well as increases in ascorbate, alphatocopherols, and stress-responsive flavones apigenin, and apigenin-7-oglucoside, isovitexin, kaempferol 3-O-beta-glucoside, luteolin-7-O-glucoside, and vitexin. In certain embodiments, elevated plastochromanol-8 levels in plant stems can serve as a biochemical marker that can be used to identify or monitor plastid perturbation target gene suppression. In particular, plastochromanol-8 levels in stems of plants subjected to plastid perturbation target gene suppression can be compared to the levels in control plants that have not been subjected to such suppression to identify or monitor plastid perturbation target gene suppression. It is further contemplated that in certain embodiments, a combination of both molecular, biochemical, and traditional methods can be used to identify or monitor plastid perturbation target gene suppression in plants.

Plastid perturbation target gene suppression that results in useful epigenetic changes and useful traits can also be readily identified or monitored by assaying for characteristic DNA methylation and/or gene transcription patterns that occur in plants subject to such perturbations. In certain embodiments, characteristic DNA methylation and/or gene transcription patterns that occur in plants subject suppression of an MSH1 target gene can be monitored in a plant, a plant cell, plants, seeds, and/or processed products obtained therefrom to identify or monitor effects mediated by suppression of other target plant plastid perturbation genes. Such plant plastid perturbation genes that include, but are not limited to, genes provided herewith in the sequence listing and Table 1 are expected to give rise to the characteristic DNA methylation and/or gene transcription patterns that occur in plants subject suppression of an MSH1 target gene. Such characteristic DNA methylation and/or gene transcription patterns that occur in plants or seeds subjected suppression of an MSH1 target gene include, but are not limited to, those patterns disclosed herewith in Example 2. In certain embodiments, first generation progeny of a plant subjected to suppression of a plastid perturbation target gene will exhibit CG differentially methylated regions (DMR) of various discrete chromosomal regions that include, but are not limited to, regions that encompass the MSH1 locus. In certain embodiments, a CG hypermethylated region that encompasses the MSH1 locus will be about 5 to about 8 MBp (mega base pairs) in length. In certain embodiments, first generation progeny of a plant subjected to suppression of a plastid perturbation target gene will also exhibit changes in plant defense and stress response gene expression. In certain embodiments, a plant, a plant cell, a seed, plant populations, seed populations, and/or processed products obtained therefrom that has been subject to suppression of a plastid perturbation target gene will exhibit pericentromeric CHG hypermethylation and CG hypermethlation of various discrete or localized chromosomal regions. Such discrete or localized hypermethylation is distinct from generalized hypermethylation across chromosomes that has been previously observed (U.S. Pat. No. 6,444,469). Such CHG hypermethylation is understood to be methylation at the sequence “CHG” where H=A, T, or C. Such CG and CHG hypermethylation can be assessed by comparing the methylation status of a sample from plants or seed that had been subjected to suppression of a plastid perturbation target gene, or a sample from progeny plants or seed derived therefrom, to a sample from control plants or seed that had not been subjected to suppression of a plastid perturbation target gene. A variety of methods that provide for suppression of plastid perturbation target gene in a plant followed by recovery of progeny plants where plastid perturbation target gene function is recovered are provided herein. In certain embodiments, such progeny plants can be recovered by downregulating expression of a plastid perturbation target gene-inhibiting transgene or by removing the plastid perturbation target gene-inhibiting transgene with a transposase. In certain embodiments of the methods provided herein, plastid perturbation target gene is suppressed in a target plant or plant cell and progeny plants that express plastid perturbation target gene are recovered by genetic techniques. In one exemplary and non-limiting embodiment, progeny plants can be obtained by selfing a plant that is heterozygous for the transgene that provides for plastid perturbation target gene segregation. Selfing of such heterozygous plants (or selfing of heterozygous plants regenerated from plant cells) provides for the transgene to segregate out of a subset of the progeny plant population. Where a plastid perturbation target gene is suppressed by use of a recessive mutation in an endogenous plastid perturbation target gene can, in yet another exemplary and non-limiting embodiment, be crossed to wild-type plants that had not been subjected to plastid perturbation and then selfed to obtain progeny plants that are homozygous for a functional, wild-type plastid perturbation target gene allele. In other embodiments, a plastid perturbation target gene is suppressed in a target plant or plant cell and progeny plants that express the plastid perturbation target gene are recovered by molecular genetic techniques. Non limiting and exemplary embodiments of such molecular genetic techniques include: i) downregulation of an plastid perturbation target gene suppressing transgene under the control of a regulated promoter by withdrawal of an inducer required for activity of that promoter or introduction of a repressor of that promoter; or, ii) exposure of the an plastid perturbation target gene suppressing transgene flanked by transposase recognition sites to the cognate transposase that provides for removal of that transgene.

In order to restore plastid perturbation target gene expression, such as PPD3or MSH1 function, a plant heterozygous for a suppressing transgene can be selfed, backcrossed, or outcrossed to identify progeny that are not suppressed for target gene function. In order to restore plastid or MSH1 function for a plant homozygous for the suppressing transgene or mutation in MSH1, the plant is backcrossed or outcrossed, and then selfed, backcrossed, or outcrossed to identify progeny that are not suppressed for target gene function. Double haploid methods can be applied to progeny of a plant not suppressed for the target gene or its subsequent selfed, backcrossed, or outcrossed generations (S1-Sn, F2-Fn or the equivalent outcross or backcross generation), preferably the S1-S6, F1-F6, or equivalent generations if outcrossed or backcrossed, to provide epigenetically homozygous lines that exhibit useful traits, improved epigenetic stability, and lack the suppressing gene (see U.S. Provisional Application No. 61/930,602).

In certain embodiments of the methods provided herein, progeny plants derived from plants where plastid perturbation target gene expression was suppressed that exhibit male sterility, dwarfing, variegation, and/or delayed flowering time and express functional plastid perturbation target gene are obtained and maintained as independent breeding lines or as populations of plants. It has been found that such phenotypes appear to sort, so that it is feasible to select a cytoplasmic male sterile plant displaying normal growth rate and no variegation, for example, or a stunted, male fertile plant that is highly variegated. We refer to this phenomenon herein as discrete variation (V_{D}). Exemplary and non-limiting illustrations of this phenomenon as it occurs in selfed plant populations that have lost an MSH1 plastid perturbation target gene-inhibiting transgene by segregation have been disclosed (WO 2012/151254, incorporated herein by reference in its entirety). It is further contemplated that such individual lines that exhibit discrete variation (V_{D}) can be obtained by any of the aforementioned genetic techniques, molecular genetic techniques, or combinations thereof.

Individual lines obtained from plants where plastid perturbation target gene expression was suppressed that exhibit discrete variation (V_{D}) can be crossed to other plants to obtain progeny plants that lack the phenotypes associated with discrete variation (V_{D}) (i.e. male sterility, dwarfing, variegation, and/or delayed flowering time). In certain embodiments, progeny of such outcrosses can be selfed to obtain individual progeny lines that exhibit significant phenotypic variation. Such phenotypic variation that is observed in these individual progeny lines derived from outcrosses of plants where plastid perturbation target gene expression was suppressed and that exhibit discrete variation to other plants is herein referred to as “quantitative variation” (V_{Q}). Certain individual progeny plant lines obtained from the outcrosses of plants where plastid perturbation target gene expression was suppressed to other plants can exhibit useful phenotypic variation where one or more traits are improved relative to either parental line and can be selected. Useful phenotypic variation that can be selected in such individual progeny lines includes, but is not limited to, increases in fresh and dry weight biomass relative to either parental line. An exemplary and non-limiting illustration of this phenomenon as it occurs in F2 progeny of outcrosses of plants that exhibit discrete variation to plants that do not exhibit discrete variation is provided in U.S. Patent Application Publication No. 2012/0284814, which is incorporated herein by reference in its entirety.

Individual lines obtained from plants where plastid perturbation target gene expression was suppressed that exhibit discrete variation (V_{D}) can also be selfed to obtain progeny plants that lack the phenotypes associated with discrete variation (V_{D}) (i.e. male sterility, dwarfing, variegation, and/or delayed flowering time). Recovery of such progeny plants that lack the undesirable phenotypes can in certain embodiments be facilitated by removal of the transgene or endogenous locus that provides for plastid perturbation target gene suppression. In certain embodiments, progeny of such selfs can be used to obtain individual progeny lines or populations that exhibit significant phenotypic variation. Certain individual progeny plant lines or populations obtained from selfing plants where plastid perturbation target gene expression was suppressed can exhibit useful phenotypic variation where one or more traits are improved relative to the parental line that was not subjected to plastid perturbation target gene suppression and can be selected. Useful phenotypic variation that can be selected in such individual progeny lines includes, but is not limited to, increases in fresh and dry weight biomass relative to the parental line.

In certain embodiments, an outcross of an individual line exhibiting discrete variability can be to a plant that has not been subjected to plastid perturbation target gene suppression but is otherwise isogenic to the individual line exhibiting discrete variation. In certain exemplary embodiments, a line exhibiting discrete variation is obtained by suppressing plastid perturbation target gene in a given germplasm and can outcrossed to a plant having that same germplasm that was not subjected to plastid perturbation target gene suppression. In other embodiments, an outcross of an individual line exhibiting discrete variability can be to a plant that has not been subjected to plastid perturbation target gene suppression but is not isogenic to the individual line exhibiting discrete variation. Thus, in certain embodiments, an outcross of an individual line exhibiting discrete variability can also be to a plant that comprises one or more chromosomal polymorphisms that do not occur in the individual line exhibiting discrete variability, to a plant derived from partially or wholly different germplasm, or to a plant of a different heterotic group (in instances where such distinct heterotic groups exist). It is also recognized that such an outcross can be made in either direction. Thus, an individual line exhibiting discrete variability can be used as either a pollen donor or a pollen recipient to a plant that has not been subjected to plastid perturbation target gene suppression in such outcrosses. In certain embodiments, the progeny of the outcross are then selfed to establish individual lines that can be separately screened to identify lines with improved traits relative to parental lines. Such individual lines that exhibit the improved traits are then selected and can be propagated by further selfing. An exemplary and non-limiting illustration of this procedure where F2 progeny of outcrosses of plants that exhibit discrete variation to plants that do not exhibit discrete variation are obtained is provided in WO 2012/151254, which is incorporated herein by reference in its entirety. Such F2 progeny lines are screened for desired trait improvements relative to the parental plants and lines exhibiting such improvements are selected.

In certain embodiments, sub-populations of plants comprising the useful traits and epigenetic changes induced by suppression of the plastid perturbation target gene can be selected and bred as a population. Such populations can then be subjected to one or more additional rounds of selection for the useful traits and/or epigenetic changes to obtain subsequent sub-populations of plants exhibiting the useful trait. Any of these sub-populations can also be used to generate a seed lot. In an exemplary embodiment, plastid perturbed plants exhibiting an Msh1-dr phenotype can be selfed or outcrossed to obtain an F1 generation. A bulk selection at the F1, F2, and/or F3 generation can thus provide a population of plants exhibiting the useful trait and/or epigenetic changes or a seed lot. In certain embodiments, it is also anticipated that populations of progeny plants or progeny seed lots comprising a mixture of inbred an hybrid germplasms can be derived from populations comprising hybrid germplasm (i.e. plants arising from cross of one inbred line to a distinct inbred line). Seed lots thus obtained from these exemplary method or other methods provided herein can comprise seed wherein at least 25%, 50%, 60%, 70%, 80%, 90%, or 95% of progeny plants grown from the seed exhibit a useful trait. The selection would provide the most robust and vigorous of the population for seed lot production. Seed lots produced in this manner could be used for either breeding or sale. In certain embodiments, a seed lot comprising seed wherein at least 25%, 50%, 60%, 70%, 80%, 90%, or 95% of progeny plants grown from the seed exhibit a useful trait associated with one or moreepigenetic changes, wherein the epigenetic changes are associated with CG hyper-methylation and/or CHG hyper-methylation at one or more nuclear chromosomal loci in comparison to a control plant that does not exhibit the useful trait, and wherein the seed or progeny plants grown from said seed that is epigenetically heterogenous are obtained. A seed lot obtainable by these methods can include at least 100, 500, 1000, 5000, or 10,000 seeds.

Altered chromosomal loci that can confer useful traits can also be identified and selected by performing appropriate comparative analyses of reference plants that do not exhibit the useful traits and test plants obtained from a parental plant or plant cell that had been subjected to plastid perturbation target gene suppression and obtaining either the altered loci or plants comprising the altered loci. It is anticipated that a variety of reference plants and test plants can be used in such comparisons and selections. In certain embodiments, the reference plants that do not exhibit the useful trait include, but are not limited to, any of: a) a wild-type plant; b) a distinct subpopulation of plants within a given F2 population of plants of a given plant line (where the F2 population is any applicable plant type or variety); c) an F1 population exhibiting a wild type phenotype (where the F1 population is any applicable plant type or variety); and/or, d) a plant that is isogenic to the parent plants or parental cells of the test plants prior to suppression of plastid perturbation target gene in those parental plants or plant cells (i.e. the reference plant is isogenic to the plants or plant cells that were later subjected to plastid perturbation target gene suppression to obtain the test plants). In certain embodiments, the test plants that exhibit the useful trait include, but are not limited to, any of: a) any non-transgenic segregants that exhibit the useful trait and that were derived from parental plants or plant cells that had been subjected to transgene mediated plastid perturbation target gene suppression, b) a distinct subpopulation of plants within a given F2 population of plants of a given plant line that exhibit the useful trait (where the F2 population is any applicable plant type or variety); (c) any progeny plants obtained from the plants of (a) or (b) that exhibit the useful trait; or d) a plant or plant cell that had been subjected to plastid perturbation target gene suppression that exhibit the useful trait.

In general, an objective of these comparisons is to identify differences in the small RNA profiles and/or methylation of certain chromosomal DNA loci between test plants that exhibit the useful traits and reference plants that do not exhibit the useful traits. Altered loci thus identified can then be isolated or selected in plants to obtain plants exhibiting the useful traits.

In certain embodiments, altered chromosomal loci can be identified by identifying small RNAs that are up or down regulated in the test plants (in comparison to reference plants). This method is based in part on identification of altered chromosomal loci where small interfering RNAs direct the methylation of specific gene targets by RNA-directed DNA methylation (RdDM). The RNA-directed DNA methylation (RdDM) process has been described (Chinnusamy V et al. Sci China Ser C-Life Sci. (2009) 52(4): 331-343). Any applicable technology platform can be used to compare small RNAs in the test and reference plants, including, but not limited to, microarray-based methods (Franco-Zorilla et al. Plant J. 2009 59(5):840-50), deep sequencing based methods (Wang et al. The Plant Cell 21:1053-1069 (2009)), and the like.

In certain embodiments, altered chromosomal loci can be identified by identifying histone proteins associated with a locus and that are methylated or acylated in the test plants (in comparison to reference plants). The analysis of chromosomal loci associated with methylated or acylated histones can be accomplished by enriching and sequencing those loci using antibodies that recognize methylated or acylated histones. Identification of chromosomal regions associated with methylation or acetylation of specific lysine residues of histone H3 by using antibodies specific for H3K4me3, H3K9ac, H3K27me3, and H3K36me3 has been described (Li et al., Plant Cell 20:259-276, 2008; Wang et al. The Plant Cell 21:1053-1069 (2009).

In certain embodiments, altered chromosomal loci can be identified by identifying chromosomal regions (genomic DNA) that has an altered methylation status in the test plants (in comparison to reference plants). An altered methylation status can comprise either the presence or absence of methylation in one or more chromosomal loci of a test plant comparison to a reference plant. Any applicable technology platform can be used to compare the methylation status of chromosomal loci in the test and reference plants. Applicable technologies for identifying chromosomal loci with changes in their methylation status include, but not limited to, methods based on immunoprecipitation of DNA with antibodies that recognize 5-methylcytidine, methods based on use of methylation dependent restriction endonucleases and PCR such as McrBC-PCR methods (Rabinowicz, et al. Genome Res. 13: 2658-2664 2003; Li et al., Plant Cell 20:259-276, 2008), sequencing of bisulfite-converted DNA (Frommer et al. Proc. Natl. Acad. Sci. U.S.A. 89 (5): 1827-31; Tost et al. BioTechniques 35 (1): 152-156, 2003), methylation-specific PCR analysis of bisulfate treated DNA (Herman et al. Proc. Natl. Acad. Sci. U.S.A. 93 (18): 9821-6, 1996), deep sequencing based methods (Wang et al. The Plant Cell 21:1053-1069 (2009)), methylation sensitive single nucleotide primer extension (MsSnuPE; Gonzalgo and Jones Nucleic Acids Res. 25 (12): 2529-2531, 1997), fluorescence correlation spectroscopy (Umezu et al. Anal Biochem. 415(2):145-50, 2011), single molecule real time sequencing methods (Flusberg et al. Nature Methods 7, 461-465), high resolution melting analysis (Wojdacz and Dobrovic (2007) Nucleic Acids Res. 35 (6): e41), and the like.

Methods for introducing various chromosomal modifications that can confer a useful trait into a plant, as well as the plants, plant parts, and products of those plant parts are also provided herein. Chromosomal alterations and/or chromosomal mutations induced by suppression of plastid perturbation target gene can be identified as described herein. Once identified, chromosomal modifications including, but not limited to, chromosomal alterations, chromosomal mutations, or transgenes that provide for the same genetic effect as the chromosomal alterations and/or chromosomal mutations induced by suppression of plastid perturbation target gene can be introduced into host plants to obtain plants that exhibit the desired trait. In this context, the “same genetic effect” means that the introduced chromosomal modification provides for an increase and/or a reduction in expression of one or more endogenous plant genes that is similar to that observed in a plant that has been subjected to plastid perturbation target gene suppression and exhibits the useful trait. In certain embodiments where an endogenous gene is methylated in a plant subjected to plastid perturbation target gene suppression and exhibits both reduced expression of that gene and a useful trait, chromosomal modifications in other plants that also result in reduced expression of that gene and the useful trait are provided. In certain embodiments where an endogenous gene is demethylated in a plant subjected to plastid perturbation target gene suppression and exhibits both increased expression of that gene and a useful trait, chromosomal modifications in other plants that also result in increased expression of that gene and that useful trait are provided.

In certain embodiments, the chromosomal modification that is introduced is a chromosomal alteration. Chromosomal alterations including, but not limited to, a difference in a methylation state can be introduced by crossing a plant comprising the chromosomal alteration to a plant that lacks the chromosomal alteration and selecting for the presence of the alteration in F1, F2, or any subsequent generation progeny plants of the cross. In still other embodiments, the chromosomal alterations in specific target genes can be introduced by expression of a siRNA or hairpin RNA targeted to that gene by RNA directed DNA methylation (Chinnusamy V et al. Sci China Ser C-Life Sci. (2009) 52(4): 331-343; Cigan et al. Plant J 43 929-940, 2005; Heilersig et al. (2006) Mol Genet Genomics 275 437-449; Miki and Shimamoto, Plant Journal 56(4):539-49; Okano et al. Plant Journal 53(1):65-77, 2008).

In certain embodiments, the chromosomal modification is a chromosomal mutation. Chromosomal mutations that provide for reductions or increases in expression of an endogenous gene of a chromosomal locus can include, but are not limited to, insertions, deletions, and/or substitutions of nucleotide sequences in a gene. Chromosomal mutations can result in decreased expression of a gene by a variety of mechanisms that include, but are not limited to, introduction of missense codons, frame-shift mutations, premature translational stop codons, promoter deletions, mutations that disrupt mRNA processing, and the like. Chromosomal mutations that result in increased expression of a gene include, but are not limited to, promoter substitutions, removal of negative regulatory elements from the gene, and the like. Chromosomal mutations can be introduced into specific loci of a plant by any applicable method. Applicable methods for introducing chromosomal mutations in endogenous plant chromosomal loci include, but are not limited to, homologous double stranded break repair (Wright et al., Plant J. 44, 693, 2005; D'Halluin, et al., Plant Biotech. J. 6:93, 2008), non-homologous end joining or a combination of non-homologous end joining and homologous recombination (reviewed in Puchta, J. Exp. Bot. 56, 1, 2005; Wright et al., Plant J. 44, 693, 2005), meganuclease-induced, site specific double stranded break repair (WO/06097853A1, WO/06097784A1, WO/04067736A2, U.S. 20070117128A1), and zinc finger nuclease mediated homologous recombination (WO 03/080809, WO 05/014791, WO 07014275, WO 08/021207). In still other embodiments, desired mutations in endogenous plant chromosomal loci can be identified through use of the TILLING technology (Targeting Induced Local Lesions in Genomes) as described (Henikoff et al., Plant Physiol. 2004, 135:630-636).

In other embodiments, chromosomal modifications that provide for the desired genetic effect can comprise a transgene. Transgenes that can result in decreased expression of an gene by a variety of mechanisms that include, but are not limited to, dominant-negative mutants, a small inhibitory RNA (siRNA), a microRNA (miRNA), a co-suppressing sense RNA, and/or an anti-sense RNA and the like. U.S. patents incorporated herein by reference in their entireties that describe suppression of endogenous plant genes by transgenes include U.S. Pat. No. 7,109,393, U.S. Pat. No. 5,231,020 and U.S. Pat. No. 5,283,184 (co-suppression methods); and U.S. Pat. No. 5,107,065 and U.S. Pat. No. 5,759,829 (antisense methods). In certain embodiments, transgenes specifically designed to produce double-stranded RNA (dsRNA) molecules with homology to the endogenous gene of a chromosomal locus can be used to decrease expression of that endogenous gene. In such embodiments, the sense strand sequences of the dsRNA can be separated from the antisense sequences by a spacer sequence, preferably one that promotes the formation of a dsRNA (double-stranded RNA) molecule. Examples of such spacer sequences include, but are not limited to, those set forth in Wesley et al., Plant J., 27(6):581-90 (2001), and Hamilton et al., Plant J., 15:737-746 (1998). Vectors for inhibiting endogenous plant genes with transgene-mediated expression of hairpin RNAs are disclosed in U.S. Patent Application Nos. 20050164394, 20050160490, and 20040231016, each of which is incorporated herein by reference in their entirety.

Transgenes that result in increased expression of a gene of a chromosomal locus include, but are not limited to, a recombinant gene fused to heterologous promoters that are stronger than the native promoter, a recombinant gene comprising elements such as heterologous introns, 5′ untranslated regions, 3′ untranslated regions that provide for increased expression, and combinations thereof. Such promoter, intron, 5′ untranslated, 3′ untranslated regions, and any necessary polyadenylation regions can be operably linked to the DNA of interest in recombinant DNA molecules that comprise parts of transgenes useful for making chromosomal modifications as provided herein.

Exemplary promoters useful for expression of transgenes include, but are not limited to, enhanced or duplicate versions of the viral CaMV35S and FMV35S promoters (U.S. Pat. No. 5,378,619, incorporated herein by reference in its entirety), the cauliflower mosaic virus (CaMV) 19S promoters, the rice Act1 promoter and the Figwort Mosaic Virus (FMV) 35S promoter (U.S. Pat. No. 5,463,175; incorporated herein by reference in its entirety). Exemplary introns useful for transgene expression include, but are not limited to, the maize hsp70 intron (U.S. Pat. No. 5,424,412; incorporated by reference herein in its entirety), the rice Act1 intron (McElroy et al., 1990, The Plant Cell, Vol. 2, 163-171), the CAT-1 intron (Cazzonnelli and Velten, Plant Molecular Biology Reporter 21: 271-280, September 2003), the pKANNIBAL intron (Wesley et al., Plant J. 2001 27(6):581-90; Collier et al., 2005, Plant J 43: 449-457), the PIV2 intron (Mankin et al. (1997) Plant Mol. Biol. Rep. 15(2): 186-196) and the “Super Ubiquitin” intron (U.S. Pat. No. 6,596,925, incorporated herein by reference in its entirety; Collier et al., 2005, Plant J 43: 449-457). Exemplary polyadenylation sequences include, but are not limited to, and Agrobacterium tumor-inducing (Ti) plasmid nopaline synthase (NOS) gene and the pea ssRUBISCO E9 gene polyadenylation sequences.

Methods for producing a plant exhibiting new combinations of altered chromosomal loci useful for breeding comprising the steps of: (a) crossing a plant comprising altered chromosomal loci induced by MSH1 suppression to produce progeny; and, (b) assaying the DNA methylation of said progeny to identify and select individuals with new combinations of altered chromosomal loci are provided herein. In some embodiments altered chromosomal loci are selected from the group consisting of MSH1, pericentromeric regions, CG enhanced genes, CG depleted genes, transposable elements, transposable elements containing genes, and transposable elements in pericentromeric regions. In certain embodiments DNA methylation of altered chromosomal loci occurs at CHG or CHH sites within a DNA region selected from the group consisting of MSH1, pericentromeric regions, transposable elements, transposable elements containing genes, and transposable elements in pericentromeric regions. In certain embodiments DNA methylation of altered chromosomal loci occurs at CG sequences near or within one or more CG altered genes.

Methods for producing a plant exhibiting new combinations of altered chromosomal loci useful for breeding comprising the steps of: (a) crossing a plant comprising altered chromosomal loci induced by MSH1 suppression to produce progeny; and, (b) assaying one or more sRNAs of said progeny to identify and select individuals with new combinations of altered chromosomal loci are also provided. In certain embodiments one or more sRNAs assayed have sequence homology to the group consisting of MSH1, pericentromeric regions, CG enhanced genes, CG depleted genes, transposable elements, transposable elements containing genes, and transposable elements in pericentromeric regions.

Methods for identifying a plant with altered chromosomal loci useful for plant breeding comprising the steps of: (a) assaying DNA methylation of one or more plants comprising altered chromosomal loci induced by MSH1 suppression; and, (b) identifying one or more plants from step (a) comprising one or more altered chromosomal loci selected from the group consisting of MSH1, pericentromeric regions, CG enhanced genes, CG depleted genes, transposable elements, transposable elements containing genes, and transposable elements in pericentromeric regions are provided. In certain embodiments DNA methylation of altered chromosomal loci occurs at CHG or CHH at DNA sequences selected from the group consisting of MSH1, pericentromeric regions, transposable elements, transposable elements containing genes, and transposable elements in pericentromeric regions. In certain embodiments DNA methylation of altered chromosomal loci occurs at CG sequences near or within one or more CG altered genes.

Methods for identifying a plant with altered chromosomal loci useful for plant breeding comprising the steps of: (a) assaying one or more sRNAs of one or more plants comprising altered chromosomal loci induced by MSH1 suppression; and, (b) identifying one or more plants from step (a) comprising one or more increases or decreases in one or more sRNAs with homology at DNA sequences selected from the group of altered chromosomal loci consisting of MSH1, pericentromeric regions, CG enhanced genes, CG depleted genes, transposable elements, transposable elements containing genes, and transposable elements in pericentromeric regions are provided.

Methods for producing a plant exhibiting new combinations of altered chromosomal loci useful for breeding comprising the steps of: (a) selfing a plant comprising altered chromosomal loci induced by MSH1 suppression to produce progeny; and, (b) assaying the DNA methylation at altered chromosomal loci of said progeny to identify and select individuals with new combinations of altered chromosomal loci are provided. In certain embodiments altered chromosomal loci are selected from the group consisting of MSH1, pericentromeric regions, CG enhanced genes, CG depleted genes, transposable elements, transposable elements containing genes, and transposable elements in pericentromeric regions. In certain embodiments DNA methylation of altered chromosomal loci occurs at CHG or CHH sites within a DNA region selected from the group consisting of MSH1, pericentromeric regions, transposable elements, transposable elements containing genes, and transposable elements in pericentromeric regions. In certain embodiments DNA methylation of altered chromosomal loci occurs at CG sequences near or within one or more CG altered genes.

Methods for producing a plant exhibiting new combinations of altered chromosomal loci useful for breeding comprising the steps of: (a) selfing a plant comprising altered chromosomal loci induced by MSH1 suppression to produce progeny; and, (b) assaying one or more sRNAs of said progeny to identify and select individuals with new combinations of altered chromosomal loci are also provided. In certain embodiments one or more sRNAs assayed have sequence homology to the group of altered chromosomal loci consisting of MSH1, pericentromeric regions, CG enhanced genes, CG depleted genes, transposable elements, transposable elements containing genes, and transposable elements in pericentromeric regions.

Methods for selecting a plant comprising one or more altered chromosomal loci useful for plant breeding comprising the steps of: a) comparing the DNA methylation status of one or more nuclear chromosomal regions in a reference plant to one or more corresponding nuclear chromosomal regions in a candidate plant, wherein said candidate plant or its progenitor was obtained by suppression of MSH1; and, b) selecting a candidate plant comprising one or more nuclear chromosomal regions present in the candidate plant with a DNA methylation status that is distinct from the DNA methylation status in the reference plant, thereby selecting a plant comprising one or more altered chromosomal loci useful for plant breeding are also provided.

Methods for selecting a plant comprising one or more altered chromosomal loci useful for plant breeding comprising the steps of: a) comparing one or more sRNAs with homology to one or more nuclear chromosomal regions in a reference plant to one or more sRNAs from corresponding nuclear chromosomal regions in a candidate plant, wherein said candidate plant or its progenitor was obtained by suppression of MSH1; and, b) selecting a candidate plant comprising one or more sRNA with abundances or sequences that are distinct from the sRNAs in the reference plant, thereby selecting a plant comprising one or more altered chromosomal loci useful for plant breeding are provided herein.

In certain embodiments of the methods, the DNA methylation status comprises at least one nucleotide position or region of CG hypermethylation, CHG hypermethylation, or CHH hypermethylation. In certain embodiments of the methods, the DNA methylation status comprises at least one nucleotide position or region of CG hypomethylation, CHG hypomethylation, or CHH hypomethylation. In certain embodiments of the methods, the DNA methylation status comprises hypermethylation and hypomethylation in chromosomal regions comprising sequences selected from the group of CG, CHG, and CHH DNA sequences.

In certain embodiments vegetatively or clonally propagated plant materials are derived from any of the aforementioned methods. Such vegetatively or clonally propagated plant materials can also be screened and/or selected for useful traits. Also provided herein are methods where a sexually reproducing plant or plant population comprising useful altered chromosomal loci is vegetatively or clonally propagated, and a plant or a plant population derived therefrom is then used to produce seed or a seed lot.

In certain embodiments of any of the aforementioned methods, the plant is a crop plant. In certain embodiments the crop plant is from the group consisting of corn, wheat, rice, sorghum, millet, tomato, potato, soybean, tobacco, cotton, canola, alfalfa, rapeseed, sugar beets, and sugarcane.

In certain embodiments of any of the aforementioned methods, the plants include, but are not limited to those from, millet, sorghum, maize, cotton, canola, wheat, barley, flax, oat, rye, turf grass, sugarcane, alfalfa, banana, broccoli, cabbage, carrot, cassava, cauliflower, celery, citrus, a cucurbit, eucalyptus, garlic, grape, onion, lettuce, pea, peanut, pepper, potato, poplar, pine, sunflower, safflower, soybean, strawberry, sugar beet, sweet potato, tobacco, cassava, cauliflower, celery, citrus, cotton, a cucurbit, eucalyptus, garlic, grape, onion, lettuce, pea, peanut, pepper, potato, poplar, pine, sunflower, safflower, strawberry, sugar beet, sweet potato, tobacco, cassava, cauliflower, celery, citrus, cucurbits, eucalyptus, garlic, grape, onion, lettuce, pea, peanut, pepper, poplar, pine, sunflower, safflower, soybean, strawberry, sugar beet, tobacco, Jatropha, Camelina, and Agave.

In general, methods provided herewith: a) introduce DNA methylation changes in plants and measure the changes in DNA methylation in said plants and/or their progeny; b) select said plants and/or their progeny for increased or decreased DNA methylation, either by measuring DNA methylation directly or as inferred by measuring sRNA levels from the corresponding DNA region, at DNA regions of at least one of the group of regions consisting of MSH1, pericentromeric regions, CG enhanced genes, CG depleted genes, transposable elements, transposable elements containing genes, and transposable elements in pericentromeric regions. In certain embodiments, first or later generation progeny of a plant subjected to MSH1 suppression will exhibit CG differentially methylated positions or regions of various discrete chromosomal regions that include, but are not limited to, regions that encompass the MSH1 locus. In certain embodiments, a CG hypermethylated region that encompasses the MSH1 locus will be up to about 8 Mbp (mega base pairs) in length. In certain embodiments, a plant, a plant cell, a seed, plant populations, seed populations, and/or processed products obtained from a progenitor that has been subject to MSH1 suppression will exhibit pericentromeric CHG, CHH, and/or CG hypermethlation of various discrete or localized chromosomal regions. Such discrete or localized hypermethylation is distinct from generalized hypermethylation across chromosomes that has been previously observed (U.S. Pat. No. 6,444,469).

In general, changes in DNA methylation are mostly accompanied by changes in small RNA (sRNA) profiles, particularly sRNAs of 20 to 24 nucleotides in length and microRNAs (Bond et. al., Trends Cell Biol. 2014 Feb. 24(2):100-7; Bologna et al., Annu Rev. Plant Biol. 2014 Feb. 26; Hu et al., Biochem Biophys Res Commun. 2014 Feb. 21; 444(4):676-81.), making assaying sRNA levels an alternative or complementary method for measuring changes in DNA methylation levels. Accordingly, an objective is to identify differences in one or more sRNAs derived from certain altered chromosomal loci between candidate plants and isogenic reference plants not derived from MSH1 suppressed plants. Altered chromosomal loci thus identified can then be isolated or selected in plants to obtain plants useful for plant breeding to develop improved traits selected from the group consisting of improved yield, delayed flowering, non-flowering, increased biotic stress resistance, increased abiotic stress resistance, enhanced lodging resistance, enhanced growth rate, enhanced biomass, enhanced tillering, enhanced branching, delayed flowering time, and delayed senescence.

In certain embodiments, altered chromosomal loci can be identified by identifying sRNAs that are up or down regulated in the candidate plants in comparison to reference plants. These methods are based in part on identification of altered chromosomal loci where small interfering sRNAs direct the methylation of specific gene targets by RNA-directed DNA methylation (RdDM). The RNA-directed DNA methylation (RdDM) process has been described (Chinnusamy V et al. Sci China Ser C-Life Sd. (2009) 52(4): 33 1-343; Bond et. al., Trends Cell Biol. 2014 Feb. 24(2):100-7). Any applicable technology platform can be used to compare small RNAs in the test and reference plants, including, but not limited to: microarray-based methods (Franco-Zorilla et al. Plant J. 200959(5):840-50); deep sequencing based methods (Wang et al. The Plant Cell 21:1053-1069(2009); Wei et al., Proc Natl Acad Sci USA. 2014 Feb. 19, 111(10): 3877-3882; Zhai et al., Methods. 2013 Jun. 28. pii: S1046-2023(13)00237-5. doi: 10.1016/j.ymeth.2013.06.025 or J. Zhai et al., Methods (2013), http://dx.doi.org/10.1016/j.ymeth.2013.06.025); U.S. Pat. No. 7,550,583; U.S. Pat. No. 8,399,221; U.S. Pat. No. 8,399,222; U.S. Pat. No. 8,404,439; U.S. Pat. No. 8,637,276; Rosas-Cárdenas et al., (2011) Plant Methods 2011, 7:4; Moyano et al., BMC Genomics. 2013 Oct. 11; 14:701; Eldem et al., PLoS One. 2012; 7(12):e50298; Barber et al., Proc Natl Acad Sci USA. 2012 Jun. 26; 109(26):10444-9; Gommans et al., Methods Mol Biol. 2012; 786:167-78; and the like.

DNA methylation and sRNAs corresponding to these regions can change in progeny plants when two parent plants are crossed. Tomato progeny plants from a cross displayed transgressive sRNAs that were more abundant in the progeny than in either parent (Shivaprasad et al., EMBO J. 2012 Jan. 18; 31(2):257-66). A cross between two maize lines, B73 and Mo17, yielded paramutation type switches of the DNA methylation pattern of one parent chromosome being switched to that of the other parental chromosome at the corresponding loci (Regulski et al., Genome Res. 2013 October; 23(10):1651-62). A cross between Arabidopsis plants produced progeny wherein the DNA methylation patterns of one parental chromosome were imposed onto the other parental chromosome, either gaining or losing DNA methylation levels (Greaves et al., Proc Natl Acad Sci USA. 2014 Feb. 4; 111(5):2017-22). These non-limiting examples indicate DNA methylation patterns can be more complex than just additive patterns from both parents. Accordingly, an objective is to identify new combinations of altered chromosomal loci in progeny plants that have new patterns of DNA methylation and/or of sRNA profiles. New combinations of altered chromosomal loci can result both from segregation of altered chromosomal loci in the progeny as well as due to changes in DNA methylation and sRNA profiles due to transgressive, paramutation type switching, and other biological processes. In certain embodiments, altered chromosomal loci are derived from a parental plant subjected to suppression of MSH1. In certain embodiments, altered chromosomal loci are derived from the formation of new patterns of DNA methylation and sRNA levels from the interaction of altered chromosomal loci derived from a parental plant subjected to suppression of MSH1 with chromosomal loci from a second plant. Said second plant can be from a parental plant subjected to suppression of MSH1 or from a parental plant not subjected to suppression of MSH1. Crossing parental lines both previously subjected to MSH1 suppression and containing different groupings of altered chromosomal loci provides a method of creating new combinations of altered chromosomal loci.

In certain embodiments, altered chromosomal loci can be identified by identifying chromosomal regions (genomic DNA) that have an altered methylation status in the test plants (in comparison to a reference plant). An altered methylation status can comprise either the presence or absence of methylation in one or more chromosomal loci of a test plant in comparison to a reference plant. Any applicable technology can be used to compare the methylation status of chromosomal loci in the test and reference plants. Applicable technologies for identifying chromosomal loci with changes in their methylation status include, but not limited to, methods based on immunoprecipitation of DNA with antibodies that recognize 5-methyl-cytidine, methods based on use of methylation dependent restriction endonucleases and PCR such as McrBC-PCR methods (Rabinowicz, et al. Genome Res. 13: 2658-2664 2003; Li et al., Plant Cell 20:259-276, 2008), sequencing of bisulfite-converted DNA (Frommer et al. Proc. Nat!. Acad. Sci. U.S.A. 89 (5): 1827-31; Tost et al. BioTechniques 35 (1): 152-156, 2003), methylation-pericentromeric regions specific PCR analysis of bisulfite treated DNA (Herman et al. Proc. Natl. Acad. Sci. U.S.A. 93 (18): 9821-6, 1996), deep sequencing based methods (Wang et al. The Plant Cell 21:1053-1069 (2009)), methylation sensitive single nucleotide primer extension (MsSnuPE; Gonzalgo and Jones Nucleic Acids Res. 25 (12): 2529-2531, 1997), fluorescence correlation spectroscopy (Umezu et al. Anal Biochem. 415(2):145-50, 2011), single molecule real time sequencing methods (Flusberg et al. Nature Methods 7, 461-465), high resolution melting analysis (Wojdacz and Dobrovic (2007) Nucleic Acids Res. 35 (6): e41), and the like.

Additional applicable technologies for identifying chromosomal loci with changes in their DNA methylation status include, but not limited to, the preparation, amplification and analysis of Methylome libraries as described in U.S. Pat. No. 8,440,404; using Methylation-specific binding proteins as described in U.S. Pat. No. 8,394,585; determining the average DNA methylation density of a locus of interest within a population of DNA fragments as described in U.S. Pat. No. 8,361,719; by methylation-sensitive single nucleotide primer extension (Ms-SNuPE), for determination of strand-specific methylation status at cytosine residues as described in U.S. Pat. No. 7,037,650; a method for detecting a methylated CpG-containing nucleic acid present in a specimen by contacting the specimen with an agent that modifies unmethylated cytosine and amplifying the CpG-containing nucleic acid using CpG-specific oligonucleotide primers as described in U.S. Pat. No. 6,265,171; an improved method for the bisulfite conversion of DNA for subsequent analysis of DNA methylation as described in U.S. Pat. No. 8,586,302; for treating genomic DNA samples with sodium bisulfite to create methylation-dependent sequence differences, followed by detection with fluorescence-based quantitative PCR techniques as described in U.S. Pat. No. 8,323,890; a method for retaining methylation pattern in globally amplified DNA as described in U.S. Pat. No. 7,820,385; a method for detecting cytosine methylations DNA as described in U.S. Pat. No. 8,241,855; a method for quantification of methylated DNA as described in U.S. Pat. No. 7,972,784; a highly sensitive method for the detection of cytosine methylation patterns as described in U.S. Pat. No. 7,229,759; additional methods for detecting DNA methylation changes are described in U.S. Pat. No. 7,943,308 and U.S. Pat. No. 8,273,528.

Plant centromeres are responsible for normal chromosomal segregation during mitosis and meiosis. Flanking the centromeres are the pericentromeric regions which facilitate centromere function. Centromeres are primarily composed of centromeric satellite repeated sequences and centromeric retrotransposons. In Arabidopsis, a 180-bp satellite repeat forms the main repeating centromeric sequence. Centromeric satellite repeats are mostly specific to the centromeric regions with a few copies that generally are not present as long tandem repeats elsewhere in the genome. An exception is that a limited amount of centromeric satellite repeats can also be found in the flanking pericentromeric regions. Centromeric regions bind the specialized centromeric histones such as CENH3 and the like.

Accordingly, a functional description of pericentromeric regions is heterochromatic regions containing abundant repeated sequences, transposable elements, and retrotransposons that physically flank the centromeric regions. Pericentromeric regions are often rich in mono and dimethylated H3K9 heterochromatin regions and can contain active genes. At the sequence level, a functional definition for pericentromeric sequences are repeated sequences other than the centromeric repeats and that contain transposable elements and retrotransposons embedded in said repeated pericentromeric sequences. When available, chromosomal positioning information about the location of sequences that are located adjacent to the centromere strengthens the identification of pericentromeric sequences.

Transposable elements of both class I (long terminal repeat [LTR]-retrotransposons) and class II (DNA transposons of different superfamilies) are abundantly present in plant genomes (Kidwell 2002 Genetica 115:49-63; Kapitonov and Jurka Nat Rev Genet. 2008 May; 9(5):411-2; Wicker Nat Rev Genet. 2007 December; 8(12):973-82). They can be identified by various software programs as described in Lerat (Heredity (Edinb). 2010 June; 104(6):520-33). Repbase Update (RU) is a database of prototypic sequences representing repetitive DNA including transposable elements from different eukaryotic species. Candidate sequences available from a variety of assay methods such as microarrays and next generation sequencing such as Illumina can be compared to known transposable element sequences as described above to identify most known transposable elements in a plant genome.

The aforementioned methods are useful for producing plants with new combinations of altered chromosomal loci and/or identifying plants with useful combinations of altered chromosomal loci. These plants can be further breed and/or screened and selected for useful traits in a manner consistent with plant breeding practices. In certain embodiments, the screened and selected trait is improved plant yield. In certain embodiments, such yield improvements are improvements in the yield of a plant line relative to one or more parental line(s) under non-stress conditions. Non-stress conditions comprise conditions where water, temperature, nutrients, minerals, and light fall within typical ranges for cultivation of the plant species. Such typical ranges for cultivation comprise amounts or values of water, temperature, nutrients, minerals, and light that are neither insufficient nor excessive.

Plant lines and plant populations obtained by the methods provided herein can be screened and selected for a variety of useful traits by using a wide variety of techniques. In particular embodiments provided herein, individual progeny plant lines or populations of plants obtained from the selfs or outcrosses of plants where plastid perturbation target gene expression was suppressed to other plants are screened and selected for the desired useful traits.

In certain embodiments, the screened and selected trait is improved plant yield. In certain embodiments, such yield improvements are improvements in the yield of a plant line relative to one or more parental line(s) under non-stress conditions. Non-stress conditions comprise conditions where water, temperature, nutrients, minerals, and light fall within typical ranges for cultivation of the plant species. Such typical ranges for cultivation comprise amounts or values of water, temperature, nutrients, minerals, and/or light that are neither insufficient nor excessive. In certain embodiments, such yield improvements are improvements in the yield of a plant line relative to parental line(s) under abiotic stress conditions. Such abiotic stress conditions include, but are not limited to, conditions where water, temperature, nutrients, minerals, and/or light that are either insufficient or excessive. Abiotic stress conditions would thus include, but are not limited to, drought stress, osmotic stress, nitrogen stress, phosphorous stress, mineral stress, heat stress, cold stress, and/or light stress. In this context, mineral stress includes, but is not limited to, stress due to insufficient or excessive potassium, calcium, magnesium, iron, manganese, copper, zinc, boron, aluminum, or silicon. In this context, mineral stress includes, but is not limited to, stress due to excessive amounts of heavy metals including, but not limited to, cadmium, copper, nickel, zinc, lead, and chromium.

Improvements in yield in plant lines obtained by the methods provided herein can be identified by direct measurements of wet or dry biomass including, but not limited to, grain, lint, leaves, stems, or seed. Improvements in yield can also be assessed by measuring yield related traits that include, but are not limited to, 100 seed weight, a harvest index, and seed weight. In certain embodiments, such yield improvements are improvements in the yield of a plant line relative to one or more parental line(s) and can be readily determined by growing plant lines obtained by the methods provided herein in parallel with the parental plants. In certain embodiments, field trials to determine differences in yield whereby plots of test and control plants are replicated, randomized, and controlled for variation can be employed (Giesbrecht F G and Gumpertz M L. 2004. Planning, Construction, and Statistical Analysis of Comparative Experiments. Wiley. New York; Mead, R. 1997. Design of plant breeding trials. In Statistical Methods for Plant Variety Evaluation. eds. Kempton and Fox. Chapman and Hall. London.). Methods for spacing of the test plants (i.e. plants obtained with the methods of this invention) with check plants (parental or other controls) to obtain yield data suitable for comparisons are provided in references that include, but are not limited to, any of Cullis, B. et al. J. Agric. Biol. Env. Stat. 11:381-393; and Besag, J. and Kempton, R A. 1986. Biometrics 42: 231-251.). Other useful traits that can be obtained by the methods provided herein include various seed quality traits including, but not limited to, improvements in either the compositions or amounts of oil, protein, or starch in the seed. Still other useful traits that can be obtained by methods provided herein include, but are not limited to, increased biomass, non-flowering, male sterility, digestability, seed filling period, maturity (either earlier or later as desired), reduced lodging, and plant height (either increased or decreased as desired).

In certain embodiments, the screened and selected trait is improved resistance to biotic plant stress relative to the parental lines. Biotic plant stress includes, but is not limited to, stress imposed by plant fungal pathogens, plant bacterial pathogens, plant viral pathogens, insects, nematodes, and herbivores. In certain embodiments, screening and selection of plant lines that exhibit resistance to fungal pathogens including, but not limited to, an Alternaria sp., an Ascochyta sp., a Botrytis sp.; a Cercospora sp., a Colletotrichum sp., a Diaporthe sp., a Diplodia sp., an Erysiphe sp., a Fusarium sp., Gaeumanomyces sp., Helminthosporium sp., Macrophomina sp., a Nectria sp., a Peronospora sp., a Phakopsora sp., Phialophora sp., a Phoma sp., a Phymatotrichum sp., a Phytophthora sp., a Plasmopara sp., a Puccinia sp., a Podosphaera sp., a Pyrenophora sp., a Pyricularia sp, a Pythium sp., a Rhizoctonia sp., a Scerotium sp., a Sclerotinia sp., a Septoria sp., a Thielaviopsis sp., an Uncinula sp, a Venturia sp., and a Verticillium sp. is provided. In certain embodiments, screening and selection of plant lines that exhibit resistance to bacterial pathogens including, but not limited to, an Erwinia sp., a Pseudomonas sp., and a Xanthamonas sp. is provided. In certain embodiments, screening and selection of plant lines that exhibit resistance to insects including, but not limited to, aphids and other piercing/sucking insects such as Lygus sp., lepidoteran insects such as Armigera sp., Helicoverpa sp., Heliothis sp., and Pseudoplusia sp., and coleopteran insects such as Diabroticus sp. is provided. In certain embodiments, screening and selection of plant lines that exhibit resistance to nematodes including, but not limited to, Meloidogyne sp., Heterodera sp., Belonolaimus sp., Ditylenchus sp., Globodera sp., Naccobbus sp., and Xiphinema sp. is provided.

Other useful traits that can be obtained by the methods provided herein include various seed quality traits including, but not limited to, improvements in either the compositions or amounts of oil, protein, or starch in the seed. Still other useful traits that can be obtained by methods provided herein include, but are not limited to, increased biomass, non-flowering, male sterility, digestability, seed filling period, maturity (either earlier or later as desired), reduced lodging, and plant height (either increased or decreased as desired).

In addition to any of the aforementioned traits, particularly useful traits for sorghum that can be obtained by the methods provided herein also include, but are not limited to: i) agronomic traits (flowering time, days to flower, days to flower-post rainy, days to flower-rainy; ii) fungal disease resistance (sorghum downy mildew resistance—glasshouse, sorghum downy mildew resistance-field, sorghum grain mold, sorghum leaf blight resistance, sorghum rust resistance; iii) grain related trait: (Grain dry weight, grain number, grain number per square meter, Grain weight over panicle. seed color, seed luster, seed size); iv) growth and development stage related traits (basal tillers number, days to harvest, days to maturity, nodal tillering, plant height, plant height-postrainy); v) infloresence anatomy and morphology trait (threshability); vi) Insect damage resistance (sorghum shoot fly resistance-post-rainy, sorghum shoot fly resistance-rainy, sorghum stem borer resistance); vii) leaf related traits (leaf color, leaf midrib color, leaf vein color, flag leaf weight, leaf weight, rest of leaves weight); viii) mineral and ion content related traits (shoot potassium content, shoot sodium content); ix) panicle related traits (number of panicles, panicle compactness and shape, panicle exertion, panicle harvest index, panicle length, panicle weight, panicle weight without grain, panicle width); x) phytochemical compound content (plant pigmentation); xii) spikelet anatomy and morphology traits (glume color, glume covering); xiii) stem related trait (stem over leaf weight, stem weight); and xiv) miscellaneous traits (stover related traits, metabolised energy, nitrogen digestibility, organic matter digestibility, stover dry weight).

### Examples

The following examples are included to demonstrate various embodiments. However, those of skill in the art should, in light of the present disclosure, appreciate that many changes can be made in the specific embodiments which are disclosed and still obtain a like or similar result without departing from the spirit and scope of the invention.

### Example 1

### MSH1 is Localized to a Special Plastid Type and is Associated with PPD3

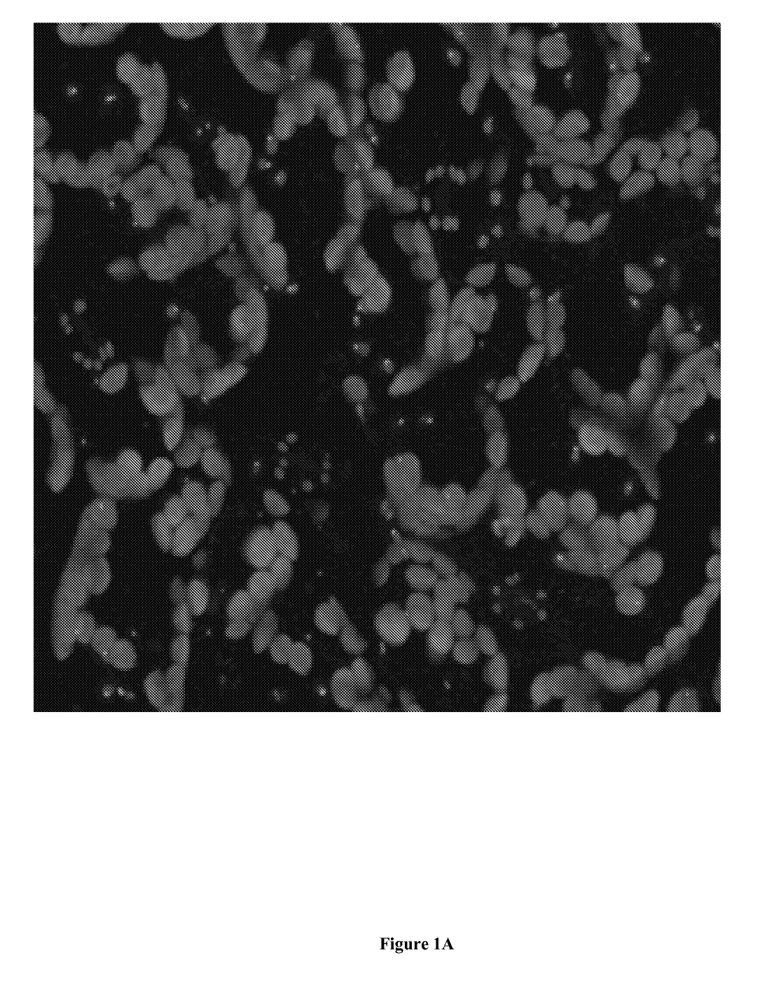

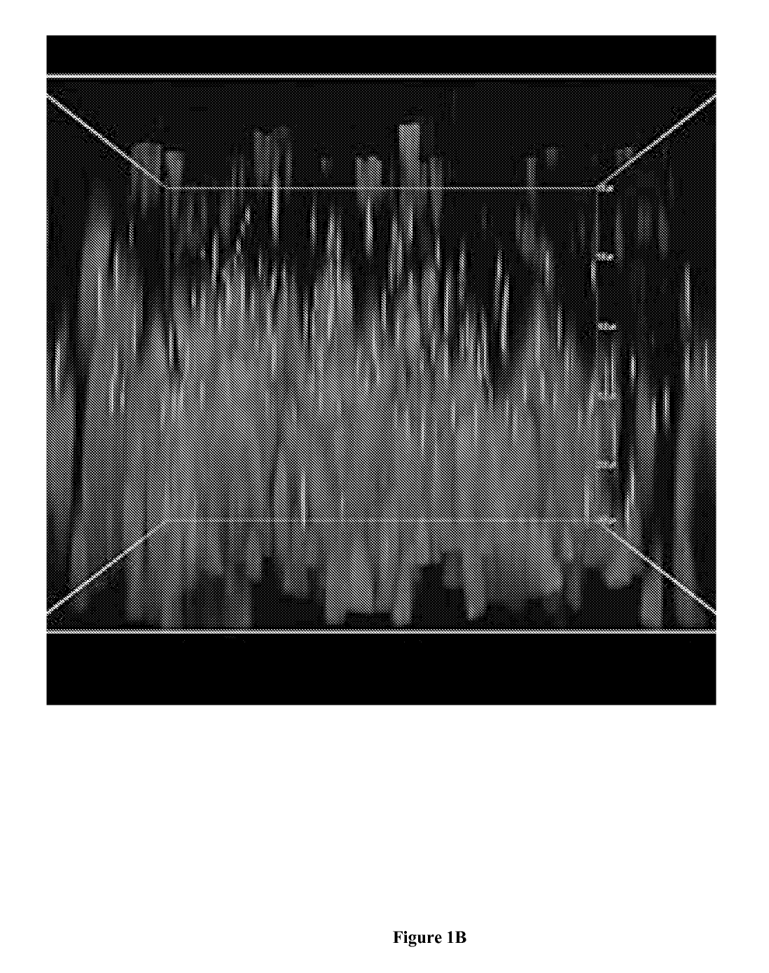

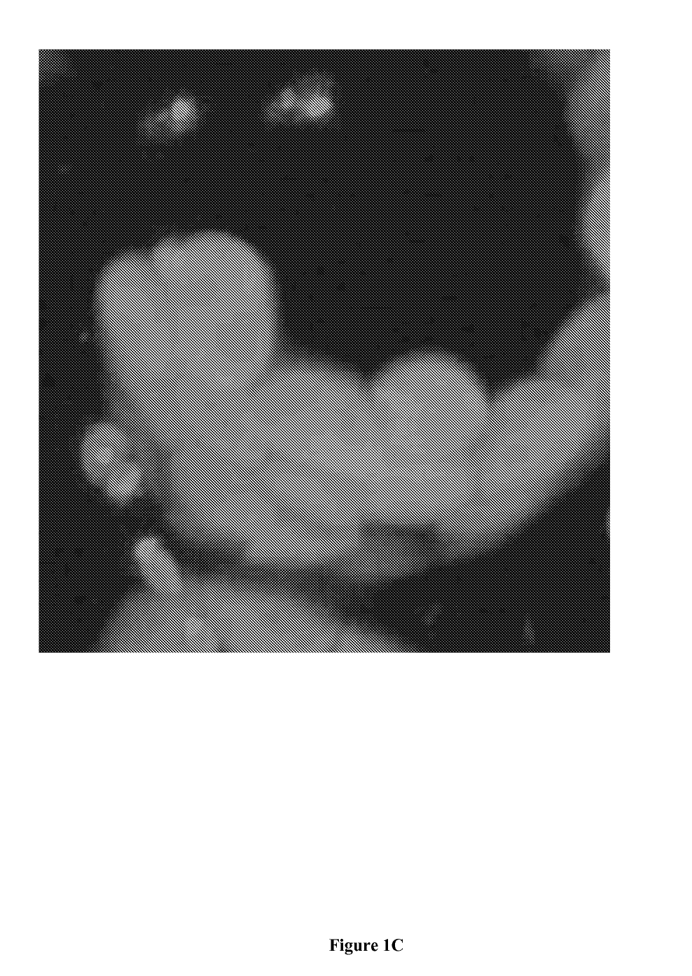

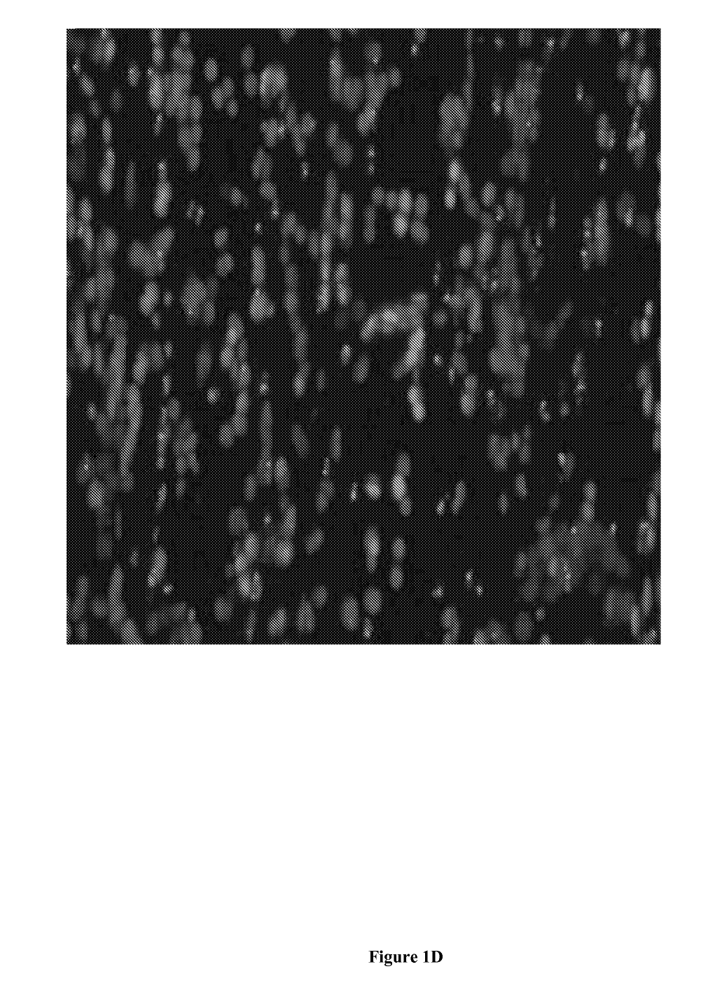

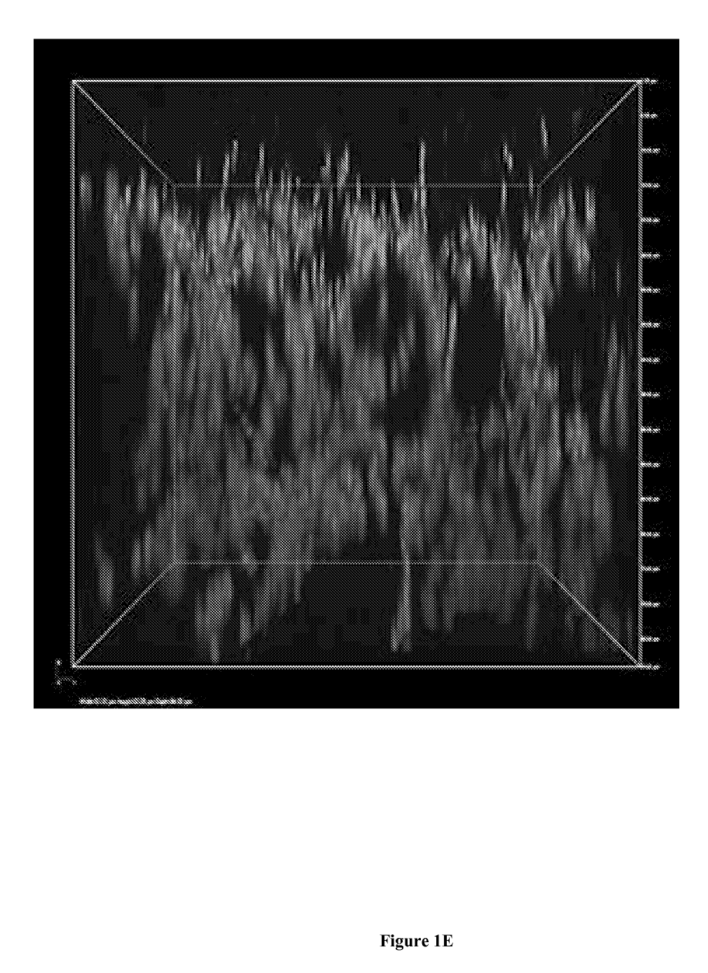

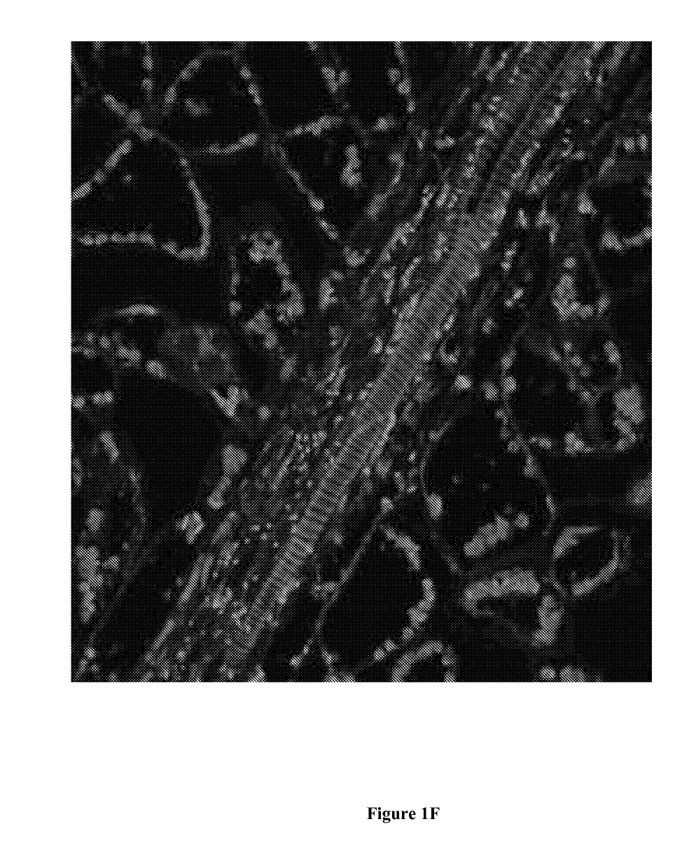

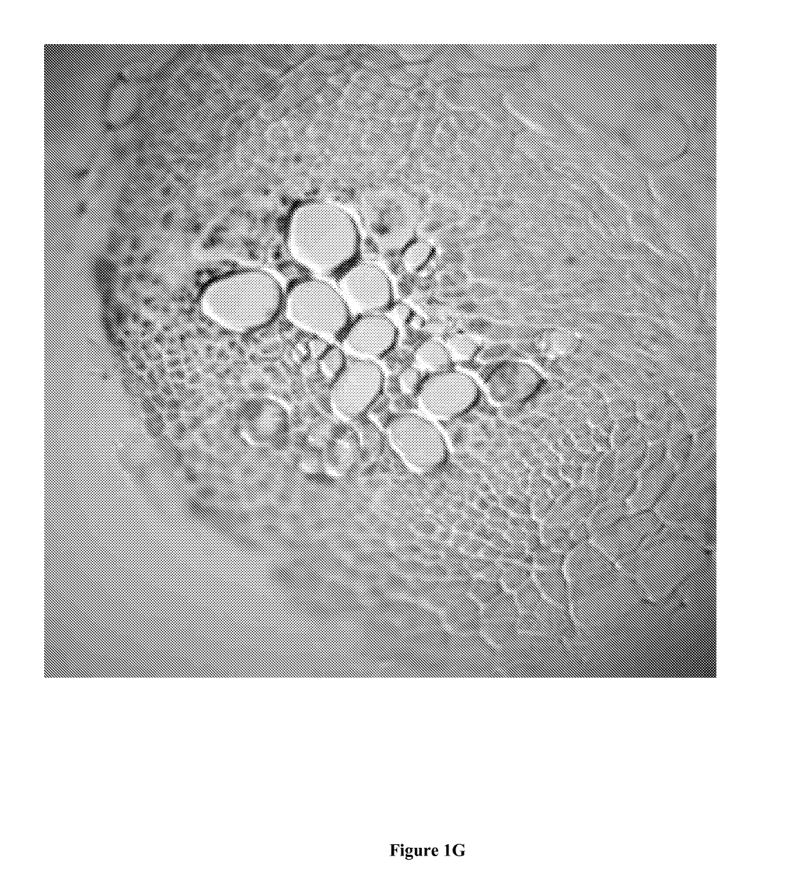

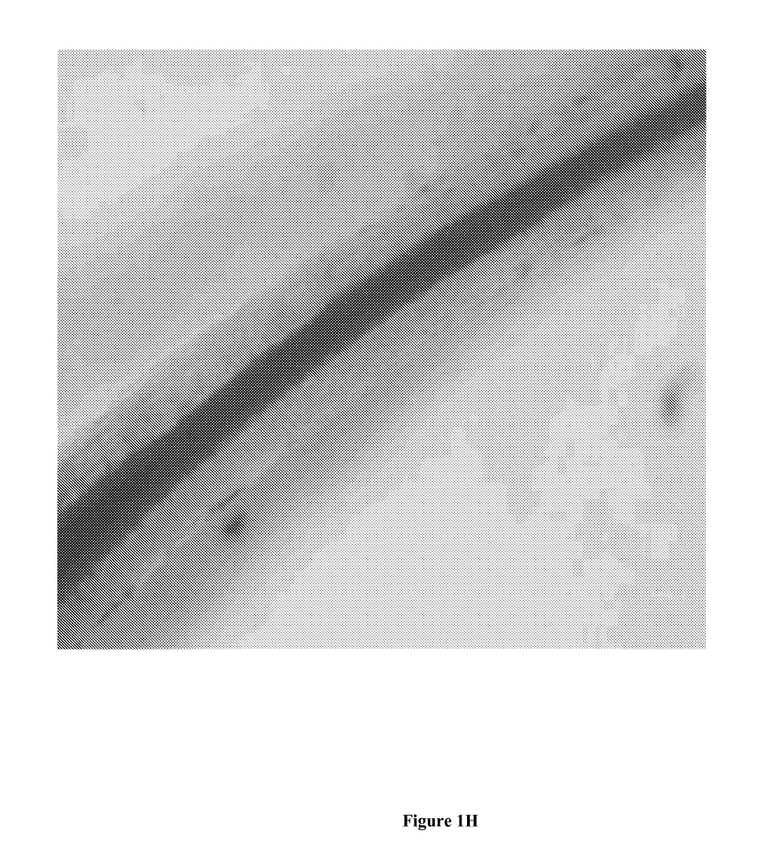

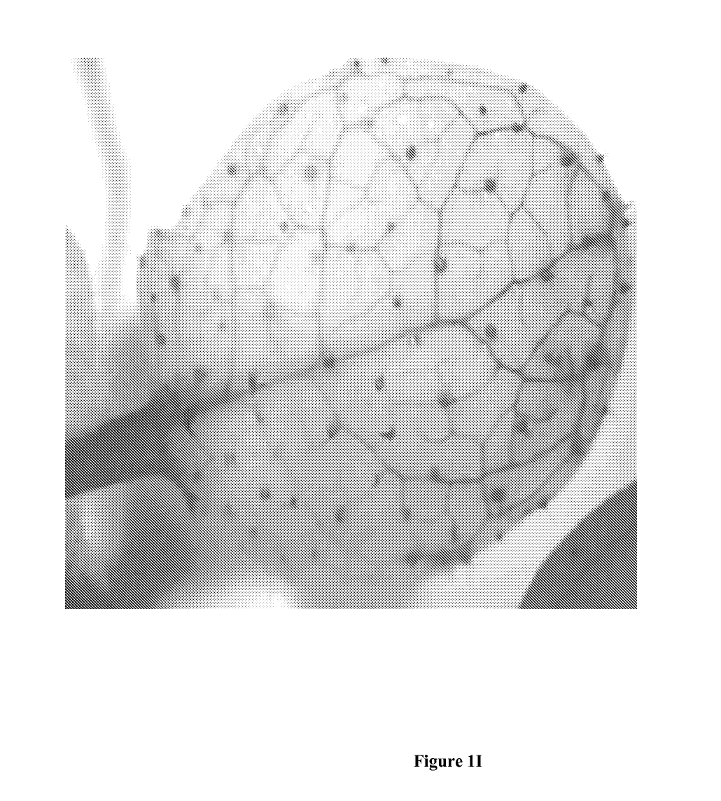

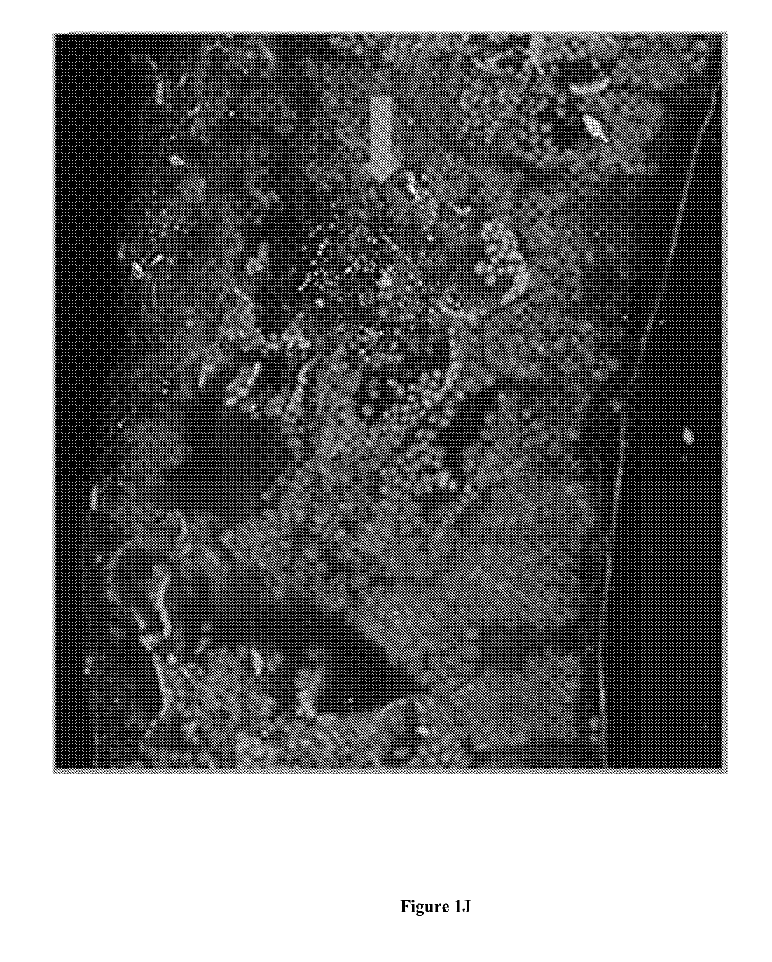

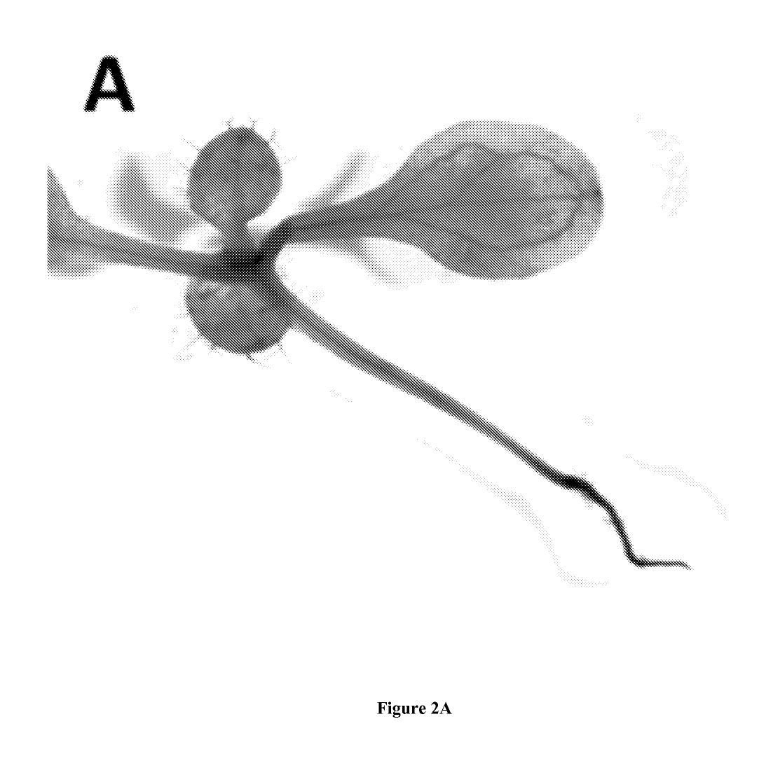

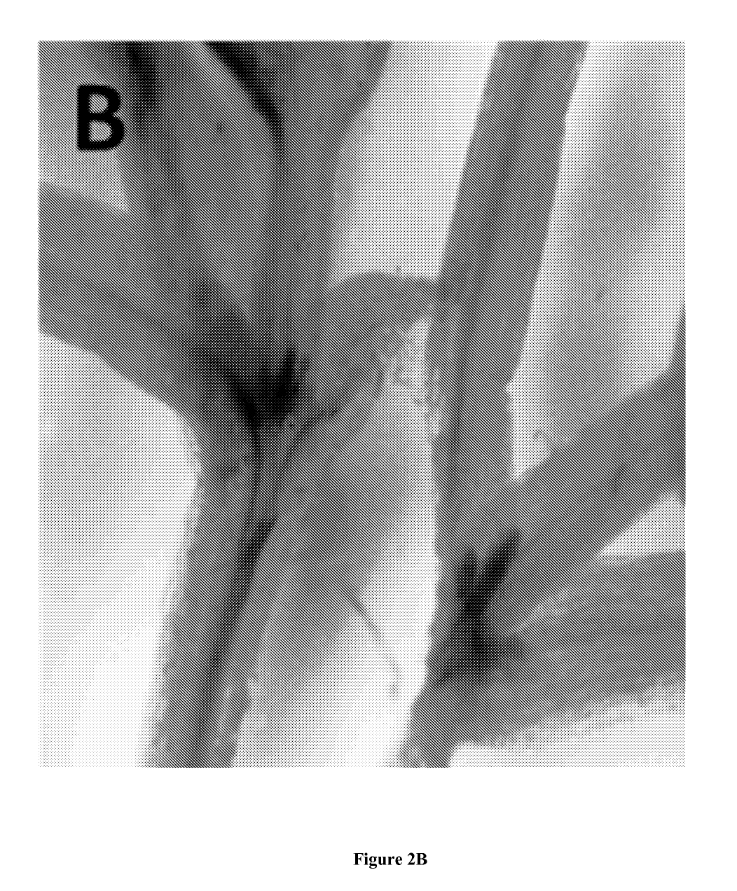

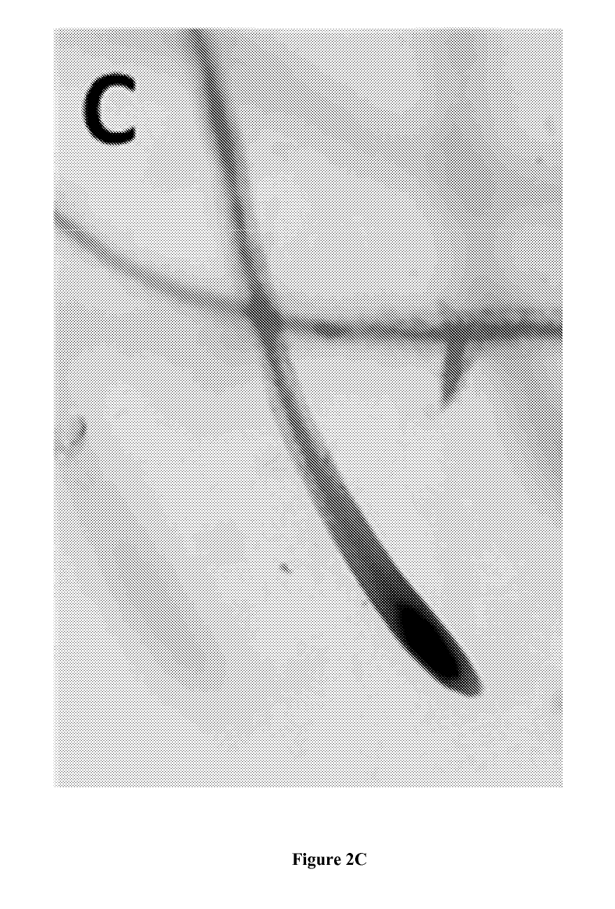

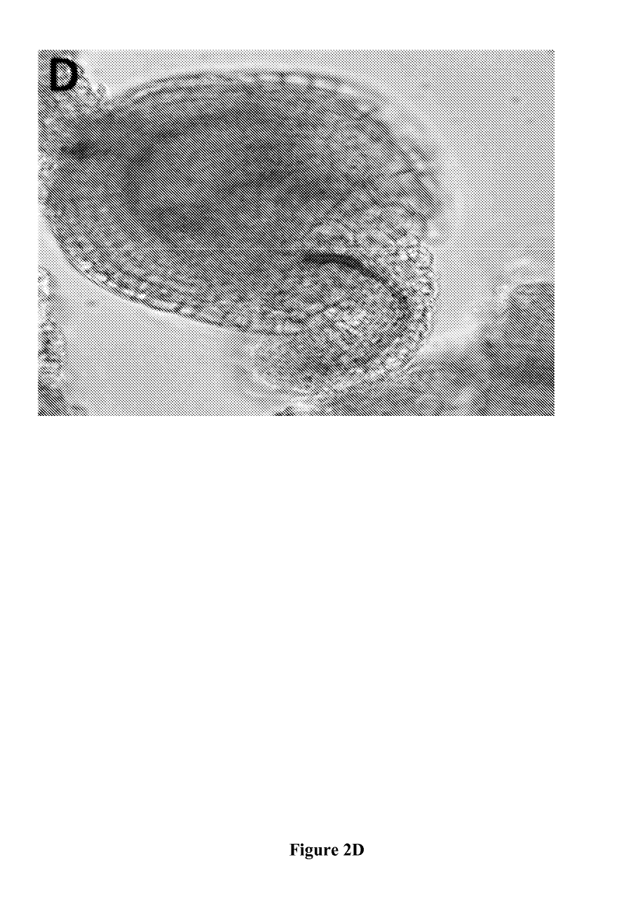

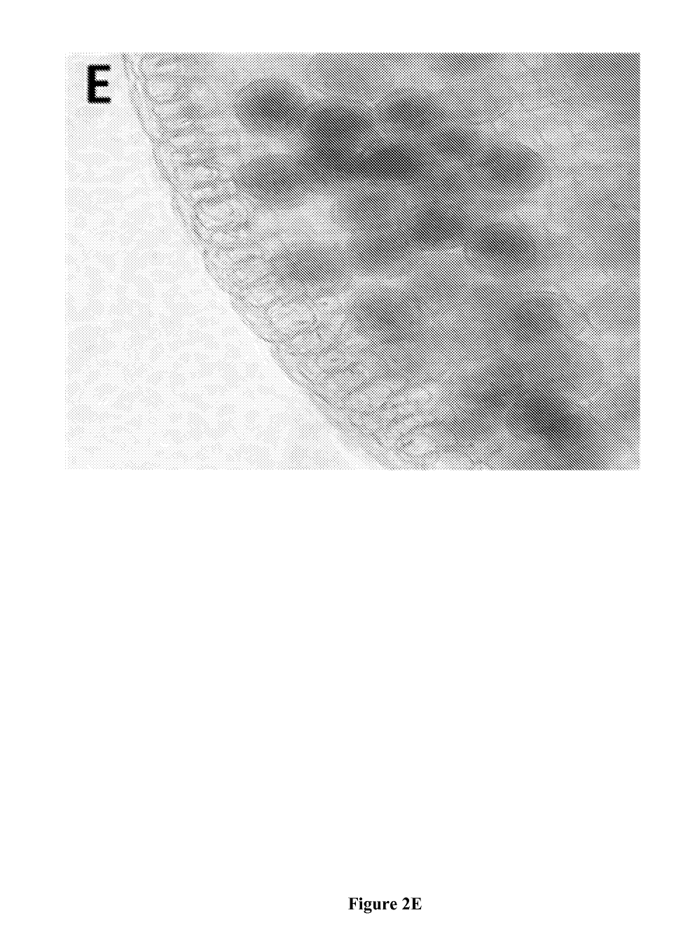

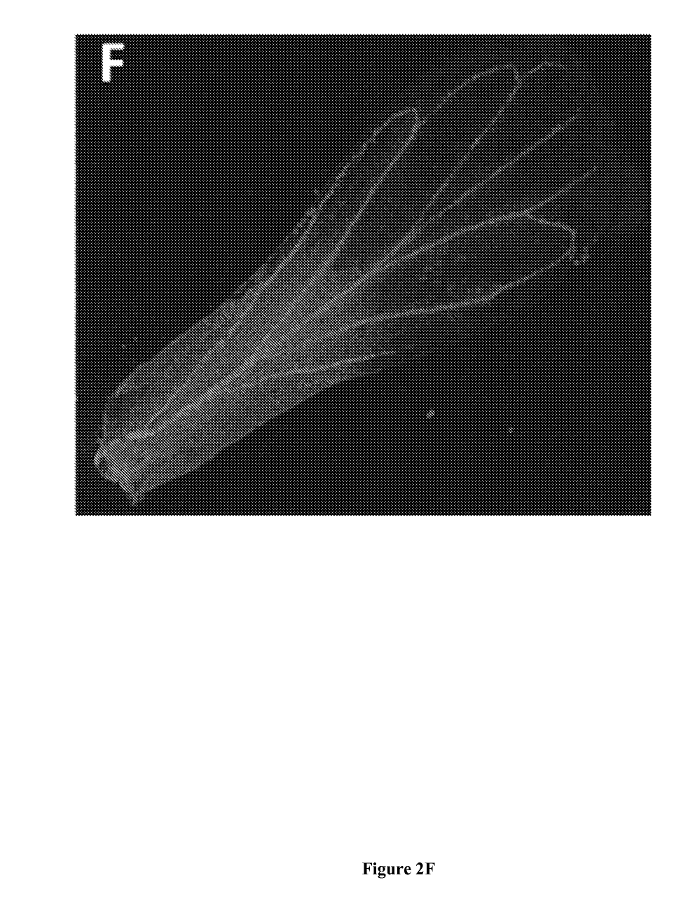

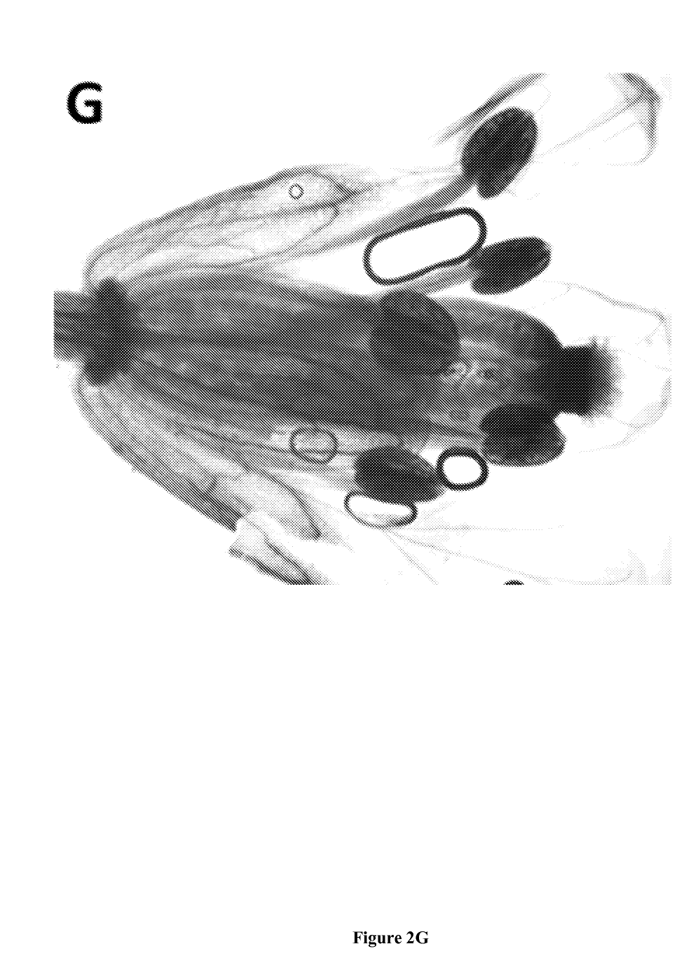

Earlier studies of MSH1 showed that the protein functions in both mitochondria and plastids. To further investigate the role of MSH1 in plastids, the MSH1 promoter and full-length gene were fused to GFP and stably transformed to Arabidopsis ecotype Col-0. While MSH1-GFP signal was detected in nearly all plant tissues throughout development, the spatial pattern of expression appeared to be largely restricted to epidermal cells, vascular parenchyma, meristems and reproductive tissues (FIGS. 1 and 2). This expression pattern was confirmed with gene constructions that included only the MSH1 promoter fused to uidA to assess GUS expression. These experiments demonstrated that the unusual spatial pattern for MSH1 accumulation is directed by the gene's promoter.

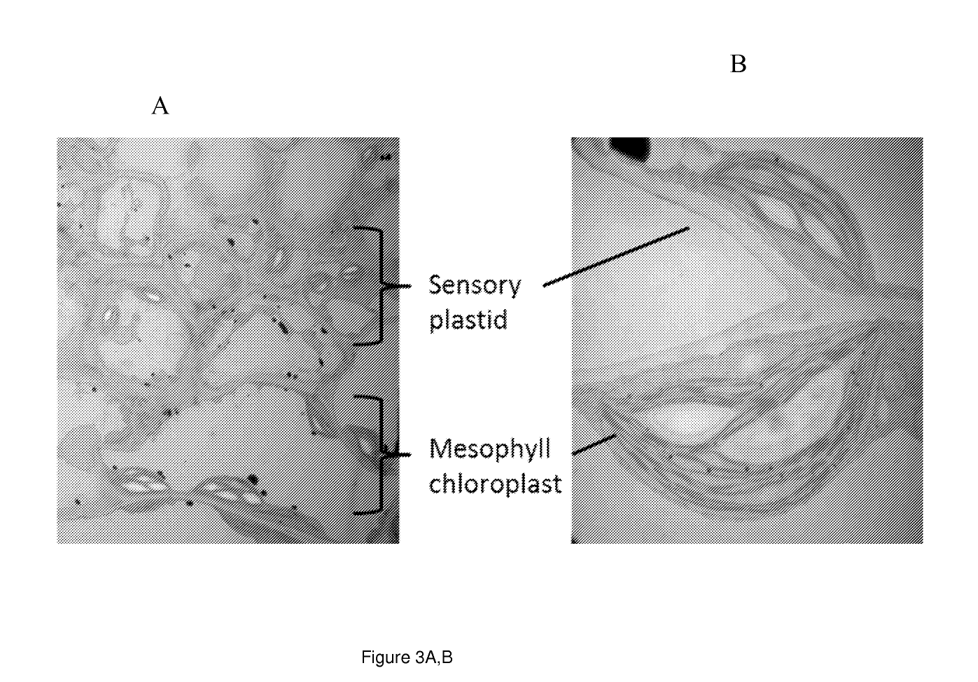

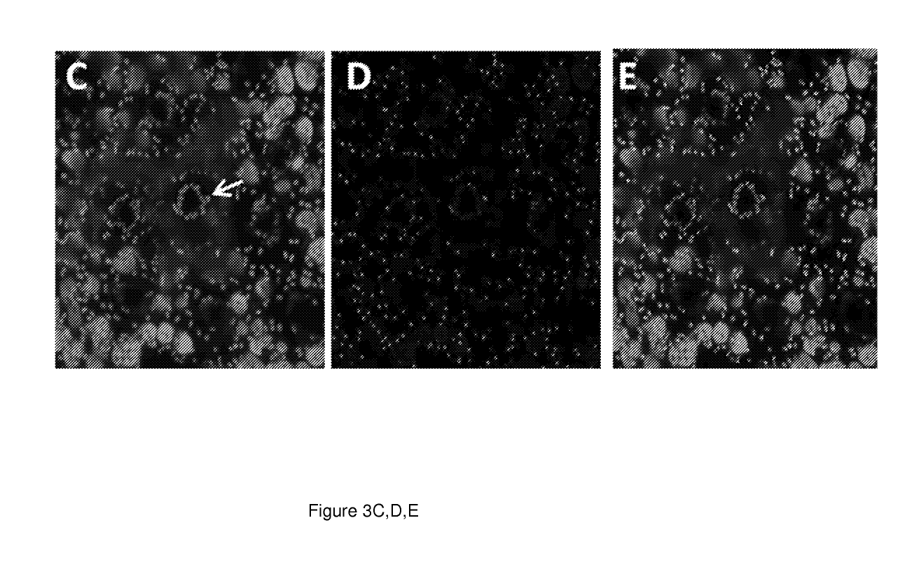

Analysis by laser scanning confocal microscopy suggested that in the leaf lamina region, GFP signal resided only on the upper surface of cells. However, nearing the midrib, the signal was detected in nearly all cell layers (FIG. 1B, E). At higher resolution, one is able to observe GFP as punctate signals from within plastid structures that are visibly smaller than mesophyll chloroplasts (FIG. 1C). The size difference was more readily estimated by electron microscopy, where these smaller plastids approximate 30-40% the size of the mesophyll chloroplasts in neighboring cells (FIG. 3).

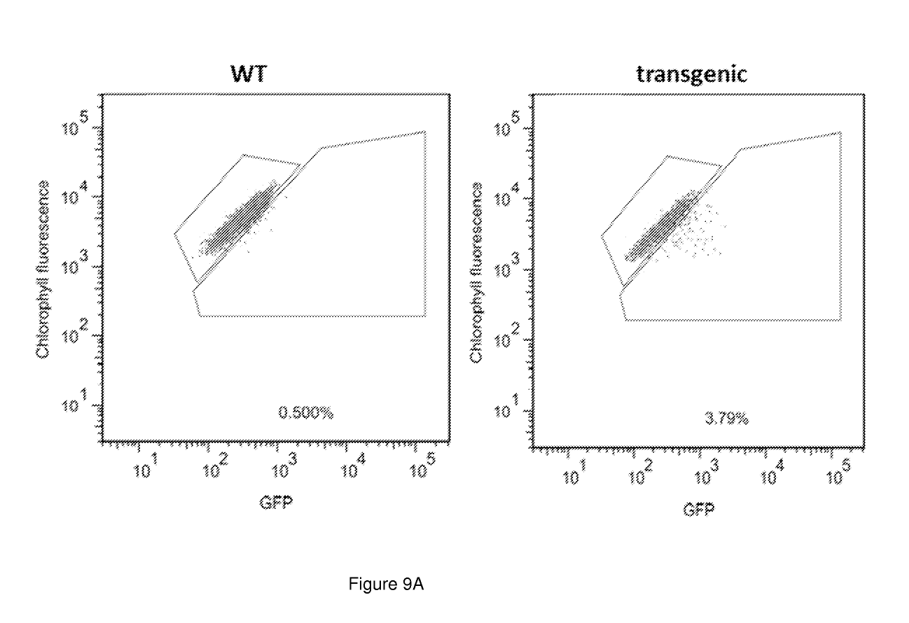

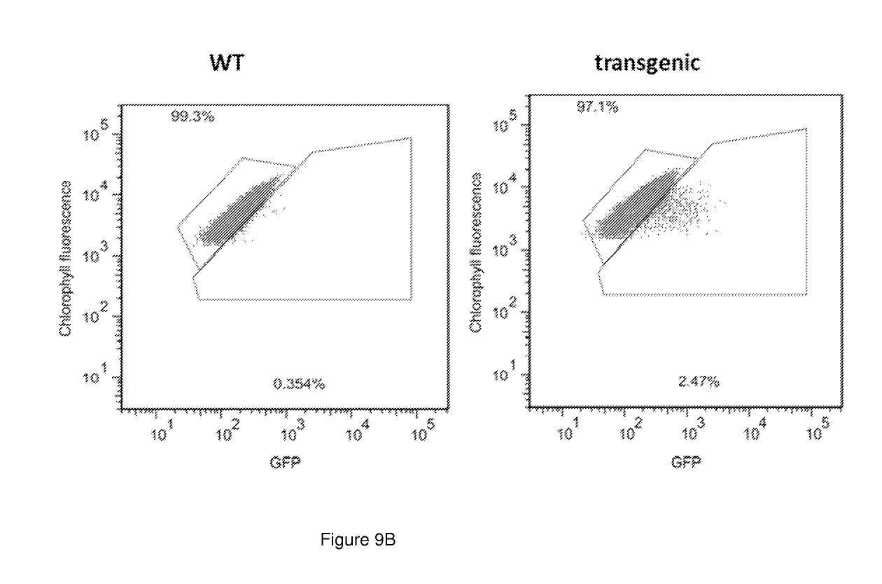

The smaller, MSH1-associated plastids display less extensive thylakoid membrane and granal stacking, and contained far fewer visible plastoglobuli than did mesophyll chloroplasts (FIG. 3B). While their autofluorescence signal was lower than mesophyll chloroplasts, they contained abundant starch. MSH1 expression has been shown previously to be modulated by abiotic stress (Shedge et al. 2010, Xu et al. 2011), and so we have termed these unusual MSH1-associated organelles ‘sensory’ plastids. To learn whether these organelles, and their unusual association with MSH1, can be generalized to other plant species, we stably transformed the Arabidopsis MSH1-GFP gene construct to tobacco (Nicotiana tabacum L). Confocal microscopy in tobacco revealed a similar pattern of smaller organelles in the epidermal cells, as well as a seemingly specialized association by MSH1 to these organelles (FIG. 3C-E). In both Arabidopsis and tobacco, crude plastid preparations were analyzed by fluorescence-activated cell sorting (FACS) to estimate the fraction of plastids that contain MSH1. Results from these experiments suggest that MSH1-containing sensory plastids comprise approximately 2-3% of the total intact plastids isolated from leaves (FIG. 9A-B).

MSH1 Resides on the Thylakoid Membrane and Interacts with Photosynthetic Components

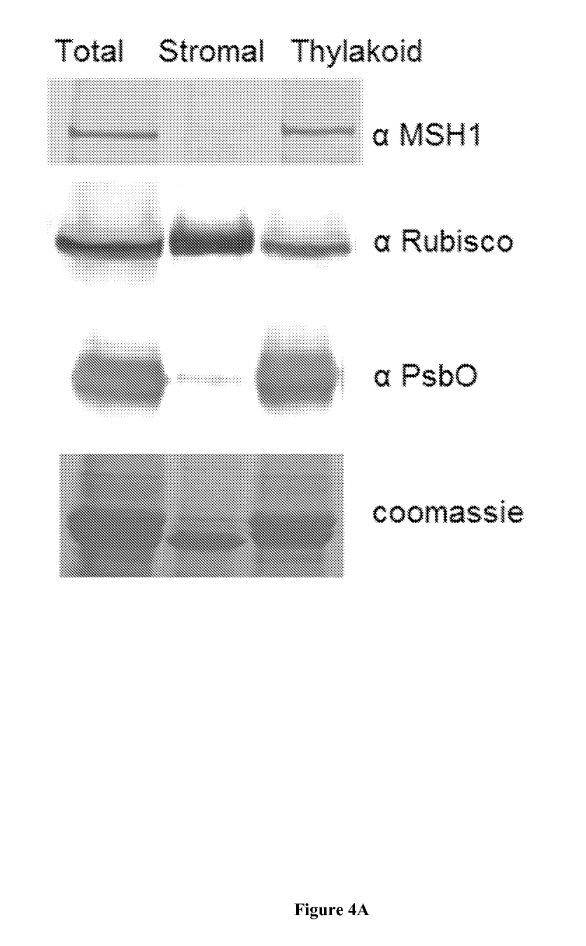

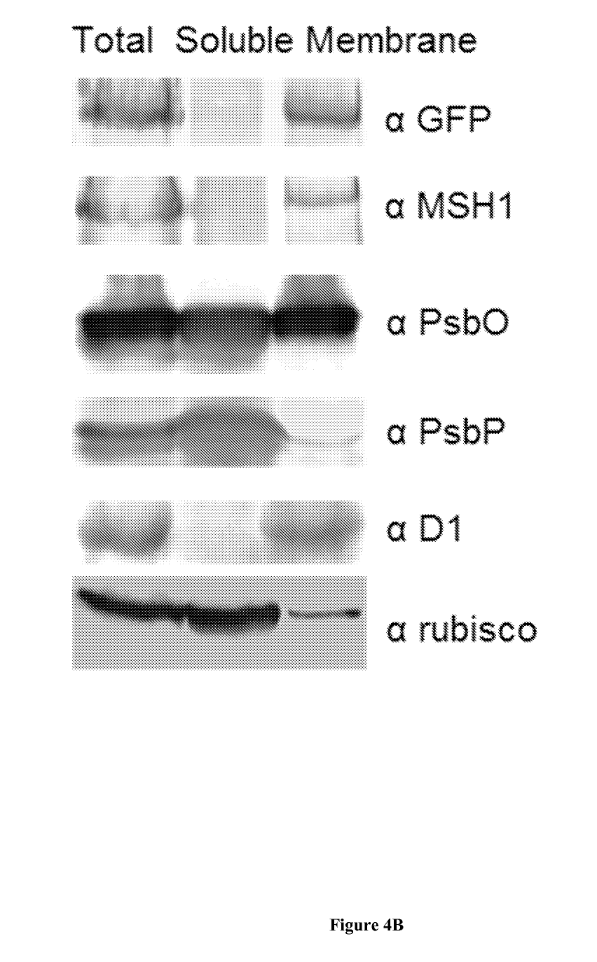

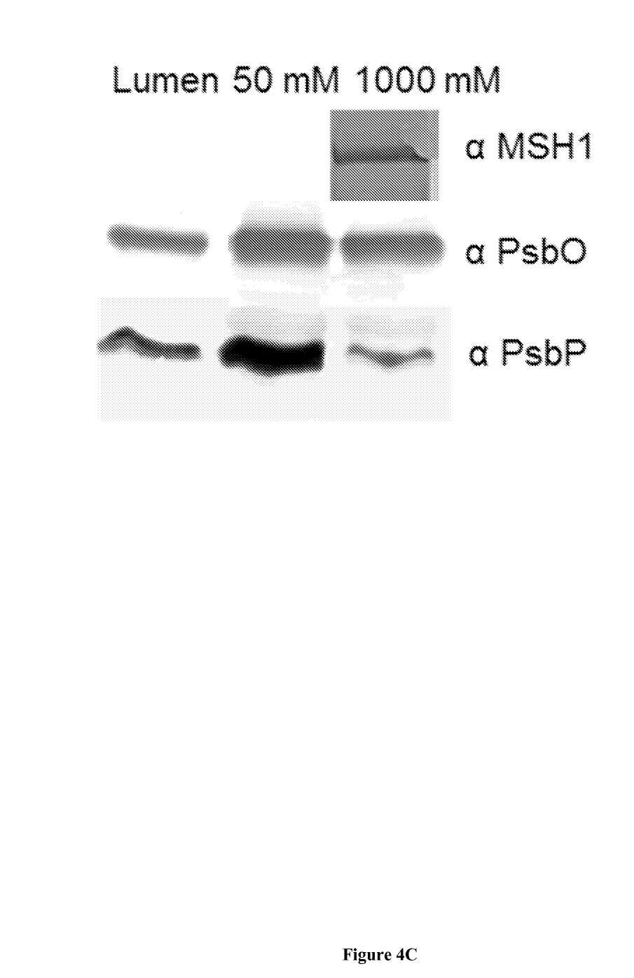

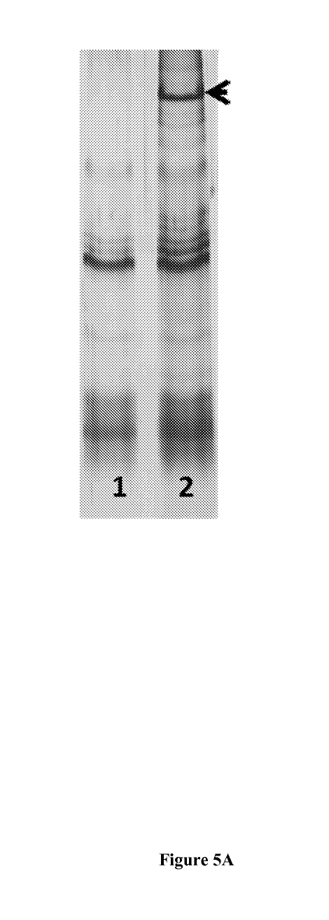

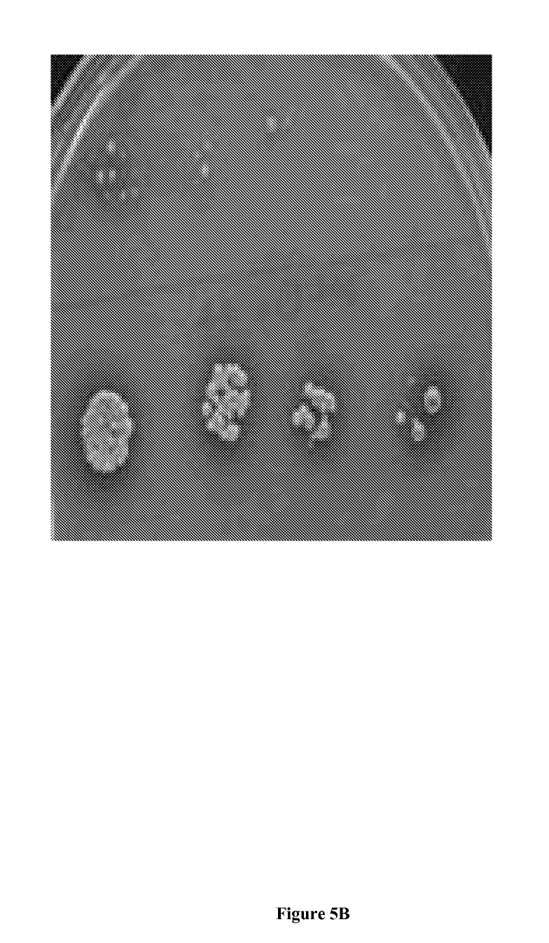

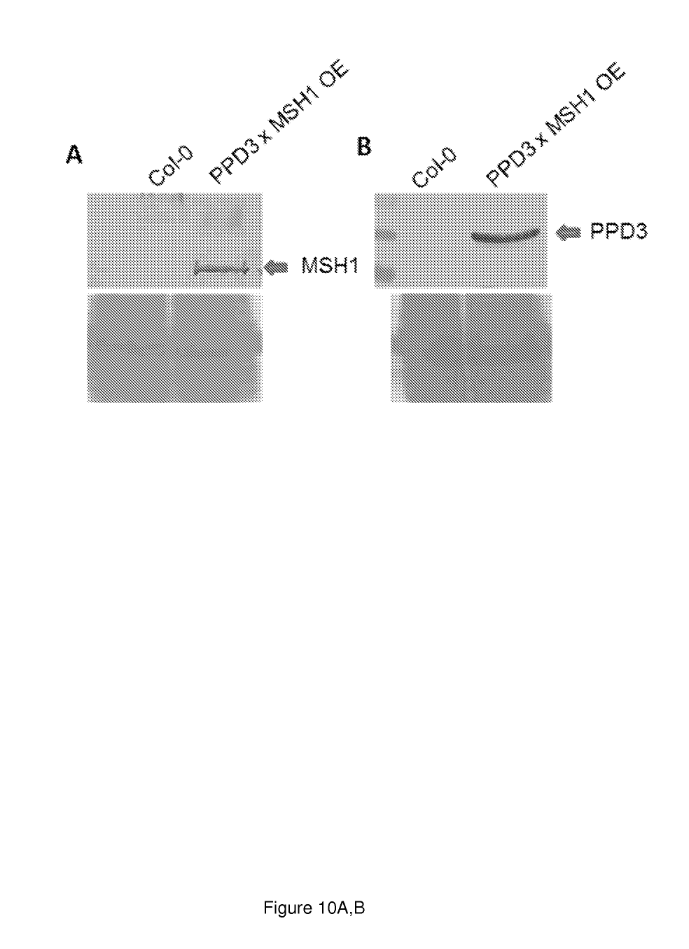

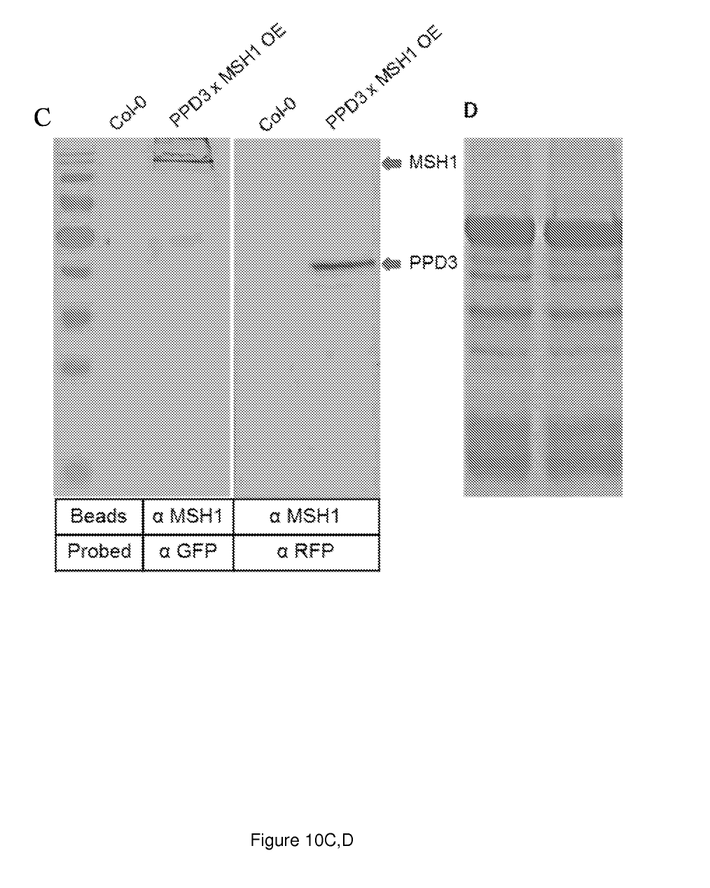

The punctate GFP signal observed within the sensory plastids suggests that MSH1 is likely compartmented within the organelle. Because the MSH1 protein is in low abundance, we opted to carry out cell fractionation experiments in an Arabidopsis stable MSH1-GFP transformant that is expressed under the control of the native promoter. Plastid fractionations resulted in co-purification of MSH1 with the thylakoid membrane (FIG. 4). This association persisted with mild detergent or salt washes, implying that the protein may be membrane-associated. To investigate possible MSH1 protein partners within the plastid, we carried out yeast 2-hybrid and co-immunoprecipitation experiments. Yeast 2-hybrid studies, with full-length MSH1 as bait in multiple matings, identified sixteen genes as putative interactors. Of these, three were selected for further investigation based on their plastid localization and consistent reproducibility in subsequent one-on-one matings. Two of the three plastid proteins, PsbO1 and PsbO2, are members of the photosystem II oxygen evolving complex, and the third, PPD3, is a 27.5 kDa PsbP domain-containing protein also thought to reside in the lumen (Ifuku et al. 2010). CoIP experiments with MSH1 did not produce PsbO1 or PsbO2, but did produce PPD3 (FIG. 10A-D), as well as two additional components of the photosynthetic apparatus, PsbA (D1) and PetC. Since PsbA and PetC were not identified by yeast 2-hybrid screening, we introduced these into one-on-one matings with MSH1, producing weak signals for positive interaction (FIG. 5B). MSH1 can be subdivided to six intervals based on cross-species protein alignments (Abdelnoor et al. 2006), with domain 1 containing a DNA binding domain, Domain V containing an ATPase domain and Domain VI encoding a GIY-YIG endonuclease domain. We subcloned MSH1 in accordance with these intervals, and conducted yeast 2-hybrid matings with each MSH1 domain as bait. From these experiments, we observed positive interaction with PPD3 at Domains 2, 3, and 6. All other putative partners produced positive interaction with Domain 3 (FIG. 5C). While Domain 3-4 appears to be bordered on both sides by short hydrophobic intervals, it is not clear whether MSH1 may span or anchor to the thylakoid membrane.

MSH1 and PPD3 are coexpressed and appear to be functional interactors.

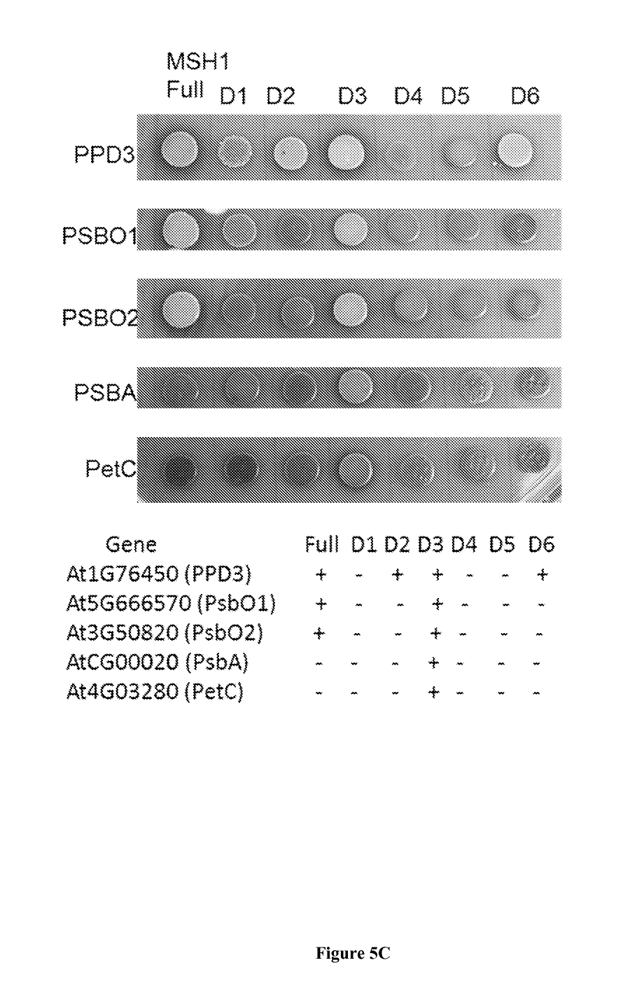

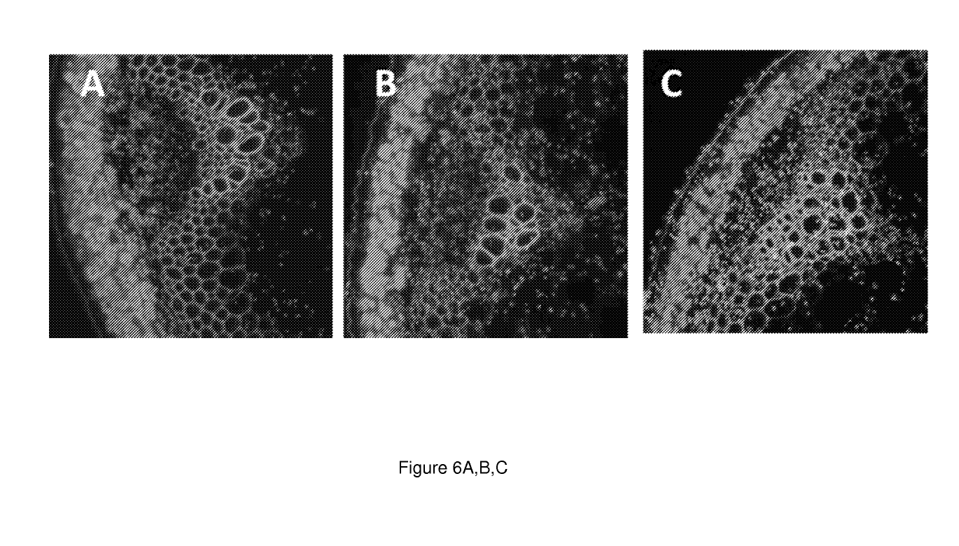

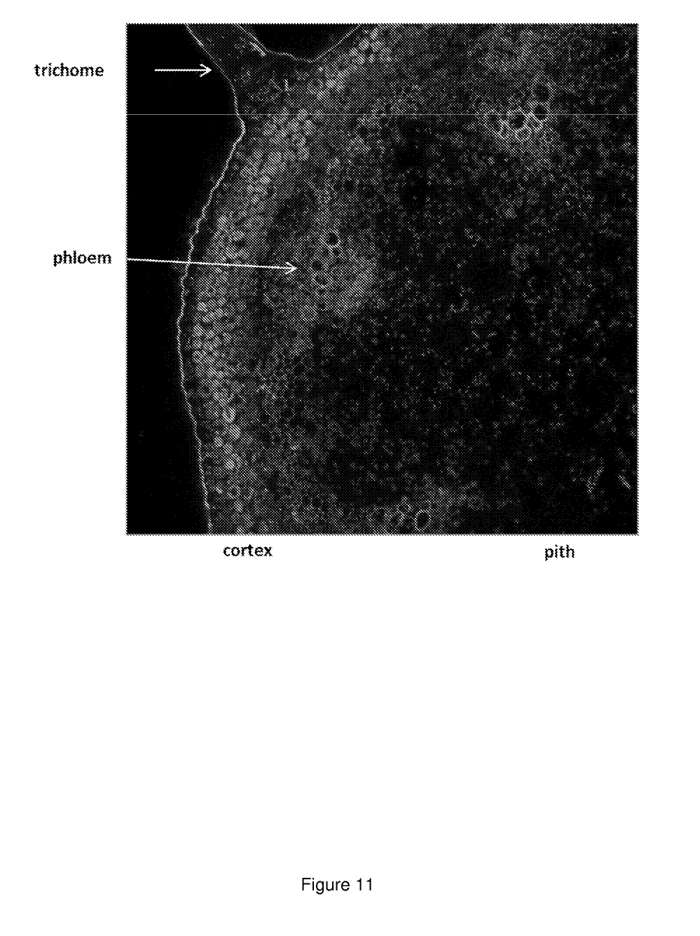

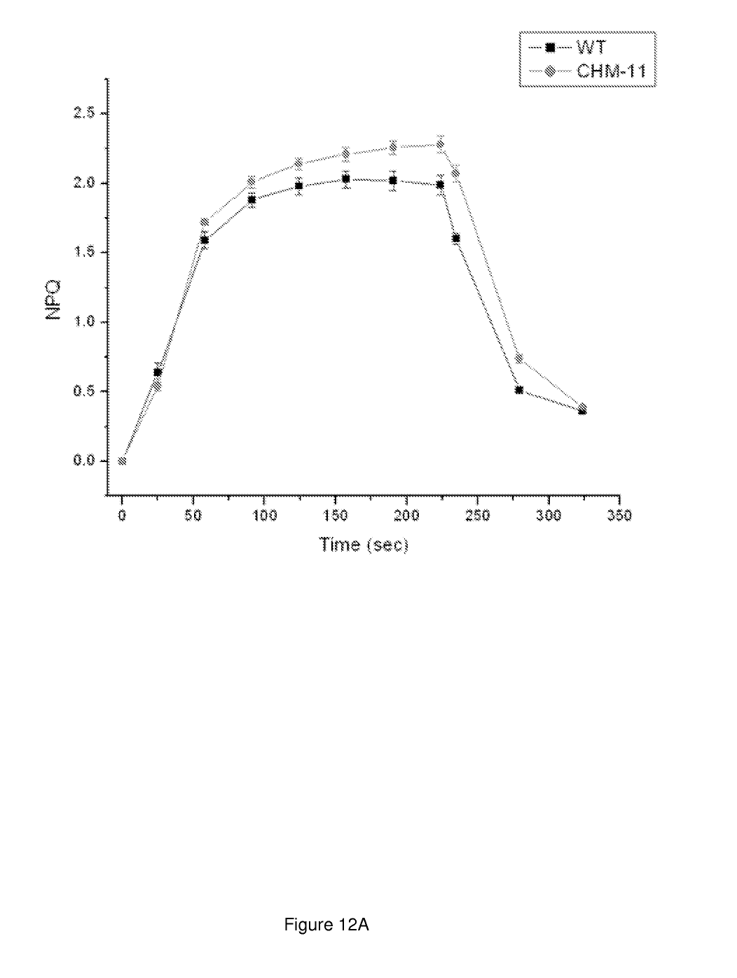

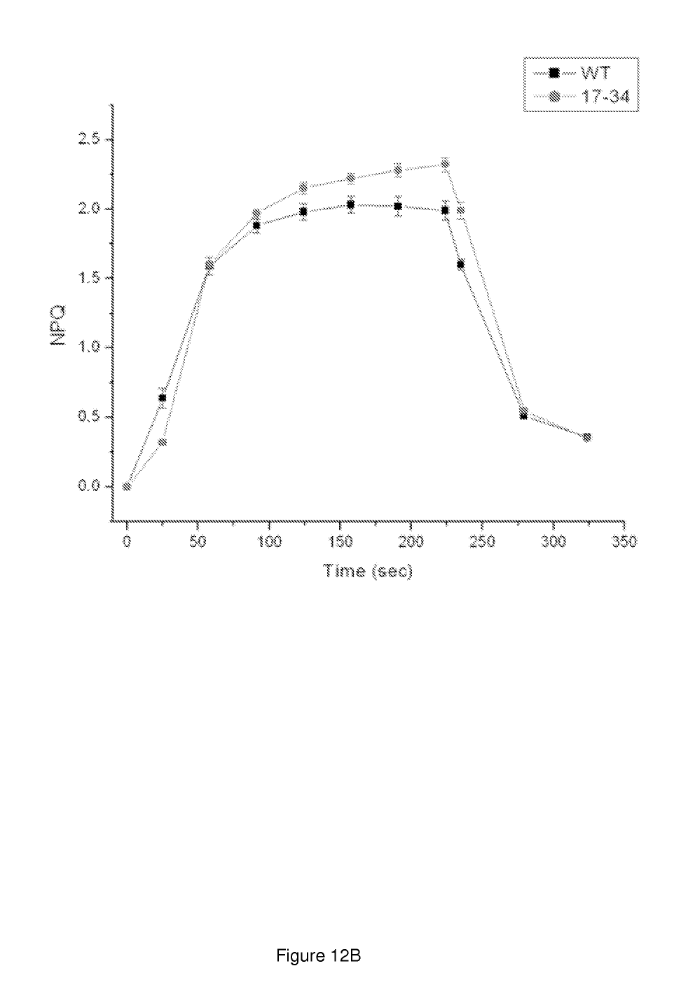

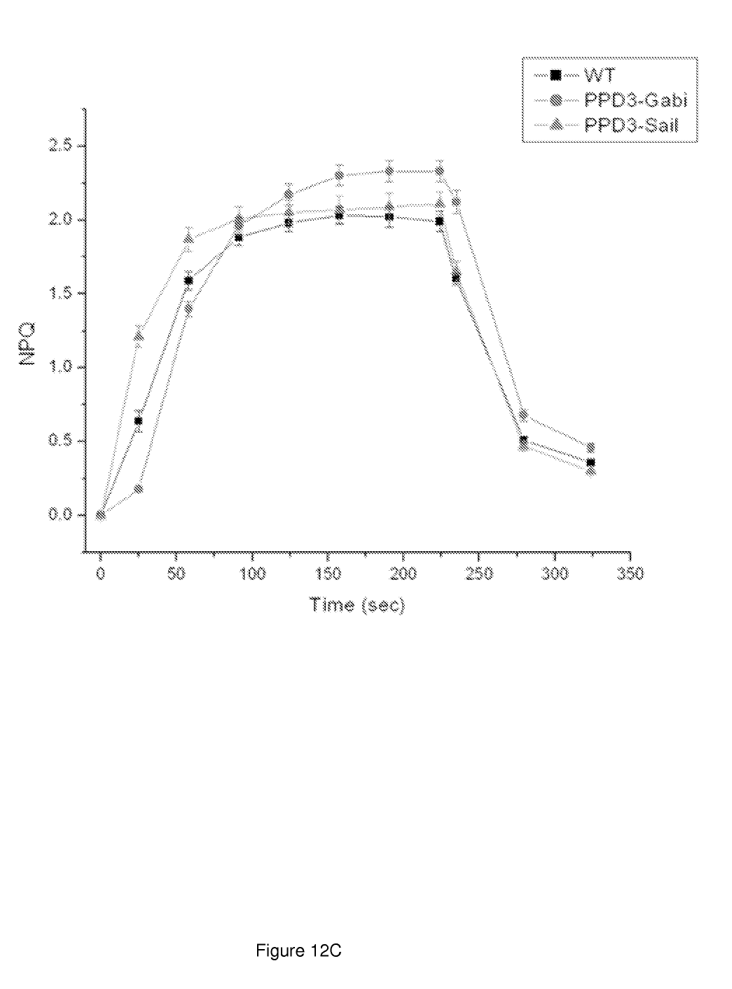

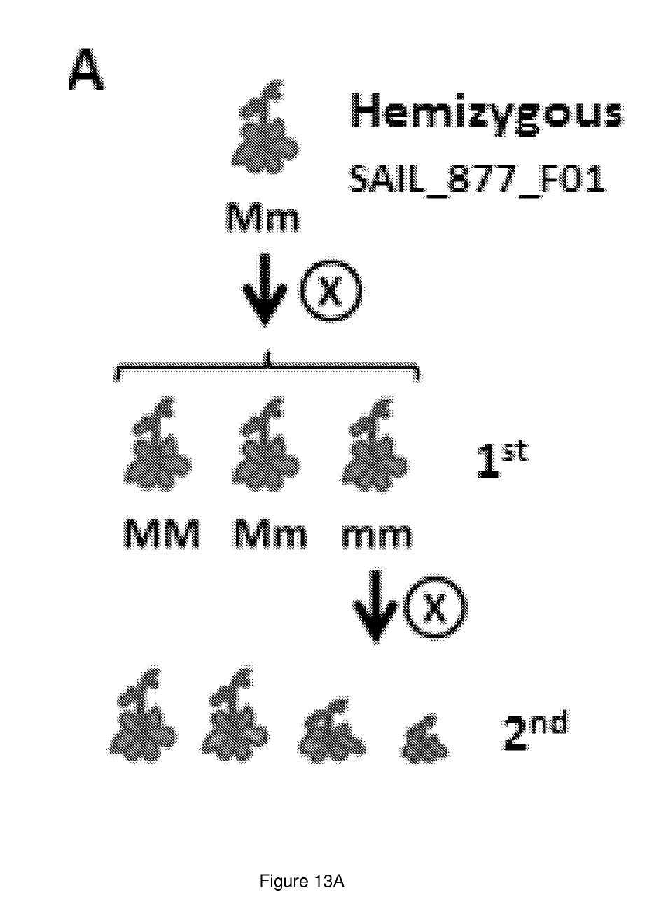

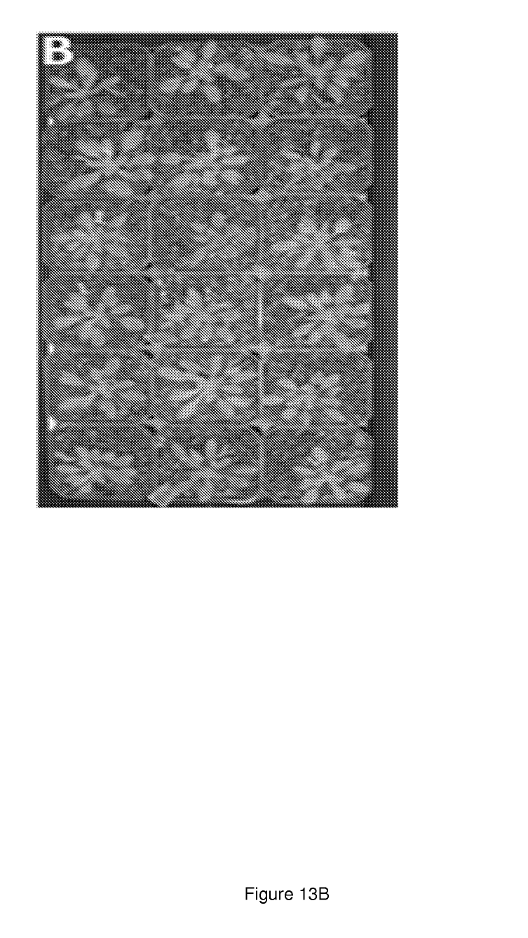

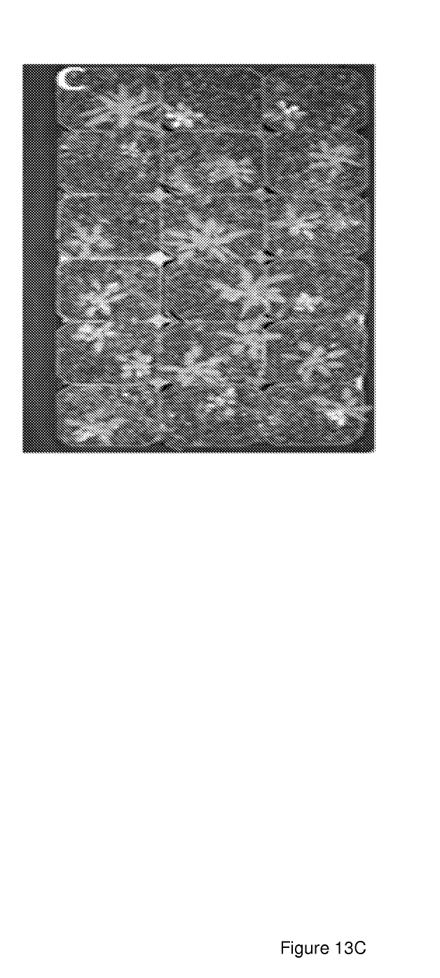

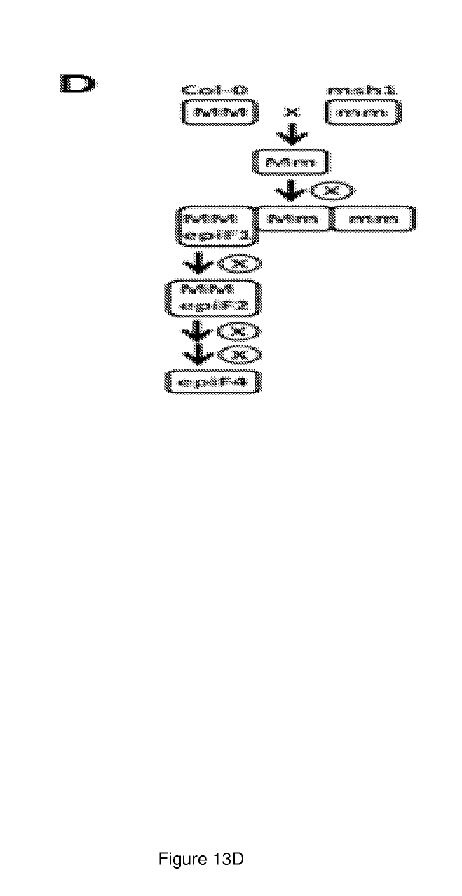

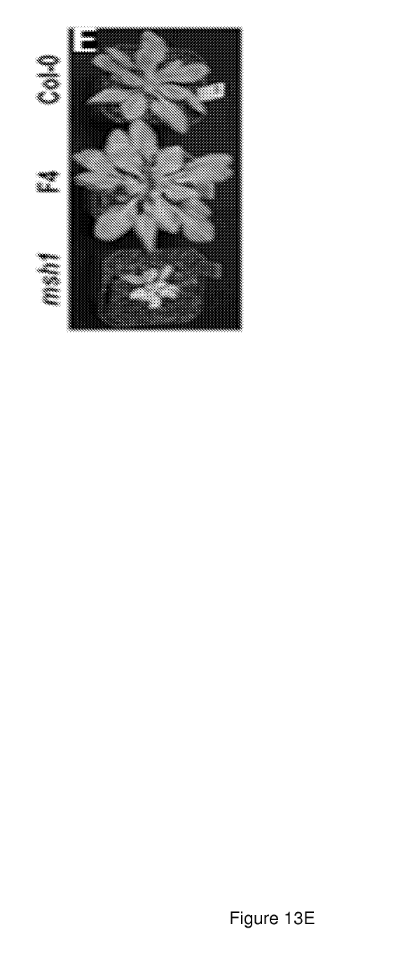

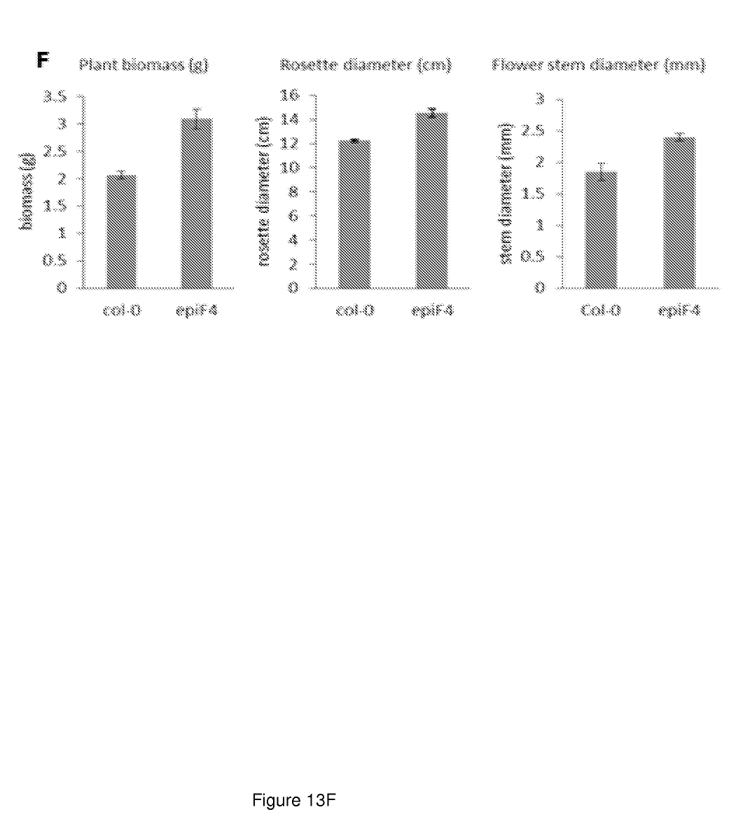

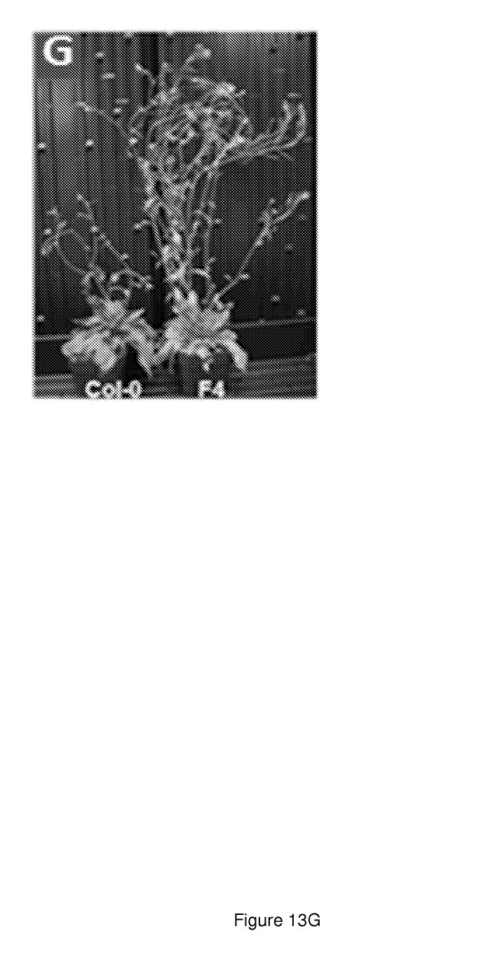

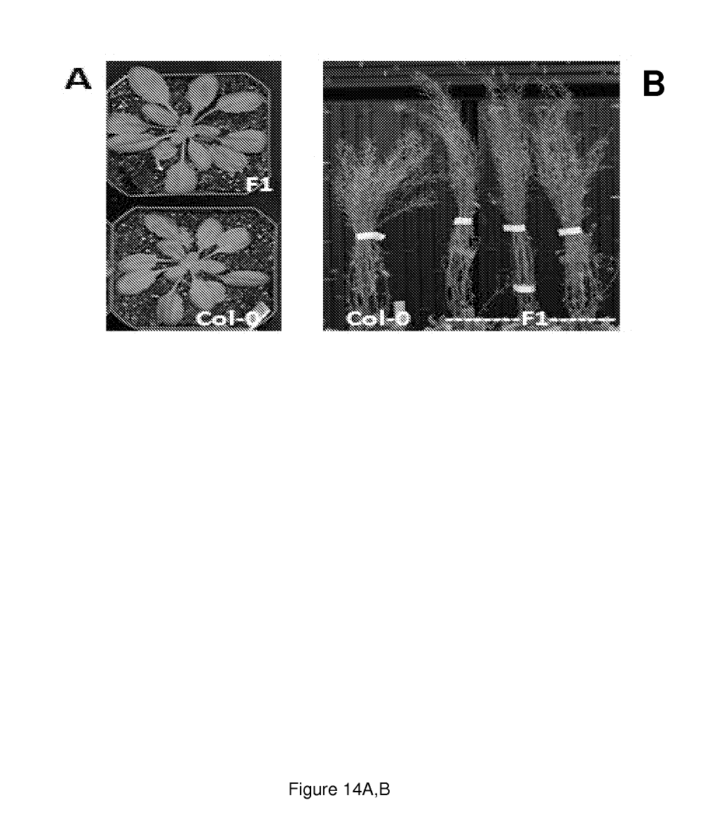

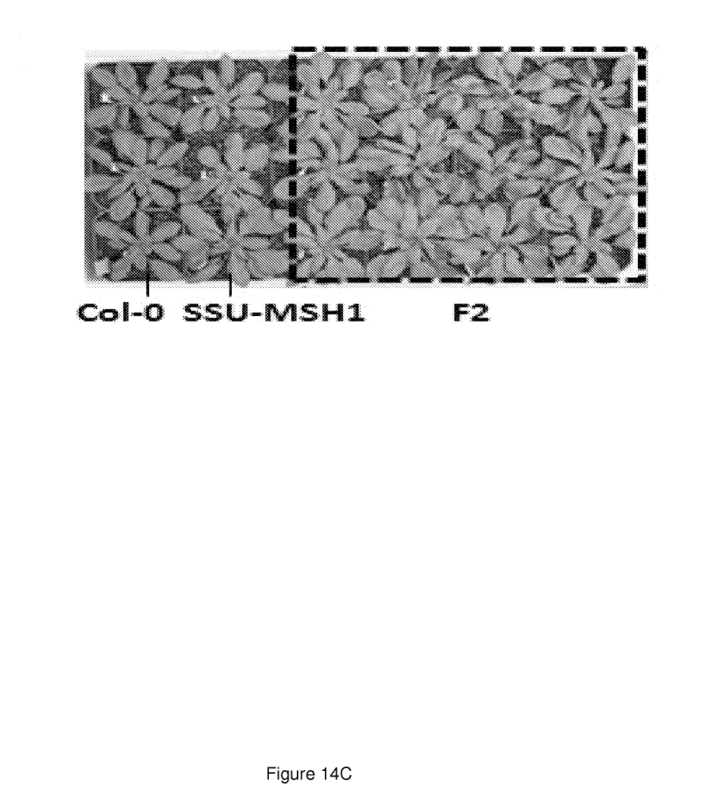

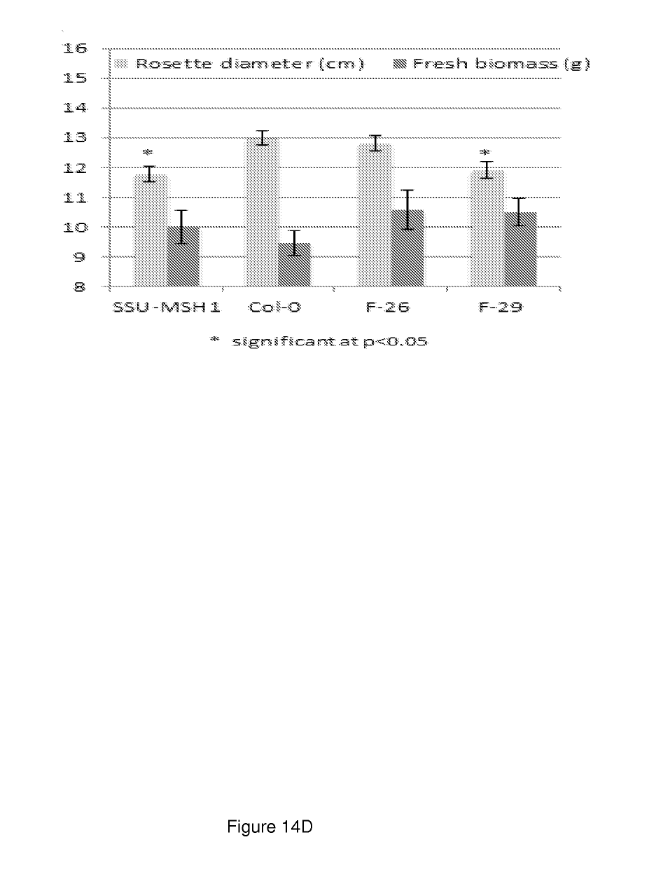

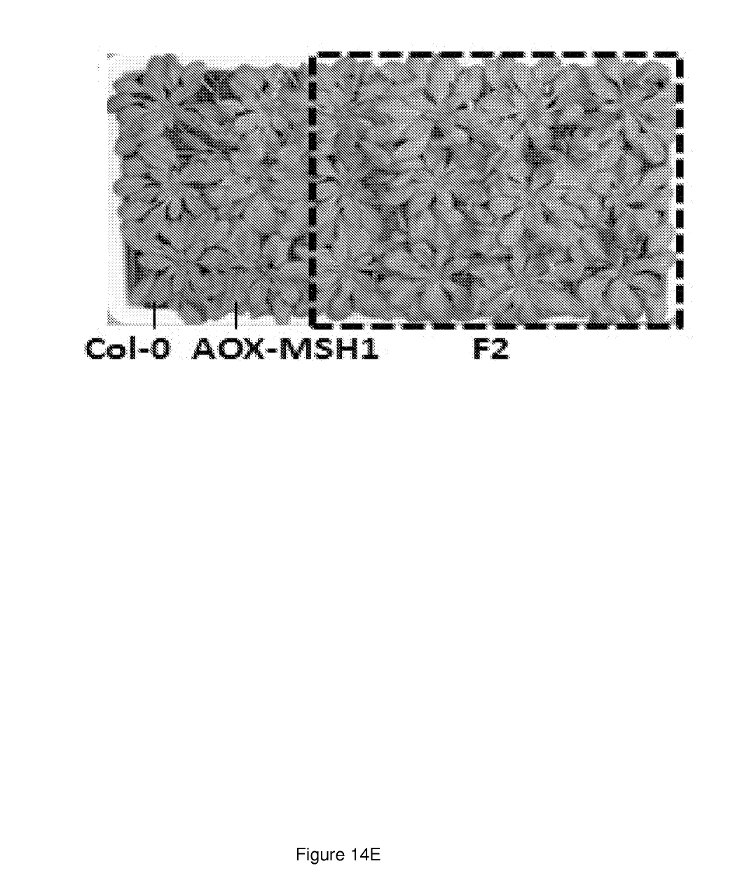

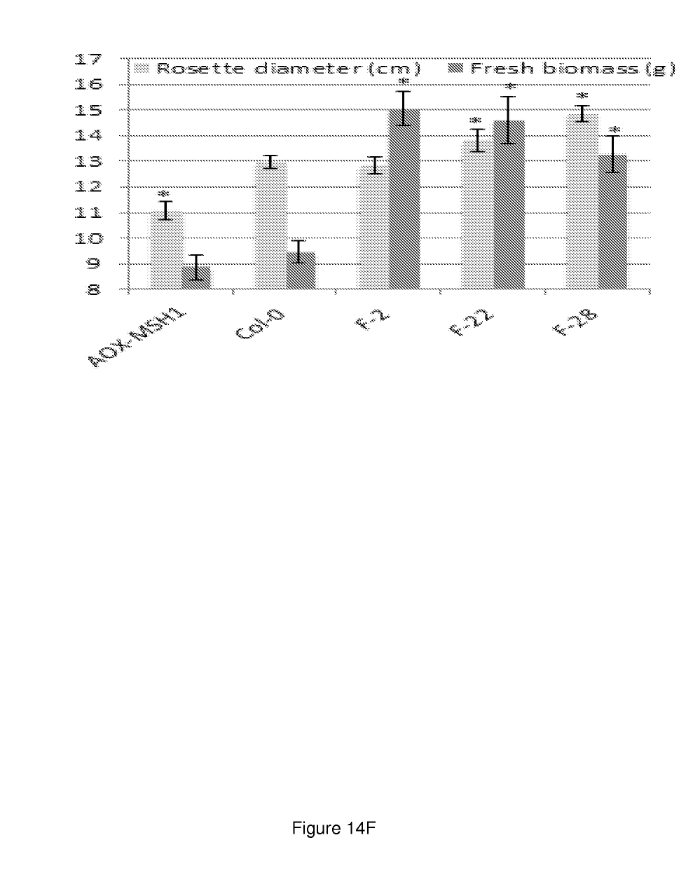

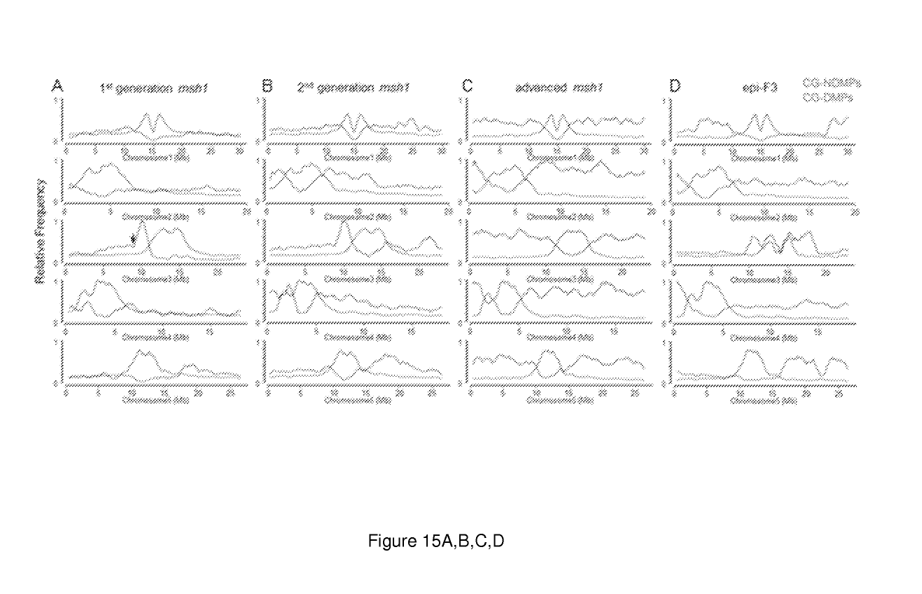

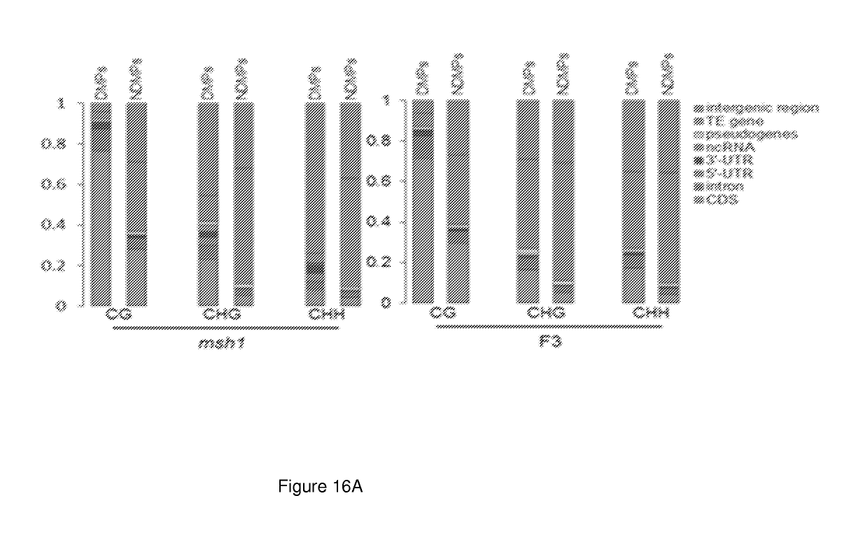

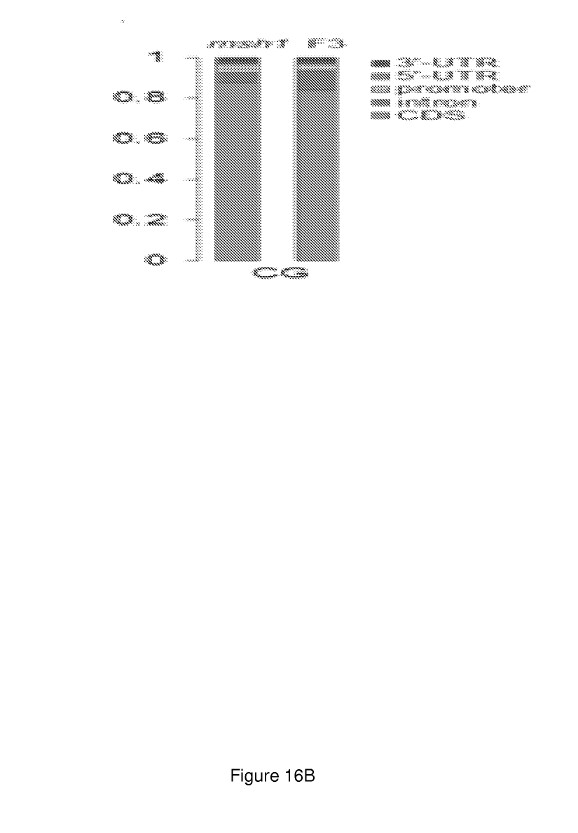

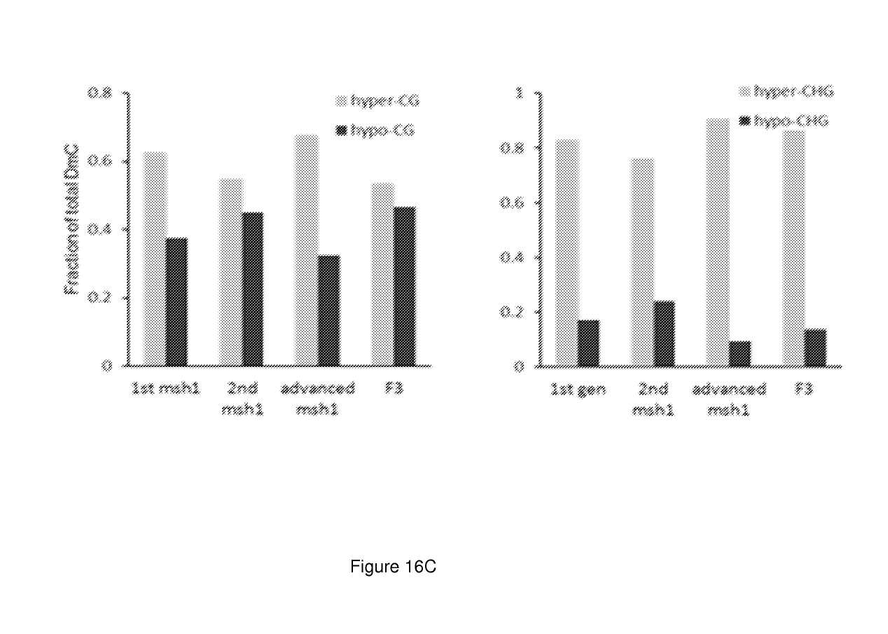

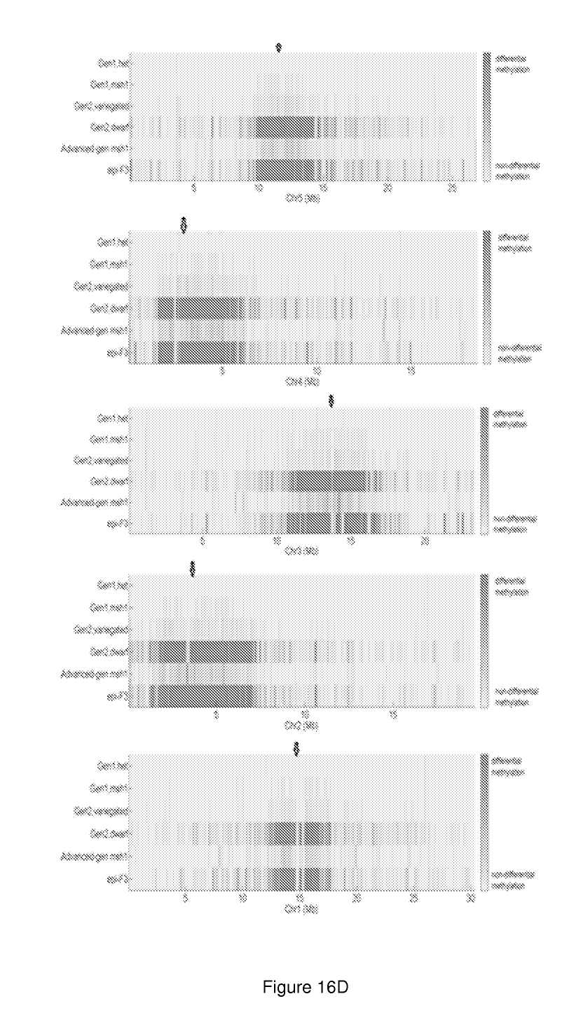

The most convincing MSH1 protein interactions data from coIP and yeast 2-hybrid experiments was derived for PPD3, a protein of unknown function. Consequently, we pursued this candidate in more detail. Full-length PPD3-GFP fusion constructs were developed to test the expression and localization pattern of PPD3. We observed, by laser scanning confocal microscopy, that PPD3 also localized to small sized plastids within the epidermal layer and the vascular parenchyma (FIG. 6). This was in contrast, for example, to PsbO2, which localized predominantly to mesophyll plastids, but also to the vascular bundle plastids (FIG. 11).

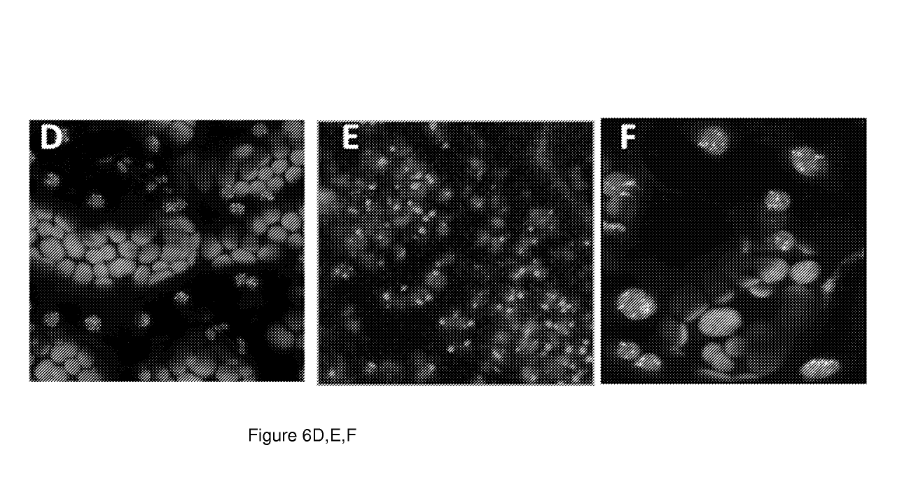

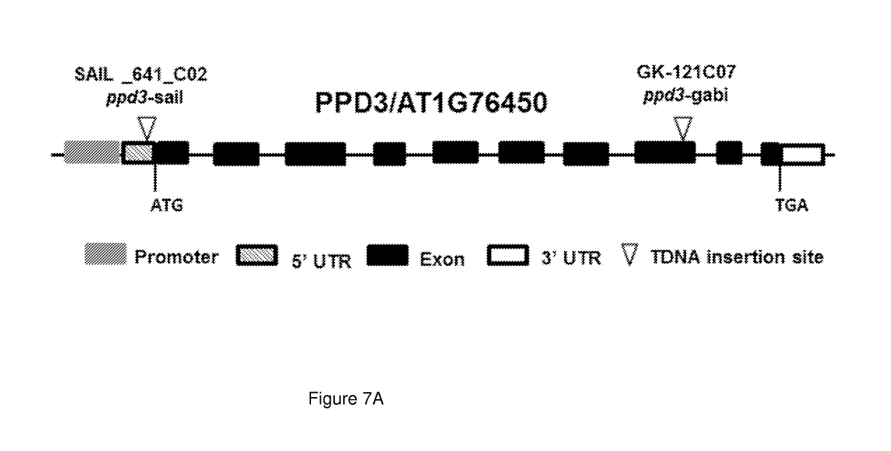

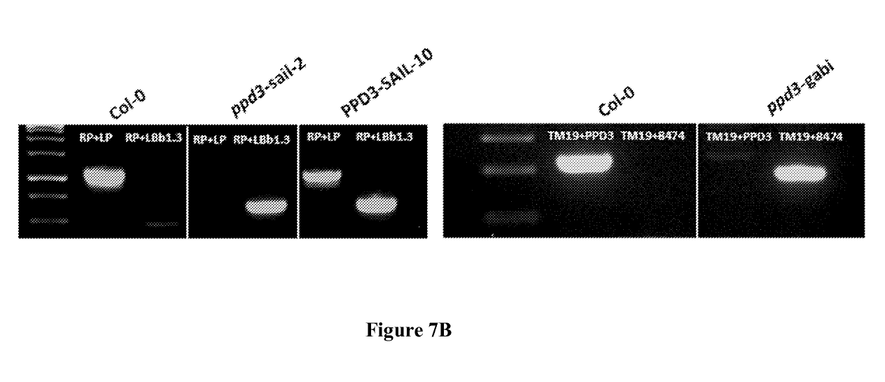

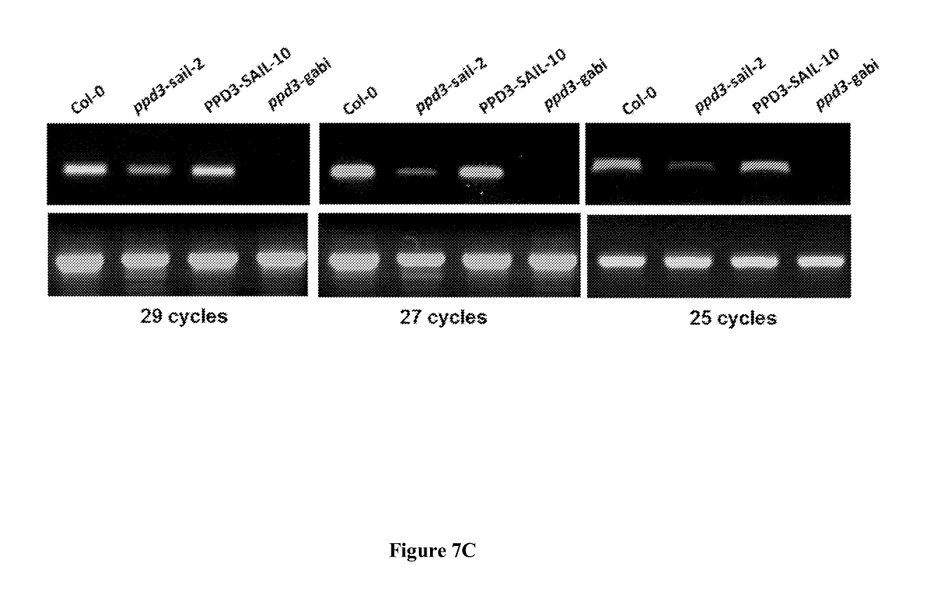

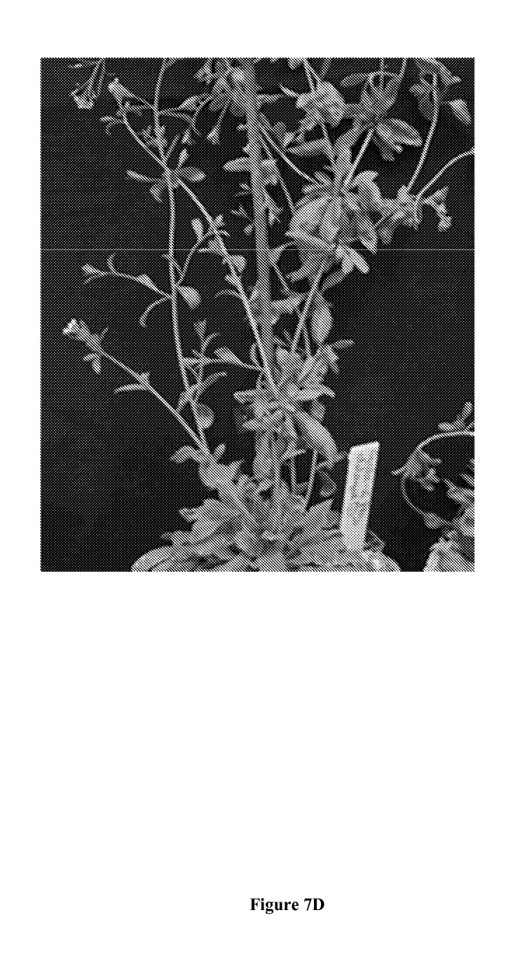

Three TDNA insertion mutants were obtained for PPD3 in Arabidopsis, located at three sites in the gene, one in an exon, one intronic, and one in the promoter (FIG. 7A). While the promoter mutant, ppd3-Sail2, reduced expression of the gene, the exon mutant ppd3-gabi produced the strongest effect on expression and also on phenotype. Growth of the ppd3-gabi mutant at 10-hour day length produced aerial rosettes and extended, woody growth that is reminiscent of what we observe in MSH1-dr lines (FIG. 7D).

MSH1 and PPD3 mutants both give rise to similar plastid redox changes.

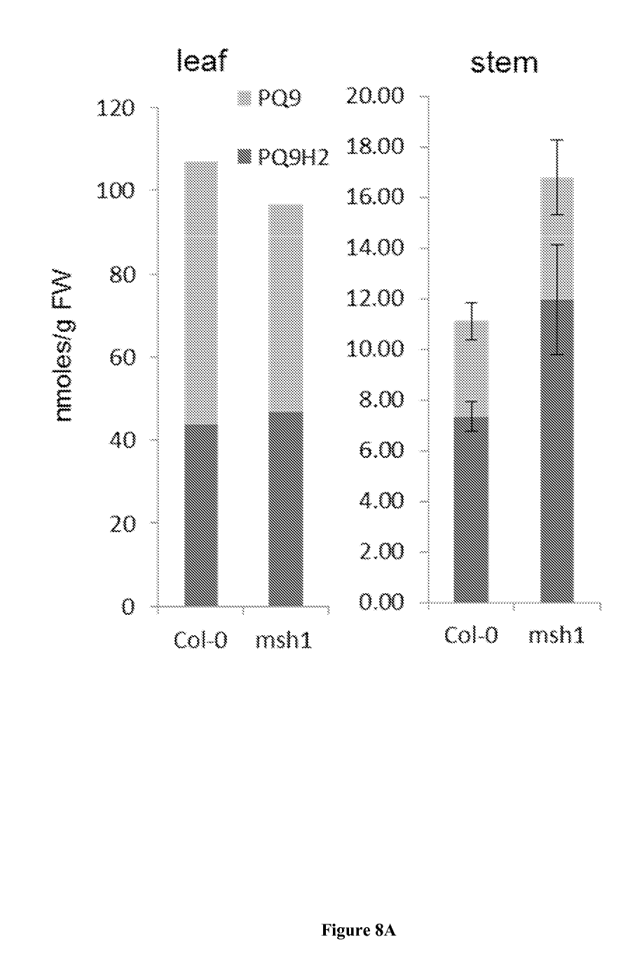

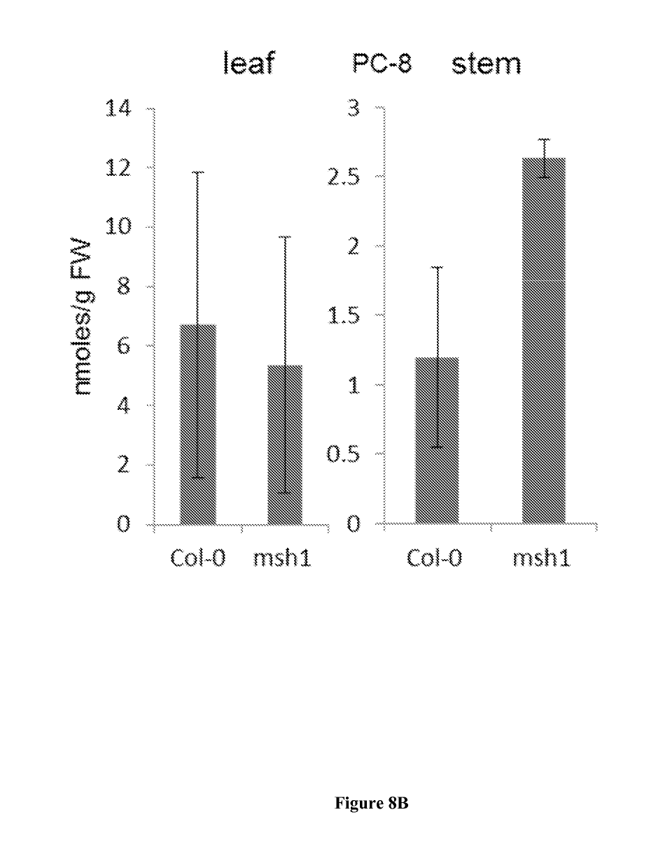

No significant differences between wildtype and msh1 mutant were apparent in amounts, oxidation rates and reduction rates of the cytochrome b6/f complex or P700, and no major defects were observed in fluorescence induction curves for assessing the efficiency of PSII closure (data not shown). However, the msh1 mutant displayed higher plastoquinone levels, in more highly reduced state, than in wildtype (FIG. 8A). This effect was more pronounced in the stem, where MSH1 expression and sensory plastids are also expected to be highest, but was less evident in the leaf. Plastochromanol −8 levels were also higher in the stem of the msh1 mutant, relative to wildtype (FIG. 8B). These observations imply that redox status of the mutant is altered. What is intriguing about these results, is that they are more pronounced in the stem than in the leaf, consistent with the hypothesis that sensory plastids, where MSH1 functions, show the most significant effects of MSH1 disruption, perhaps comprising a transmissible signal within the plant.

The msh1 mutant effect on plastid redox properties was also evident in enhanced non-photochemical quenching rates in the light, followed by slower decay rates in the dark (FIG. 12A-C). A nearly identical effect was measured in the ppd3 mutants, consistent with a likely functional interaction between MSH1 and PPD3.

### Example 2

### Methylation in MSH1 Suppressed Plants

A plant's phenotype is comprised of both genetic and non-genetic influences. Control of epigenetic effects, thought to be influenced by environment, is not well defined. Transgenerational epigenetic phenomena are thought to be important to a plant's ability to pre-condition progeny for abiotic stress tolerance. MSH1 is a mitochondrial and plastid protein, and MSH1 gene disruption leads to enhanced abiotic stress and altered development. Genome methylation changes occur immediately following disruption of MSH1, changes that are most pronounced in plants displaying the altered developmental phenotype. These developmental changes are inherited independent of MSH1 in subsequent generations, and lead to enhanced growth vigor via reciprocal crossing to wildtype, implying that loss of MSH1 function leads to programmed epigenetic changes.

Plant phenotypes respond to environmental change, an adaptive capacity that is, at least in part, trans-generational. Genotype×environment interaction in plant populations involves both genetic and epigenetic factors to define a plant's phenotypic range of response. The epigenetic aspect of this interplay is generally difficult to measure. Previously we showed that depletion of a single nuclear-encoded protein, MSH1, from the plastid causes dramatic and heritable changes in development. The changes are fully penetrant in the progeny of these plants. Here we show that crossing these altered plants with isogenic wild type restores normal growth and produces a range of phenotypic variation with markedly enhanced vigor that is heritable. In Arabidopsis, these growth changes are accompanied by redistribution of DNA methylation and extensive gene expression changes. MSH1 mutation results in very early changes in both CG and CHG methylation that drive toward hypermethylation, with pronounced changes in pericentromeric regions, and with apparent association to developmental reprogramming. Crosses to wildtype result in a significant redistribution of DNA methylation within the genome. Variation in growth observed in this study is non-genetic, suggesting that plastid perturbation by MSH1 depletion constitutes a novel means of inducing epigenetic changes in plants.

Evidence exists in support of a link between environmental sensing and epigenetic changes in plants and animals (1-3). Trans-generational heritability of these changes remains a subject of investigation (4-5), but studies in Arabidopsis indicate that it is feasible to establish new and stable epigenetic states (6-7). Much of what has been learned in plants derives from studies exploiting Arabidopsis DNA methylation mutants to disrupt the genomic methylation architecture of the plant and provide evidence of epigenomic variation in plant adaptation (8). In maize and Arabidopsis, heritable DNA methylation differences are observed among inbred lines (9) and resulting hybrids that may be related to heterosis (10). In natural Arabidopsis populations, epiallelic variation is highly dynamic and found largely as CG methylation within gene-rich regions of the genome (11-12).

Here we demonstrate that loss of MSH1 results in a pattern of early methylome changes in the genome that are most pronounced in plants that demonstrate developmental reprogramming. These effects involve heritable pericentromeric CHG and localized CG hypermethylation. These genome methylation changes may underlie the trans-generational nature of non-genetic phenotypes observed with MSH1 depletion.

A genetic strategy for organelle perturbation involves mutation or RNAi suppression of MUTS HOMOLOG 1 (MSH1). MSH1 is a mitochondrial- and chloroplast-targeted protein unique to plants and involved in organelle genome stability (13, 14). MSH1 disruption also effects developmental reprogramming (MSH1-dr) (15). A range in MSH1-dr phenotype intensity occurs, and the changes in transcript and metabolite patterns seen in MSH1-dr selections are characteristic of plant abiotic stress responses (14-15).

FIG. 1 shows the crossing process used in this study. Arabidopsis experiments were carried out in the inbred ecotype Columbia-O. Crossing wildtype Col-0 with the msh1 mutant results in a heritable, enhanced growth phenotype that, by the F3 generation (epi-F3), produces markedly larger rosettes and stem diameter, early flowering, and enhanced plant vigor (FIGS. 1E-G).

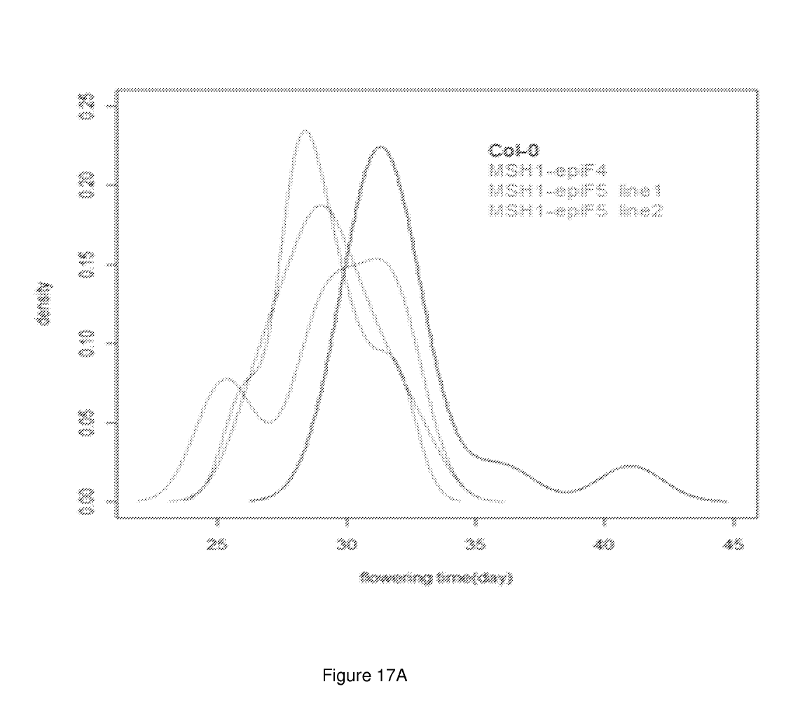

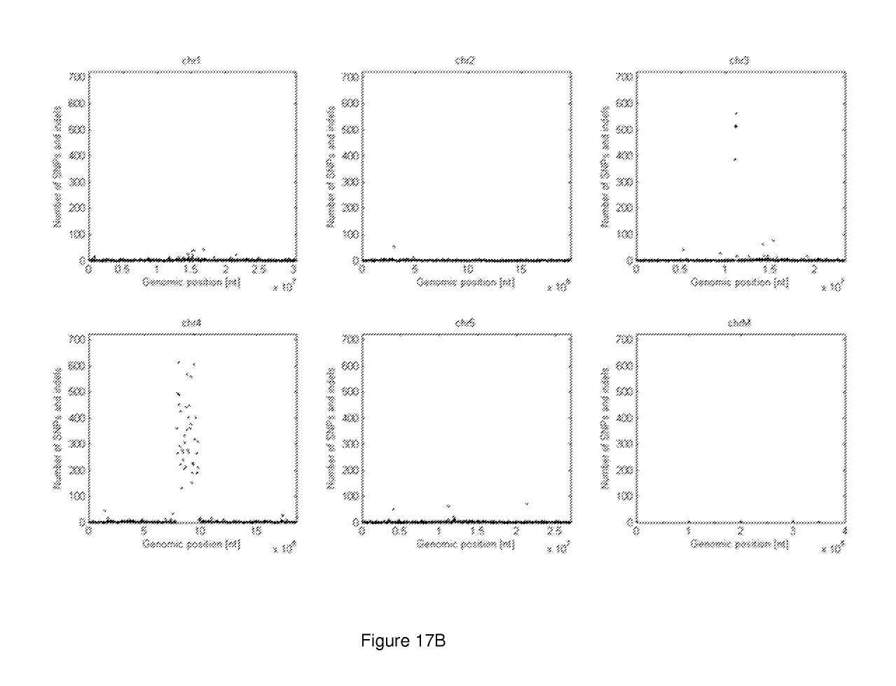

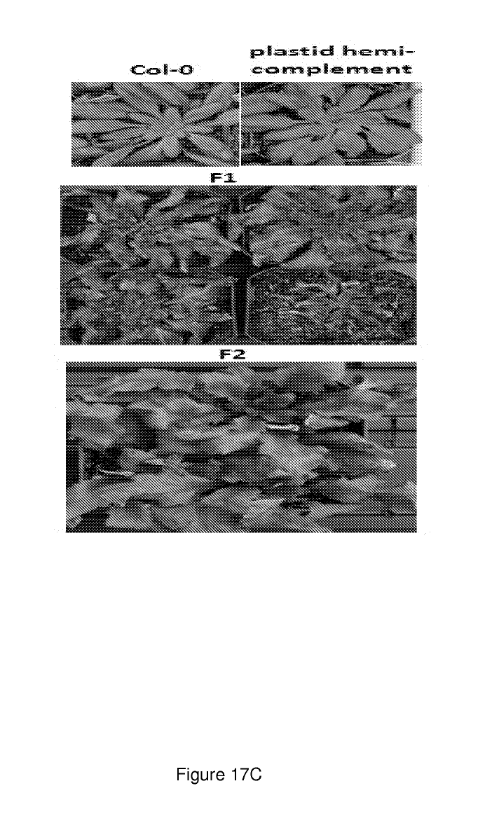

To test whether the Arabidopsis genome, with msh1 mutation, has undergone genomic rearrangement to account for the rapid developmental reprogramming, paired-end genome-wide sequencing, alignment and de novo partial assembly of the mutant genome was conducted. The longstanding chm1-1 mutant, first identified over 30 years ago, was used for these experiments, providing the best opportunity to test for any evidence of genome instability caused by MSH1 mutation. The analysis produced 14,416 contigs (n50=40,761 bp) containing 118.5 Mbp; mapping these contigs against Col-0 covers 72 Mbp. Alignment of paired-end reads to the Col-0 public reference sequence produced 95% alignment and identified 12,771 SNPs and indels, with one 2-Mbp interval, on chromosome 4, accounting for 8,582 (FIG. 17B). The chm1-1 mutant used in this study is a Col-0 mutant once crossed to Ler (13). Comparing SNPs and indels in the chromosome 4 region with those in a recent study of Ler×Col-0 (16) accounts for 5060 of 6985 SNPs (72%) and 1073 of 1597 indels (67%), consistent with an Ler introgressed segment. Of the remaining 4188 SNP/indels, 72% (2996) reside in non-genic regions. This SNP mutation rate is likely consistent with natural SNP frequencies (11), suggesting that no significant, unexplained genome alterations were detected in the msh1 mutant.

Altered plant development in Arabidopsis msh1 is conditioned by chloroplast changes (15). We found that the enhanced growth in MSH1-epiF2 lines also appeared to emanate from these organelle effects. Arabidopsis MSH1 hemi-complementation lines, derived by introducing a mitochondrial- versus chloroplast-targeted MSH1 transgene to msh1 (14), distinguish mitochondrial and chloroplast contributions to the phenomenon. Chloroplast hemi-complementation lines (SSU-MSH1) crossed as female to wild type (Col-0) produced F1 phenotypes resembling wild type (FIG. 2, Table 3), although 10% to 77% of independent F1 progenies showed slow germination, slow growth, leaf curling and delayed flowering (FIG. 17C). The curling phenotype may be a mitochondrial effect; it resembles altered salicylic acid pathway regulation, which has shown epigenetic influence (17). In F1 progeny from crosses to the mitochondrial-complemented line (AOX-MSH1), over 30% showed enhanced growth, larger rosette diameter, thicker floral stems and earlier flowering time, resembling MSH1-epiF3 phenotypes (FIG. 2; Table 3). These results were further confirmed in derived F2 populations (FIG. 2), and imply that growth enhancement arises from the MSH1-dr phenomenon.

Arabidopsis wild type, first-, second- and advanced-generation msh1 mutants, and msh1-epiF3 plants, all Col-0, were investigated for methylome variation. Bisulfite treatment and genomic DNA sequence analysis (18) was carried out on progeny from an MSH1/msh1 heterozygous T-DNA insertion line, producing first generation msh1/msh1, MSH1/msh1, and MSH1/MSH1 full-sib progeny segregants for comparison (FIG. 1A). All first-generation plants appeared normal, with only very mild variegation visible on the leaves of the msh1/msh1 segregants (FIG. 1B). These lines were compared to two second-generation msh1/msh1 lines from a parallel lineage (FIG. 1C), one a normal-growth, variegated line and one a dwarfed dr line. The advanced-generation mutant is chm1-1, with which we have carried out all of our previous studies. Methylation changes between the first-generation msh1 mutant and its wild type MSH1/MSH1 sib involved 20 CG differentially methylated regions (DMRs) (Table below). The CG DMRs were clustered on Chromosome 3, forming a peak adjacent to the MSH1 gene (FIG. 3). Whether proximity of this peak to MSH1 has functional significance or is mere coincidence is not yet known.

TABLE 2

| | CpG | | CHG | | CHH | |
|---|---|---|---|---|---|---|
| Lines | DMP | DMR | DMP | DMR | DMP | DMR |
| Gen 1, het | 6664 | 8 | 349 | 0 | 359 | 8 |
| Gen1, msh1 | 11073 | 20 | 1176 | 0 | 887 | 16 |
| Gen2, variegated | 28860 | 111 | 2885 | 4 | 1631 | 28 |
| Gen2, dwarf | 29680 | 103 | 39307 | 867 | 4625 | 45 |
| Advanced-gen msh1 | 61046 | 1001 | 5519 | 21 | 571 | 2 |

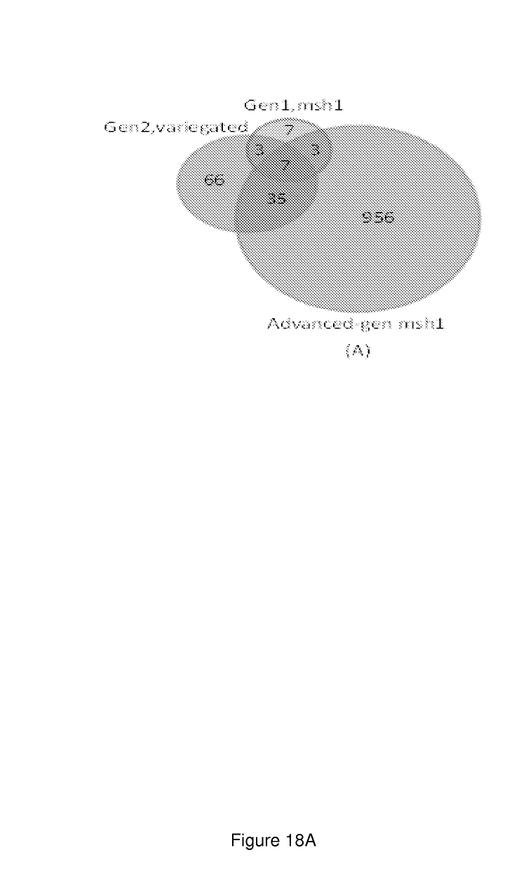

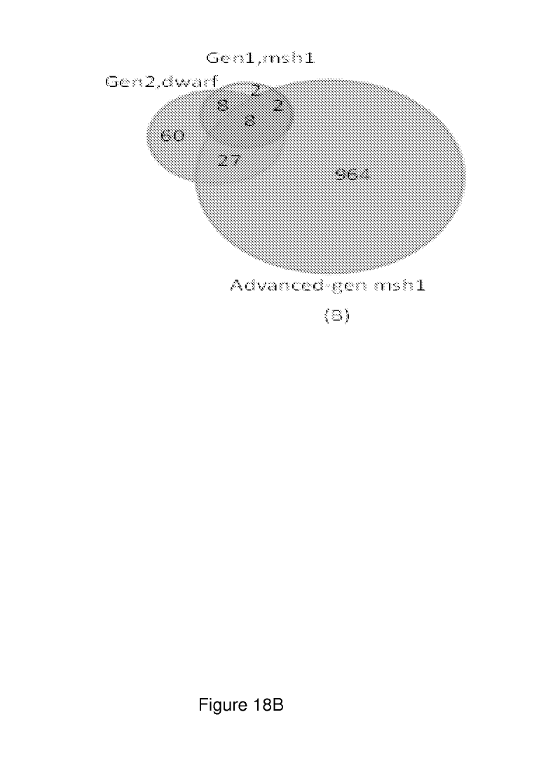

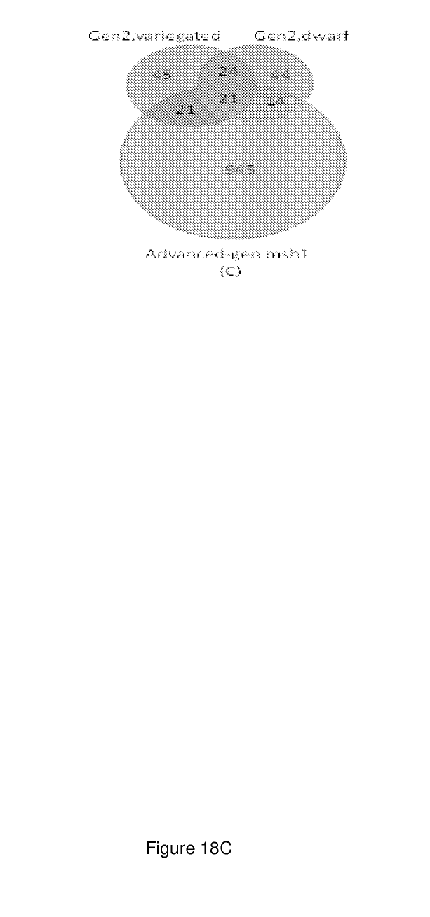

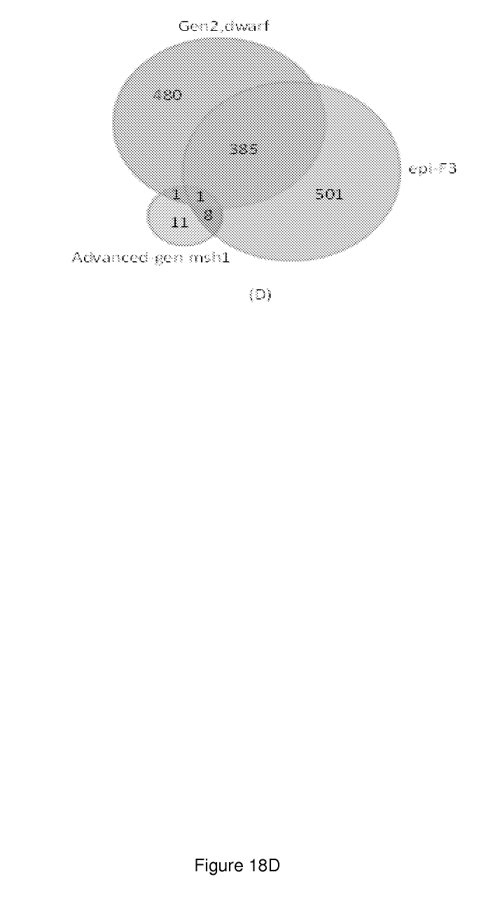

By generation 2, the variegated, normal growth line displayed 111 CG DMRs and the dwarfed, dr line displayed 103, both retaining the DMR peak on Chromosome 3 (Table immediately above, FIG. 3). Of the 20 CG DMRs observed in generation 1, 10 were retained in the variegated line and 16 were present in the dwarfed dr line (FIG. 18A-D). CHG differential methylation varied markedly in the generation 2 lines, with 4 CHG DMRs in the variegated line versus 867 CHG DMRs in the dwarfed dr line (Table immediately above). The advanced-generation msh1 mutant, compared to Col-0, showed 1001 CG DMRs, of which 56 were shared with early generation lines. Whereas the advanced-generation msh1 mutant showed 21 CHG DMRs with significant overlap to those CHG DMRs seen in early generation, the epi-F3 line showed 385 CHG DMRs (43%) with significant overlap to those seen in the dwarf line of generation 2 (FIG. 18A-D). As negative control for background, we compared the MSH1/msh1 (het) first-generation segregant to the same MSH1/MSH1 first-generation segregant used in the above comparisons, revealing only 6664 CG DMPs and 8 DMRs (Table immediately above).

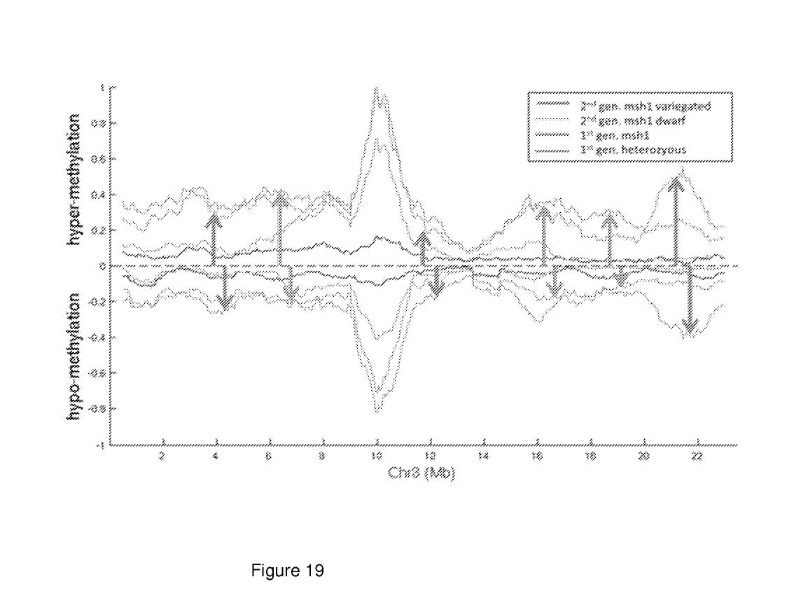

CG changes in methylation were largely in gene body regions (FIG. 4A-B). While CG DMRs generally include both loss and gain of methylation by a coordinated activity of both DNA methyl transferases and DNA glycosylases to maintain DNA methylation balance in the genome (11, 12), a disturbance in this balance is particularly evident in the second- and advanced-generation msh1 mutant lines (FIG. 4C, FIG. 19). This tendency toward hypermethylation is also particularly pronounced for CHG DMRs from generation 1 to advanced (FIG. 4C). Comparison of Col-0 and the epiF3 line, derived from crossing an early generation (gen 3) line to Col-0, showed over 2000 CG DMRs with interspersed genomic intervals of hypermethylation (FIG. 3). In the epiF3 line, methylation changes are dramatically redistributed in the genome, presumably the consequence of recombination following the cross to wildtype (FIG. 3).

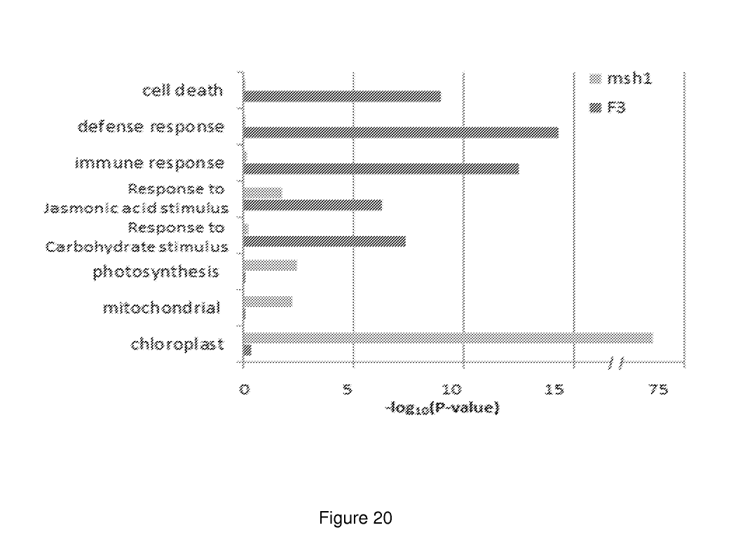

Gene expression changes in msh1 occurred for plant defense and stress response networks, while the epi-F3 lines showed predominant changes in expression of regulatory, protein turnover and several classes of kinase genes (FIG. 20). These data reflect formation of two strikingly distinct and rapid plant transitions, from wildtype to msh1-dr, and from msh1-dr to epi-F3 enhanced growth, as evidenced by plant growth phenotype, methylome and transcriptome data.

CG DMPs occurred mostly in gene coding regions, resembling natural epigenetic variation (11, 12), and gene-associated CG DMPs were located within gene bodies (FIG. 4). Non-differential methylation distributions in wildtype Col-0 versus MSH1-epiF3 and msh1, seen as blue lines in FIG. 3, showed good correspondence to that reported by an earlier Arabidopsis study of natural methylation variation in Col-0 (11). The striking differences were seen in distribution of differential methylation. The Becker et al. (11) analysis of natural variation showed fairly uniform distribution of CG differential methylation spanning each chromosome, which was also the case for advanced-generation msh1, similarly maintained by serial self-pollination (FIG. 3).

What distinguished advanced-generation msh1 methylation from that previously reported in Col-0 was the striking tendency toward hypermethylation, comprising 88% of the DMRs and 70% of total DMPs, which is not observed in natural variation patterns (11). First- and second-generation msh1 showed discrete regions of differential methylation, reflective of msh1 changes with greatly reduced background “noise” (FIG. 3). Particularly intriguing was the observation of CHG hypermethylation changes in the second-generation dwarfed dr segregants but not observed in the full-sib variegated, normal growth segregants. These changes are concentrated in pericentromeric regions of the chromosome. The second generation following msh1 depletion is the point at which the developmental reprogramming phenotype, involving dwarfing, delayed maturity transition and flowering, and woody perennial growth at short day length, is fully evident in over 20% of the plants (15). We are investigating the possible association of these pericentromeric changes with development of the dr phenotype and the derived MSH1-epiF3 enhanced growth phenotype. The hemi-complementation data suggest that development of the MSH1-dr phenotype is prerequisite to the enhanced growth effects that follow crossing to wildtype.

MSH1-epiF3 lines are developed by crossing early-generation msh1 to wild type and self-pollinating the F1 two generations. These enhanced growth lines showed hypomethylation at 33% of DMRs and 45% of total DMPs. Intervals of differential methylation were redistributed in the genome following crosses to wildtype (FIG. 3, red line), a phenomenon that may prove useful for future mapping of growth enhancing determinants.

Gene expression patterns in wildtype, msh1-dr, and enhanced growth epiF3 lines show profound changes in only one or two generations with the altered expression of MSH1. Natural reprogramming of the epigenome in plants can occur during reproductive development (19-20), when MSH1 expression is most pronounced (21). MSH1 steady state transcript levels decline markedly in response to environmental stress (14, 22). These observations suggest that MSH1 participates in environmental sensing to allow the plant to dramatically alter its growth. MSH1 suppression is a previously unrecognized process for altering plant phenotype, and may act through epigenetic remodeling to relax genetic constraint on phenotype in response to environmental change (23).

The near-identical MSH1-dr phenotypes in six different plant species (15) indicate that changes observed with MSH1 suppression are non-stochastic, programmed effects. The phenotypic transition to msh1-dr is accompanied by a significant alteration in methylome pattern that, likewise, appears non-stochastic. At least two pronounced methylome changes occur immediately upon mutation of msh1, a concentration of CG differential methylation on Chromosome 3 adjacent to and encompassing MSH1, and heritable pericentromeric CHG hyper-methylation changes in second-generation plants displaying the msh1-dr phenotype and epiF3 lines showing enhanced growth.

Crossing msh1-dr and Col-0, each with differing methylome patterns, results in redistribution of DMRs within the epi-F3 genome. Enhanced growth capacity of the resulting progeny may be the consequence of a phenomenon akin to heterosis or transgressive segregation (24, 25). Pericentromeric intervals of a chromosome tend to retain heterozygosity and have been suggested to contribute disproportionately to heterosis (26).

### Methods

Plant materials and growth conditions. Arabidopsis Col-0 and msh1 mutant lines were obtained from the Arabidopsis stock center and grown at 12 hr day length at 22° C. MSH1-epi F3 lines were derived by crossing MSH1-dr lines with wild type plants and self-pollinating two generations. Arabidopsis plant biomass and rosette diameters were measured for 4-week-old plants. Arabidopsis flowering time was measured as date of first visible flower bud appearance. For hemi-complementation crosses, mitochondrial (AOX-MSH1) and plastid (SSU-MSH1) complemented homozygous lines were crossed to Col-0 wildtype plants. Each F1 plant was genotyped for transgene and wildtype MSH1 allele and harvested separately. Three F2 families from AOX-MSH1×Col-0 and two F2 families from SSU-MSH1×Col-0 were evaluated for growth parameters. All families were grown under the same conditions, and biomass, rosette diameter and flowering time were measured. Two-tailed Student t-test was used to calculate p-values.

Bisulfite treatment of DNA for PCR analysis. Arabidopsis genomic DNA was bisulfite treated using the MethylEasy Xceed kit according to manufacturer's instructions. PCR was performed using primers listed in Table 4, and the PCR products were cloned (Topo TA cloning kit, Invitrogen) and DNA-sequenced. Sequence alignment was performed using the T-Coffee multiple sequence alignment server (27).

Bisulfite treated genomic library construction and sequencing. Arabidopsis genomic DNA (15 ug) prepared from Col-0, msh1 and epi-F3 plants was sonicated to peak range 200 bp to 600 bp. Sonicated DNA (12 ug) was treated with Mung Bean Nuclease (New England Biolabs), phenol/chloroform extracted and ethanol precipitated. Mung Bean Nuclease-treated genomic DNA (3 ug) was end-repaired and 3′ end-adenylated with Illumina (San Diego Calif.) Genomic DNA Samples Prep Kit. The adenylated DNA fragment was ligated to methylation adapters (Illumina). Samples were column purified and fractionated in agarose. A fraction of 280 bp to 400 bp was gel purified with the QIAquick Gel Purification kit (Qiagen, Valencia, Calif.). Another 3 ug of Mung Bean Nuclease treated genomic DNA was used to repeat the process, and the two fractions pooled and subjected to sodium bisulfite treatment with the MethylEasy Xceed kit (Human Genetic Signatures Pty Ltd, North Ryde, Australia). Three independent library PCR enrichments were carried out with 10 ul from total 30 ul bisulfate treated DNA as input template. The PCR reaction mixture was 10 ul DNA, 5 ul of 10× pfuTurbo Cx buffer, 0.7 ul of PE1.0 primer, 0.7 ul PE2.0 primer, 0.5 ul of dNTP (25 mM), 1 ul of PfuTurbo Cx Hotstart DNA Polymerase (Stratagene, Santa Clara, Calif.), and water to total volume 50 ul. PCR parameters were 950C for 2 min, followed by 12 cycles of 950 C 30 sec, 650 C 30 sec and 720 C 1 min, then 720 C for 5 min. PCR product was column-purified and equal volumes from each reaction were pooled to final concentration of 10 nM.

Libraries were DNA sequenced on the Illumina Genome Analyzer II with three 36-cycle TruSeq sequencing kits v5 to read 116 nucleotides of sequence from a single end of each insert (V8 protocol).

DNA Sequence analysis and identification of differentially methylated cytosines (DMCs).

FASTQ files were aligned to the TAIR10 reference genome using Bismark (28), which was also used to determine the methylation state of cytosines. One mismatch was allowed in the first 50 nucleotides of the read. Bismark only retains reads that can be uniquely mapped to a location in the genome. Genomic regions with highly homologous sequences at other locations of the genome were filtered out.

Only cytosine positions identified as methylated in at least two reads for at least one of the genotypes and sequenced at least four times in each of the genotypes were used for the identification of DMCs. For these cytosine positions, the number of reads indicating methylation or non-methylation for each genotype was tabulated using R (http://www.r-project.org). Fisher's exact test was carried out for testing differential methylation at each position. Adjustment for multiple testing over the entire genome was done as suggested in Storey and Tibshirani (29) and a false discovery rate (FDR) of 0.05 was used for identifying differentially methylated CG cytosines. A less stringent threshold was used for identifying differentially methylated cytosines of CHG and CHH, i.e. adjustment for multiple testing was done for cytosines where a p-value smaller than 0.05 and a false discovery rate (FDR) of 0.035 was used. Methylome sequence data were uploaded to the Gene Expression Omnibus with accession number GSE36783.

Mapping DMCs to Genomic Context and Identifying Differentially Methylated Regions (DMRs)

TAIR10 annotation (ftp://ftp.arabidopsis.org/home/tair/Genes/TAIR10_genome_release/TAIR10_gff3) was used to determine the counts for DMCs or non-differentially methylated cytosines in gene coding regions, 5′-UTRs, 3′-UTRs, introns, pseudogenes, non-coding RNAs, transposable element genes, and intergenic regions. Intergenic regions were defined as regions not corresponding to any annotated feature.

For each methylation context (CG, CHG, CHH), the genome was scanned for regions enriched in DMCs using a 1-kb window in 100-bp increments. Windows with at least four DMCs were retained and overlapping windows were merged into regions. Regions with at least 10 DMCs were retained with the boundary trimmed to the furthest DMCs in the region.

Microarray analysis. Microarray experiments were carried out as described previously (14). Total RNA was extracted from 8-week-old Col-0 and MSH1-epiF3 Arabidopsis plants using TRIzol (Invitrogen) extraction procedures followed by purification on RNeasy columns (Qiagen). Three hybridizations were performed per genotype with RNA extractions from single plants for each microarray chip. Samples were assayed on the Affymetrix GeneChip oligonucleotide 22K ATH1 array (Affymetrix) according to the manufacturer's instructions. Expression data from Affymetrix GeneChips were normalized using the Robust Multichip Average method (30). Tests for differential expression between genotypes were performed with the limma package (31). The false discovery rate is controlled at 0.1 for identifying differentially expressed genes. Gene ontology analysis is carried out using DAVID v6.7 (32). The microarray data have been deposited at the Gene Expression Omnibus with accession number GSE43993.

Genome sequencing, de-novo genome assembly and SNP analysis of msh1. Genome sequencing was carried out at the Center for Genomics and Bioinformatics at Indiana University. The 20 nM dilutions were made for DNA samples prepared from mutant msh1 and one epiF5 line. Preparation of single stranded DNA used 5 ul 20 nM dilution and 5 ul 0.2N NaOH inclubated for 5 min and diluted with 990 ul Illumina HT1 Hyb buffer for 100 pM ssDNA stocks. 100 ul of 100 pM stock, 397 ul Ht1 buffer and 3 ul PhiX 10 nM ssDNA control were loaded to the flowcell of the Illumina MiSeq and processing was according to manufacturer's instructions.

Raw paired-end reads (mate 1: 300 bp; mate 2: 230 bp) were quality trimmed with a Phred quality threshold of 20 and reads with a subsequent length of less than 50 bases were removed. Illumina TruSeq adapter (index 22) was trimmed (prefixed with ‘A’ user for adapter ligation), removing from the adapter match to the 3′ end of the read. A second pass of adapter trimming without the ‘A’ prefix was done to remove adapter dimers. Ambiguous bases were trimmed from the 5′ and 3′ end of reads, and those reads with more than 1% number of ambiguous bases were completely removed. A second pass of quality filtering was performed, again with bases lower than a Phred quality score of 20 being trimmed, and reads of less than 50 bases being removed. A PhiX (RefSeq: NC_{—}001422) spike-in was removed by mapping the reads via bowtie233 (version 2.0.6) against the PhiX genome and filtering out any hits from the FASTQ files via a custom Perl script (available upon request). The resulting FASTQ files were synchronized, such that only full mate-pairs remained, while orphans (only one mate exists) were stored in an separate file. Cutadapt (33) (version 1.2.1) was used for the adapter removal, and the NGS-QC toolkit (34) (version 2.3) and fastq_quality_trimmer (35) (part of FASTX Toolkit 0.0.13.2) were used for the removal of ambiguous bases and quality filtering, respectively.

The msh1 genome was assembled using Velvet (36) with a kmer value of 83, an insert length of 400 bases, a minimum contig length of 200 bases, and the short paired (the PE reads) and a short read (the orphans) FASTQ files. The expected coverage (−exp_cov) and coverage cutoff (−cov_cutoff) were determined manually to be 25 and 8, respectively, by inspecting the initial weighted coverage of the first assembly. Resulting contigs were mapped back to Col-0 via blastn (37)(version 2.2.26+) using an e-value of 10-20 and coverage was determined with a custom Perl script (available upon request).

For the SNP and indel detection between msh1 and Col-0, the PE reads were aligned against the TAIR10 reference version of the Col-0 genome sequence via the short read aligner Bowtie2 (38) using the very-sensitive option and allowing one mismatch per seed (−N 1). Only the best alignment was reported and stored in a SAM file. The SAM file was processed via samtools mpileup (39)(version 0.1.18) and subsequently filtered by a minimum read depth of 20, a minimum mapping quality of 30, and a minimum SNP or indel Phred quality score of 30 (p 0.001).

The SNPs and small indels were compared to supplementary data files from Lu et al. (16) with custom made Perl scripts (available upon request). The msh1 genome sequence data has been uploaded to the Short Read Archive under sample number SAMN0919714.

Table 3. Analysis of phenotype data from individual Arabidopsis F₂ families derived by crossing hemi-complementation lines×Col-0 wildtype. SSU-MSH1 refers to lines transformed with the plastid-targeted form of MSH1; AOX-MSH1 refers to lines containing the mitochondrial-targeted form of the MSH1 transgene. In all genetic experiments using hemi-complementation, presence/absence of the transgene was confirmed with a PCR-based assay.

TABLE 3

| | Rosette diameter | | | | | Fresh biomass | | | | |
|---|---|---|---|---|---|---|---|---|---|---|
| | Mean | | Std. | Std. | p- | Mean | | Std. | Std. | p- |
| | (cm) | N | Error | Dev | value | (g) | N | Error | Dev | value |
| AOX-MSH1 | 11.07 | 36 | 0.37 | 2.23 | <0.001 | 8.86 | 10 | 0.47 | 1.33 | NS |
| SSU-MSH1 | 11.76 | 18 | 0.26 | 1.10 | <0.001 | 10 | 10 | 0.55 | 1.55 | NS |
| Col-0 | 12.98 | 42 | 0.24 | 1.59 | — | 9.45 | 10 | 0.43 | 1.36 | — |
| F-2 (AOX- | 12.83 | 21 | 0.34 | 1.57 | NS | 15.07 | 10 | 0.66 | 2.07 | <0.001 |
| MSH1 × Col-0) | | | | | | | | | | |
| F-22 (AOX- | 13.82 | 21 | 0.42 | 1.92 | <0.10 | 14.62 | 10 | 0.92 | 2.24 | <0.001 |
| MSH1 × Col-0) | | | | | | | | | | |
| F-28 (AOX- | 14.85 | 21 | 0.31 | 1.42 | <0.001 | 13.27 | 10 | 0.70 | 1.99 | <0.001 |
| MSH1 × Col-0) | | | | | | | | | | |
| F-26 (SSU- | 12.82 | 20 | 0.25 | 1.12 | NS | 10.57 | 10 | 0.66 | 1.74 | NS |
| MSH1 × Col-0) | | | | | | | | | | |
| F-29 (SSU- | 11.9 | 21 | 0.27 | 1.25 | <0.001 | 10.5 | 10 | 0.45 | 1.19 | NS |
| MSH1 × Col-0) | | | | | | | | | | |

†P values are based on two-tailed Student t-test comparing to Col-0
NS = Not Significant

TABLE 4
Primers used in the study

| | |
|---|---|
| Primer name | Sequence |

For bisulfite sequencing:

| | |
|---|---|
| AT5G67120RING-F | 5′-TTTTTAGGAATTATTGAGTATTATTGA-3′ |
| | (SEQ ID NO: 42) |
| AT5G67120RING-R | 5′-AAATAAAAATCATACCCACATCCC-3′ |
| | (SEQ ID NO: 43) |
| AT1G20690SWI-F | 5′-TGTTGAATTATTAAGATATTTAAGAT-3′ |
| | (SEQ ID NO: 44) |
| AT1G20690SWI-R | 5′-TCAACCAATAAAAATTACCATCTAC-3′ |
| | (SQ ID NO: 45) |
| AT3g271501stMir2- | 5′- |
| F | TAAGTTTTTTTTAAGAGTTTGTATTTGTAT-3′ |
| | (SEQ ID NO: 46) |
| AT3g271501stMir2- | 5′-TAAAAATAATCAAAACCTAACTTAC-3′ |
| R | (SEQ ID NO: 47) |
| AT3g271502ndMir2- | 5′-ATTGTTTATTAAATGTTTTTTAGTT-3′ |
| F | (SEQ ID NO: 48) |
| AT3g271502ndMir2- | 5′-CTAACAATTCCCAAAACCCTTATC-3′ |
| R | (SEQ ID NO: 49) |

For PCR assay of MSH1-RATAi transgene:

| | |
|---|---|
| RNAi-F | 5′-GTGTACTCATCTGGATCTGTATTG-3′ |
| | (SEQ ID NO: 50) |
| RNAi-R | 5′-GGTTGAGGAGCCTGAATCTCTGAAC-3′ |
| | (SEQ ID NO: 51) |

### REFERENCES FOR EXAMPLE 2

1. Bonasio, R., Tu, S. & Reinberg, D. (2010) Molecular signals of epigenetic states. Science 33: 612-616
2. Mirouze, M. & Paszkowski, J. (2011) Epigenetic contribution to stress adaptation in plants. Curr Opin Plant Biol. 14:267-274
3. Dowen, R. H. et al. (2012) Widespread dynamic DNA methylation in response to biotic stress. Proc. Natl. Acad. Sci. USA 109: E2183-2191
4. Youngson, N. A. & Whitelaw, E. (2008) Transgenerational epigenetic effects. Annu. Rev. Genom. Human Genet 9: 233-257
5. Paszkowski, J. & Grossniklaus, U. (2011) Selected aspects of transgenerational epigenetic inheritance and resetting in plants. Curr. Opin. Plant Biol. 14: 195-203
6. Reinders, J. et al. (2009) Compromised stability of DNA methylation and transposon immobilization in mosaic Arabidopsis epigenomes. Genes Dev. 23: 939-950
7. Johannes, F. et al. (2009) Assessing the impact of transgenerational epigenetic variation on complex traits. PLoS Genet. 5: e1000530
8. Roux, F. et al. (2011) Genome-wide epigenetic perturbation jump-starts patterns of heritable variation found in nature. Genetics 188: 1015-1017.
9. Eichten, S. R. et al. (2011) Heritable epigenetic variation among maize inbreds. PLoS Genet. 7: e1002372.
10. Shen, H. et al. (2012) Genome-wide analysis of DNA methylation and gene expression changes in two Arabidopsis ecotypes and their reciprocal hybrids. Plant Cell 24: 875-892
11. Becker, C. et al. (2011) Spontaneous epigenetic variation in the Arabidopsis thaliana methylome. Nature 480: 245-249
12. Schmitz, R. J. et al. (2011) Transgenerational epigenetic instability is a source of novel methylation variants. Science 334: 369-373
13. Abdelnoor, R. V. et al. (2003) Substoichiometric shifting in the plant mitochondrial genome is influenced by a gene homologous to MutS. Proc. Natl. Acad. Sci. USA 100: 5968-5973
14. Xu, Y.-Z. et al. (2011) MutS HOMOLOG1 is a nucleoid protein that alters mitochondrial and plastid properties and plant response to high light. Plant Cell 23: 3428-3441
15. Xu, Y.-Z. et al. (2012) The chloroplast triggers developmental reprogramming when MUTS HOMOLOG1 is suppressed in plants. Plant Physiol. 159: 710-720
16. Lu, P. et al. (2012) Analysis of Arabidopsis genome-wide variations before and after meiosis and meiotic recombination by resequencing Landsberg erecta and all four products of a single meiosis. Genome Res. 22: 508-518
17. Stokes, T. L., Kunkel, B. N. & Richards, E. J. (2002) Epigenetic variation in Arabidopsis disease resistance. Genes Dev 16: 171-182
18. Lister, R. et al. (2008) Highly integrated single-base resolution maps of the epigenome in Arabidopsis. Cell 133: 523-36
19. Hsieh, T.-F., et al. (2009) Genome-wide demethylation of Arabidopsis endosperm. Science 324: 1451-1454
20. Gehring, M., Bubb, K. L. & Henikoff, S. (2009) Extensive demethylation of repetitive elements during seed development underlies gene imprinting. Science 324: 1447-1451
21. Shedge, V., Arrieta-Montiel, M. P., Christensen, A. C. & Mackenzie, S. A. (2007) Plant mitochondrial recombination surveillance requires unusual RecA and MutS homologs. Plant Cell 19: 1251-1264
22. Shedge, V., Davila, J., Arrieta-Montiel, M. P., Mohammed, S. & Mackenzie S. A. (2010) Extensive rearrangement of the Arabidopsis mitochondrial genome elicits cellular conditions for thermotolerance. Plant Physiol. 152: 1960-1970
23. Kalisz, S. & Kramer, E. M. (2008) Variation and constraint in plant evolution and development. Hered. 100: 171-177
24. Greaves, I., Groszmann, M., Dennis, E. S. & Peacock, W. J. (2012) Trans-chromosomal methylation. Epigenetics 7:800-805
25. Shivaprasad, P. V., Dunn, R. M., Santos, B. A., Bassett, A. & Baulcombe, D. C. (2012) Extraordinary transgressive phenotypes of hybrid tomato are influenced by epigenetics and small silencing RNAs. EMBO J 31: 257-266
26. McMullen M. D., et al. (2009) Genetic properties of the maize nexted association mapping population. Science 7: 737-740
27. Notredame, C., Higgins, D. G. & Heringa, J. (2000) T-Coffee: A novel method for fast and accurate multiple sequence alignment. J Mol. Biol. 302: 205-217
28. Krueger, F. & Andrews, S. R. (2011) Bismark: a flexible aligner and methylation caller for Bisulfite-Seq applications. Bioinformatics 27:1571-1572
29. Storey, J. D. & Tibshirani, R. (2003) Statistical significance for genome-wide studies. Proc. Natl. Acad. Sci. USA 100: 9440-9445
30. Bolstad, B., Irizarry, R. A., Astrand, M. & Speed T. (2003) A comparison of normalization methods for high density oligonucleotide array data based on bias and variance. Bioinformatics 19: 195-193
31. Smyth, G. K. (2004) Linear models and empirical Bayes methods for assessing differential expression in microarray experiments. Stat. Appl. Genet. Mol. Biol. 3: Article 3
32. Huang, D. W., Sherman, B. T. & Lempicki, R. A. (2009) Systematic and integrative analysis of large gene lists using DAVID Bioinformatics Resources. Nat. Protoc. 4:44-57
33. Martin M. (2011) Cutadapt removes adapter sequences from high-throughput sequencing reads. EMBnet Journal, Vol 17, No 1.
34. Langmead, B. & Salzberg, S. NGS QC Toolkit: A toolkit for quality control of next generation sequencing data. PLoS ONE 7(2): e30619
35. Harmon Lab. FASTX-Toolkit. On the interne at “hannonlab.cshl.edu/fastx_toolkit/”
36. Zerbino D R, McEwen G K, Margulies E H, Birney E. (2009) Pebble and Rock Band: Heuristic Resolution of Repeats and Scaffolding in the Velvet Short-Read de Novo Assembler. PLoS ONE 4(12): e8407
37. Camacho, C. et al. (2012) BLAST+: architecture and applications. BMC Bioinformatics 10, 421 (2009). Fast gapped-read alignment with Bowtie 2. Nat. Methods 9: 357-359
38. Li, H. et al. (2009) The Sequence alignment/map (SAM) format and SAMtools. Bioinformatics 25: 2078-2079

### Example 3

### Summary Tables of Nucleic Acid Sequences and SEQ ID NO

TABLE 5
Nucleotide Sequences of SEQ ID NO:1-54 provided in the Sequence Listing

| Internet Accession | SEQ ID | |
|---|---|---|
| Information | NO | Comments |
| The Arabidopsis Information Resource | 1 | Arabidopsis |
| (TAIR) | | MSH1 |
| 1009043787 | | Full length cDNA (DNA |
| on the internet (world wide web) at | | sequence) |
| arabidopsis.org | | |
| The Arabidopsis Information Resource | 2 | Arabidopsis |
| (TAIR) | | MSH1 Protein (amino acid |
| 1009118392 | | sequence) |
| on the internet (world wide web) at | | |
| arabidopsis.org | | |
| NCBI AY856369 | 3 | Soybean MSH1 |
| on the world wide web at | | >gi\|61696668\|gb\|AY856369.1\| |
| ncbi.nlm.nih.gov/nuccore | | Glycine max DNA mismatch |
| | | repair protein (MSH1) |
| | | complete cds; (DNA sequence) |
| NCBI Accession | 4 | Zea mays MSH1 |
| AY856370 | | gi\|61696670\|gb\|AY856370.1\| |
| on the world wide web at | | Zea mays DNA mismatch |
| ncbi.nlm.nih.gov/nuccore | | repair protein (MSH1), |
| | | complete cds; (DNA sequence) |
| NCBI Accession | 5 | Tomato MSH1 |
| AY866434.1 | | >gi\|61696672\|gb\|AY866434.1\| |
| on the world wide web at | | Lycopersicon esculentum DNA |
| ncbi.nlm.nih.gov/nuccore | | mismatch repair protein |
| | | (MSH1), partial cds; (DNA |
| | | sequence) |
| NCBI | 6 | Sorghum MSH1 |
| XM002448093.1 | | >gi\|242076403:1-3180 |
| on the world wide web at | | Sorghum bicolor hypothetical |
| ncbi.nlm.nih.gov/nuccore | | protein; (DNA sequence) |
| Os04g42784.1 | 7 | Rice (Oryza sativa) MSH1 |
| Rice Genome Annotation Project - MSU | | coding sequence (DNA |
| Rice Genome Annotation (Osal) | | sequence) |
| Release 6.1 | | |
| Internet address | | |
| rice.plantbiology.msu.edu/index.shtml | | |
| Brachypodium | 8 | Brachypodium |
| Bradi5g15120.1 | | MSH1 coding region (DNA |
| On the world wide web at | | sequence) |
| gramene.org/Brachypodium_distachyon/ | | |
| Gene/Summary?db=core;g=BRADI5G1 | | |
| 5120;r=5:18500245- | | |
| 18518223;t=BRADI5G15120.1 | | |
| GSVIVT01027931001 | 9 | Vitis Vinifera |
| On the world wide web at | | MSH1 cDNA (DNA sequence) |
| genoscope.cns.fr/spip/Vitis-vinifera- | | |
| e.html | | |
| Cucsa.255860.1 | 10 | Cucumber (Cucumis sativa) |
| On the internet (world wide web) at | | MSH1 coding sequence; (DNA |
| phytozome.net/ | | sequence) |
| GenBank Accession | 11 | Cotton (Gossypium hirsutum) |
| ES831813.1 | | MSH1 partial cDNA sequence |
| on the world wide web at | | (EST); (DNA sequence) |
| ncbi.nlm.nih.gov/nucest | | |
| Oryza_sativa_msh1_2000up | 12 | Oryza_sativa_msh1_Promoter |
| >Rice-LOC_Os04g42784 | | and 5′ UTR |
| Solanum_lycopersicum_2000up | 13 | Solanum_lycopersicum msh1 |
| >Tomato-Solyc09g090870.2 | | promoter and 5′ UTR |
| Sorghum_bicolor_MSH1_2000up_Phyt | 14 | Sorghum bicolor msh1 |
| ozome>Sb06g021950 | | promoter and 5′ UTR |
| Arabidopsis-Col0-MSH1 | 15 | Arabidopsis-Col0-MSH1 |
| | | promoter and 5′ UTR |
| >gi\|145337631\|ref\|NM_106295.3\| | 16 | Arabidopsis PPD3 coding |
| Arabidopsis thaliana photosystem II | | region |
| reaction center PsbP family protein | | |
| cDNA, complete cds | | |
| >gi\|297839518\|ref\|XM_002887595.1\| | 17 | Arabidopsis PPD3 coding |
| Arabidopsis lyrata subsp. lyrata | | region |
| hypothetical protein, cDNA | | |
| >gi\|449522158\|ref\|XM_004168047.1\| | 18 | Cucumis sativus PPD3 coding |
| PREDICTED: Cucumis sativus psbP domain- | | region |
| containing protein 3, chloroplastic-like | | |
| (LOC101211525), cDNA | | |
| >gi\|255539323\|ref\|XM_002510681.1\| | 19 | Ricinus communis PPD3 |
| Ricinus communis conserved | | coding region |
| hypothetical protein cDNA | | |
| >gi\|359491869\|ref\|XM_002273296.2\| | 20 | Vitis vinifera PPD3 coding |
| PREDICTED: Vitis vinifera psbP domain- | | region |
| containing protein 3, chloroplastic-like | | |
| (LOC100263326), cDNA | | |
| >gi\|357467178\|ref\|XM_003603826.1\|Medicago | 21 | Medicago truncatula PPD3 coding |
| truncatula PsbP domain-containing protein | | region |
| (MTR_3g116110) cDNA, complete cds | | |
| >gi\|224083365\|ref\|XM_002306962.1\|Populus | 22 | Populus trichocarpa PPD3 coding |
| trichocarpa predicted protein, cDNA | | region |
| >gi\|388521576\|gb\|BT149056.1\| Lotus | 23 | Lotus japonicus PPD3 coding |
| japonicus clone JCVI-FLLj-8L12 | | region |
| unknown cDNA | | |
| gi\|470131466\|ref\|XM_004301567.1\| | 24 | Fragaria vesca PPD3 coding |
| PREDICTED: Fragaria vesca subsp. | | region |
| vesca psbP domain-containing protein 3, | | |
| chloroplastic-like (LOC101302662), | | |
| mRNA | | |
| >gi\|356517169\|ref\|XM_003527214.1\| | 25 | Glycine max PPD3 coding |
| PREDICTED: Glycine max psbP | | region |
| domain-containing protein 3, | | |
| chloroplastic-like (LOC100805637), | | |
| mRNA | | |
| Solanum lycopersicum psbP domain- | 26 | Solanum lycopersicum PPD3 |
| containing protein 3, chloroplastic-like | | coding region |
| (LOC101247415), mRNA | | |
| >gi\|502130964\|ref\|XM_004500773.1\| | 27 | Cicer arietinum PPD3 coding |
| PREDICTED: Cicer arietinum psbP domain- | | region |
| containing protein 3, chloroplastic-like | | |
| (LOC101499898), transcript variant X2, mRNA | | |
| >gi\|241989846\|dbj\|AK330387.1\| Triticum | 28 | Triticum aestivum PPD3 |
| aestivum cDNA, clone: SET4_F09, cultivar: | | coding region |
| Chinese Spring | | |
| >gi\|115477245\|ref\|NM_001068754.1\| | 29 | Oryza sativa PPD3 coding |
| Oryza sativa Japonica Group | | region |
| Os08g0512500 (Os08g0512500) | | |
| mRNA, complete cds | | |
| >gi\|357141873\|ref\|XM_003572329.1\| | 30 | Brachypodium distachyon |
| PREDICTED: Brachypodium | | PPD3 coding region |
| distachyon psbP domain-containing | | |
| protein 3, chloroplastic-like | | |
| (LOC100840022), mRNA | | |
| >gi\|242383886\|emb\|FP097685.1\| | 31 | Phyllostachys edulis PPD3 |
| Phyllostachys edulis cDNA clone: | | coding region |
| bphylf043n24, full insert sequence | | |
| >gi\|326512571\|dbj\|AK368438.1\| | 32 | Hordeum vulgare PPD3 coding |
| Hordeum vulgare subsp. vulgare mRNA | | region |
| for predicted protein, partial cds, clone: | | |
| NIASHv2073K06 | | |
| >gi\|195613363\|gb\|EU956394.1\| Zea | 33 | Zea mays PPD3 coding region |
| mays clone 1562032 thylakoid lumen | | |
| protein mRNA, complete cds | | |
| >gi\|242082240\|ref\|XM_002445844.1\| | 34 | Sorghum bicolor PPD3 coding |
| Sorghum bicolor hypothetical protein, | | region |
| mRNA | | |
| >gi\|514797822\|ref\|XM_004973837.1\| | 35 | Setaria italica PPD3 coding |
| PREDICTED: Setaria italica psbP | | region |
| domain-containing protein 3, | | |
| chloroplastic-like (LOC101754517), | | |
| mRNA | | |
| >gi\|270145042\|gb\|BT111994.1\| Picea glauca | 36 | Picea glauca PPD3 coding |
| clone GQ03308_J01 mRNA sequence | | region |
| >gi\|215274040\|gb\|EU935214.1\| Arachis diogoi | 37 | Arachis diogoi PPD3 coding |
| clone AF1U3 unknown mRNA | | region |
| >gi\|168003548\|ref\|XM_001754423.1\| | 38 | Physcomitrella patens PPD3 |
| Physcomitrella patens subsp. patens | | coding region |
| predicted protein | | |
| (PHYPADRAFT_175716) mRNA, | | |
| complete cds | | |
| >gi\|302809907\|ref\|XM_002986600.1\| | 39 | Selaginella moellendorffii |
| Selaginella moellendorffii hypothetical | | PPD3 coding region |
| protein, mRNA | | |
| >gi\|330318510\|gb\|HM003344.1\| | 40 | Camellia sinensis PPD3 coding |
| Camellia sinensis clone U10BcDNA | | region |
| 3162 | | |
| Zea_mays_2000up_phytozome | 41 | Zea mays Msh1 promoter and |
| >GRMZM2G360873 | | 5′ UTR |
| AT5G67120RING-F | 42 | primer |
| AT5G67120RING-R | 43 | primer |
| AT1G20690SWI-F | 44 | primer |
| AT1G20690SWI-R | 45 | primer |
| AT3g271501stMir2-F | 46 | primer |
| AT3g271501stMir2-R | 47 | primer |
| AT3g271502ndMir2-F | 48 | primer |
| AT3g271502ndMir2-R | 49 | primer |
| RNAi-F | 50 | primer |
| RNAi-R | 51 | primer |
| upstream_1 kb\| photosystem II | 52 | Arabidopsis thaliana PPD3 |
| reaction center PsbP family protein | | promoter |
| mRNA | | |
| upstream_1 kb\|Oryza sativa Japonica | 53 | Oryza sativa PPD3 promoter |
| Group Os08g0512500 | | |
| (Os08g0512500) mRNA | | |
| upstream_1 kb\|PREDICTED: | 54 | Solanum lycopersicum |
| Solanum lycopersicum psbP domain- | | PPD3 promoter |
| containing protein 3, chloroplastic- | | |
| like | | |

Sequence Listing is provided herewith as a computer readable form (CRF) named “46589_{—}133998_SEQ_LST.txt” and is incorporated herein by reference in its entirety. This sequence listing contains SEQ ID NO:1-57 that are referred to herein.

### Example 4

### MSH1 Alters the Epigenome at Specific Nuclear Regions

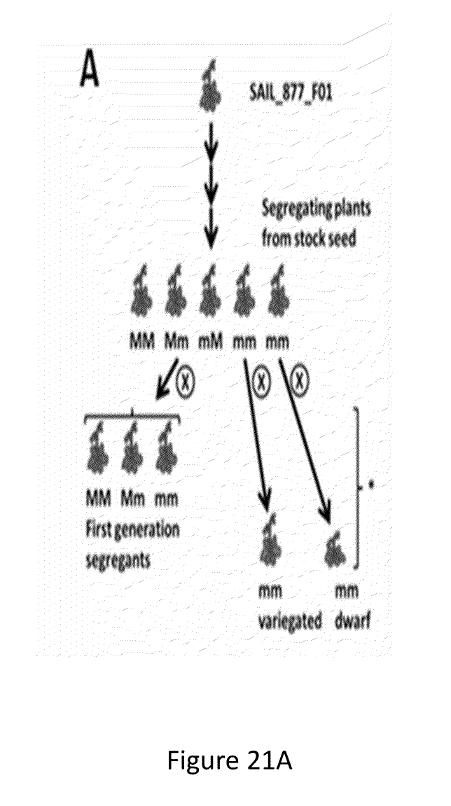

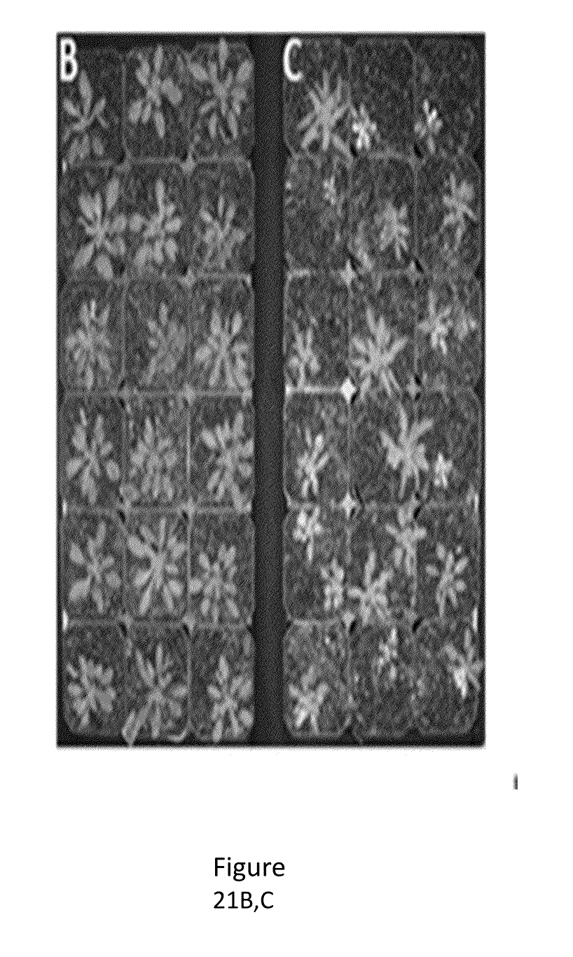

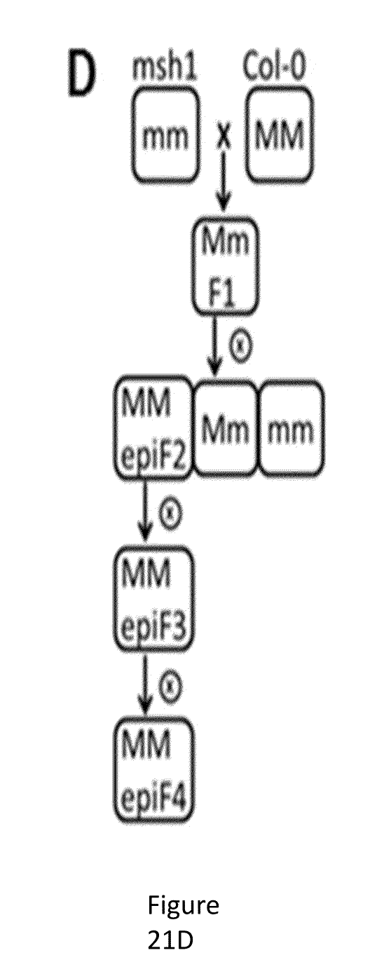

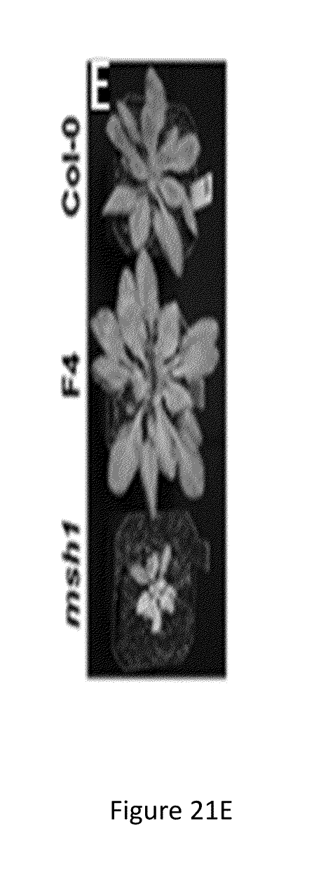

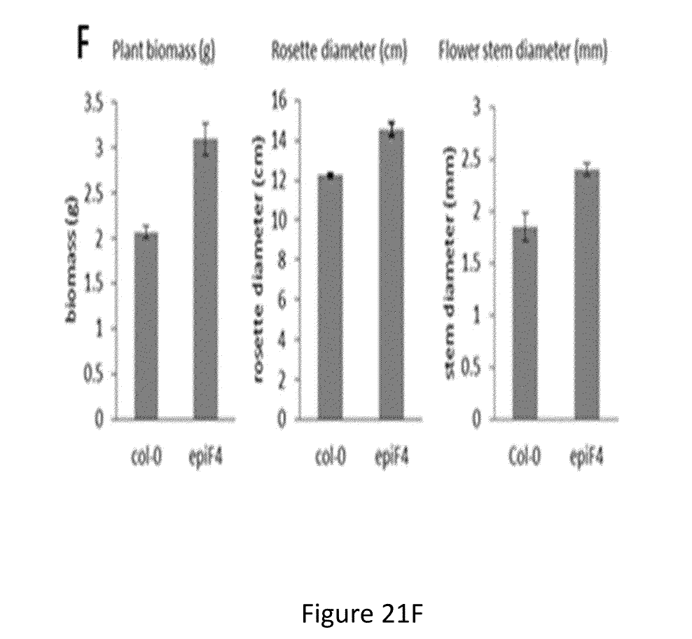

To investigate the heritability of MSH1-derived phenotypes in Arabidopsis, we carried out crossing experiments (FIG. 21). Crossing of wild type Columbia-0 (Col-0) with the msh1 mutant chm1-1, which contains a point mutation¹⁰, resulted in an enhanced growth phenotype. By the F₃ generation, these enhanced-vigor plants exhibited markedly larger rosettes and stem diameter and early flowering (FIG. 21), similar to observations in sorghum^{1,12}.

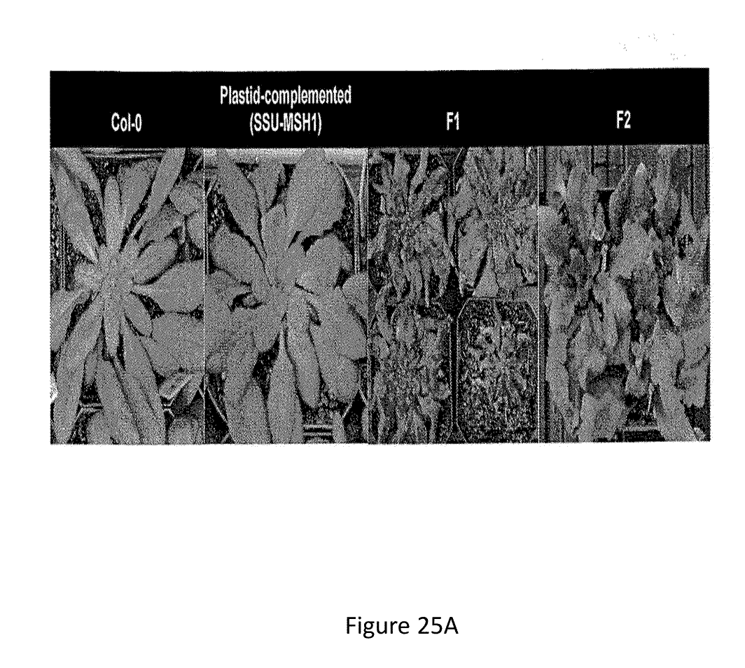

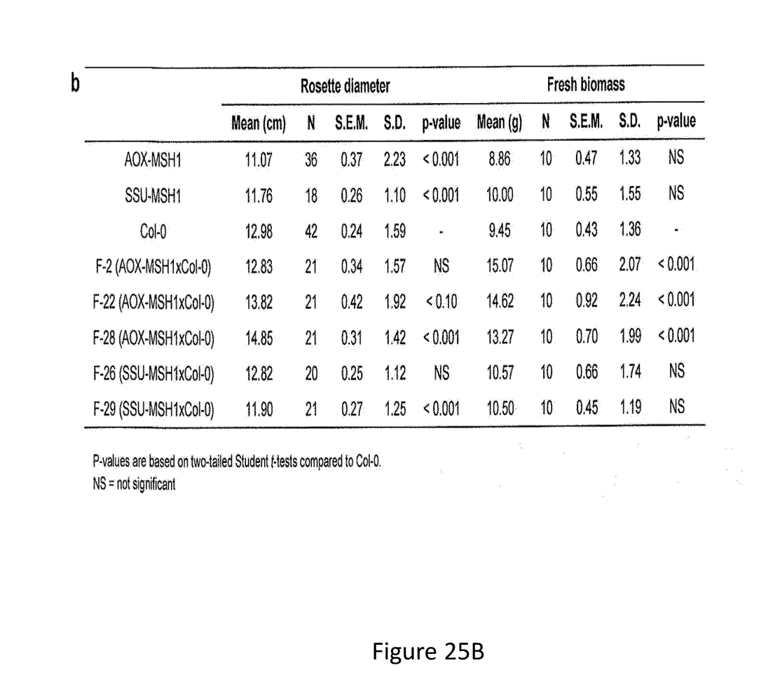

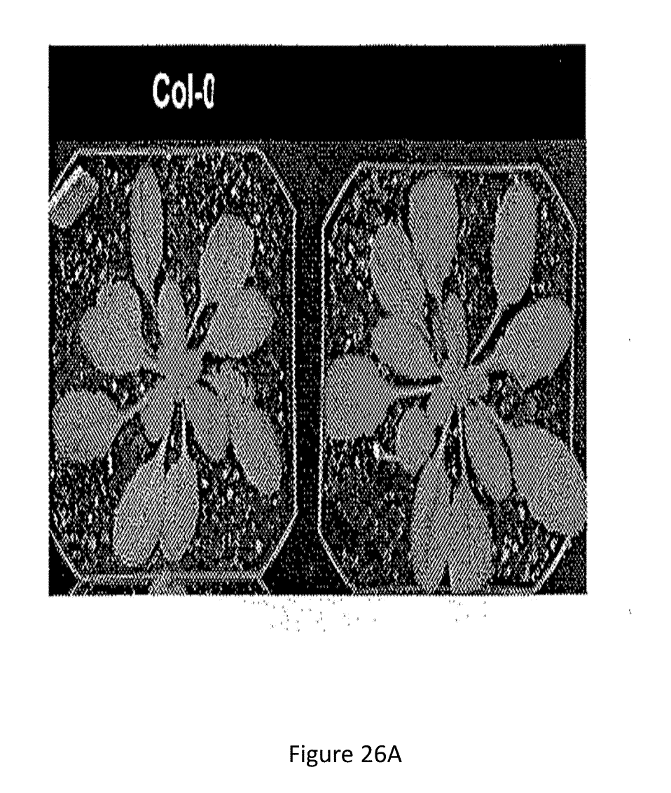

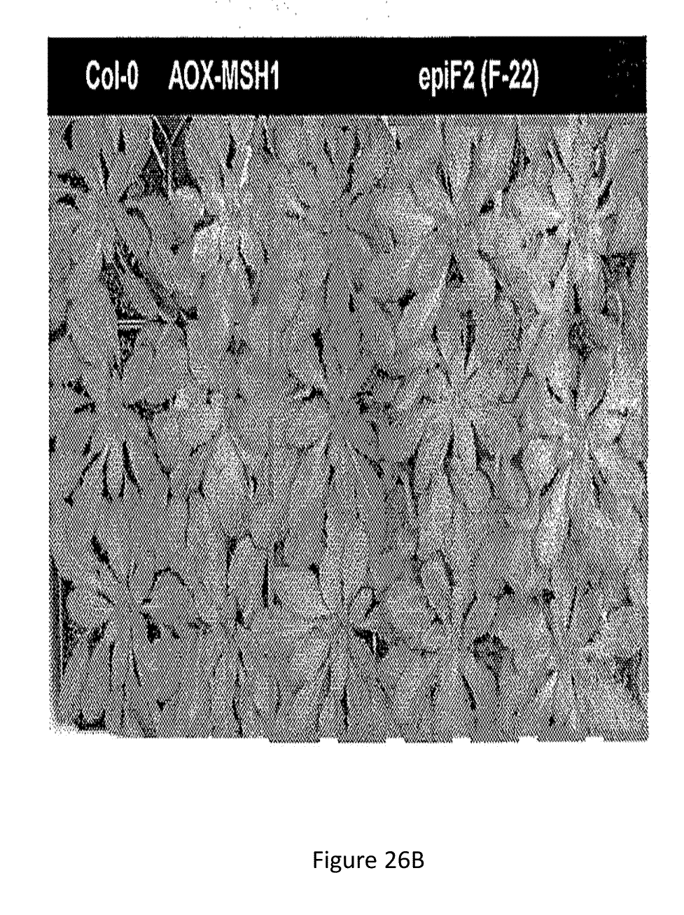

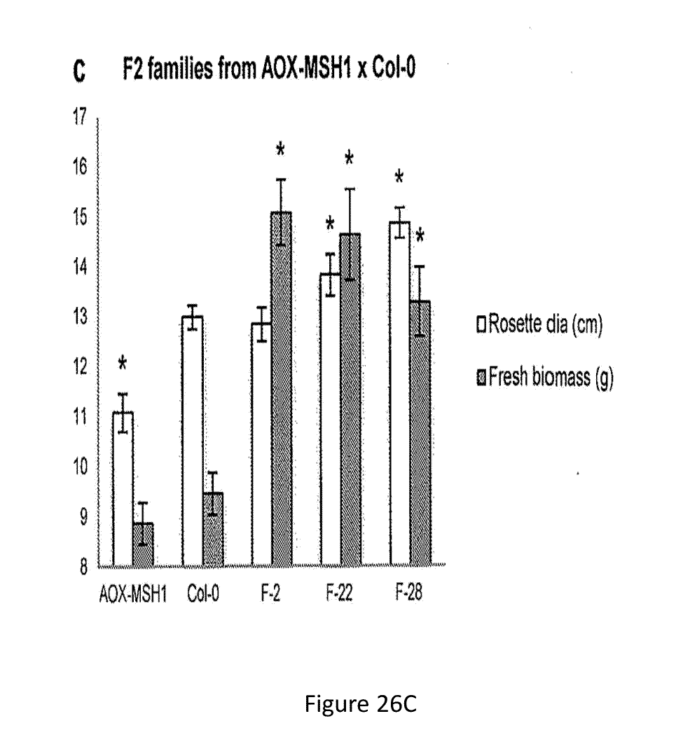

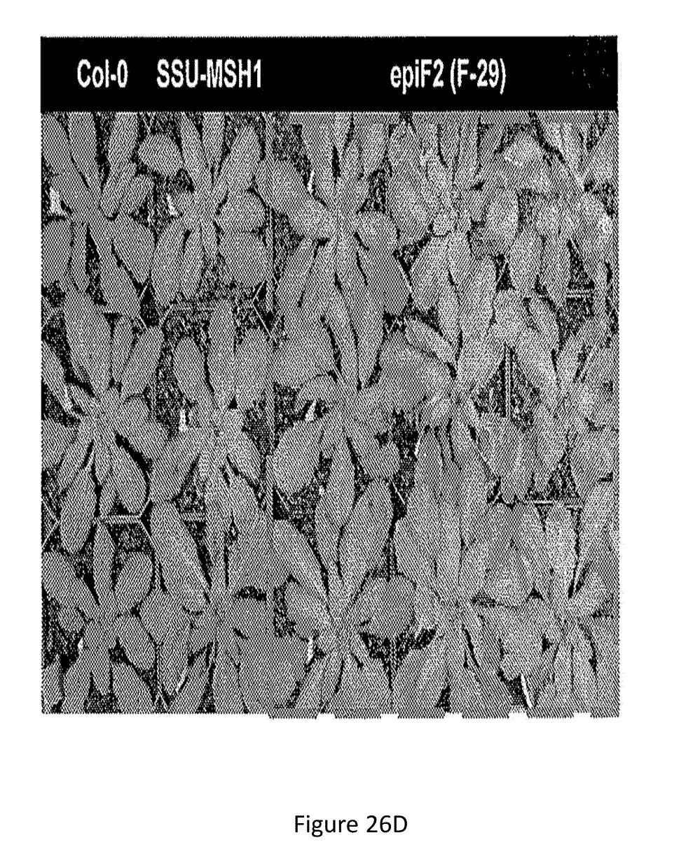

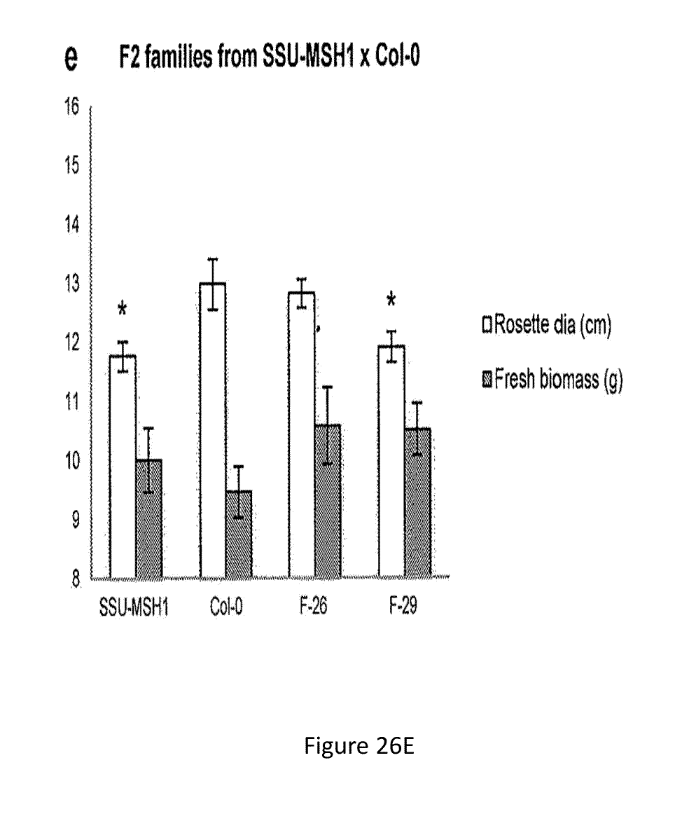

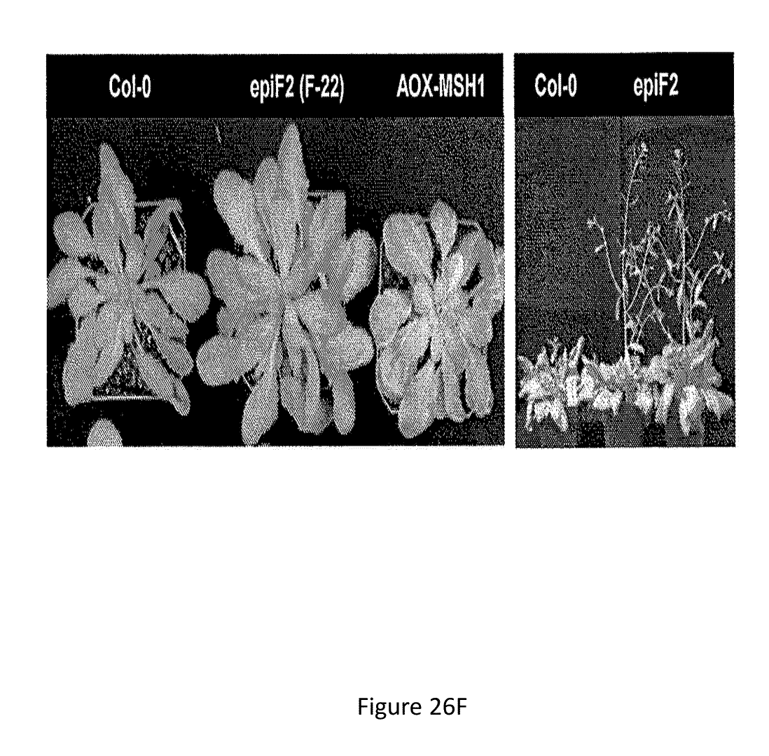

Since altered plant development in Arabidopsis msh1 is conditioned by plastid changes′, we tested whether the enhanced growth vigor in F₂ lines also emanated from these plastid effects. Arabidopsis MSH1 hemi-complementation lines, derived by introducing a mitochondrial- versus chloroplast-targeted MSH1 transgene to the msh1 mutant¹¹, distinguish mitochondrial and plastid contributions to the phenomenon. Plastid hemi-complementation lines crossed as female to Col-0 resulted in a normal phenotype for some F₁ progeny, but with 10% to 77% showing slow germination, leaf curling and delayed flowering (FIG. 25A). The altered phenotypes may be due to mitochondrial changes. In F, progeny from crosses to the mitochondrial-complemented line, over 30% showed enhanced growth, larger rosette diameter, and earlier flowering time, closely resembling F₄ phenotypes from chm1-1×Col-0 (FIGS. 25A, and 26A). These results were further confirmed in derived F₂ populations (FIGS. 25B, 26B-E), indicating that msh1-deprived plastids are necessary for the growth vigor changes seen after crossing.

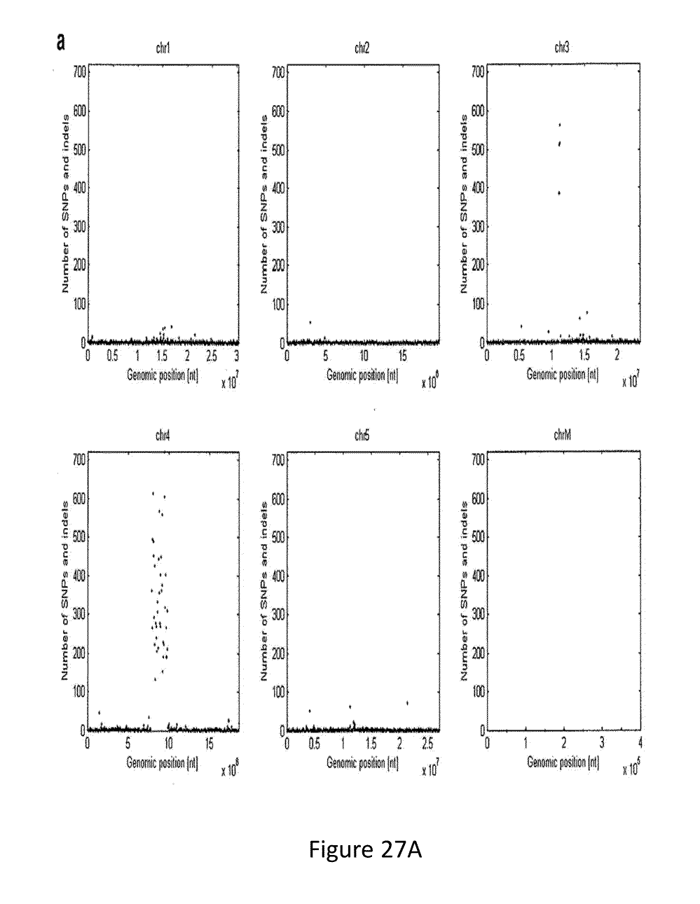

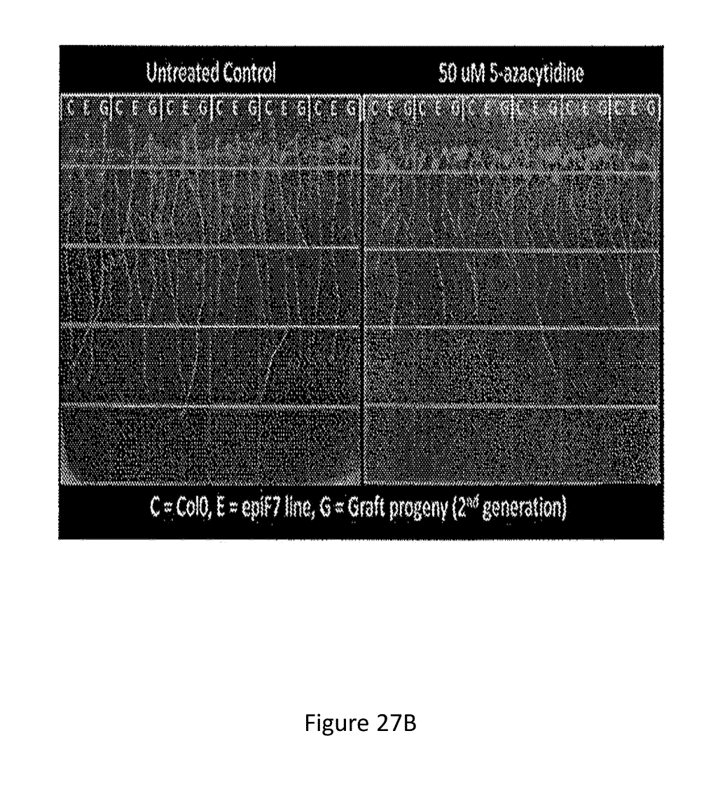

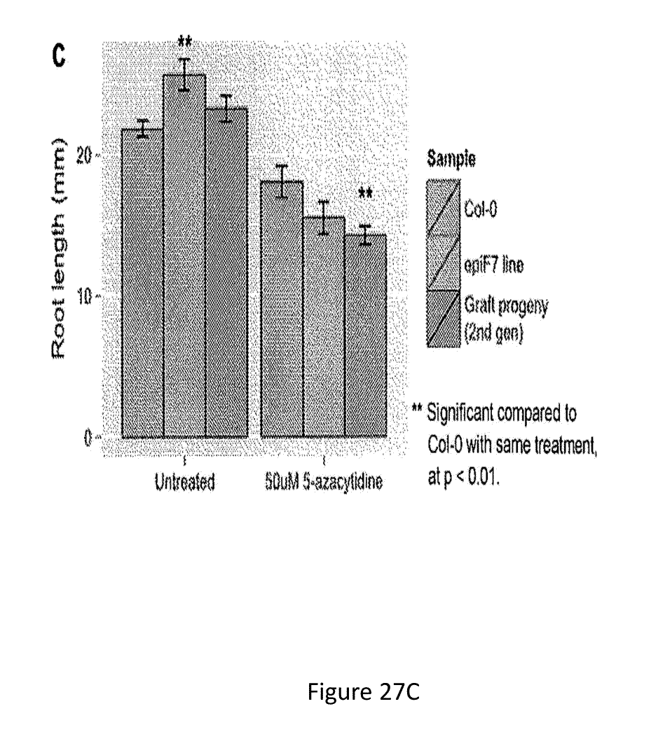

Sequencing and alignment of the chm1-1 genome produced no evidence of illegitimate recombination or rearrangement to account for the novel phenotypic variation (FIG. 27A-C). To assess whether msh1-mediated growth changes were epigenetic, we performed bisulfite sequencing on material derived from early generation msh1 T-DNA mutants (FIG. 21A), thereby minimizing generational DNA methylation noise. A segregating Salk T-DNA line was obtained and a heterozygous individual was self-pollinated to yield MSH1 +/+(wild-type segregant), MSH1 +/− heterozygotes, and msh1 −/− (considered first generation), each of which was included in bisulfite sequencing. Additional msh1 −/− plants were self-pollinated to create second generation msh1 mutants, which recapitulated the variable phenotypes seen in chm1-1; individuals showing variegation (msh1 gen2 variegated) and dwarfing (msh1 gen2 dwarf) were included in bisulfite sequencing.

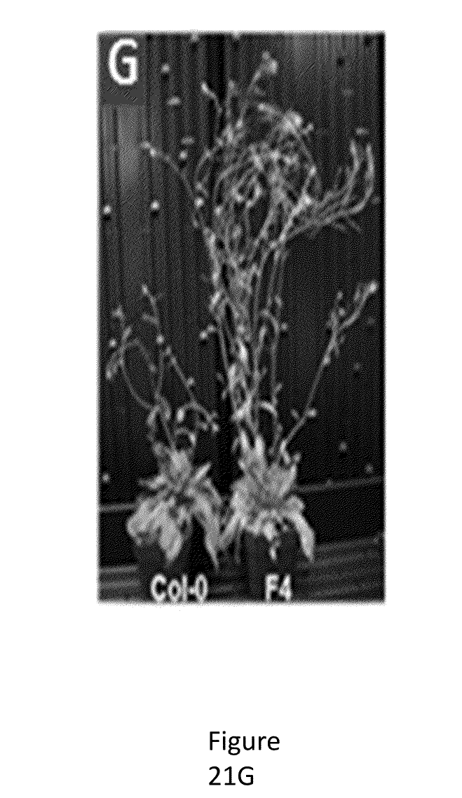

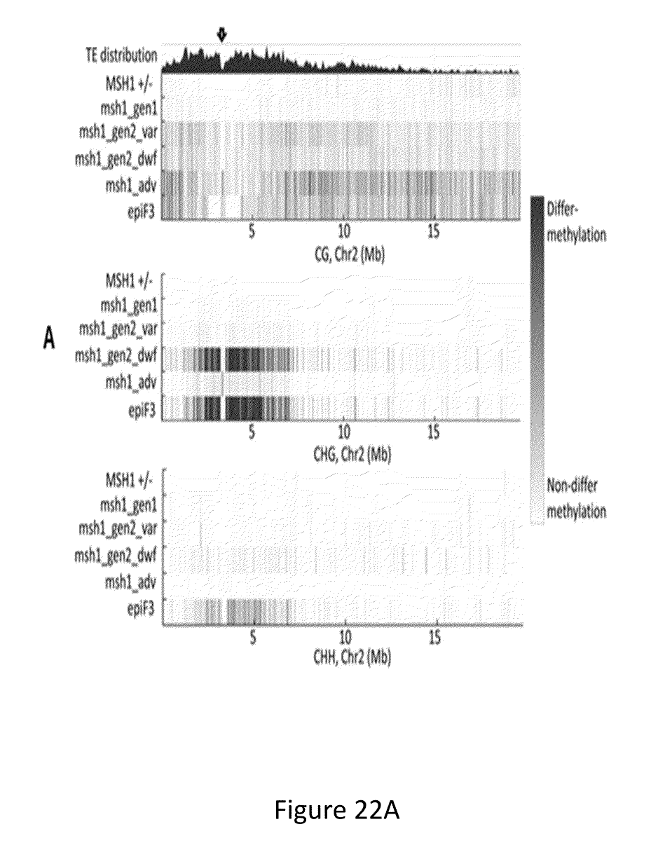

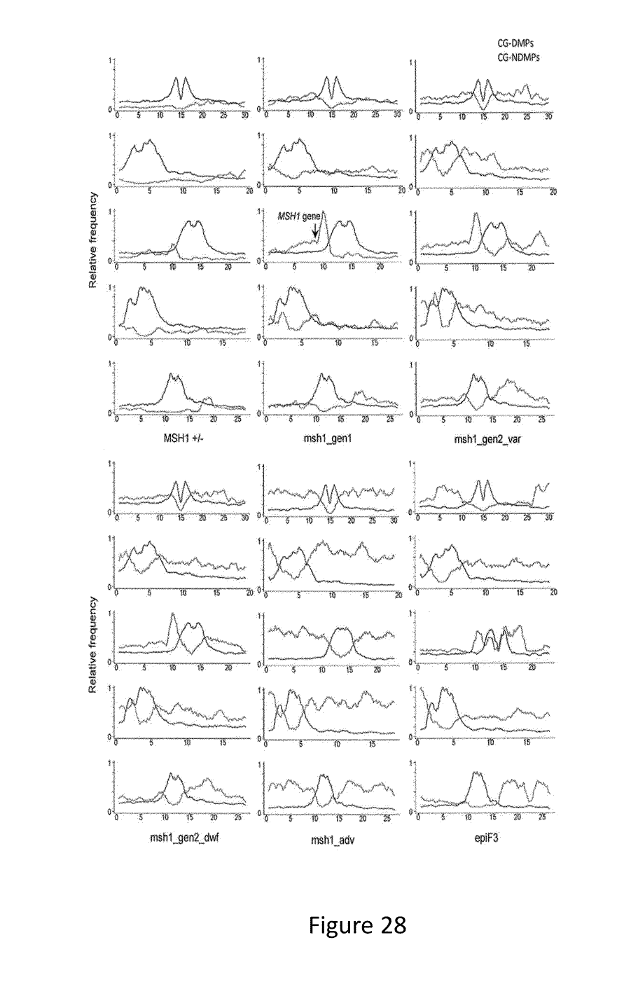

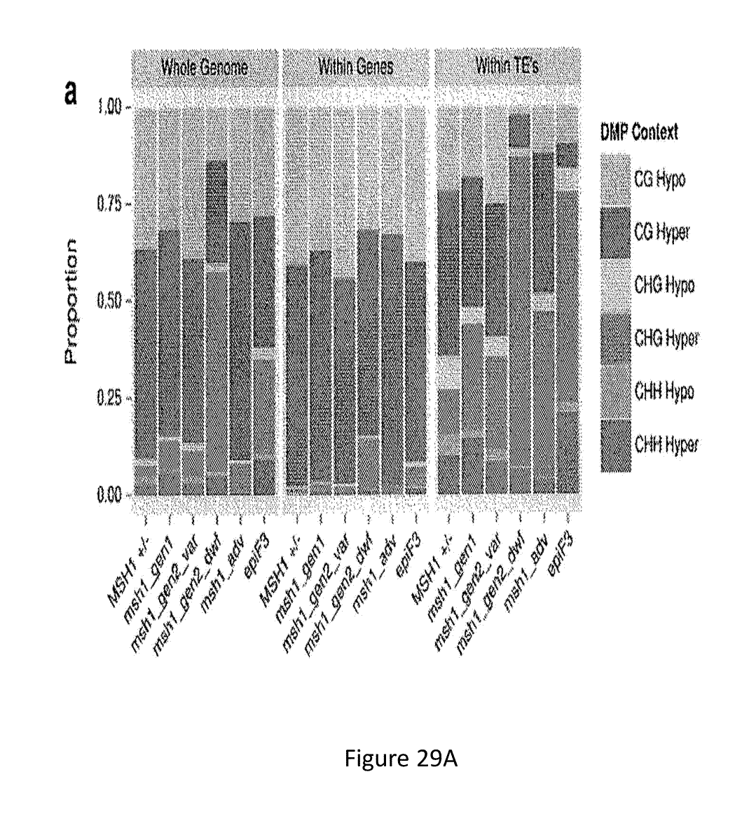

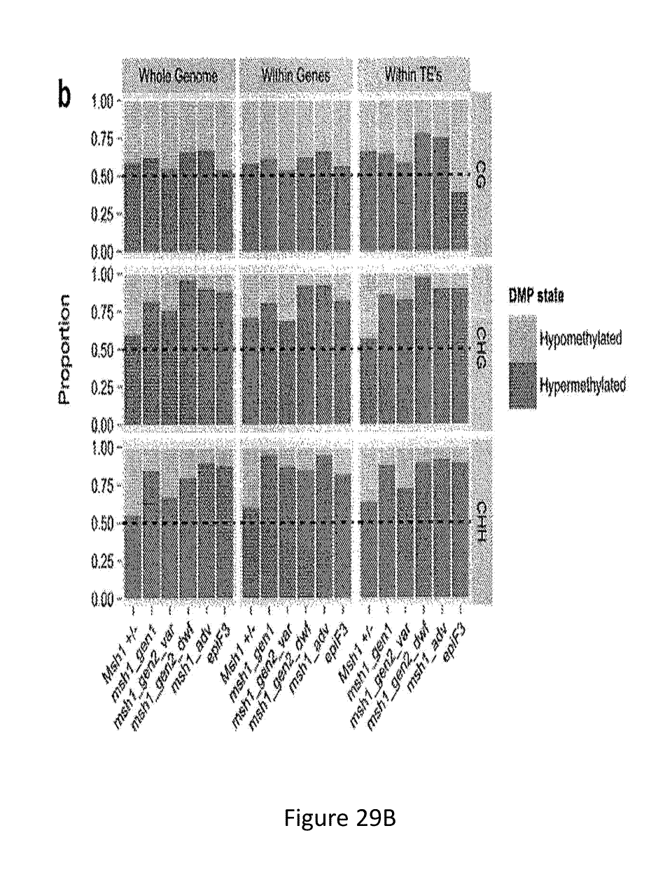

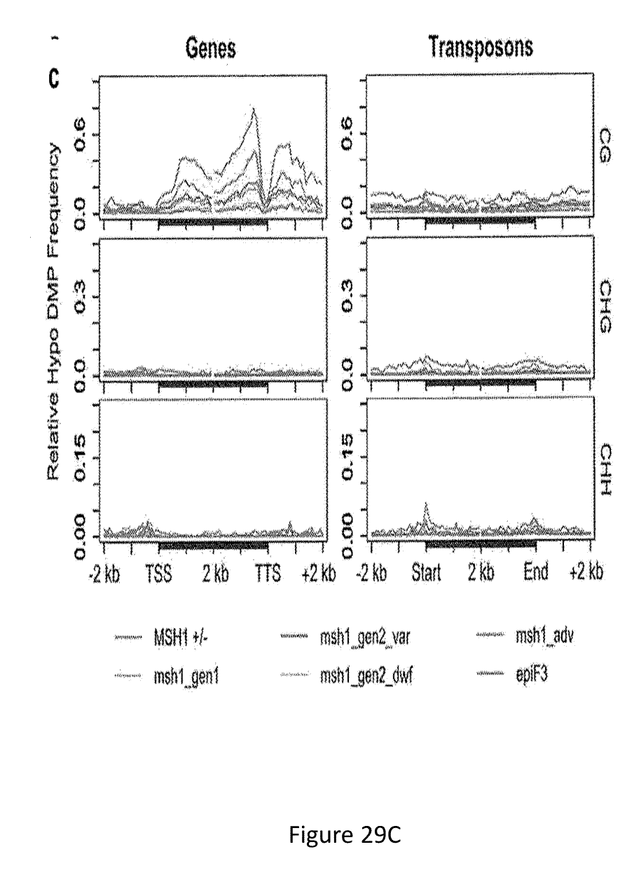

Methylome analysis was first conducted based on pair-wise comparisons of each msh1 mutant to wild type. Generally, we observed increasing numbers of pair-wise CG differentially methylated positions (DMPs) along chromosome arms (FIG. 22A) a proportion of which is likely due to unavoidable stochastic generational changes (FIG. 22B, C; FIG. 28). However, a surprising concentration of CG-DMPs was seen stretching nearly 2 Mb along chromosome 3, centered on the 10-Mb mark in all samples (FIG. 28). CG methylation changes in this region were most apparent starting in the first generation of msh1 −/−. The MSH1+/− heterozygote and all msh1 −/− mutants showed a preference for CG hypermethylation over hypomethylation compared to the wild-type segregant, in both genes and transposons (FIG. 29A-C).

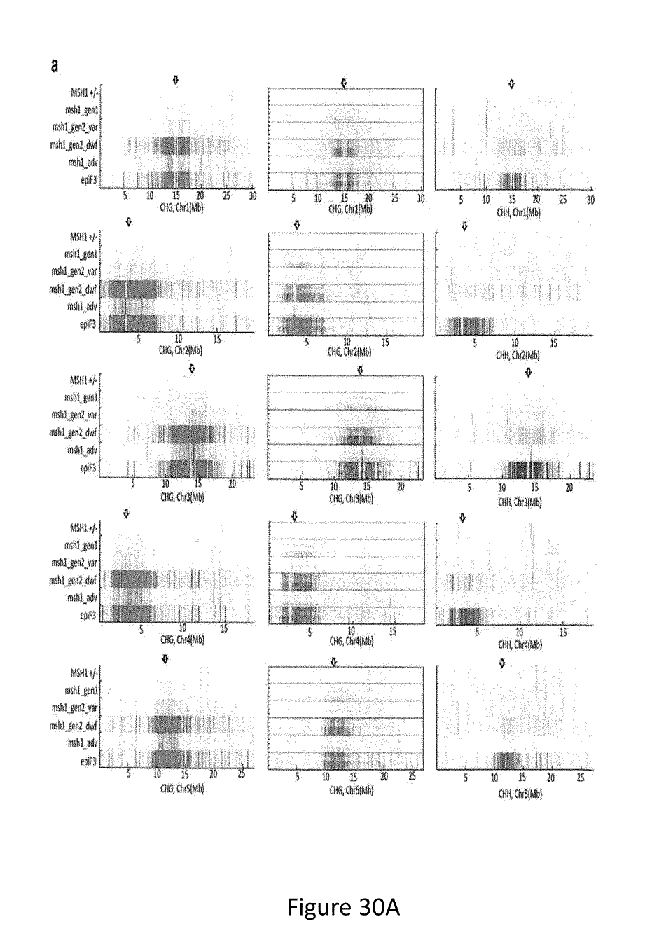

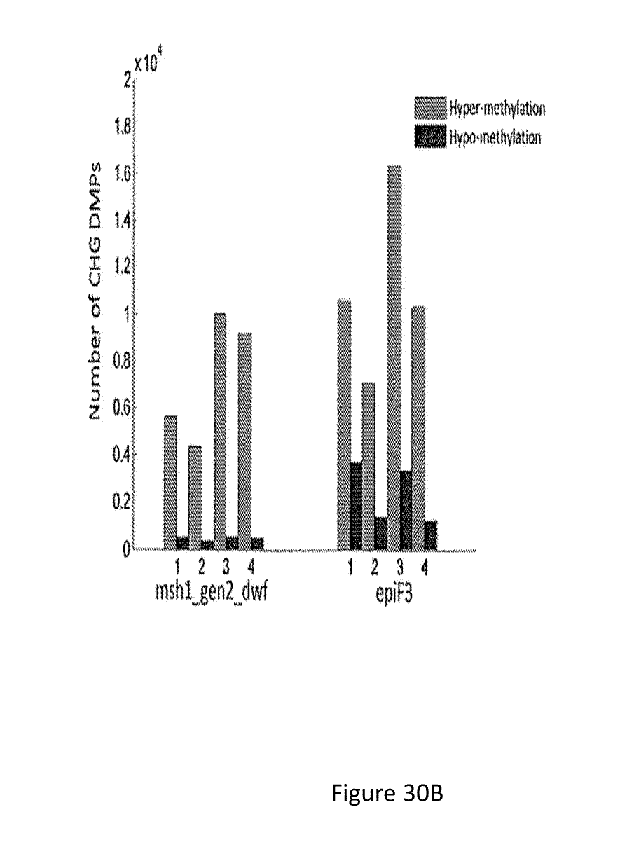

Similar to CG methylation, non-CG-DMPs trended toward hypermethylation in all msh1 −/− mutants; again, methylation changes begin within the first generation and prior to emergence of altered phenotype, although non-CG hypermethylation was most pronounced in the msh1 gen2 dwarf (FIG. 22B; FIG. 29A-C). The vast majority of non-CG-DMPs are located in transposons around pericentromeric regions (FIG. 22, FIG. 29A-C, FIG. 30A-B). Within transposons, non-CG-DMPs are generally enriched around TE boundaries (FIG. 22C, FIG. 29C). A minority of CHG-DMPs were also found in genes, with the greatest number occurring in the msh1 gen2 dwarf samples, possibly a consequence of methylation spreading from nearby silent chromatin.

Having observed non-random methylation differences between the near-isogenic msh1 mutants and their wild type siblings, we next performed bisulfite sequencing of two epiF₃ individuals from an enhanced growth line, and two wild type Col-0 individuals from stock seed. From the longstanding chm1-1 line (msh1 advanced), two individuals with mild phenotype were selected for bisulfite sequencing. As expected, due to greater generational distance, both the msh1 advanced and epiF₃ lines displayed numerous genic pair-wise CG-DMPs relative to wild-type Col-0 (FIG. 22b, FIG. 28). Whereas CG-DMPs tended to be hypermethylated in genes and transposons in msh1 advanced plants and in epiF₃ genes, a contrast was seen in epiF₃ transposons, where CG-DMPs were hypomethylated (FIG. 29B). Furthermore, while non-CG-DMPs in both msh1 advanced and epiF₃ tended to be hypermethylated in both genes and transposons (FIG. 29B), the absolute number of hypermethylated CHG-DMPs in the epiF₃ was much greater, and similar to the number observed in the msh1 gen2 dwarf (FIG. 22B).

Inducible pericentromeric CHG hypermethylation is not common in Arabidopsis methylation mutants¹³, crosses or natural populations^{14,15}. EpiF₃ samples also contained disproportionately high levels of hypermethylated CHH-DMPs, most located within transposons (FIG. 29A and FIG. 30A-B). Because the msh1 advanced line, similar to epiF₃ in generational distance from stock Col-0, did not contain these patterns, the changes were considered nonstochastic. The epiF₃ enhanced growth line displays a unique pattern of CG hypomethylation and non-CG hypermethylation around transposons, suggesting a recent history of silencing release and reestablishment.

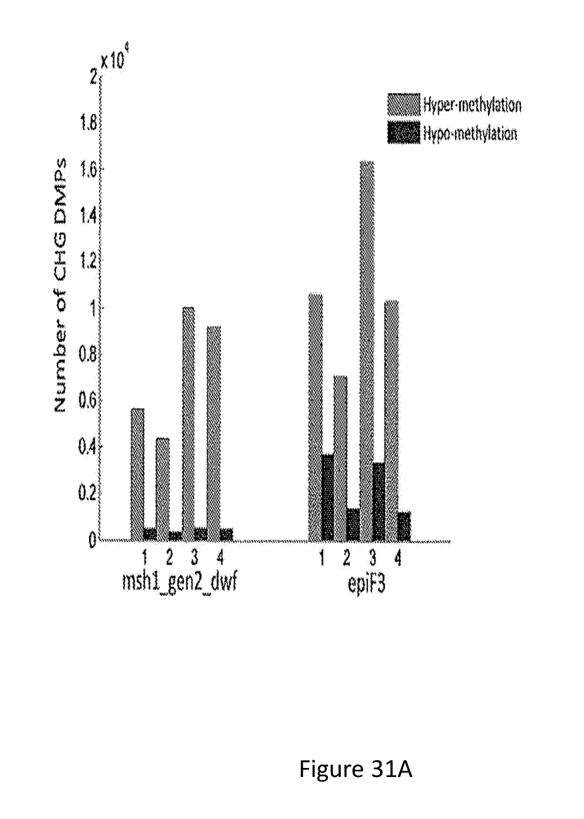

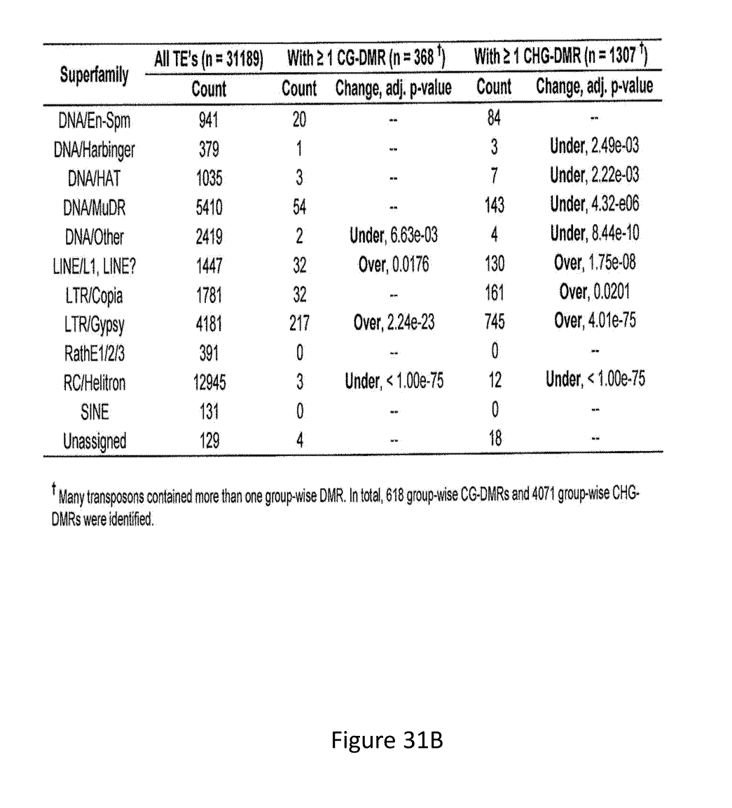

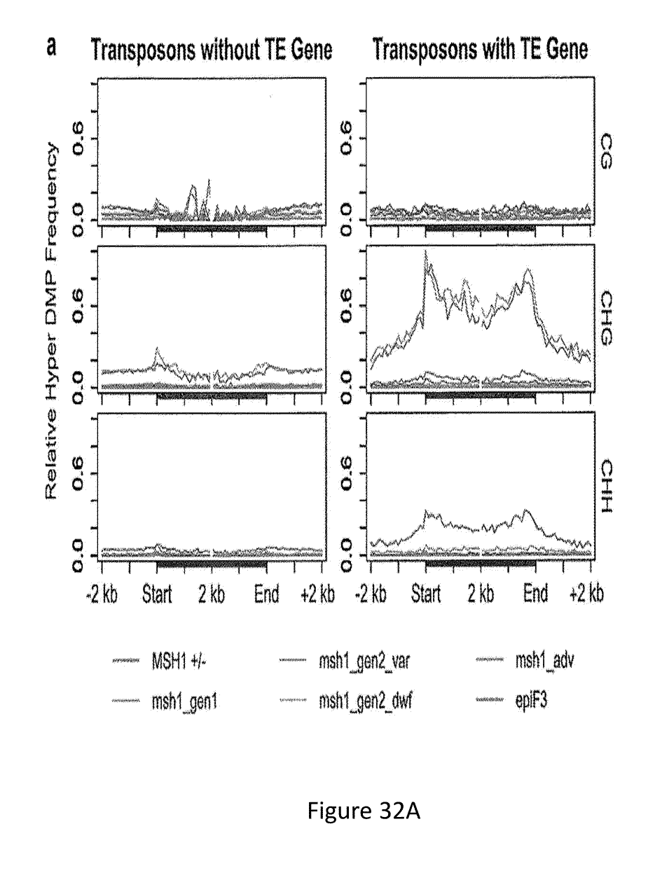

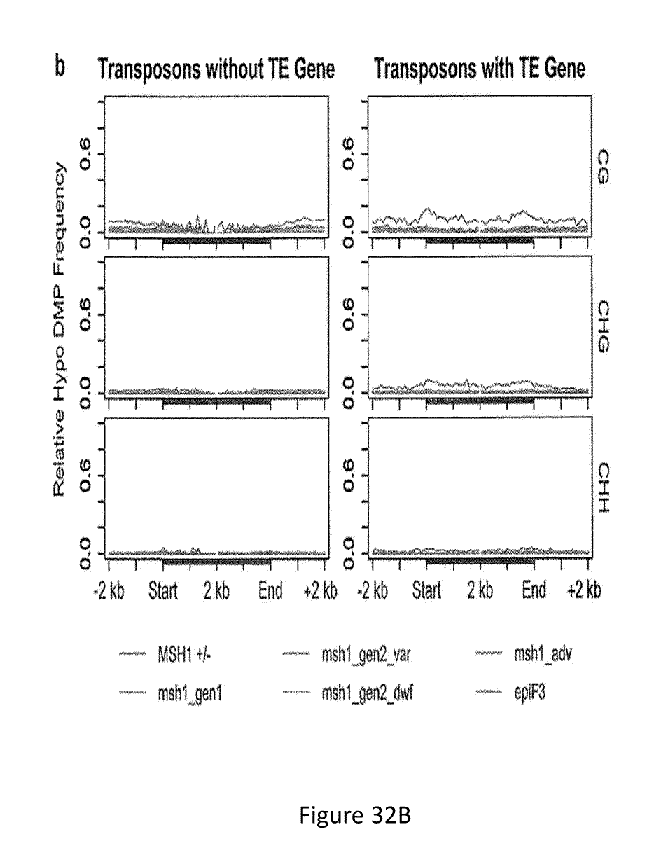

To detect discriminatory genome-wide patterns and perform multivariate analysis, we analyzed the methylome on the basis of group-wise differentially methylated regions (DMRs) between all msh1 mutants and all wild-type samples, identified by BiSeq. 456 of 618 CG-DMRs and 3506 of 4071 CHG-DMRs mapped to transposons. Gypsy-like retrotransposons were heavily over-represented in both contexts (FIG. 31A-B). Additionally, 82.5% of DMR-associated transposons are annotated as containing a transposable element gene, a highly significant enrichment compared to all annotated transposons (Fisher's exact test, p<2.2 e-16). In fact, we found that after separating transposons that contain or overlap a TE gene, these selected transposons had higher concentrations of pair-wise CHG-DMPs in the msh1 gen2 dwarf and epiF₃ compared to transposons not associated with a TE gene (FIG. 32A-B). The epiF₃ also exhibited CHH hypermethylation and CG hypomethylation in transposons containing a TE gene. These results indicate that epigenetic modulation of TE genes is likely a key consequence of MSH1 loss.

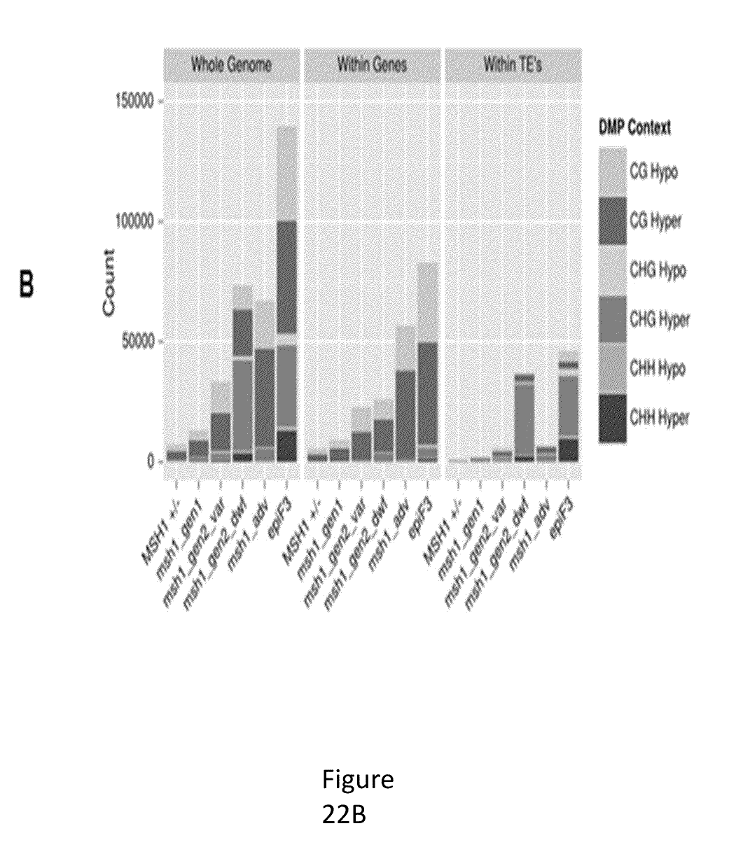

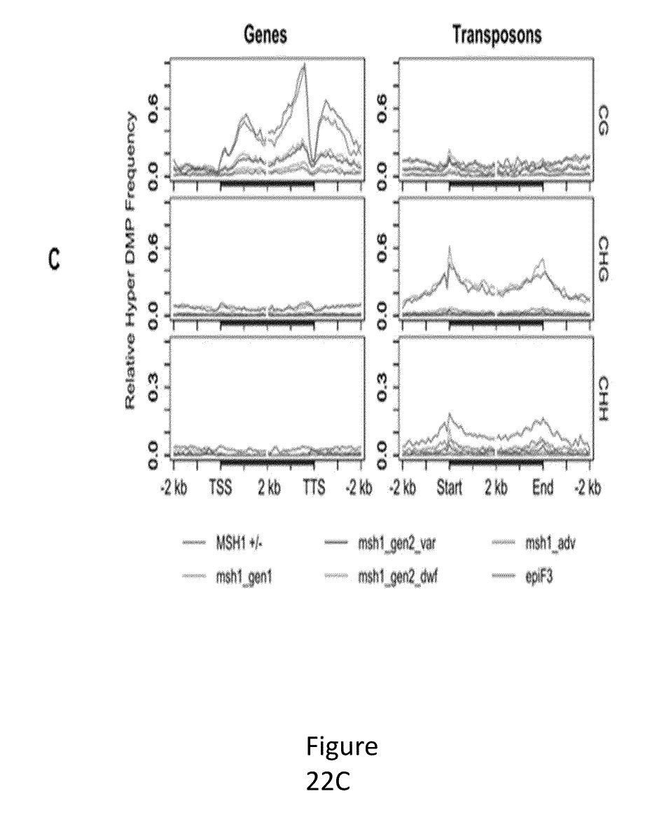

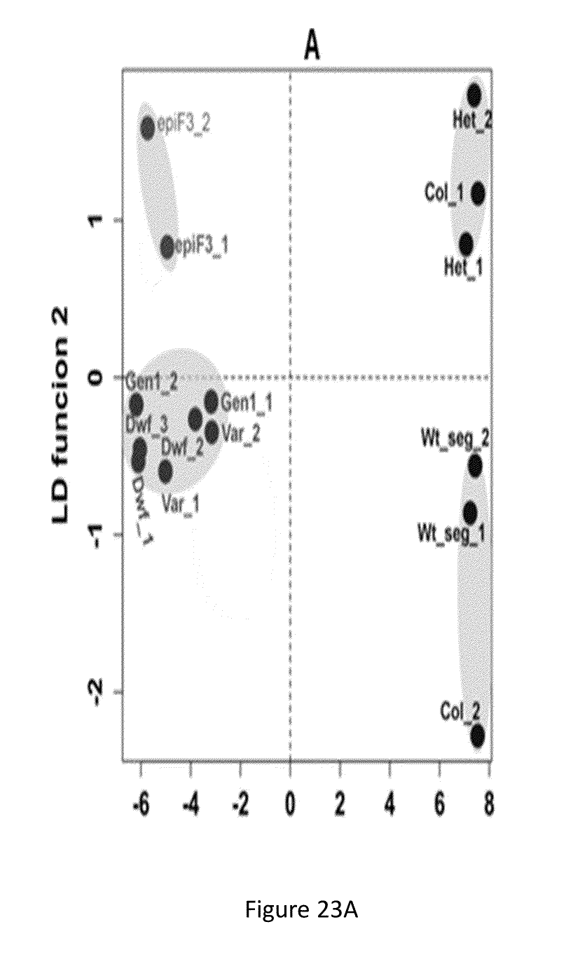

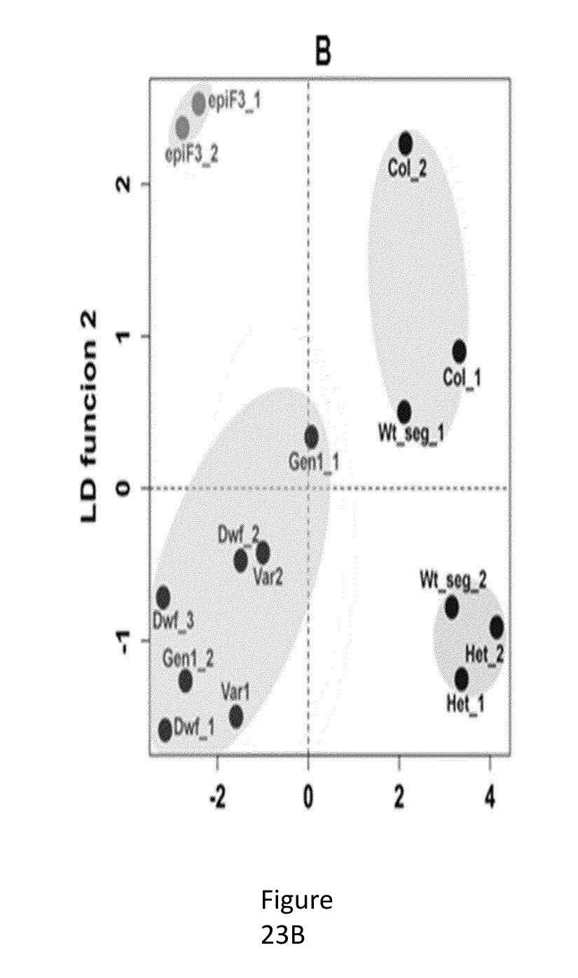

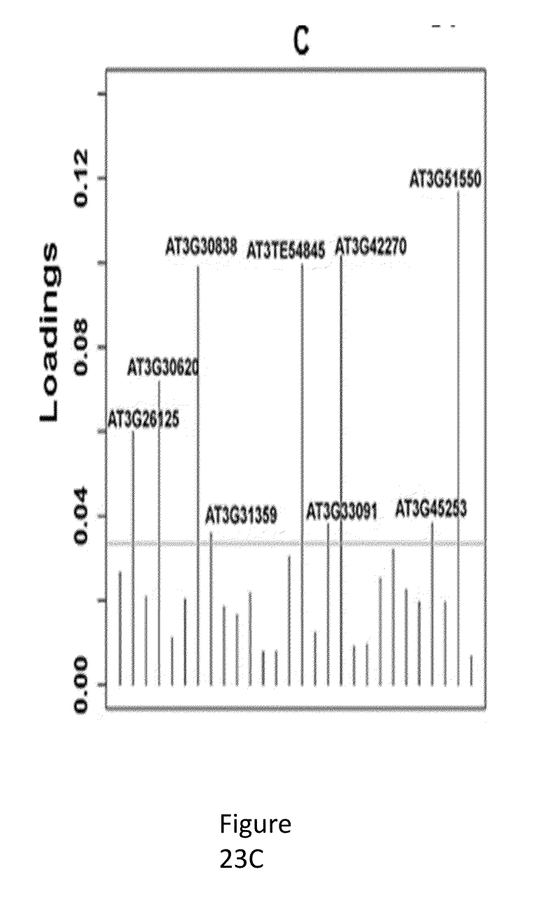

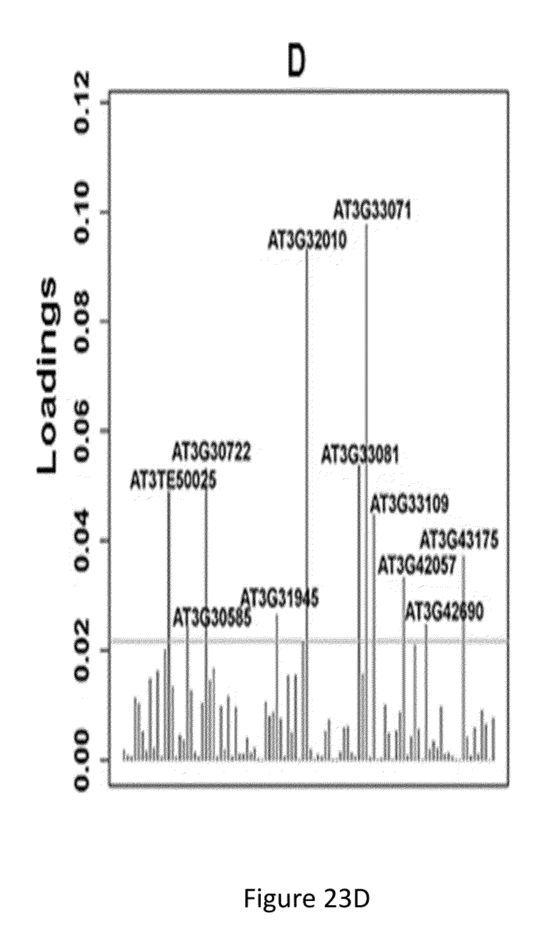

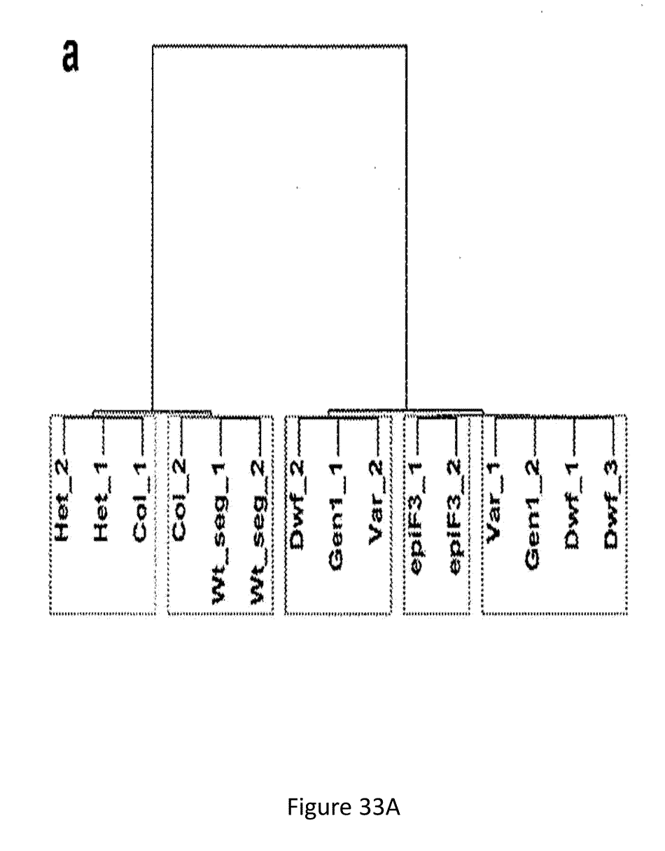

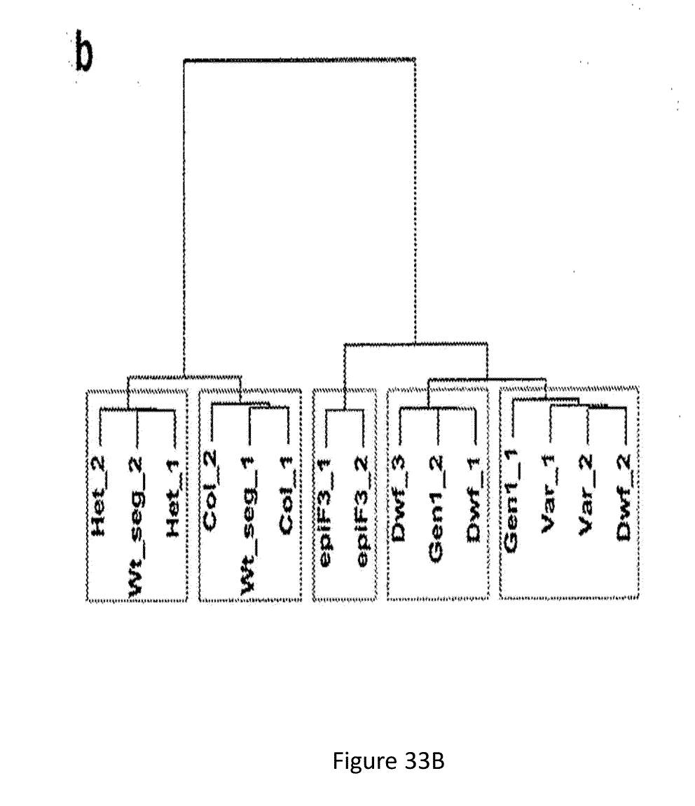

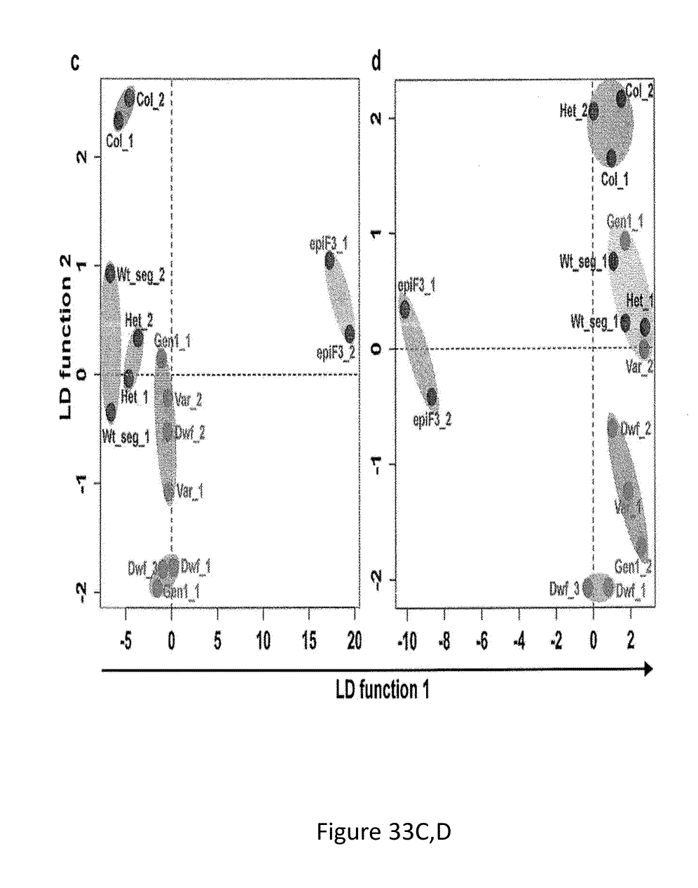

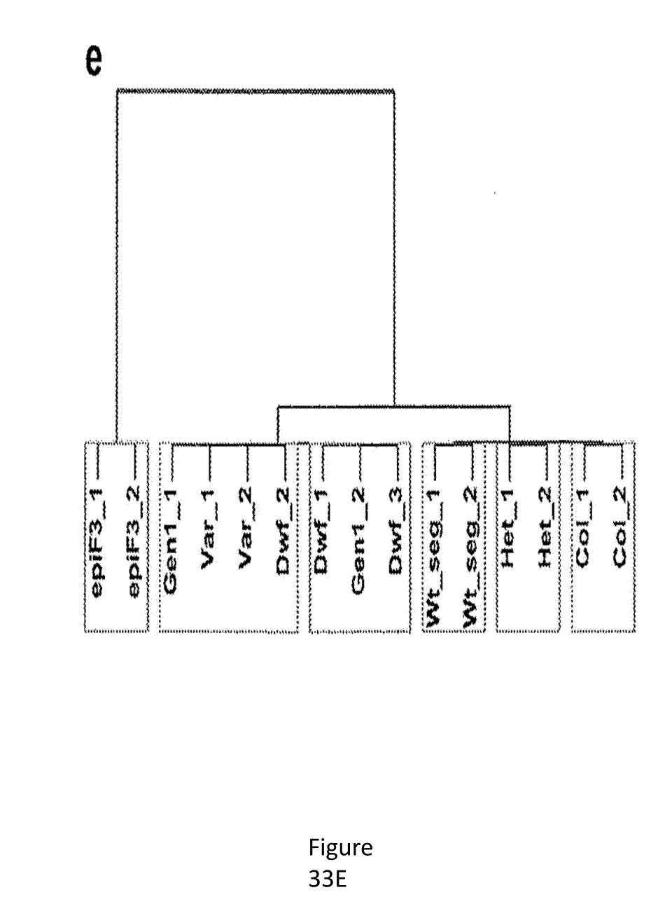

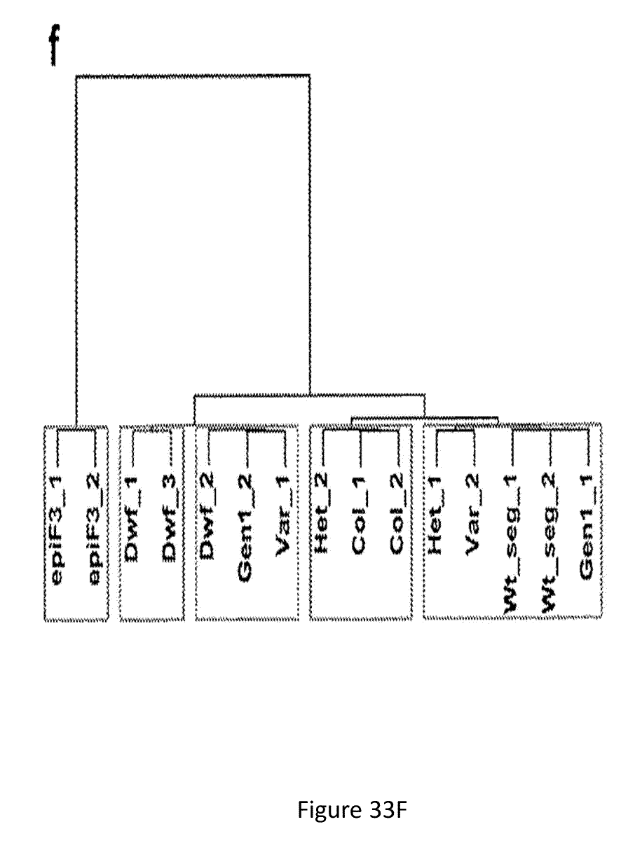

Significant genome-wide methylation differences between subsets of samples were confirmed by multivariate statistical analyses. Methylation levels in group-wise DMRs across all samples were considered as variables and reduced using principal component analysis (PCA). Subsequent application of linear discriminant analysis (LDA) revealed the existence of genome-wide CG and CHG methylation patterns able to discriminate between epiF3, msh1 mutants, and wild type (FIG. 23A, B and FIG. 33A, B). While not all group-wise DMRs carried discriminatory information (FIG. 23C, D), signals carried by the samples were sufficient to reliably split the samples into subsets. MSH1 +/− heterozygotes clustered with wild types, while all msh1 −/− mutants clustered together and epiF₃ samples formed a separate cluster, suggesting that epigenetic reprogramming occurs in msh1 −/− plants and again following crossing to generate epi-lines. Multivariate analyses using methylated regions found by tiling windows were consistent with those including group-wise DMRs (FIG. 33C-F).

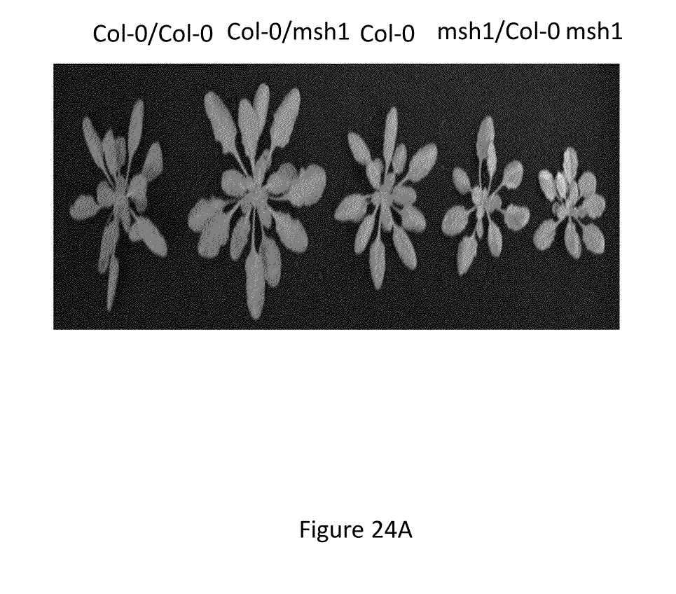

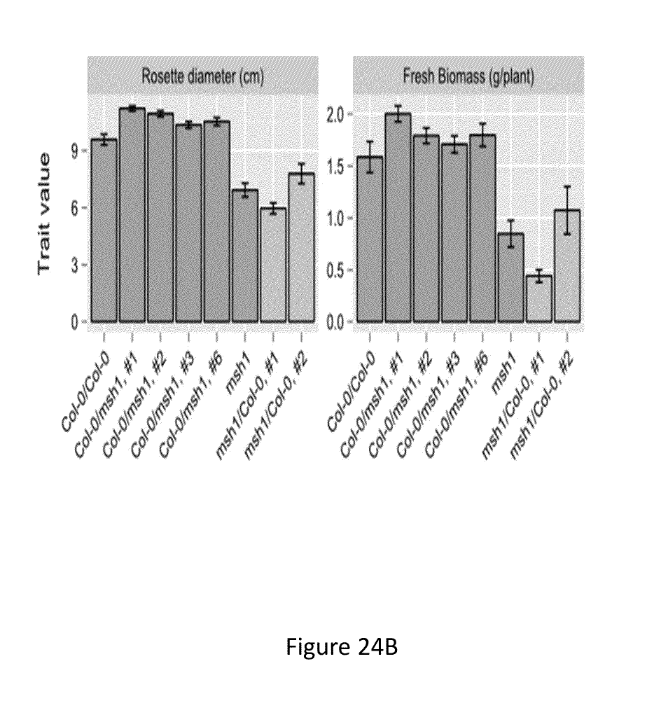

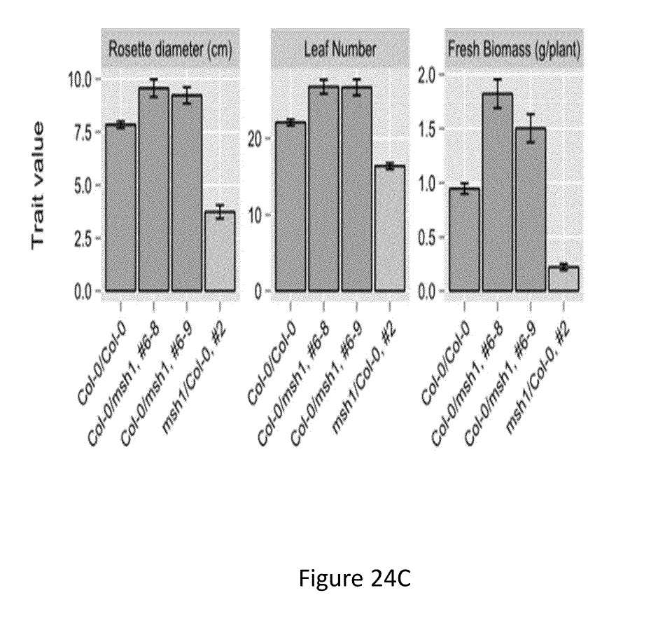

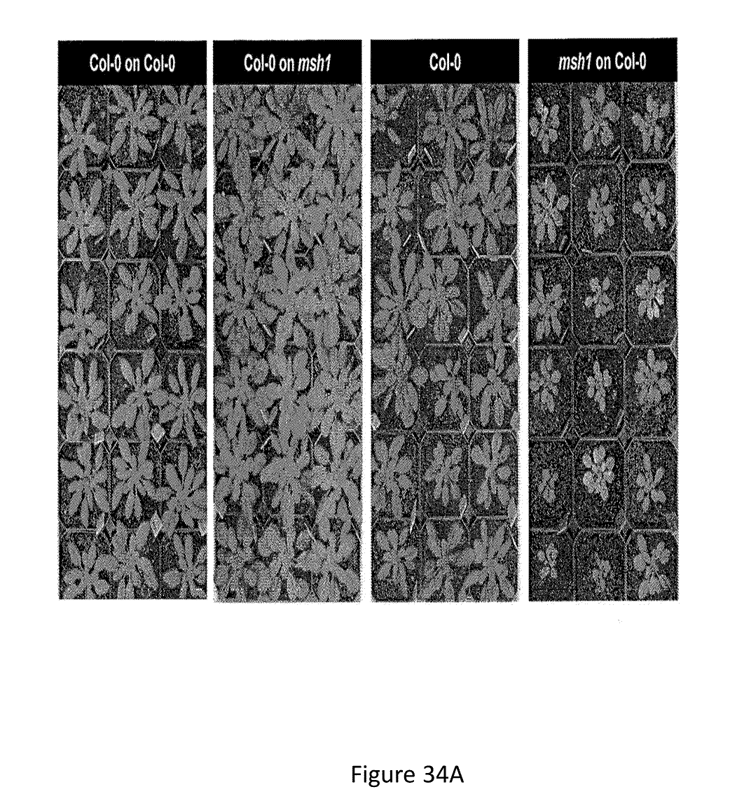

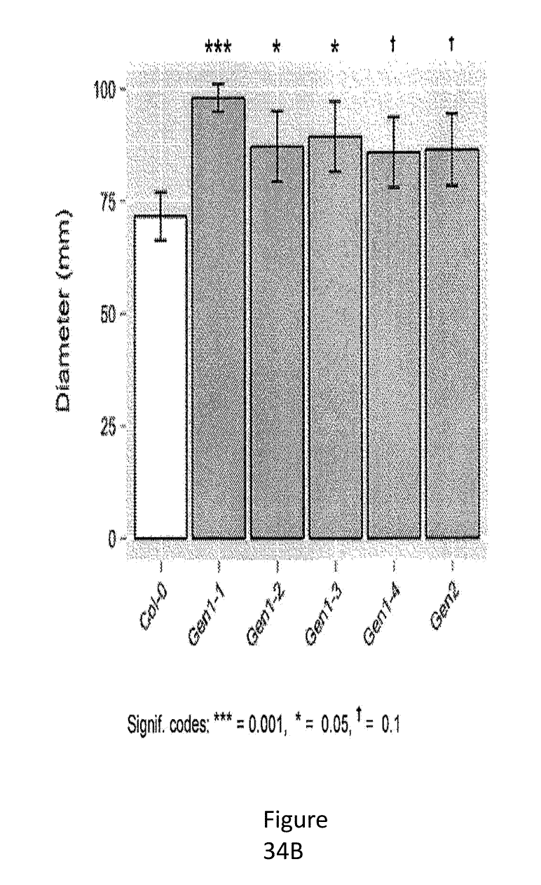

Because MSH1 down-regulation produces epigenetic changes, we tested whether enhanced growth could be transmitted through grafting or suppressed by chemical inhibition of DNA methylation. Using a root growth assay, we observed restoration of epi-F3 seedlings to wild type growth levels when seedlings were treated with 5-azacytidine (FIG. 27B, C), implicating DNA methylation in the enhanced growth phenotype. Moreover, when floral stem grafts between Col-0 and msh1 mutants were generated using msh1 as the rootstock, plants from first generation seed had an enhanced growth phenotype reminiscent of epi-lines produced through crossing (FIG. 24). This effect was not seen when msh1 was used as scion. Progeny from the graft-derived enhanced vigor plants retained growth vigor, indicating that the graft effects are heritable for at least two generations. The grafting results were observed in separate experiments using chm1-1 and Salk msh1 T-DNA lines (FIG. 34A-B).

Under normal conditions MSH1 expression is highest in reproductive tissues¹⁶, and steady state transcript levels decline markedly in response to environmental stress^{11,17}. One possibility is that MSH1 participates in environmental sensing, presumably via the plastid. MSH1 down-regulation triggers a process for altering plant phenotype via epigenetic remodeling, which could be a means to relax genetic constraint on phenotype following environmental change¹⁸. We have observed similar phenotypes from loss of MSH1 in six different plant species¹, indicating that these changes are part of a programmed response. Enhanced growth following crossing indicates that msh1-induced epigenetic reprogramming has special consequences when mutants are crossed to plants with unmodified epigenomes, perhaps resembling heterosis^{19,20}. The role of transposons in this phenomenon requires further investigation, but studies of stress in diverse organisms imply an association between transposons, stress responses^{21,22}, and phenotypic plasticity²³.

### Methods for Example 4

### Plant Materials and Growth Conditions

Arabidopsis Col-0 and msh1 mutant lines were obtained from the Arabidopsis stock center and grown at 12 hr day length at 22° C. The segregating T-DNA insertion line, SAIL_{—}877_F01, was genotyped using forward (ACGGAAAAAGTTCTTTCCAGG; SEQ ID NO:55) and reverse (GCTTTCCATCGGCTAGGTTAG; SEQ ID NO:56) primers for MSH1 (At3G24320) together with SAIL primer LB3 (TAGCATCTGAATTTCATAACCAATCTCGATACAC; SEQ ID NO:57). Seed from individual plants segregating for the T-DNA insertion in MSH1 was collected from heterozygous and null msh1 mutant plants. Progeny from a single heterozygous parent were grown to produce wild type segregants, heterozygote segregants and first generation msh1 mutant segregants. Second generation msh1 mutants were derived from individual first generation msh1 mutant plants. The advanced generation chm1-1 mutant was described previously²⁴. MSH1 first-generation, second-generation and epi-lines were derived as shown in FIG. 21. Arabidopsis plant measurements and leaf material used for DNA methylome analysis were conducted on 4-5 week-old plants prior to bolting. Arabidopsis flowering time was measured as date of first visible flower bud appearance. For hemi-complementation crosses, mitochondrial (AOX-MSH1) and plastid (SSU-MSH1) complemented homozygous lines were crossed to Col-0 wild type plants. Each F₁ plant was genotyped for transgene and wild type MSH1 allele and harvested separately. Three F2 families from AOX-MSH1×Col-0 and two F2 families from SSU-MSH1×Col-0 were evaluated for growth parameters. All families were grown under the same conditions, and biomass, rosette diameter and flowering time were measured. Two-tailed Student t-test was used to calculate p-values.

Genome Sequencing, De Novo Genome Assembly and SNP Analysis of Msh1.

Genome sequencing was carried out at the Center for Genomics and Bioinformatics at Indiana University. The 20 nM dilutions were made for DNA samples prepared from mutant msh1 and one epiF5 line. Preparation of single stranded DNA used 5 μl 20 nM dilution and 5 μl 0.2 N NaOH inclubated for 5 min and diluted with 990 μl Illumina HT1 Hyb buffer for 100 pM ssDNA stocks. 100 μl of 100 pM stock, 397 μl Ht1 buffer and 3 μl PhiX 10 nM ssDNA control were loaded to the flowcell of the Illumina MiSeq and processing was according to manufacturer's instructions.

Raw paired-end reads (mate 1: 300 bp; mate 2: 230 bp) were quality trimmed with a Phred quality threshold of 20 and reads with a subsequent length of less than 50 bases were removed. Illumina TruSeq adapter (index 22) was trimmed (prefixed with ‘A’ user for adapter ligation), removing from the adapter match to the 3′ end of the read. A second pass of adapter trimming without the ‘A’ prefix was done to remove adapter dimers. Ambiguous bases were trimmed from the 5′ and 3′ end of reads, and those reads with more than 1% number of ambiguous bases were completely removed. A second pass of quality filtering was performed, again with bases lower than a Phred quality score of 20 being trimmed, and reads of less than 50 bases being removed. A PhiX (RefSeq: NC_{—}001422) spike-in was removed by mapping the reads via bowtie2²⁵ (version 2.0.6) against the PhiX genome and filtering out any hits from the FASTQ files via a custom Perl script (available upon request). The resulting FASTQ files were synchronized, such that only full mate-pairs remained, while orphans (only one mate exists) were stored in an separate file. Cutadapt²⁶ (version 1.2.1) was used for the adapter removal, and the NGS-QC toolkit²⁷ (version 2.3) and fastq_quality_trimmer²⁸ (part of FASTX Toolkit 0.0.13.2) were used for the removal of ambiguous bases and quality filtering, respectively.

The msh1 genome was assembled using Velvet²⁹ with a kmer value of 83, an insert length of 400 bases, a minimum contig length of 200 bases, and the short paired (the PE reads) and a short read (the orphans) FASTQ files. The expected coverage (−exp_cov) and coverage cutoff (−cov_cutoff) were determined manually to be 25 and 8, respectively, by inspecting the initial weighted coverage of the first assembly. Resulting contigs were mapped back to Col-0 via blastn³° (version 2.2.26+) using an e-value of 10⁻²⁰ and coverage was determined with a custom Perl script (available upon request).

For the SNP and indel detection between msh1 and Col-0, the PE reads were aligned against the TAIR10 reference version of the Col-0 genome sequence via the short read aligner bowtie2 using the very-sensitive option and allowing one mismatch per seed (−N 1). Only the best alignment was reported and stored in a SAM file. The SAM file was processed via samtools mpileup³¹ (version 0.1.18) and subsequently filtered by a minimum read depth of 20, a minimum mapping quality of 30, and a minimum SNP or indel Phred quality score of 30 (p<=0.001). The SNPs and small indels were compared to supplementary data files from Lu et al.³² with custom made Perl scripts (available upon request). The msh1 genome sequence data has been uploaded to the Short Read Archive under sample number SAMN0919714.

Bisulfite Treated Genomic Library Construction and Sequencing.

Arabidopsis genomic DNA (15 μg) prepared from Col-0, msh1 (chm1-1) and epi-F3 plants was sonicated to peak range 200 bp to 600 bp. Sonicated DNA (12 μg) was treated with Mung Bean Nuclease (New England Biolabs), phenol/chloroform extracted and ethanol precipitated. Mung Bean Nuclease-treated genomic DNA (3 μg) was end-repaired and 3′ end-adenylated with Illumina (San Diego Calif.) Genomic DNA Samples Prep Kit. The adenylated DNA fragment was ligated to methylation adapters (Illumina). Samples were column purified and fractionated in agarose. A fraction of 280 bp to 400 bp was gel purified with the QIAquick Gel Purification kit (Qiagen, Valencia, Calif.). Another 3 pl of Mung Bean Nuclease treated genomic DNA was used to repeat the process, and the two fractions pooled and subjected to sodium bisulfite treatment with the MethylEasy Xceed kit (Human Genetic Signatures Pty Ltd, North Ryde, Australia). Three independent library PCR enrichments were carried out with 10 μl from total 30 μl bisulfate treated DNA as input template. The PCR reaction mixture was 10 μl DNA, 5 μl of 10× pfuTurbo Cx buffer, 0.7 μl of PE1.0 primer, 0.7 μl PE2.0 primer, 0.5 μl of dNTP (25 mM), 1 μl of PfuTurbo Cₓ Hotstart DNA Polymerase (Stratagene, Santa Clara, Calif.), and water to total volume PCR parameters were 95° C. for 2 min, followed by 12 cycles of 95° C. 30 sec, 65° C. 30 sec and 72° C. 1 min, then 72° C. for 5 min. PCR product was column-purified and equal volumes from each reaction were pooled to final concentration of 10 nM. Libraries were DNA sequenced on the Illumina Genome Analyzer II with three 36-cycle TruSeq sequencing kits v5 to read 116 nucleotides of sequence from a single end of each insert (V8 protocol). Early generation msh1 T-DNA insertion line methylomes were generated at the University of California Los Angeles according to methods published previouslyl³.

Identification and Annotation of Pair-Wise DMPs.

FASTQ files were aligned to the TAIR10 reference genome using Bismark³³, which was also used to determine the methylation state of cytosines. One mismatch was allowed in the first 50 nucleotides (when the read length is 116) or 35 nucleotides (when the read length is 51, as in the case of early generation msh1 T-DNA insertion lines) of the read. Only reads that were uniquely mapped to a location in the genome were retained. Genomic regions with highly homologous sequences at other locations of the genome were filtered out.

Cytosines were considered for DMP identification if they were covered by four or more reads in each of the genotypes, and covered by two or more reads as methylated cytosines in at least one genotype. For these cytosine positions, the number of reads indicating methylation or non-methylation for each genotype was tabulated. Fisher's exact test was carried out for testing differential methylation between two genotypes at each position. Adjustment for multiple testing over the entire genome was done according to Storey and Tibshirani³⁴ and a false discovery rate (FDR) of 0.05 was used for identifying differentially methylated cytosines. Cytosines which were not identified as DMPs were considered as NDMPs. A less stringent threshold was used for identifying differentially methylated cytosines of CHG and CHH; adjustment for multiple testing was done for cytosines where a p-value smaller than 0.05 and a false discovery rate (FDR) of 0.035 was used. Methylome sequence data have been uploaded to the Gene Expression Omnibus with accession number GSE36783.

Annotation from TAIR10 was used to determine the counts for pair-wise DMPs or non-differentially methylated positions in genes, transposons, transposable element genes, or other features. For plots of pair-wise DMP distributions across features, the distance between each DMP and the boundary of its nearest gene and transposon was calculated. For each sample, DMP frequencies within non-overlapping 100 bp bins were computed from −2 kb to +2 kb relative to feature start and ends. Bin frequencies were normalized to the proportion of DMPs with mapped features having a length sufficient to cover each corresponding bin, then scaled as a proportion of the maximum bin frequency across all samples and contexts, as well as across feature types depending on comparison (genes and transposons, or transposons with and without TE genes).

Identifying Group-Wise DMRs and Subsequent Multivariate Analyses.

Statistically significant CG and CHG group-wise DMRs were detected using the R-package BiSeq³⁵. Each sample was represented as a vector in the N-dimensional space formed by the N means of group-wise DMR methylation levels detected in the previous step. Multivariate statistical analyses of the vector-samples was performed using the R-package adegenet^{36,37}. Partitioning of samples into subsets was performed by principal component analysis (PCA) followed by linear discriminant analysis (LDA). PCA was first applied to the data set to reduce its dimensionality. The four first PCA components were then used to perform the LDA. The LDA sample's coordinates of two linear discriminant functions were used to perform the hierarchical clustering of the two-dimensional vector-samples by using the R-package cluster³⁸. Ward's minimum variance method³⁹ was used as agglomerative hierarchical clustering procedure with the squared Euclidean distance. Alternative multivariate analyses, without relying on DMRs or DMPs, were also performed. In this case, the methylation levels in tiling windows of 340 bp with at least 20 covered cytosine sites were obtained using the R-package methylKit⁴⁰. Next, each sample was represented as a vector in the N-dimensional space formed by the N methylation regions and the steps of PCA, LDA and hierarchical clustering were performed.

Grafting Experiments.

Wedge-Cleft grafting was performed when primary inflorescence meristems reached 5 to 10 cm above rosettes and floral buds became visible⁴¹. Silicone tubing was used to secure the wedge grafts to help maintain contact between scion and root stock. Graft junctions were further sealed with stretched parafilm to prevent desiccation. Grafted plants were kept in a mist chamber for 1-2 weeks days until scions started to grow, after which plants were slowly acclimatized to normal growth conditions. Additional floral shoots were removed to promote growth of the primary grafted floral stem. Each grafted scion was harvested separately, giving rise to generation one progeny. Single plants from generation one progeny were allowed to self-pollinate to produce generation two progeny.

5-Azacytidine Treatment and Root Length Assays.

Treatment with azacytidine can be used to nullify methylation effects⁴². Methylation inhibition assay was performed on wild type Col-0 (C), an advanced epiF7 line (E), and the 2^{nd} generation progeny of a Col-0/msh1 graft (G). All seeds were bleach sterilized then sown on half-strength MS media containing 1% sucrose and 25 μL DMSO (untreated solvent control), alternating between lines as shown in FIG. 27b. Plates were placed vertically in a growth chamber maintained at 12 hour day-light cycle and temperature of 22° C. At 3 days post-germination, half of the seedlings were transferred to similar half-strength MS plates containing 1% sucrose and 5-azacytidine at a final concentration of 50 μM. Ten days after moving to growth chamber, plates were scanned and root lengths were measured using ImageJ. Three replicates were conducted for a total sample size of 18 for each line and treatment combination. Similar abolishment of enhanced epi-line root length phenotype was seen in two additional independent experiments where seedlings were directly germinated on half-strength MS media containing 30 μM 5-azacytidine or DMSO solvent control.

Additional Information

The raw and processed microarray data are deposited at the Gene Expression Omnibus (GEO) under accession number GSE43993. Methylome sequence data are deposited at GEO under accession number GSE36783. The genome sequence data has been uploaded to the Short Read Archive under sample number SAMN0919714. Reprints and permissions information is available on the internet (world wide web) at “nature.com/reprints”.

### REFERENCES FOR EXAMPLE 4

1. Xu, Y.-Z. et al. The chloroplast triggers developmental reprogramming when MUTS HOMOLOG1 is suppressed in plants. Plant Physiol. 159, 710-720 (2012).
2. Bonasio, R., Tu, S. & Reinberg, D. Molecular signals of epigenetic states. Science 33, 612-616 (2010).
3. Mirouze, M. & Paszkowski, J. Epigenetic contribution to stress adaptation in plants. Curr Opin Plant Biol. 14, 267-274 (2011).
4. Dowen, R. H. et al. Widespread dynamic DNA methylation in response to biotic stress. Proc. Natl. Acad. Sci. USA 109, E2183-2191 (2012).
5. Youngson, N. A. & Whitelaw, E. Transgenerational epigenetic effects. Annu. Rev. Genom. Human Genet 9, 233-257 (2008).
6. Paszkowski, J. & Grossniklaus, U. Selected aspects of transgenerational epigenetic inheritance and resetting in plants. Curr. Opin. Plant Biol. 14, 195-203 (2011).
7. Reinders, J. et al. Compromised stability of DNA methylation and transposon immobilization in mosaic Arabidopsis epigenomes. Genes Dev. 23, 939-950 (2009).
8. Cortijo, S et al. Mapping the epigenetic basis of complex traits. Science. 5, epub ahead of print (2014).
9. Roux, F. et al. Genome-wide epigenetic perturbation jump-starts patterns of heritable variation found in nature. Genetics 188, 1015-1017 (2011).
10. Abdelnoor, R. V. et al. Substoichiometric shifting in the plant mitochondrial genome is influenced by a gene homologous to MutS. Proc. Natl. Acad. Sci. USA 100, 5968-5973 (2003).
11. Xu, Y.-Z. et al. MutS HOMOLOG1 is a nucleoid protein that alters mitochondrial and plastid properties and plant response to high light. Plant Cell 23, 3428-3441 (2011).
12. Santa Maria, R., et al. MSH1-induced non-genetic variation provides a source of phenotypic diversity in Sorghum bicolor. Submitted.
13. Stroud, H., et al. Comprehensive analysis of silencing mutants reveals complex regulation of the Arabidopsis methylome. Cell 152, 352-364 (2013).
14. Becker, C. et al. Spontaneous epigenetic variation in the Arabidopsis thaliana methylome. Nature 480, 245-249 (2011).
15. Schmitz, R. J. et al. Transgenerational epigenetic instability is a source of novel methylation variants. Science 334, 369-373 (2011).
16. Shedge, V., Arrieta-Montiel, M. P., Christensen, A. C. & Mackenzie, S. A. Plant mitochondrial recombination surveillance requires unusual RecA and MutS homologs. Plant Cell 19, 1251-1264 (2007).
17. Shedge, V., Davila, J., Arrieta-Montiel, M. P., Mohammed, S. & Mackenzie S. A. Extensive rearrangement of the Arabidopsis mitochondrial genome elicits cellular conditions for thermotolerance. Plant Physiol. 152, 1960-1970 (2010).
18. Kalisz, S. & Kramer, E. M. Variation and constraint in plant evolution and development. Hered. 100, 171-177 (2008).
19. Greaves, I., Groszmann, M., Dennis, E. S. & Peacock, W. J. Trans-chromosomal methylation. Epigenetics 7, 800-805 (2012).
20. Shivaprasad, P. V., Dunn, R. M., Santos, B. A., Bassett, A. & Baulcombe, D.C. Extraordinary transgressive phenotypes of hybrid tomato are influenced by epigenetics and small silencing RNAs. EMBO J 31, 257-266 (2012).
21. Wheeler, B. S. Small RNAs, big impact: small RNA pathways in transposon control and their effect on the host stress response. Chromosome Res. 21, 587-600 (2013).
22. Ito, H. Small RNAs and regulation of transposons in plants. Genes Genet Syst. 88, 3-7 (2013).
23. Zhang, C. C, Yuan, W-Y, Zhang, Q-F. RPL1: A gene involved in epigenetic processes regulates phenotypic plasticity in rice. Mol. Plant 5, 482-493 (2012).
24. Redei, G. P. Extra-chromosomal mutability determined by a nuclear gene locus in Arabidopsis. Mutat. Res. 18, 149-162 (1973).
25. Langmead, B. & Salzberg, S. Fast gapped-read alignment with Bowtie 2. Nat. Methods 9, 357-359 (2012).
26. Martin M. Cutadapt removes adapter sequences from high-throughput sequencing reads. EMBnet Journal. 17, 1 (2011).
27. Patel R. K., Jain M. NGS QC Toolkit: A toolkit for quality control of next generation sequencing data. PLoS ONE 7(2): e30619 (2012).
28. Hannon Lab. FASTX-Toolkit. http://hannonlab.cshl.edu/fastx_toolkit/29. Zerbino D. R., McEwen G. K., Margulies E. H., Birney E. Pebble and Rock Band: Heuristic Resolution of Repeats and Scaffolding in the Velvet Short-Read de Novo Assembler. PLoS ONE 4(12): e8407 (2009).
30. Camacho, C. et al. BLAST+: architecture and applications. BMC Bioinformatics 10, 421 (2009).
31. Li, H. et al. The Sequence alignment/map (SAM) format and SAMtools. Bioinformatics 25, 2078-2079 (2009).
32. Lu, P. et al. Analysis of Arabidopsis genome-wide variations before and after meiosis and meiotic recombination by resequencing Landsberg erecta and all four products of a single meiosis. Genome Res. 22, 508-518 (2012).
33. Krueger, F. & Andrews, S. R. Bismark: a flexible aligner and methylation caller for Bisulfite-Seq applications. Bioinformatics 27, 1571-1572 (2011).
34. Storey, J. D. & Tibshirani, R. Statistical significance for genome-wide studies. Proc. Natl. Acad. Sci. USA 100, 9440-9445 (2003).
35. Hebestreit, K., Dugas, M. & Klein, H.-U. Detection of significantly differentially methylated regions in targeted bisulfite sequencing data. Bioinformatics 29, 1647-53 (2013).
36. Jombart, T. & Ahmed, I. adegenet 1.3-1: new tools for the analysis of genome-wide SNP data. Bioinformatics 27, 3070-1 (2011).
37. Jombart, T. adegenet: a R package for the multivariate analysis of genetic markers. Bioinformatics 24, 1403-5 (2008).
38. Maechler, M., Rousseeuw, P., Anja Struyf, M. H. & Hornik, K. cluster: Cluster Analysis Basics and Extensions. R package version 1.15.1. (2013).
39. Joe H. Ward, J. Hierarchical Grouping to Optimize an Objective Function. J. Am. Stat. Assoc. 58, 236-244 (1963).
40. Akalin, A. et al. methylKit: a comprehensive R package for the analysis of genome-wide DNA methylation profiles. Genome Biol. 13, R87 (2012).
41. Nisar, N., Verma, S., Pogson, B. J. & Cazzonelli, C. I. Inflorescence stem grafting made easy in Arabidopsis. Plant Methods 8(1):50. (2012).
42. Boyko, A. et al. Transgenerational adaptation of Arabidopsis to stress requires DNA methylation and the function of Dicer-like proteins. PLoS ONE. 5(3):e95149. (2010).

### Example 5

### Method for Selecting a Plant Comprising One or More Altered Chromosomal Loci Useful for Plant Breeding

Methods for suppressing MSH1 and constructs for doing so are described in U.S. Patent Application Publication No., 2012/0284814, U.S. Provisional 61/882,140, and U.S. Provisional 61/901,349, which are each incorporated herein by reference in their entireties. An RNAi hairpin vector directed against an endogenous MSH1 for the specific plant targeted is used herein. A transgenic plant containing the MSH1 hairpin construct is produced by transformation methods known to those skilled in the art, such as Agrobacterium-mediated transformation methods or particle gun transformation methods known to be effective for the plant species to be transformed. A transgenic plant containing said MSH1 hairpin construct is identified and the knockdown of the endogenous MSH1 mRNA is confirmed by Northern blot or a quantitative PCR analysis of RNA isolated from said plant. Progeny from said plant are obtained and screened by PCR of their isolated DNA to find progeny lacking the transgene. One or more progeny lacking a transgene and derived from a MSH1 suppressed parent, are either self pollinated or outcrossed. These resulting progeny are candidate plants for altered chromosomal loci due to their descent from a progenitor plant suppressed for MSH1.

DNA methylation analysis by Illumina high throughput DNA sequencing of bisulfite treated DNA will identify CG, CHG, and CHH sites with 5-methyl-cytosine modifications in the genome, and will identify a frequency of methylation at each of these sites, with higher genome sequence coverage levels providing better quality data (with sequence coverage of several multiples of the genome size, preferably at least 20×, where 1× is the amount of sequence equivalent to the genome size of the species). Comparison of the DNA methylation levels between the isogenic parental plant prior to MSH1 suppression and the candidate plant identifies chromosomal regions with differences in DNA methylation levels as described in Example 4. Comparison of the DNA methylation patterns of the MSH1 suppressed parental plant, and its progeny that were ancestors to the candidate plant provides additional comparisons to increase identification of altered chromosomal regions with increased or decreased DNA methylation levels as described in Example 4. Increased or decreased DNA methylation levels selected from the group consisting of MSH1, pericentromeric regions, CG enhanced genes, CG depleted genes, transposable elements, transposable elements containing genes, and transposable elements in pericentromeric regions are preferable for identifying altered chromosomal loci. This method of identification of altered chromosomal loci can be applied to any plant, preferably plants with established genome sequences.

Measurement of sRNA levels for the plants described above at the steps of measuring DNA methylation above, also identifies altered chromosomal loci as indicated by their altered sRNA levels. sRNA levels are measured by using Illumina procedures and kits for constructing sRNA libraries. Said sRNA libraries are sequenced on an Illumina high throughput DNA sequencing system such as the HiSeq 2500, at sufficient sequencing coverage such as 0.1 to 10 M or more reads per sample, preferably 40 M sequence reads per sample. Comparison of the sRNA sequences and abundances between the reference plant (a parental plant prior to MSH1 suppression) and the candidate plant identifies altered chromosomal regions producing altered sRNA levels by this method. Candidate plants with one or more altered chromosomal loci as determined by DNA methylation or sRNA changes are selected and are useful for plant breeding.

### Example 6

### Method for Producing a Plant Exhibiting New Combinations of Altered Chromosomal Loci Useful for Breeding

The methods described in Examples 4 and 5 for identifying altered chromosomal loci with altered DNA methylation or sRNAs in progeny are applied to progeny from a cross of two parents, wherein at least one parent has a progenitor plant subjected to MSH1 suppression. The DNA methylation and/or sRNAs of one or more said progeny are compared to the DNA methylation and/or sRNAs of either parent, each compared separately to said progeny, wherein said progeny can be from the cross or from later plant generations derived from the cross. Increased or decreased DNA methylation levels, or increased or decreased sRNA levels derived from the following regions, are selected from the group consisting of MSH1, pericentromeric regions, CG enhanced genes, CG depleted genes, transposable elements, transposable elements containing genes, and transposable elements in pericentromeric regions are preferable for identifying altered chromosomal loci. This method of identification of altered chromosomal loci can be applied to any plant, preferably plants with established genome sequences. Candidate plants with new combinations of altered chromosomal loci as determined by DNA methylation or sRNA changes are selected and are useful for plant breeding.

### Example 7

### Method for Producing a Plant from a Selfed Plant Exhibiting New Combinations of Altered Chromosomal Loci Useful for Breeding

The methods described in Examples 4 and 5 for identifying altered chromosomal loci with altered DNA methylation or sRNAs in progeny are applied to progeny from a selfed plant which is derived from a progenitor plant subjected to MSH1 suppression. The DNA methylation and/or sRNAs of one or more said progeny are compared to the DNA methylation and/or sRNAs of the parent plant, wherein said progeny can be from initial progeny of from later plant generations. Increased or decreased DNA methylation levels, or increased or decreased sRNA levels derived from the following regions, are selected from the group consisting of MSH1, one or more pericentromeric regions, CG enhanced genes, CG depleted genes, transposable elements, transposable elements containing genes, transposable elements in pericentromeric regions, and transposable elements containing genes in pericentromeric regions are preferable for identifying altered chromosomal loci. This method of identification of altered chromosomal loci can be applied to any plant, preferably plants with established genome sequences. Candidate plants with new combinations of altered chromosomal loci as determined by DNA methylation or sRNA changes are selected and are useful for plant breeding.

### Example 8

### Methods Applicable to all Crops

All of the above Examples 4-7 are suitable for application to all plant and all crop plants (with suitable methods of MSH1 suppression such as RNAi constructs and transformation methods specific for each plant species), including, but not limited to, the following crops: corn, wheat, rice, sorghum, millet, tomato, potato, soybean, tobacco, cotton, canola, alfalfa, rapeseed, sugar beets, and sugarcane.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A method for producing a plant exhibiting a useful trait comprising the steps of
(a) perturbing plastid function in a first parental plant or plant cell, wherein the perturbing does not comprise direct suppression of MSH1 gene expression;
(b) screening a population of progeny plants obtained from the parental plant or plant cell for the useful trait, wherein plastid function has been recovered in at least a portion of the progeny plants; and,
(c) selecting one or more progeny plants that exhibit(s) the useful trait and have recovered plastid function, wherein the trait exhibits nuclear inheritance.

2. The method of claim 1, wherein the perturbed plastid function is selected from the group consisting of a sensor, photosystem I, photosystem II, NAD(P)H dehydrogenase (NDH) complex, cytochrome b6f complex, and plastocyanin function.

3. The method of claim 2, wherein the photosystem II function and/or sensor function is perturbed by suppressing expression of a gene selected from the group consisting of a PPD3 gene, a PsbO-1, a PsbO-2, PsbY, PsbW, PsbX, PsbR, PsbTn, PsbP1, PsbP2, PsbS, PsbQ-1, PsbQ-2, PPL1, PSAE-1, LPA2, PQL1, PQL2 and a PQL3 gene.

4. The method of claim 1, wherein the plastid function is selectively inhibited in cells containing sensory plastids.

5. The method of claim 4, wherein the selective inhibition is effected with a transgene comprising a promoter that is selectively expressed in cells containing sensory plastids and that is operably linked to a sequence that perturbs plastid function.

6. The method of claim 5, wherein the promoter is an MSH1 promoter or a PPD3 promoter.

7.-14. (canceled)

15. The method of claim 1, wherein the method further comprises the step of producing seed from: i) a selfed progeny plant or plants; ii) an out-crossed progeny plant or plants; or, iii) both of a selfed and an out-crossed progeny plant or plants.

16. The method of claim 1, wherein the method further comprises the step of producing seed from: (i) a selfed progeny plant or plants selected in step (c); or from (ii) an out-crossed progeny plant or plants selected in step (c).

17. The method of claim 1, wherein the method comprises: (i) outcrossing or selfing the first parental plant or progeny thereof to obtain an F1 generation of plants, wherein the first parental plant or progeny thereof exhibits one or more Msh1-dr traits; (ii) screening the population of plants obtained from the outcross for the presence of the useful trait and the absence of Msh1-dr traits; (iii) selecting a population of plants exhibiting the useful trait and recovered plastid function; and (iv) obtaining seed from the selected population of step (iii) or, optionally, repeating steps (iii) and (iv) on a population of plants grown from the seed obtained from the selected population.

18.-48. (canceled)

49. A recombinant DNA construct comprising a promoter that is selectively expressed in cells containing sensory plastids and that is operably linked to a heterologous sequence that perturbs plastid function.

50. The recombinant DNA construct of claim 49, wherein the promoter is selected from the group consisting of a Msh1 promoter and a PPD3 promoter.

51.-56. (canceled)

57. A method for producing a seed lot comprising:
(i) selecting a first sub-population of plants exhibiting a useful trait associated with an epigenetic change at one or more nuclear chromosomal loci and recovered plastid function from a first population of plants that are segregating for the useful trait; and
(ii) obtaining a seed lot from the first selected sub-population of step (i) or, optionally, repeating steps (i) and (ii) on a second population of plants grown from the seed obtained from the first selected sub-population of plants.

58. The method of claim 57, wherein the epigenetic change was induced by plastid perturbation.

59. The method of claim 58, wherein the epigenetic change was induced by suppressing expression of a gene selected from the group consisting of a Msh1, PPD3 gene, a PsbO-1, a PsbO-2, PsbY, PsbW, PsbX, PsbR, PsbTn, PsbP1, PsbP2, PsbS, PsbQ-1, PsbQ-2, PPL1, PSAE-1, LPA2, PQL1, PQL2, and a PQL3 gene.

60. The method of claim 57, wherein the epigenetic change is associated with CG hyper-methylation and/or CHG and/or CHH hyper-methylation at one or more nuclear chromosomal loci in comparison to a control plant that does not exhibit the useful trait.

61. (canceled)

62. The method of claim 57, wherein a plurality of plants in the first sub-population exhibit heritable CHG and/or CHH hyper-methylation of one or more regions comprising pericentromeric, transposable element, or repeated sequences.

63. The method of claim 57, wherein at least 25%, 50%, 60%, 70%, 80%, 90%, or 95% of progeny plants grown from the seed lot obtained in step (ii) exhibit the useful trait associated with an epigenetic change.

64. The method of claim 63, wherein the seed or progeny plants grown from the seed comprise a mixture of inbred and hybrid germplasm that is epigenetically heterogenous.

65. A seed lot produced by the method of claim 57.

66. A seed lot comprising seed wherein at least 25%, 50%, 60%, 70%, 80%, 90%, or 95% of progeny plants grown from the seed exhibit a useful trait associated with one or more epigenetic changes induced by suppression of MSH1, wherein the epigenetic changes are associated with CG hyper-methylation and/or CHG and/or CHH hyper-methylation at one or more nuclear chromosomal loci in comparison to a control plant that does not exhibit the useful trait, and wherein the seed or progeny plants grown from said seed that is epigenetically heterogenous.

67. The seed lot of claim 66, wherein the useful trait is selected from the group consisting of increased yield, male sterility, non-flowering, increased biotic stress resistance, increased abiotic stress resistance, enhanced lodging resistance, enhanced growth rate, enhanced biomass, enhanced tillering, enhanced branching, delayed flowering time, and delayed senescence in comparison to a control plant that lacks the epigenetic change(s).

68. The seed lot of claim 66, wherein said seed comprise a mixture of inbred and hybrid germplasm.

69. A method for producing a plant exhibiting new combinations of altered chromosomal loci useful for breeding comprising the steps of:
(a) crossing or selfing a plant comprising altered chromosomal loci induced by MSH1 suppression to produce progeny; and,
(b) assaying the progeny of step (a) to identify and select individuals with new combinations of altered chromosomal loci, thereby producing a plant exhibiting new combinations of altered chromosomal loci useful for breeding.

70. (canceled)

71. The method of claim 69, wherein the DNA methylation of one or more altered chromosomal loci occurs at CHG or CHH sites within a DNA region selected from the group consisting of MSH1, pericentromeric regions, transposable elements, transposable elements containing genes, and transposable elements in pericentromeric regions is assayed.

72. The method of claim 69, wherein one or more sRNAs having sequence homology to one or more regions selected from the group consisting of MSH1, pericentromeric regions, CG enhanced genes, CG depleted genes, transposable elements, transposable elements containing genes, and transposable elements in pericentromeric regions are assayed.

73.-74. (canceled)

75. A method for identifying a plant with altered chromosomal loci useful for plant breeding comprising the steps of:
(a) assaying one or more plants comprising altered chromosomal loci induced by MSH1 suppression; and,
(b) identifying one or more plants from step (a) comprising one or more altered chromosomal loci selected from the group consisting of MSH1, pericentromeric regions, CG enhanced genes, CG depleted genes, transposable elements, transposable elements containing genes, and transposable elements in pericentromeric regions, thereby identifying a plant with altered chromosomal loci useful for plant breeding.

76. The method of claim 75, wherein DNA methylation of one or more altered chromosomal loci occurring at CHG or CHH at DNA sequences selected from the group consisting of MSH1, pericentromeric regions, transposable elements, transposable elements containing genes, and transposable elements in pericentromeric regions is assayed.

77. The method of claim 75, wherein one or more sRNAs having sequence homology to one or more regions selected from the group consisting of MSH1, pericentromeric regions, CG enhanced genes, CG depleted genes, transposable elements, transposable elements containing genes, and transposable elements in pericentromeric regions are assayed.

78.-87. (canceled)
